SEQUENCE LISTING (129 sequences)

SEQ ID NO: 1 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 2 - protein, 305 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 3 - RNA, 972 nt, synthetic construct
  source 1..972: mol_type = other RNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataa

SEQ ID NO: 4 - RNA, 918 nt, synthetic construct
  source 1..918: mol_type = other RNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactga

SEQ ID NO: 5 - RNA, 969 nt, synthetic construct
  source 1..969: mol_type = other RNA
atgtggcccctggtcgccgccctgttgctgggctcggcatgctgcggatcagctcagctactgtttaataaaacaaaatctgtagaattcacgttttgtaacgacactgtcgtgatcccatgctttgttactaatatggaggcacaaaacaccactgaagtgtacgtgaagtggaaattcaaaggcagagacatttacacctttgacggcgccctcaacaagtccaccgtgcccactgactttagtagcgcaaaaattgaggtcagccaattactaaaaggagatgcctctttgaagatggacaagagcgatgctgtcagccacacagggaactacacttgtgaagtaacagagttaacccgcgaaggtgaaacgatcatcgagctgaagtatcgagtggtgtcctggttttctccgaacgagaatatccttatcgtaattttcccaattttcgctatcctcctgttctggggccagtttggtatcaagacactcaaatatcggtccggtgggatggatgagaagacaattgccctgcttgttgctggactcgtgatcaccgtcatcgtgattgttggggccatccttttcgtcccaggggagtacagcctgaagaatgctacgggcctgggattaattgtgacctctacagggatactcatcctgcttcactactatgtgttcagtaccgcgattggactgacctccttcgtcattgccatattggtgattcaggtgatagcctacatcctcgccgtggttggcctgagtctctgtatcgcggcgtgcatacccatgcatggccctcttctgatttcagggttgagtatcctcgcactagcacagttgctgggactggtttatatgaaatttgtggcctccaaccagaagactatacagcctcctaggaaggctgtagaggagcccctgaatgcattcaaggaatcaaaaggcatgatgaatgatgaa

SEQ ID NO: 6 - protein, 21 aa, synthetic construct
MALPVTALLLPLALLLHAARP

SEQ ID NO: 7 - protein, 20 aa, synthetic construct
METDTLLLWVLLLWVPGSTG

SEQ ID NO: 8 - protein, 22 aa, synthetic construct
MLLLVTSLLLCELPHPAFLLIP

SEQ ID NO: 9 - protein, 45 aa, synthetic construct
TTTPAPRPPTPAPTIASQPLSLRPEACRPAAGGAVHTRGLDFACD

SEQ ID NO: 10 - protein, 39 aa, synthetic construct
IEVMYPPPYLDNEKSNGTIIHVKGKHLCPSPLFPGPSKP

SEQ ID NO: 11 - protein, 42 aa, synthetic construct
AAAIEVMYPPPYLDNEKSNGTIIHVKGKHLCPSPLFPGPSKP

SEQ ID NO: 12 - protein, 12 aa, synthetic construct
ESKYGPPCPPCP

SEQ ID NO: 13 - protein, 12 aa, synthetic construct
ESKYGPPCPSCP

SEQ ID NO: 14 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 24 aa, synthetic construct
IYIWAPLAGTCGVLLLSLVITLYC

SEQ ID NO: 16 - protein, 27 aa, synthetic construct
FWVLVVVGGVLACYSLLVTVAFIIFWV

SEQ ID NO: 17 - protein, 28 aa, synthetic construct
MFWVLVVVGGVLACYSLLVTVAFIIFWV

SEQ ID NO: 18 - protein, 42 aa, synthetic construct
KRGRKKLLYIFKQPFMRPVQTTQEEDGCSCRFPEEEEGGCEL

SEQ ID NO: 19 - protein, 41 aa, synthetic construct
RSKRSRLLHSDYMNMTPRRPGPTRKHYQPYAPPRDFAAYRS

SEQ ID NO: 20 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 23 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 24 - protein, 6 aa, synthetic construct
QDISKY

SEQ ID NO: 25 - skipped

SEQ ID NO: 26 - protein, 9 aa, synthetic construct
QQGNTLPYT

SEQ ID NO: 27 - protein, 18 aa, synthetic construct
GSTSGSGKPGSGEGSTKG

SEQ ID NO: 28 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 29 - protein, 8 aa, synthetic construct
GVSLPDYG

SEQ ID NO: 30 - protein, 7 aa, synthetic construct
IWGSETT

SEQ ID NO: 31 - protein, 14 aa, synthetic construct
AKHYYYGGSYAMDY

SEQ ID NO: 32 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 33 - protein, 15 aa, synthetic construct
GGGGSGGGGSGGGGS

SEQ ID NO: 34 - DNA, 1467 bp, synthetic construct
  source 1..1467: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 35 - protein, 489 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - DNA, 1458 bp, synthetic construct
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 37 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - DNA, 1383 bp, synthetic construct
  source 1..1383: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 39 - protein, 461 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - DNA, 1467 bp, synthetic construct
  source 1..1467: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 41 - protein, 489 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 246 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 43 - protein, 106 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 44 - protein, 10 aa, synthetic construct
RASSSVNYMD

SEQ ID NO: 45 - protein, 7 aa, synthetic construct
ATSNLAS

SEQ ID NO: 46 - protein, 9 aa, synthetic construct
QQWSFNPPT

SEQ ID NO: 47 - protein, 122 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 48 - protein, 5 aa, synthetic construct
SYNMH

SEQ ID NO: 49 - protein, 17 aa, synthetic construct
AIYPGNGDTSYNQKFKG

SEQ ID NO: 50 - protein, 246 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 51 - protein, 124 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 52 - protein, 10 aa, synthetic construct
GDSVSSNSAA

SEQ ID NO: 53 - protein, 9 aa, synthetic construct
TYYRSKWYN

SEQ ID NO: 54 - protein, 14 aa, synthetic construct
AREVTGDLEDAFDI

SEQ ID NO: 55 - protein, 107 aa, synthetic construct
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 56 - protein, 6 aa, synthetic construct
QTIWSY

SEQ ID NO: 57 - skipped

SEQ ID NO: 58 - protein, 9 aa, synthetic construct
QQSYSIPQT

SEQ ID NO: 59 - protein, 246 aa, synthetic construct
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 60 - protein, 124 aa, synthetic construct
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSS

SEQ ID NO: 61 - protein, 10 aa, synthetic construct
GDSVSSNSVA

SEQ ID NO: 62 - protein, 9 aa, synthetic construct
TYYRSTWYN

SEQ ID NO: 63 - protein, 14 aa, synthetic construct
AREVTGDLEDAFDI

SEQ ID NO: 64 - protein, 107 aa, synthetic construct
DIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 65 - protein, 6 aa, synthetic construct
QTIWSY

SEQ ID NO: 66 - skipped

SEQ ID NO: 67 - protein, 9 aa, synthetic construct
QQSYSIPQT

SEQ ID NO: 68 - protein, 246 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 69 - protein, 111 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 70 - protein, 15 aa, synthetic construct
RASESVSVIGAHLIH

SEQ ID NO: 71 - protein, 7 aa, synthetic construct
LASNLET

SEQ ID NO: 72 - protein, 9 aa, synthetic construct
LQSRIFPRT

SEQ ID NO: 73 - protein, 117 aa, synthetic construct
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 74 - protein, 5 aa, synthetic construct
DYSIN

SEQ ID NO: 75 - protein, 17 aa, synthetic construct
WINTETREPAYAYDFRG

SEQ ID NO: 76 - protein, 8 aa, synthetic construct
DYSYAMDY

SEQ ID NO: 77 - protein, 246 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 78 - protein, 111 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 79 - protein, 15 aa, synthetic construct
RASESVTILGSHLIY

SEQ ID NO: 80 - protein, 7 aa, synthetic construct
LASNVQT

SEQ ID NO: 81 - protein, 9 aa, synthetic construct
LQSRTIPRT

SEQ ID NO: 82 - protein, 117 aa, synthetic construct
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 83 - protein, 5 aa, synthetic construct
HYSMN

SEQ ID NO: 84 - protein, 17 aa, synthetic construct
RINTESGVPIYADDFKG

SEQ ID NO: 85 - protein, 8 aa, synthetic construct
DYLYSLDF

SEQ ID NO: 86 - protein, 121 aa, synthetic construct
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 87 - protein, 8 aa, synthetic construct
GFTFSSYA

SEQ ID NO: 88 - protein, 8 aa, synthetic construct
ISGSGDYI

SEQ ID NO: 89 - protein, 14 aa, synthetic construct
AKEGTGANSSLADY

SEQ ID NO: 90 - protein, 243 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 91 - protein, 106 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 92 - protein, 6 aa, synthetic construct
QSISSY

SEQ ID NO: 93 - skipped

SEQ ID NO: 94 - protein, 8 aa, synthetic construct
QQKYDLLT

SEQ ID NO: 95 - protein, 119 aa, synthetic construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 96 - protein, 10 aa, synthetic construct
GGSISSSSYY

SEQ ID NO: 97 - protein, 7 aa, synthetic construct
ISYSGST

SEQ ID NO: 98 - protein, 11 aa, synthetic construct
ARDRGDTILDV

SEQ ID NO: 99 - DNA, 1503 bp, synthetic construct
  source 1..1503: mol_type = other DNA
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaaaatacgacctcctcacttttggcggagggaccaaggttgagatcaaaggcagcaccagcggctccggcaagcctggctctggcgagggcagcacaaagggacagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagagatcgtggagacaccatactagacgtatggggtcagggtacaatggtcaccgtcagctcattcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 100 - protein, 501 aa, synthetic construct
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 21 aa, synthetic construct
  REGION 1..3: note = A glycine-serine-glycine (GSG) linker is optionally added to the N-terminal of a 2A peptide to increase cleavage efficiency
GSGEGRGSLLTCGDVEENPGP

SEQ ID NO: 102 - protein, 22 aa, synthetic construct
  REGION 1..3: note = A glycine-serine-glycine (GSG) linker is optionally added to the N-terminal of a 2A peptide to increase cleavage efficiency
GSGATNFSLLKQAGDVEENPGP

SEQ ID NO: 103 - protein, 23 aa, synthetic construct
  REGION 1..3: note = A glycine-serine-glycine (GSG) linker is optionally added to the N-terminal of a 2A peptide to increase cleavage efficiency
GSGQCTNYALLKLAGDVESNPGP

SEQ ID NO: 104 - protein, 25 aa, synthetic construct
  REGION 1..3: note = A glycine-serine-glycine (GSG) linker is optionally added to the N-terminal of a 2A peptide to increase cleavage efficiency
GSGVKQTLNFDLLKLAGDVESNPGP

SEQ ID NO: 105 - protein, 7 aa, synthetic construct
  VARIANT 5..7: note = One or more optional glycine-serine-glycine (GSG) sequences can be included for cleavage efficiency
RRRRGSG

SEQ ID NO: 106 - protein, 7 aa, synthetic construct
  VARIANT 5..7: note = One or more optional glycine-serine-glycine (GSG) sequences can be included for cleavage efficiency
RKRRGSG

SEQ ID NO: 107 - protein, 15 aa, synthetic construct
  VARIANT 5..7: note = One or more optional glycine-serine-glycine (GSG) sequences can be included for cleavage efficiency
  VARIANT 13..15: note = One or more optional glycine-serine-glycine (GSG) sequences can be included for cleavage efficiency
RKRRGSGTPDPWGSG

SEQ ID NO: 108 - skipped

SEQ ID NO: 109 - RNA, 12 nt, synthetic construct
  source 1..12: mol_type = other RNA
gttttagagcta

SEQ ID NO: 110 - skipped

SEQ ID NO: 111 - RNA, 66 nt, synthetic construct
  source 1..66: mol_type = other RNA
tagcaagttaaaataaggctagtccgttatcaacttgaaaaagtggcaccgagtcggtgctttttt

SEQ ID NO: 112 - skipped

SEQ ID NO: 113 - RNA, 17 nt, synthetic construct
  source 1..17: mol_type = other RNA
gtttsagagctatgctg

SEQ ID NO: 114 - skipped

SEQ ID NO: 115 - RNA, 71 nt, synthetic construct
  source 1..71: mol_type = other RNA
cagcatagcaagttsaaataaggctagtccgttatcaacttgaaaaagtggcaccgagtcggtgctttttt

SEQ ID NO: 116 - skipped

SEQ ID NO: 117 - RNA, 14 nt, synthetic construct
  source 1..14: mol_type = other RNA
gttttagtactctg

SEQ ID NO: 118 - skipped

SEQ ID NO: 119 - RNA, 64 nt, synthetic construct
  source 1..64: mol_type = other RNA
cagaatctactaaaacaaggcaaaatgccgtgtttatctcgtcaacttgttggcgagatttttt

SEQ ID NO: 120 - RNA, 88 nt, synthetic construct
  source 1..88: mol_type = other RNA
gtcgtctataggacggcgaggacaacgggaagtgccaatgtgctctttccaagagcaaacaccccgttggcttcaagatgaccgctcg

SEQ ID NO: 121 - skipped

SEQ ID NO: 122 - RNA, 23 nt, synthetic construct
  source 1..23: mol_type = other RNA
cgagcggtctgagaagtggcact

SEQ ID NO: 123 - skipped

SEQ ID NO: 124 - RNA, 20 nt, synthetic construct
  source 1..20: mol_type = other RNA
tcagggttctggatatctgt

SEQ ID NO: 125 - RNA, 20 nt, synthetic construct
  source 1..20: mol_type = other RNA
tttggagaaaaccatgaaac

SEQ ID NO: 126 - protein, 18 aa, synthetic construct
GSTSGSGKPGSGEGSTKG

SEQ ID NO: 127 - protein, 6 aa, synthetic construct
  VARIANT 2..6: note = The amino acid residues GGGGS can be repeated one to ten times
SGGGGS

SEQ ID NO: 128 - protein, 5 aa, synthetic construct
  VARIANT 1..5: note = The entire sequence of amino acids 1-5 can be repeated one to ten times
GGGGS

SEQ ID NO: 129 - protein, 4 aa, synthetic construct
  VARIANT 1..4: note = The entire sequence of amino acids 1-4 can be repeated one to six times
GGGS

(19) United States
(12) Patent Application Publication
(10) Pub. No.: US 2025/0313861 A1
(43) Pub. Date: Oct. 9, 2025

(54) METHODS OF ENGINEERING ALLOGENEIC T CELLS WITH A TRANSGENE IN A TCR LOCUS AND ASSOCIATED COMPOSITIONS AND METHODS

(71) Applicant: Sana Biotechnology, Inc., Seattle, WA (US)

(72) Inventors: Terry J Fry, Denver, CO (US); Adam James Johnson, Shoreline, WA (US); William Dowdle, South San Francisco, CA (US)

(21) Appl. No.: 18/703,605

(22) PCT Filed: Oct. 24, 2022
(86) PCT No.: PCT/US2022/047624

§ 371 Date: Apr. 22, 2024

Related U.S. Application Data
(60) Provisional application No. 63/270,956, filed on Oct. 22, 2021.

(51) Int. Cl. C12N 15/90; C12N 5/0783; C12N 9/22; C12N 15/11

(52) U.S. Cl. CPC C12N 15/907; C12N 5/0636; C12N 9/226; C12N 15/11; C12N 2310/20; C12N 2510/00

(57) ABSTRACT

Provided herein are methods of producing a composition comprising genetically engineered cells for cell therapy, the method comprising: selecting one or more genetically engineered cells from a population of cells, and formulating the composition comprising the selected one or more genetically engineered cells for use, wherein the one or more genetically engineered cells comprise one or more genetic modifications, and wherein the one or more genetically engineered cells are selected based on a level of one or more markers on the cell surface of the one or more genetically engineered cells, as well as compositions derived therefrom.

### CROSS REFERENCE TO RELATED APPLICATIONS

This application claims priority to U.S. Provisional Application No. 63/270,956, filed Oct. 22, 2021, which is incorporated herein by reference.

### BACKGROUND

T cells play a central role in the adaptive immune response, including immune cell-mediated cell death. The use of modified T cells is an emerging cell therapy approach within the area of adoptive cell transfer (ACT). This approach involves collecting T cells from a patient (autologous) or healthy donors (allogeneic), genetically modifying or engineering these T cells, and transferring the modified or engineered T cells into the patient to treat a range of diseases. The use of allogeneic T cells has several advantages over the use of autologous T cells, as the latter suffers from challenges such as a patient having insufficient healthy T cells for harvesting and the patient experiencing disease progression, co-morbidities, or even death in the time it takes to manufacture the engineered T cells.

However, in order to make the use of allogeneic T cells in ACT feasible, the T cells must be rendered immune evasive (or hypoimmune), i.e., not be attacked by the host's immune system for being “foreign”. Engineering the T cells to contain one or more exogenous nucleic acids encoding a tolerogenic factor, such as CD47, a transmembrane protein and known marker of “self” on host cells within an organism, and optionally other modifications, enables the T cells to evade the patient's immune system. Thus, there is a growing need to efficiently manufacture such immune evasive (e.g., CD47+) T cells.

Moreover, T cells express an endogenous T cell receptor (TCR), generally consisting of a TCR alpha chain (TRAC) and a TCR beta chain (TRBC), which can form a complex with additional adaptor proteins, including CD3, to form an octameric complex. To make the use of allogeneic T cells feasible, expression of the TCR must be reduced or eliminated to prevent graft versus host disease (GVHD). Thus, there is also a need for the reliable manufacture of immune evasive T cells with endogenous TCR expression reduced or eliminated, in addition to the expression of tolerogenic factors.

### SUMMARY

The present disclosure provides methods for generating T cells, such as immune evasive allogeneic T cells, by inserting a first transgene encoding a tolerogenic factor (e.g., CD47, HLA-E, HLA-G, PD-L1, and CTLA-4) into an endogenous TCR gene locus (e.g., the TRAC and/or TRBC loci including TRBC1 and/or TRBC2) of the T cells, and selecting for T cells by CD3 depletion, TCR depletion, and/or positive selection for the tolerogenic factor. The compositions derived from such methods and methods of using said compositions are also provided. In some embodiments, the compositions and methods disclosed herein further comprise delivering a second transgene encoding a chimeric antigen receptor (CAR) (e.g., CD19 CAR, CD20 CAR, CD22 CAR, and BCMA CAR) to the T cells. In some embodiments, the methods disclosed herein further comprise reducing expression of major histocompatibility complex (MHC) class I and/or MHC class II molecules in the T cells.

Among other things, the present disclosure provides methods of producing a composition comprising genetically engineered cells. In some embodiments, a method comprises the step of selecting one or more genetically engineered cells from a population of cells based on a level of one or more markers on the cell surface of the one or more genetically engineered cells. In some embodiments, the level of the one or more markers on the cell surface comprise a level of CD3. In some embodiments, a method comprises the step of formulating the composition comprising the selected one or more genetically engineered cells for treating a disease in a subject. In some embodiments, one or more genetically engineered cells comprise one or more genetic modifications. In some embodiments, one or more genetic modifications comprise a transgene encoding a first tolerogenic factor at an insertion site at a T-cell receptor (TCR) gene locus.

The present disclosure further provides methods of selecting engineered cells suitable for use in a therapeutic product. In some embodiments, a method comprises the step of selecting one or more genetically engineered cells from a population of cells based on a level of one or more markers on the cell surface of the one or more genetically engineered cells. In some embodiments, the level of the one or more markers on the cell surface comprise a level of CD3. In some embodiments, a method comprises the step of formulating the composition comprising the selected one or more genetically engineered cells for treating a disease in a subject. In some embodiments, one or more genetically engineered cells comprise one or more genetic modifications. In some embodiments, one or more genetic modifications comprise a transgene encoding a first tolerogenic factor at an insertion site at a T-cell receptor (TCR) gene locus.

The present disclosure provides methods of treating a disease in a subject with a composition comprising genetically engineered cells. In some embodiments, a method comprises the step of selecting one or more genetically engineered cells from a population of cells based on a level of one or more markers on the cell surface of the one or more genetically engineered cells. In some embodiments, the level of the one or more markers on the cell surface comprise a level of CD3. In some embodiments, a method comprises the step of formulating the composition comprising the selected one or more genetically engineered cells for treating a disease in a subject. In some embodiments, one or more genetically engineered cells comprise one or more genetic modifications. In some embodiments, one or more genetic modifications comprise a transgene encoding a first tolerogenic factor at an insertion site at a T-cell receptor (TCR) gene locus. In some embodiments, a method comprises the step of administering the formulated composition to a subject.

The present disclosure further provides methods of producing a composition comprising engineered cells with increased purity. In some embodiments, a method comprises the step of selecting one or more genetically engineered cells from a population of cells based on a level of one or more markers on the cell surface of the one or more genetically engineered cells. In some embodiments, the level of the one or more markers on the cell surface comprise a level of CD3. In some embodiments, a method comprises the step of formulating the composition comprising the selected one or more genetically engineered cells for treating a disease in a subject. In some embodiments, one or more genetically engineered cells comprise one or more genetic modifications. In some embodiments, one or more genetic modifications comprise a transgene encoding a first tolerogenic factor at an insertion site at a T-cell receptor (TCR) gene locus. In some embodiments, at least 30%, at least 35%, at least 40%, at least 45%, at least 50%, at least 55%, at least 60%, at least 65%, at least 70%, at least 75%, at least 80%, at least 85%, at least 90%, or at least 95% of the genetically engineered cells in the formulated composition comprise the transgene encoding the first tolerogenic factor at the insertion site at the TCR gene locus.

The present disclosure also provides methods of producing a composition comprising genetically engineered cells with enhanced efficacy. In some embodiments, a method comprises the step of selecting one or more genetically engineered cells from a population of cells based on a level of one or more markers on the cell surface of the one or more genetically engineered cells. In some embodiments, the level of the one or more markers on the cell surface comprise a level of CD3. In some embodiments, a method comprises the step of formulating the composition comprising the selected one or more genetically engineered cells for treating a disease in a subject. In some embodiments, one or more genetically engineered cells comprise one or more genetic modifications. In some embodiments, one or more genetic modifications comprise a transgene encoding a first tolerogenic factor at an insertion site at a T-cell receptor (TCR) gene locus. In some embodiments, a composition with enhanced efficacy is more effective than a composition comprising cells that do not comprise the one or more genetic modifications.

Additionally, the present disclosure provides methods of producing a composition with reduced host immune response. In some embodiments, a method comprises the step of selecting one or more genetically engineered cells from a population of cells based on a level of one or more markers on the cell surface of the one or more genetically engineered cells. In some embodiments, the level of the one or more markers on the cell surface comprise a level of CD3. In some embodiments, a method comprises the step of formulating the composition comprising the selected one or more genetically engineered cells for treating a disease in a subject. In some embodiments, one or more genetically engineered cells comprise one or more genetic modifications. In some embodiments, one or more genetic modifications comprise a transgene encoding a first tolerogenic factor at an insertion site at a T-cell receptor (TCR) gene locus. In some embodiments, a composition with reduced host immune response elicits a reduced host immune response compared to a composition comprising comparable cells that do not comprise the one or more genetic modifications.

Further, the present disclosure provides methods of formulating a composition with reduced immunogenicity. In some embodiments, a method comprises the step of selecting one or more genetically engineered cells from a population of cells based on a level of one or more markers on the cell surface of the one or more genetically engineered cells. In some embodiments, the level of the one or more markers on the cell surface comprise a level of CD3. In some embodiments, a method comprises the step of formulating the composition comprising the selected one or more genetically engineered cells for treating a disease in a subject. In some embodiments, one or more genetically engineered cells comprise one or more genetic modifications. In some embodiments, one or more genetic modifications comprise a transgene encoding a first tolerogenic factor at an insertion site at a T-cell receptor (TCR) gene locus. In some embodiments, a composition with reduced immunogenicity elicits a reduced host immune response compared to a composition comprising comparable cells that do not comprise the one or more genetic modifications.

The present disclosure further provides methods of producing a composition comprising genetically engineered cells with reduced immunogenicity. In some embodiments, a method comprises the step of selecting one or more genetically engineered cells from a population of cells based on a level of one or more markers on the cell surface of the one or more genetically engineered cells. In some embodiments, the level of the one or more markers on the cell surface comprise a level of CD3. In some embodiments, a method comprises the step of formulating the composition comprising the selected one or more genetically engineered cells for treating a disease in a subject. In some embodiments, one or more genetically engineered cells comprise one or more genetic modifications. In some embodiments, one or more genetic modifications comprise a transgene encoding a first tolerogenic factor at an insertion site at a T-cell receptor (TCR) gene locus. In some embodiments, a composition with reduced immunogenicity elicits a reduced host immune response compared to a composition comprising comparable cells that do not comprise the one or more genetic modifications.

In some embodiments provided herein, a host immune response is an immune response of a subject against the one or more genetically engineered cells. In some embodiments, a reduced host immune response comprises reduced donor-specific antibodies in the subject. In some embodiments, a reduced host immune response comprises reduced IgM or IgG antibodies in the subject. In some embodiments, a reduced host immune response comprises reduced complement-dependent cytotoxicity (CDC) in the subject. In some embodiments, a reduced host immune response comprises reduced TH1 activation in the subject. In some embodiments, a reduced host immune response comprises reduced NK cell killing in the subject. In some embodiments, a reduced host immune response comprises reduced killing by whole PBMCs in the subject.

The present disclosure also provides methods of producing a composition comprising genetically engineered cells with a reduced graft versus host response. In some embodiments, a method comprises the step of selecting one or more genetically engineered cells from a population of cells based on a level of one or more markers on the cell surface of the one or more genetically engineered cells. In some embodiments, the level of the one or more markers on the cell surface comprise a level of CD3. In some embodiments, a method comprises the step of formulating the composition comprising the selected one or more genetically engineered cells for treating a disease in a subject. In some embodiments, one or more genetically engineered cells comprise one or more genetic modifications. In some embodiments, one or more genetic modifications comprise a transgene encoding a first tolerogenic factor at an insertion site at a T-cell receptor (TCR) gene locus. In some embodiments, one or more genetically engineered cells of the composition with a reduced graft versus host response have a reduced immune response against cells of the subject as compared to a composition comprising comparable cells that do not comprise the one or more genetic modifications.

In some embodiments, one or more genetic modifications comprise an inserted transgene encoding a first tolerogenic factor. In some embodiments, a transgene encoding the first tolerogenic factor is inserted at an insertion site at a T-cell receptor (TCR) gene locus.

In some embodiments, methods provided herein comprise inserting a transgene encoding a first tolerogenic factor into an insertion site in the genome of one or more cells in the population. In some embodiments, the step of inserting comprises homology-directed repair (HDR)-mediated insertion using a genome-modifying protein.

In some embodiments, the step of inserting using a genome modifying protein comprises insertion by a CRISPR-associated transposase, prime editing, a TnpB polypeptide, or Programmable Addition via Site-specific Targeting Elements (PASTE).

In some embodiments, the step of inserting using a genome modifying protein comprises insertion by a site-directed nuclease. In some embodiments, a site-directed nuclease is selected from a zinc finger nuclease (ZFN), a TAL-effector nuclease (TALEN), or a CRISPR-Cas combination, optionally wherein the Cas is selected from a Cas9 or a Cas12. In some embodiments, a site-directed nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, and a TnpB polypeptide.

In some embodiments, the step of inserting comprises homology-directed repair (HDR)-mediated insertion using a guide RNA (gRNA) and a CRISPR-associated (Cas) nuclease. In some embodiments, a gRNA comprises a complementary region. In some embodiments, a complementary region comprises a nucleic acid sequence that is complementary to a target nucleic acid sequence within the TCR gene locus. In some embodiments, a target nucleic acid sequence comprises the insertion site.

In some embodiments, an insertion site is 25 nucleotides or less from a protospacer adjacent motif (PAM) sequence. In some embodiments, a PAM sequence is ngg, nag, ngrrt, ngrrn, nnnngatt, nnnnryac, nnagaaw, naaaac, tttv, ttn, attn, tttn, or gttn, and where (i) r=a or g, (ii) y=c or t, (iii) w=a or t, (iv) v=a or c or g, and (v) n=a, c, t, or g.

In some embodiments, the step of inserting comprises homology-directed repair (HDR)-mediated insertion using SpCas9 and the PAM is ngg or nag, where n=a, c, t, or g.

In some embodiments, the step of inserting comprises homology-directed repair (HDR)-mediated insertion using SaCas9 and the PAM is ngrrt or ngrrn, where (i) r=a or g, and (ii) n=a, c, t, or g.

In some embodiments, the step of inserting comprises homology-directed repair (HDR)-mediated insertion using NmeCas9 and the PAM is nnnngatt, wherein n=a, c, t, or g.

In some embodiments, the step of inserting comprises homology-directed repair (HDR)-mediated insertion using CjCas9 and the PAM is nnnnryac, where (i) r=a or g, (ii) y=c or t, and (iii) n=a, c, t, or g.

In some embodiments, the step of inserting comprises homology-directed repair (HDR)-mediated insertion using StCas9 and the PAM is nnagaaw, where (i) w=a or t, and (ii) n=a, c, t, or g.

In some embodiments, the step of inserting comprises homology-directed repair (HDR)-mediated insertion using TdCas9 and the PAM is naaaac, where n=a, c, t, or g.

In some embodiments, the step of inserting comprises homology-directed repair (HDR)-mediated insertion using LbCas12a and the PAM is tttv, where v=a or c or g.

In some embodiments, the step of inserting comprises homology-directed repair (HDR)-mediated insertion using AsCas12a and the PAM is tttv, where v=a or c or g.

In some embodiments, the step of inserting comprises homology-directed repair (HDR)-mediated insertion using AacCas12b and the PAM is ttn, where n=a, c, t, or g.

In some embodiments, the step of inserting comprises homology-directed repair (HDR)-mediated insertion using BhCas12b and the PAM is attn, tttn, or gttn, where n=a, c, t, or g.

In some embodiments, homology-directed repair (HDR)-mediated insertion using a site-directed nuclease is performed with an HDR efficiency equal to or greater than HDR insertion using lentivirus.

In some embodiments, the step of inserting comprises homology-directed repair (HDR)-mediated insertion using ZFN. In some embodiments, the first insertion site is 25 nucleotides or less from a zinc finger binding sequence.

In some embodiments, the step of inserting comprises homology-directed repair (HDR)-mediated insertion using TALEN. In some embodiments, the first insertion site is 25 nucleotides or less from a transcription activator-like effectors (TALE) binding sequence.

In some embodiments, step of inserting comprises homology-directed repair (HDR)-mediated insertion using a guide RNA (gRNA) and a TnpB polypeptide. In some embodiments, a gRNA comprises a complementary region. In some embodiments, a complementary region comprises a nucleic acid sequence that is complementary to a target nucleic acid sequence within the TCR gene locus. In some embodiments, a target nucleic acid sequence comprises the insertion site.

In some embodiments, an insertion site is 25 nucleotides or less from a target adjacent motif (TAM) sequence, wherein the TAM sequence is tca, ttcan, ttgatn or ataaa, and where n=a, c, t, or g.

In some embodiments, the step of inserting comprises homology-directed repair (HDR)-mediated insertion using TnpB polypeptide and the TAM is tca.

In some embodiments, the step of inserting comprises homology-directed repair (HDR)-mediated insertion using TnpB polypeptide and the TAM is ttcan, wherein n=a, c, t, or g.

In some embodiments, the step of inserting comprises homology-directed repair (HDR)-mediated insertion using TnpB polypeptide and the TAM is ttgatn, wherein n=a, c, t, or g.

In some embodiments, the step of inserting comprises homology-directed repair (HDR)-mediated insertion using TnpB polypeptide and the TAM is ataaa.

In some embodiments, an insertion site is in an exon. In some embodiments, an insertion site is in an intron. In some embodiments, an insertion site is between an intron and an exon. In some embodiments, an insertion site is in a regulatory region.

In some embodiments, a transgene encoding a first tolerogenic factor at an insertion site at a T-cell receptor (TCR) gene locus reduces expression of a functional TCR. In some embodiments, a transgene encoding a first tolerogenic factor at an insertion site at a T-cell receptor (TCR) gene locus disrupts expression of a functional TCR.

In some embodiments, a transgene encoding a first tolerogenic factor has a reverse orientation (5′ to 3′) relative to the TCR locus.

In some embodiments, a TCR locus is an endogenous TCR locus. In some embodiments, avTCR locus is or comprises: a TRAC locus, a TRBC1 locus, or a TRBC2 locus. In some embodiments, a TCR locus is or comprises a TRAC locus. In some embodiments, an insertion site is within exon 1 at the TRAC locus.

In some embodiments, the step of inserting comprises using an hTRAC gRNA comprising the nucleic acid sequence TCAGGGTTCTGGATATCTGT (SEQ ID NO: 124).

In some embodiments, a level of one or more markers on the cell surface comprises a level of a first tolerogenic factor on the cell surface of the one or more genetically engineered cells. In some embodiments, a method comprises detecting a level of the first tolerogenic factor on the cell surface of the one or more genetically engineered cells. In some embodiments, one or more genetically engineered cells are selected if the first tolerogenic factor is detected on the cell surface of the one or more genetically engineered cells.

In some embodiments, a first tolerogenic factor is or comprises A20/TNFAIP3, B2M-HLA-E, C1-Inhibitor, CCL21, CCL22, CD16, CD16 Fc receptor, CD24, CD27, CD35, CD39, CD46, CD47, CD52, CD55, CD59, CD200, CR1, CTLA4-Ig, DUX4, FasL, H2-M3 (HLA-G), HLA-C, HLA-E, HLA-E heavy chain, HLA-F, HLA-G, IDO1, IL-10, IL15-RF, IL-35, IL-39, MANF, Mfge8, PD-L1, or Serpinb9.

In some embodiments, a first tolerogenic factor is or comprises CD47. In some embodiments, the first tolerogenic factor is or comprises human CD47. In some embodiments, CD47 comprises an amino acid sequence at least 80%, at least 85%, at least 90%, at least 91%, at least 92%, at least 93%, at least 94%, at least 95%, at least 96%, at least 97%, at least 98%, at least 99%, or 100% identical to an amino acid sequence set forth in SEQ ID NO:1 or SEQ ID NO:2. In some embodiments, a transgene encoding a first tolerogenic factor is a transgene that encodes CD47 and the transgene comprises a nucleotide sequence at least 80%, at least 85%, at least 90%, at least 91%, at least 92%, at least 93%, at least 94%, at least 95%, at least 96%, at least 97%, at least 98%, at least 99%, or 100% identical to a nucleotide sequence set forth in SEQ ID NO:3 or SEQ ID NO:4.

In some embodiments, a transgene encoding a first tolerogenic factor is a transgene that encodes CD47 and the nucleotide sequence of the transgene is codon-optimized. In some embodiments, a transgene is at least 80%, at least 85%, at least 90%, at least 91%, at least 92%, at least 93%, at least 94%, at least 95%, at least 96%, at least 97%, at least 98%, at least 99%, or 100% identical to a nucleotide sequence set forth in SEQ ID NO:5.

In some embodiments, a method comprises detecting a level of CD3 on the cell surface of the one or more genetically engineered cells. In some embodiments, one or more genetically engineered cells are selected if CD3 is not present at a detectable level on the cell surface of the one or more genetically engineered cells.

In some embodiments, a level of one or more markers on the cell surface comprises a level of TCR on the cell surface of the one or more genetically engineered cells. In some embodiments, a method comprises detecting a level of TCR on the cell surface of the one or more genetically engineered cells. In some embodiments, one or more genetically engineered cells are selected if TCR is not present at a detectable level on the cell surface of the one or more genetically engineered cells.

In some embodiments, one or more genetic modifications comprise a modification at a B2M locus, a TAP I locus, a NLRC5 locus, a CIITA locus, an HLA-A locus, an HLA-B locus, an HLA-C locus, an HLA-DP locus, an HLA-DM locus, an HLA-DOA locus, an HLA-DOB locus, an HLA-DQ locus, an HLA-DR locus, a RFX5 locus, a RFXANK locus, a RFXAP locus, an NFY-A locus, an NFY-B locus, an NFY-C locus, or a combination thereof.

In some embodiments, one or more genetic modifications comprise a modification at an HLA-DM locus, an HLA-DO locus, an HLA-DP locus, an HLA-DQ locus, an HLA-DR locus, or a combination thereof. In some embodiments, a modification at the HLA-DM locus, the HLA-DO locus, the HLA-DP locus, the HLA-DQ locus, the HLA-DR locus, or a combination thereof comprises a knock-out of the HLA-DM locus, the HLA-DO locus, the HLA-DP locus, the HLA-DQ locus, the HLA-DR locus, or a combination thereof. In some embodiments, a modification at the HLA-DM locus, the HLA-DO locus, the HLA-DP locus, the HLA-DQ locus, the HLA-DR locus, or a combination thereof is a heterozygous modification. In some embodiments, a modification at the HLA-DM locus, the HLA-DO locus, the HLA-DP locus, the HLA-DQ locus, the HLA-DR locus, or a combination thereof is a homozygous modification.

In some embodiments, a method comprises modifying an HLA-DM locus, an HLA-DO locus, an HLA-DP locus, an HLA-DQ locus, an HLA-DR locus, or a combination thereof. In some embodiments, a method comprises knocking out an HLA-DM locus, an HLA-DO locus, an HLA-DP locus, an HLA-DQ locus, an HLA-DR locus, or a combination thereof.

In some embodiments, one or more genetic modifications comprise a modification at an HLA-A locus, an HLA-B locus, an HLA-C locus, or a combination thereof. In some embodiments, a modification at the HLA-A locus, the HLA-B locus, the HLA-C locus, or a combination thereof comprises a knock-out of the HLA-A locus, the HLA-B locus, the HLA-C locus, or a combination thereof. In some embodiments, a modification at the HLA-A locus, the HLA-B locus, the HLA-C locus, or a combination thereof is a heterozygous modification. In some embodiments, a modification at the HLA-A locus, the HLA-B locus, the HLA-C locus, or a combination thereof is a homozygous modification.

In some embodiments, a method comprises modifying an HLA-A locus, an HLA-B locus, an HLA-C locus, or a combination thereof. In some embodiments, a method comprises knocking out an HLA-A locus, an HLA-B locus, an HLA-C locus, or a combination thereof.

In some embodiments, one or more genetic modifications comprise a modification at a B2M locus. In some embodiments, a modification at the B2M locus comprises a knock-out of the B2M locus. In some embodiments, a modification at the B2M locus is a heterozygous modification. In some embodiments, a modification at the B2M locus is a homozygous modification.

In some embodiments, a method comprises modifying a B2M locus. In some embodiments, a method comprises knocking out the B2M locus.

In some embodiments, one or more genetic modifications comprise a modification at a CIITA locus. In some embodiments, a modification at the CIITA locus comprises a knock-out of the CIITA locus. In some embodiments, a modification at the CIITA locus is a heterozygous modification. In some embodiments, a modification at the CIITA locus is a homozygous modification.

In some embodiments, a method comprises modifying a CIITA locus. In some embodiments, a method comprises knocking out the CIITA locus.

In some embodiments, a level of one or more markers on the cell surface comprises a level of an MHC I molecule, an MHC II molecule, or both, on the cell surface of the one or more genetically engineered cells. In some embodiments, a method comprises detecting a level of the MHC I molecule, the MHC II molecule, or both, on the cell surface of the one or more genetically engineered cells. In some embodiments, one or more genetically engineered cells are selected if the MHC I molecule, the MHC II molecule, or both, are not present at a detectable level on the cell surface of the one or more genetically engineered cells.

In some embodiments, one or more genetic modifications comprise a knock-out of: ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRBC1, TRBC2, or a combination thereof. In some embodiments, a level of one or more markers on the cell surface comprises a level of ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRBC1, TRBC2, or a combination thereof, on the cell surface of the one or more genetically engineered cells. In some embodiments, a method comprises detecting a level of ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRBC1, TRBC2, or a combination thereof, on the cell surface of the one or more genetically engineered cells. In some embodiments, one or more genetically engineered cells are selected if ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRBC1, TRBC2, or a combination thereof, are not present at a detectable level on the cell surface of the one or more genetically engineered cells. In some embodiments, a protein that is involved in oxidative or ER stress is selected from the group consisting of TXNIP, PERK, IRE1α, and DJ-1 (PARK7).

In some embodiments, one or more genetic modifications comprise a second inserted transgene. In some embodiments, a second transgene encodes a chimeric antigen receptor (CAR). In some embodiments, a method comprises inserting a transgene encoding a CAR in the genome of one or more cells in the population.

In some embodiments, a transgene encoding a CAR is inserted at a safe harbor locus. In some embodiments, a transgene encoding a CAR is inserted at a TRAC locus, a TRBC1 locus, a TRBC2 locus, a B2M locus, a CIITA locus, a MICA locus, a MICB locus, or a safe harbor locus. In some embodiments, a transgene encoding a CAR is inserted at an AAVS1 locus, an ABO locus, a CCR5 locus, a CLYBL locus, a CXCR4 locus, a F3 locus, a FUT1 locus, a HMGB1 locus, a KDM5D locus, a LRP1 locus, a RHD locus, a ROSA26 locus, or a SHS231 locus. In some embodiments, a second transgene is inserted into same site as the transgene encoding the first tolerogenic factor.

In some embodiments, a second transgene and a first tolerogenic factor are encoded by two separate constructs.

In some embodiments, a second transgene and a first tolerogenic factor are encoded by a bicistronic construct.

In some embodiments, a CAR comprises a CD5-specific CAR, a CD19-specific CAR, a CD20-specific CAR, a CD22-specific CAR, a CD23-specific CAR, a CD30-specific CAR, a CD33-specific CAR, CD38-specific CAR, a CD70-specific CAR, a CD123-specific CAR, a CD138-specific CAR, a Kappa, Lambda, B cell maturation agent (BCMA)-specific CAR, a G-protein coupled receptor family C group 5 member D (GPRC5D)-specific CAR, a CD123-specific CAR, a LeY-specific CAR, a NKG2D ligand-specific CAR, a WT1-specific CAR, a GD2-specific CAR, a HER2-specific CAR, a EGFR-specific CAR, a EGFRvIII-specific CAR, a B7H3-specific CAR, a PSMA-specific CAR, a PSCA-specific CAR, a CAIX-specific CAR, a CD171-specific CAR, a CEA-specific CAR, a CSPG4-specific CAR, a EPHA2-specific CAR, a FAP-specific CAR, a FRα-specific CAR, a IL-13Rα-specific CAR, a Mesothelin-specific CAR, a MUC1-specific CAR, a MUC16-specific CAR, a ROR1-specific CAR, a C-Met-specific CAR, a CD133-specific CAR, a Ep-CAM-specific CAR, a GPC3-specific CAR, a HPV16-E6-specific CAR, a IL13Ra2-specific CAR, a MAGEA3-specific CAR, a MAGEA4-specific CAR, a MART1-specific CAR, a NY-ESO-1-specific CAR, a VEGFR2-specific CAR, a α-Folate receptor-specific CAR, a CD24-specific CAR, a CD44v7/8-specific CAR, a EGP-2-specific CAR, a EGP-40-specific CAR, a erb-B2-specific CAR, a erb-B 2,3,4-specific CAR, a FBP-specific CAR, a Fetal acethylcholine e receptor-specific CAR, a G_{D2}-specific CAR, a G_{D3}-specific CAR, a HMW-MAA-specific CAR, a IL-11Rα-specific CAR, a KDR-specific CAR, a Lewis Y-specific CAR, a L1-cell adhesion molecule-specific CAR, a MAGE-A1-specific CAR, a Oncofetal antigen (h5T4)-specific CAR, a TAG-72-specific CAR, or a CD19/CD22-bispecific CAR.

In some embodiments, a CAR comprises a CD19-specific CAR, a CD20-specific CAR, a CD22-specific CAR, a CD38-specific CAR, a CD123-specific CAR, a CD138-specific CAR, a BCMA-specific CAR, or a CD19/CD22-bispecific CAR,

In some embodiments, a level of one or more markers on the cell surface comprises a level of the CAR on the cell surface of the one or more genetically engineered cells. In some embodiments, a method comprises detecting a level of the CAR on the cell surface of the one or more genetically engineered cells. In some embodiments, one or more genetically engineered cells are selected if the CAR is detected on the cell surface of the one or more genetically engineered cells.

In some embodiments, a second transgene encodes a second tolerogenic factor. In some embodiments, a second transgene encoding the second tolerogenic factor is inserted at a TRAC locus, a TRBC1 locus, a TRBC2 locus, a B2M locus, a CIITA locus, a MICA locus, a MICB locus, a safe harbor locus, an AAVS1 locus, an ABO locus, a CCR5 locus, a CLYBL locus, a CXCR4 locus, a F3 locus, a FUT1 locus, a HMGB1 locus, a KDM5D locus, a LRP1 locus, a RHD locus, a ROSA26 locus, or a SHS231 locus. In some embodiments, a second tolerogenic factor is or comprises A20/TNFAIP3, B2M-HLA-E, C1-Inhibitor, CCL21, CCL22, CD16, CD16 Fc receptor, CD24, CD27, CD35, CD39, CD46, CD47, CD52, CD55, CD59, CD200, CR1, CTLA4-Ig, DUX4, FasL, H2-M3 (HLA-G), HLA-C, HLA-E, HLA-E heavy chain, HLA-F, HLA-G, IDO1, IL-10, IL15-RF, IL-35, IL-39, MANF, Mfge8, PD-L1, or Serpinb9.

In some embodiments, a first tolerogenic factor and a second tolerogenic factor are the same tolerogenic factor. In some embodiments, a first tolerogenic factor and the second tolerogenic factor are different tolerogenic factors.

In some embodiments, a method comprises detecting a level of the second tolerogenic factor on the cell surface of the one or more genetically engineered cells. In some embodiments, a second tolerogenic factor is expressed at a higher level than endogenous expression levels of the second tolerogenic factor in a comparable cell that does not comprise the second transgene. In some embodiments, one or more genetically engineered cells are selected if the second tolerogenic factor is detected on the cell surface of the one or more genetically engineered cells at a higher level of expression than endogenous expression levels of the second tolerogenic factor in a comparable cell that does not comprise the second transgene.

In some embodiments, one or more genetically engineered cells are selected from a population of cells based on a level of two or more markers on the cell surface of the one or more genetically engineered cells. In some embodiments, one or more genetically engineered cells are selected from a population of cells based on a level of three or more markers on the cell surface of the one or more genetically engineered cells. In some embodiments, one or more genetically engineered cells are selected from a population of cells based on a level of four or more markers on the cell surface of the one or more genetically engineered cells.

In some embodiments, each of the one or more markers on the cell surface of the one or more genetically engineered cells is associated with at least one of the one or more genetic modifications. In some embodiments, each of the one or more genetic modifications impacts the level of at least one of the one or more markers on the cell surface of the one or more genetically engineered cells.

In some embodiments, a transgene encoding the first tolerogenic factor comprises a promoter, an insulator, an enhancer, a polyadenylation (poly(A)) tail, a ubiquitous chromatin opening element, or a combination thereof. In some embodiments, a transgene encoding the CAR comprises a promoter, an insulator, an enhancer, a polyadenylation (poly(A)) tail, a ubiquitous chromatin opening element, or a combination thereof. In some embodiments, a transgene encoding the second tolerogenic factor comprises a promoter, an insulator, an enhancer, a polyadenylation (poly(A)) tail, a ubiquitous chromatin opening element, or a combination thereof.

In some embodiments, a transgene encoding the first tolerogenic factor comprises a promoter and the promoter is a constitutive promoter. In some embodiments, a transgene encoding the CAR comprises a promoter and the promoter is a constitutive promoter. In some embodiments, a transgene encoding the second tolerogenic factor comprises a promoter and the promoter is a constitutive promoter.

In some embodiments, a constitutive promoter is an EF1α, EF1α short, CMV, SV40, PGK, adenovirus late, vaccinia virus 7.5K, SV40, HSV tk, mouse mammary tumor virus (MMTV), HIV LTR, moloney virus, Esptein Barr virus (EBV), Rous sarcoma virus (RSV), UBC CAG, MND, SSFV, or ICOS promoter.

In some embodiments, a population of cells are human cells or non-human animal cells. In some embodiments, non-human animal cells are porcine, bovine or ovine cells. In some embodiments, a population of cells are human cells.

In some embodiments, a population of cells are differentiated cells derived from stem cells or progenitor cells. In some embodiments, stem cells are pluripotent stem cells. In some embodiments, pluripotent stem cells are induced pluripotent stem cells. In some embodiments, pluripotent stem cells are embryonic stem cells.

In some embodiments, a population of cells are primary cells isolated from a donor. In some embodiments, a donor is a single donor or multiple donors. In some embodiments, a donor is healthy and/or is not suspected of having a disease or condition at the time the primary cells are obtained from the donor.

In some embodiments, a population of cells are islet cells, beta islet cells, pancreatic islet cells, immune cells, B cells, T cells, natural killer (NK) cells, natural killer T (NKT) cells, macrophage cells, endothelial cells, muscle cells, cardiac muscle cells, smooth muscle cells, skeletal muscle cells, dopaminergic neurons, retinal pigmented epithelium cells, optic cells, hepatocytes, thyroid cells, skin cells, glial progenitor cells, neural cells, cardiac cells, stem cells, hematopoietic stem cells, induced pluripotent stem cells (iPSCs), mesenchymal stem cells (MSCs), embryonic stem cells (ESCs), pluripotent stem cell (PSCs), blood cells, or a combination thereof.

In some embodiments, a population of cells are T-cells. In some embodiments, T-cells are CD3+ T cells, CD4+ T cells, CDS+ T cells, naive T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T cells, effector memory T cells, effector memory T cells expressing CD45RA (TEMRA cells), tissue-resident memory (Trm) cells, virtual memory T cells, innate memory T cells, memory stem cell (Tse), γδ T cells, or a combination thereof. In some embodiments, T cells are cytotoxic T-cells, helper T-cells, memory T-cells, regulatory T-cells, tumor infiltrating lymphocytes, or a combination thereof. In some embodiments, T-cells are human T-cells.

In some embodiments, a population of cells are autologous T-cells.

In some embodiments, a population of cells are allogenic T-cells. In some embodiments, allogeneic T cells are primary T cells. In some embodiments, allogeneic T cells have been differentiated from embryonic stem cells (ESCs) or an induced pluripotent stem cells (iPSCs).

In some embodiments, a population of cells are T-cells, and wherein, after the steps of inserting the transgene encoding the first tolerogenic factor and modifying an HLA-A locus, an HLA-B locus, an HLA-C locus, or a combination thereof, at least 30%, at least 35%, at least 40%, at least 45%, at least 50%, at least 55%, at least 60%, at least 65%, at least 70%, at least 75%, at least 80%, at least 85%, at least 90%, or at least 95% of the population of T-cells each have (a) reduced cell surface expression of MHC I and/or MHC II molecules as compared to comparable T-cells that have not been genetically engineered, and (b) increased expression of the first tolerogenic factor encoded by the first transgene as compared to comparable T-cells that have not been genetically engineered.

In some embodiments, a population of cells are T-cells and the tolerogenic factor is CD47, and wherein, after the steps of inserting the transgene encoding the first tolerogenic factor and knocking out the B2M locus and/or the CIITA locus, at least 30%, at least 35%, at least 40%, at least 45%, at least 50%, at least 55%, at least 60%, at least 65%, at least 70%, at least 75%, at least 80%, at least 85%, at least 90%, or at least 95% of the population of T-cells each have (a) a B2M locus and/or a CIITA locus knocked-out, and (b) increased expression of CD47 as compared to comparable T-cells that have not been genetically engineered. In some embodiments, at least 30%, at least 35%, at least 40%, at least 45%, at least 50%, at least 55%, at least 60%, at least 65%, at least 70%, at least 75%, at least 80%, at least 85%, at least 90%, or at least 95% of the population of T-cells each have (a) and (b).

In some embodiments, a population of cells are T-cells and the first tolerogenic factor is CD47, and wherein, after the steps of inserting the transgene encoding the first tolerogenic factor and knocking out the B2M locus and/or the CIITA locus, at least 30% of the population of T-cells each have (a) reduced cell surface expression of MHC I and/or MHC II molecules as compared to T-cells that have not been genetically engineered, and (b) increased expression of CD47 as compared to comparable T-cells that have not been genetically engineered. In some embodiments, at least 30%, at least 35%, at least 40%, at least 45%, at least 50%, at least 55%, at least 60%, at least 65%, at least 70%, at least 75%, at least 80%, at least 85%, at least 90%, or at least 95% of the population of T-cells each have (a) and (b).

In some embodiments, a population of cells are T-cells and the tolerogenic factor is CD47, and wherein, after the steps of inserting the transgene encoding the first tolerogenic factor and knocking out the B2M locus and/or the CIITA locus, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the population of T-cells each have (a) reduced expression of B2M as compared to comparable T-cells that have not been genetically engineered, (b) reduced expression of CIITA as compared to comparable T-cells that have not been genetically engineered, and (c) increased expression of CD47 as compared to comparable T-cells that have not been genetically engineered. In some embodiments, at least 30%, at least 35%, at least 40%, at least 45%, at least 50%, at least 55%, at least 60%, at least 65%, at least 70%, at least 75%, at least 80%, at least 85%, at least 90%, or at least 95% of the T-cells each have (a) and (b). In some embodiments, at least 30%, at least 35%, at least 40%, at least 45%, at least 50%, at least 55%, at least 60%, at least 65%, at least 70%, at least 75%, at least 80%, at least 85%, at least 90%, or at least 95% of the population of T-cells each have (a), (b), and (c).

In some embodiments, a method comprises freezing the cells. In some embodiments, one or more genetically engineered cells are frozen after being selected based on a level of one or more markers on the cell surface of the one or more genetically engineered cells. In some embodiments, one or more genetically engineered cells are frozen after one or more genetic modifications are introduced.

In some embodiments, a method comprises thawing the cells. In some embodiments, one or more genetically engineered cells are thawed prior to one or more genetic modifications being introduced. In some embodiments, one or more genetically engineered cells are formulated in the composition after thawing. In some embodiments, one or more genetically engineered cells are formulated in the composition before thawing.

In some embodiments, a composition is suitable for use in a subject. In some embodiments, a composition is a therapeutic composition. In some embodiments, a composition is a cell therapy composition.

In some embodiments, a composition comprises a pharmaceutically acceptable additive, carrier, diluent, or excipient. In some embodiments, a composition comprises a buffered solution. In some embodiments, a composition comprises a pharmaceutically acceptable buffer. In some embodiments, a pharmaceutically acceptable buffer comprises neutral buffer saline or phosphate buffered saline.

In some embodiments, a composition comprises Plasma-Lyte A®, dextrose, dextran, sodium chloride, human serum albumin (HSA), dimethylsulfoxide (DMSO), or a combination thereof.

In some embodiments, a composition comprises a cryoprotectant.

The present disclosure provides populations of genetically engineered cells.

In some embodiments, a population of genetically engineered cells is produced by a method described herein.

In some embodiments, a population of cells have been genetically engineered to comprise a transgene encoding a first tolerogenic factor. In some embodiments, at least 30% of cells in a population have increased cell surface expression of a first tolerogenic factor as compared to a comparable cell that has not been genetically engineered. In some embodiments, at least 30%, at least 35%, at least 40%, at least 45%, at least 50%, at least 55%, at least 60%, at least 65%, at least 70%, at least 75%, at least 80%, at least 85%, at least 90%, or at least 95% of the cells have increased cell surface expression of a first tolerogenic factor as compared to a comparable cell that has not been genetically engineered.

In some embodiments, a transgene encoding the first tolerogenic factor is inserted at an insertion site at a T-cell receptor (TCR) gene locus. In some embodiments, an insertion site is in an exon. In some embodiments, an insertion site is in an intron. In some embodiments, an insertion site is between an intron and an exon. In some embodiments, an insertion site is in a regulatory region.

In some embodiments, a tolerogenic factor is CD47.

In some embodiments, at least 30% of the cells have decreased cell surface expression of a TCR as compared to a comparable cell that has not been genetically engineered. In some embodiments, at least 30%, at least 35%, at least 40%, at least 45%, at least 50%, at least 55%, at least 60%, at least 65%, at least 70%, at least 75%, at least 80%, at least 85%, at least 90%, or at least 95% of the cells have decreased cell surface expression of a TCR as compared to a comparable cell that has not been genetically engineered.

In some embodiments, a population of cells have been genetically engineered to knock-out an HLA-A locus, an HLA-B locus, an HLA-C locus, or a combination thereof.

In some embodiments, a population of cells have been genetically engineered to knock-out an HLA-DM locus, an HLA-DO locus, an HLA-DP locus, an HLA-DQ locus, an HLA-DR locus, or a combination thereof.

In some embodiments, a population of cells have been genetically engineered to knock-out a B2M locus.

In some embodiments, a population of cells have been genetically engineered to knock-out a CIITA locus.

In some embodiments, at least 30% of the cells have decreased cell surface expression of an MHC I molecule, an MHC II molecule, or both, as compared to a comparable cell that has not been genetically engineered. In some embodiments, at least 30%, at least 35%, at least 40%, at least 45%, at least 50%, at least 55%, at least 60%, at least 65%, at least 70%, at least 75%, at least 80%, at least 85%, at least 90%, or at least 95% of the cells have decreased cell surface expression of an MHC I molecule, an MHC II molecule, or both, as compared to a comparable cell that has not been genetically engineered.

In some embodiments, a population of cells have been genetically engineered to comprise a transgene encoding a CAR.

In some embodiments, at least 30% of the cells have cell surface expression of the CAR. In some embodiments, at least 30%, at least 35%, at least 40%, at least 45%, at least 50%, at least 55%, at least 60%, at least 65%, at least 70%, at least 75%, at least 80%, at least 85%, at least 90%, or at least 95% of the cells have cell surface expression of the CAR.

In some embodiments, a composition comprises a population of cells as provided herein.

The present disclosure further provides a pharmaceutical composition comprising (i) a population of cells according to any of the preceding claims, and (ii) a pharmaceutically acceptable excipient.

Additionally, the present disclosure provides methods comprising administering to a subject a population of cells as described herein, a composition as described herein, or a pharmaceutical composition as described herein.

In some embodiments, a method is a method of treating a disease in a subject.

The present disclosure also provides uses of a population of cells as described herein, a composition as described herein, or a pharmaceutical composition as described herein for use in treating a disease in a subject. In some embodiments, a population of cells as described herein is for the use in treating a disease in a subject. In some embodiments, a composition as described herein is for use in treating a disease in a subject. In some embodiments, a pharmaceutical composition as described herein is for use in treating a disease in a subject.

The present disclosure further provides uses of a population of cells as described herein, a composition as described herein, or a pharmaceutical composition as described herein in the manufacture of a medicament for the treatment of a disease.

In some embodiments, a disease is cancer. In some embodiments, a cancer is associated with CD5, CD19, CD20, CD22, CD23, CD30, CD33, CD70, Kappa, Lambda, B cell maturation agent (BCMA), G-protein coupled receptor family C group 5 member D (GPRC5D), CD123, LeY, NKG2D ligand, WT1, GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, ROR1, C-Met, CD133, Ep-CAM, GPC3, HPV16-E6, IL13Ra2, MAGEA3, MAGEA4, MART1, NY-ESO-1, VEGFR2, α-Folate receptor, CD24, CD44v7/8, EGP-2, EGP-40, erb-B2, erb-B 2,3,4, FBP, Fetal acethylcholine e receptor, G_{D2}, G_{D3}, HMW-MAA, IL-11Rα, KDR, Lewis Y, L1-cell adhesion molecule, MAGE-A1, Oncofetal antigen (h5T4), and/or TAG-72 expression.

In some embodiments, a cancer is a hematologic malignancy. In some embodiments, a hematologic malignancy is selected from the group consisting of myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

In some embodiments, a cancer is solid malignancy. In some embodiments, a solid malignancy is selected breast cancer, ovarian cancer, colon cancer, prostate cancer, epithelial cancer, renal-cell carcinoma, pancreatic adenocarcinoma, cervical carcinoma, colorectal cancer, glioblastoma, rhabdomyosarcoma, neuroblastoma, melanoma, Ewing sarcoma, osteosarcoma, mesothelioma and adenocarcinoma.

In some embodiments, a disease is an autoimmune disease. In some embodiments, an autoimmune disease is selected from the group consisting of lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

In some embodiments, a disease is diabetes mellitus. In some embodiments, diabetes is selected from the group consisting Type I diabetes, Type II diabetes, prediabetes, and gestational diabetes.

In some embodiments, a disease is a neurological disease. In some embodiments, a neurological disease is selected from the group consisting of catalepsy, epilepsy, encephalitis, meningitis, migraine, Huntington's, Alzheimer's, Parkinson's, Pelizaeus-Merzbacher disease, and multiple sclerosis.

The present disclosure also provides methods of identifying a site for inserting a first transgene at a TCR gene locus. a protospacer adjacent motif (PAM) sequence or target adjacent motif (TAM) sequence

In some embodiments, a method comprises the step of identifying a protospacer adjacent motif (PAM) sequence in a TCR gene locus. In some embodiments, a method comprises the step of identifying a PAM sequence in the 100 bp upstream of the 5′ end of a TCR gene locus. In some embodiments, a method comprises the step of identifying a PAM sequence in the 100 bp downstream of the 3′ end of a TCR gene locus.

In some embodiments, a method comprises the step of identifying a target adjacent motif (TAM) sequence in a TCR gene locus. In some embodiments, a method comprises the step of identifying a TAM sequence in the 100 bp upstream of the 5′ end of a TCR gene locus. In some embodiments, a method comprises the step of identifying a TAM sequence in the 100 bp downstream of the 3′ end of a TCR gene locus.

In some embodiments, a method comprises the step of generating a gRNA. In some embodiments, a gRNA comprises a complementary region. In some embodiments, a complementary region comprises a nucleic acid sequence that is complementary to a target nucleic acid sequence within the TCR gene locus. In some embodiments, a target nucleic acid sequence comprises a first insertion site. In some embodiments, a first insertion site is 25 nucleotides or less from a PAM sequence. In some embodiments, a first insertion site is 25 nucleotides or less from a TAM sequence.

### BRIEF DESCRIPTION OF THE DRAWING

FIG. 1 is a flow chart showing a method for generating T cells according to certain embodiments disclosed herein.

FIG. 2A shows a schematic of a TRAC locus and an exemplary AAV construct comprising an exemplary CD47 transgene (SA-CD47) for insertion at the TRAC locus.

FIG. 2B shows a schematic of a TRAC locus and an exemplary AAV construct comprising an exemplary CD47 transgene (EF1a-CD47) for insertion at the TRAC locus.

FIG. 3 shows exemplary graphs illustrating percentage of non-homologous end joining (NHEJ). These graphs illustrate that all groups demonstrated high levels of NHEJ of TRAC relative to the wild-type (WT) control. However, only the groups that included hCD47 gRNA demonstrated high levels of NHEJ of CD47 relative to the control.

FIG. 4A shows a schematic of an insertion of an exemplary CD47 transgene (SA-hCD47) at a TRAC locus and an exemplary gel with junction PCR products across the insertion site, which was used to confirm insertion of the transgene at the target (TRAC) locus.

FIG. 4B shows a schematic of an insertion of an exemplary CD47 transgene (EF1a-hCD47) at a TRAC locus and an exemplary gel with junction PCR products across the insertion site, which was used to confirm insertion of the transgene at the target (TRAC) locus.

FIG. 5A shows a schematic of an insertion of an exemplary CD47 transgene (SA-CD47) at a TRAC locus and exemplary flow cytometry data demonstrating that introduction of Cas9 and hTRAC gRNA led to a decrease in CD3 expression (indicating knock-down of TRAC).

FIG. 5B shows an exemplary graph demonstrating that introduction of SA-CD47 increased CD47 expression.

FIG. 6 shows exemplary flow cytometry data demonstrating that, in an endogenous CD47 knock-down background, introduction of Cas9 with TRAC gRNA and CD47 gRNA led to a reduction in the expression of CD47 (middle graph), which was recovered when the SA-CD47 transgene was introduced into the cells (right graph). Cells with knock-down of endogenous CD47 were used because wild-type cells (left graph) expressed high levels of CD47.

### DETAILED DESCRIPTION

While the present disclosure is capable of being embodied in various forms, the description below of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios, such as about 2, about 3, and about 4, and sub-ranges, such as about 10 to about 50, about 20 to about 100, and so forth. It also is to be understood, although not always explicitly stated, that the reagents described herein are merely exemplary and that equivalents of such are known in the art.

To the extent any materials incorporated by reference herein conflict with the present disclosure, the present disclosure controls.

### Definitions

The term “about,” as used herein when referring to a measurable value, such as an amount or concentration and the like, is meant to encompass variations of 20%, 10%, 5%, 1%, 0.5%, or even 0.1% of the specified amount.

The term “antibody” is used to denote, in addition to natural antibodies, genetically engineered or otherwise modified forms of immunoglobulins or portions thereof, including chimeric antibodies, human antibodies, humanized antibodies, or synthetic antibodies. The antibodies may be monoclonal or polyclonal antibodies. In those embodiments wherein an antibody is an immunogenically active portion of an immunoglobulin molecule, the antibody may include, but is not limited to, a single chain variable fragment antibody (scFv), disulfide linked Fv, single domain antibody (sdAb), VHH antibody, antigen-binding fragment (Fab), Fab′, F(ab′)2 fragment, or diabody. An scFv antibody is derived from an antibody by linking the variable regions of the heavy (VH) and light (VL) chains of the immunoglobulin with a short linker peptide. Similarly, a disulfide linked Fv antibody can be generated by linking the VH and VL using an interdomain disulfide bond. On the other hand, sdAbs consist of only the variable region from either the heavy or light chain and usually are the smallest antigen-binding fragments of antibodies. A VHH antibody is the antigen binding fragment of heavy chain only. A diabody is a dimer of scFv fragment that consists of the VH and VL regions noncovalent connected by a small peptide linker or covalently linked to each other. The antibodies disclosed herein, including those that comprise an immunogenically active portion of an immunoglobulin molecule, retain the ability to bind a specific antigen.

The term “antigen” refers to an immunogenic molecule that provokes an immune response. This immune response may involve antibody production, activation of specific immunologically competent cells, or both. An antigen may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can also be produced by cells that have been modified or genetically engineered to express an antigen.

A “binding domain,” also referred to as a “binding region,” refers to an antibody or portion thereof that possesses the ability to specifically and non-covalently associate, unite, or combine with a target. A binding domain includes any naturally occurring, synthetic, semi-synthetic, or recombinantly produced binding partner for a biological molecule, a molecular complex, or other target of interest. Exemplary binding domains include receptor ectodomains, ligands, scFvs, disulfide linked Fvs, sdAbs, VHH antibodies, Fab fragments, Fab′ fragments, F(ab′)2 fragments, diabodies, or other synthetic polypeptides selected for their specific ability to bind to a biological molecule, a molecular complex, or other target of interest.

The term “chimeric antigen receptor (CAR),” also known as chimeric T cell receptor or artificial T cell receptor, refers to an artificially engineered receptor that combines both antigen-binding and T cell activating functions. CARs may include an extracellular portion comprising a binding domain, such as one obtained or derived from an antibody (e.g., an scFv). The extracellular portion may be linked through a transmembrane domain to one or more intracellular signaling or effector domains. CARs can optionally contain an intracellular costimulatory domain(s). See, e.g., Sadelain et al., 2013; see also Harris & Kranz, 2016; Stone et al., 2014. CARs can be introduced to be expressed on the surface of a T cell, so that the T cell can target and kill target cells (e.g., cancer cells) that express the antigen the CAR is designed to bind.

The term “codon-optimized” or “codon optimization” when referring to a nucleotide sequence is based on the discovery that the frequency of occurrence of synonymous codons (i.e., codons that code for the same amino acid) in coding nucleotide is biased in different species. Such codon degeneracy allows an identical polypeptide to be encoded by a variety of nucleotide sequences. Codon optimization refers to the process of substituting certain codons in a coding nucleotide sequence with synonymous codons based on the host cell's preference without changing the resulting polypeptide sequence. A variety of codon optimization methods is known in the art, and include, for example, methods disclosed in at least U.S. Pat. Nos. 5,786,464 and 6,114,148.

The term “comparable”, as used herein, refers to two or more agents, entities, situations, sets of conditions, etc. that may not be identical to one another but that are sufficiently similar to permit comparison there between so that conclusions may reasonably be drawn based on differences or similarities observed. Persons of ordinary skill in the art will understand, in context, what degree of identity is required in any given circumstance for two or more such agents, entities, situations, sets of conditions, etc. to be considered comparable.

The term “complementarity determining regions (CDRs)” is synonymous with “hypervariable region” or “HVR,” and is known in the art to refer to sequences of amino acids within antibody variable regions, which, in general, confer antigen specificity and/or binding affinity and are separated from one another in primary structure by framework sequence. In some cases, framework amino acids can also contribute to binding. In general, there are three CDRs in each variable region. Variable domain sequences can be aligned to a numbering scheme (e.g., Kabat, EU, international ImMunoGeneTics Information System® (IMGT®), and Aho), which can allow equivalent residue positions to be annotated and for different molecules to be compared using the Antibody Numbering and Antigen Receptor Classification (ANARCI) software tool (2016, Bioinformatics 15:298-300).

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of introducing a specific nucleic acid sequence into a cell or into another nucleic acid sequence, or as a means of transporting another nucleic acid molecule. Vectors may be, for example, plasmids, cosmids, viruses, an RNA vector, or a linear or circular DNA or RNA molecule that may include chromosomal, non-chromosomal, semi-synthetic, or synthetic nucleic acid molecules. Exemplary vectors are those capable of autonomous replication (episomal vector), capable of delivering a polynucleotide to a cell genome (e.g., viral vector), or capable of expressing nucleic acid molecules to which they are linked (expression vectors).

The term “epitope” includes any molecule, structure, amino acid sequence, or protein determinant that is recognized and specifically bound by a cognate binding molecule, such as an antibody or a T cell receptor, or other binding molecule, domain, or protein.

The term “expression” refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “host cell” as used herein refers to a cell or microorganism targeted for genetic modification by introduction of a construct or vector carrying a nucleotide sequence for expression of a protein or polypeptide of interest. In certain embodiments, when the protein to be expressed includes a CAR, the host cell is usually a T cell.

The term “hypoimmunogenicity,” “hypoimmunogeneic,” “hypoimmunogenic,” “hypoimmunity,” or “hypoimmune” is used interchangeably to describe a cell being less prone to immune rejection by a subject into which such cell is transplanted. For example, relative to an unaltered or unmodified wild-type cell, such a hypoimmunogenic cell may be about 2.5%, 5%, 10%, 20%, 30%, 40%, 50%, 60%, 70%, 80%, 90%, 95%, 97.5%, 99% or more less prone to immune rejection by a subject into which such cell is transplanted. In some examples described herein, genome editing technologies are used to modulate the expression of MHC I and/or MHC II genes, and thus, to generate a hypoimmunogenic cell. In other examples described herein, a tolerogenic factor is introduced into a cell and when expressed can modulate or affect the ability of the cell to be recognized by host immune system and thus confer hypoimmunogenicity. Hypoimmunogenicity of a cell can be determined by evaluating the cell's ability to elicit adaptive and innate immune responses. Such immune response can be measured using assays recognized by those skilled in the art, for example, by measuring the effect of a hypoimmunogenic cell on T cell proliferation, T cell activation, T cell killing, NK cell proliferation, NK cell activation, and macrophage activity. Hypoimmunogenic cells may undergo decreased killing by T cells and/or NK cells upon administration to a subject or show decreased macrophage engulfment compared to an unmodified or wildtype cell. In some cases, a hypoimmunogenic cell elicits a reduced or diminished immune response in a recipient subject compared to a corresponding unmodified wild-type cell. In some cases, a hypoimmunogenic cell is nonimmunogenic or fails to elicit an immune response in a recipient subject.

An “intracellular signaling domain” or “effector domain” is an intracellular portion or domain of a CAR or receptor that can directly or indirectly promote a biological or physiological response in a cell when receiving an appropriate signal. In certain embodiments, an effector domain is from a protein or portion thereof or protein complex that receives a signal when bound to a target or cognate molecule, or when the protein or portion thereof or protein complex binds directly to a target or cognate molecule and triggers a signal from the effector domain.

The term “nucleic acid” or “polynucleotide” refers to a polymeric compound including covalently linked nucleotides comprising natural subunits (e.g., purine or pyrimidine bases). Purine bases include adenine and guanine, and pyrimidine bases include uracil, thymine, and cytosine. Nucleic acid molecules include polyribonucleic acid (RNA) and polydeoxyribonucleic acid (DNA), which includes cDNA, genomic DNA, and synthetic DNA, either of which may be single- or double-stranded. A nucleic acid molecule encoding an amino acid sequence includes all nucleotide sequences that encode the same amino acid sequence.

The term “operably linked” refers to the association of two or more nucleic acid molecules on a single nucleic acid fragment so that the function of one is affected by the other.

The term “safe harbor locus” refers to a gene locus that allows safe expression of a transgene or an exogenous gene. Safe harbors or genomic safe harbors are sites in the genome able to accommodate the integration of new genetic material in a manner that permits the newly inserted genetic elements to: (i) function predictably and (ii) do not cause alterations of the host genome posing a risk to the host cell or organism. Exemplary “safe harbor” loci include a CCR5 gene, a CXCR4 gene, a PPP1R12C (also known as AAVS1) gene, an albumin gene, and a Rosa gene.

The term “safety switch” refers to a system for controlling the expression of a gene or protein of interest that, when downregulated or upregulated, leads to clearance or death of the cell, e.g., through recognition by the host's immune system. A safety switch can be designed to be or include an exogenous molecule administered to prevent or mitigate an adverse clinical event. A safety switch can be engineered by regulating the expression on the DNA, RNA and protein levels. A safety switch may include a protein or molecule that allows for the control of cellular activity in response to an adverse event. In some embodiments, a safety switch refers to an agent (e.g., protein, molecule, etc.) that binds a specific cell and targets it for cell death or elimination. In some instances, the safety switch is a blockade agent that binds a target protein on the surface of a target cell, which in turn, triggers an immune response. In one embodiment, the safety switch is a “kill switch” that is expressed in an inactive state and is fatal to a cell expressing the safety switch upon activation of the switch by a selective, externally provided agent. In one embodiment, the safety switch gene is cis-acting in relation to the gene of interest in a construct. Activation of the safety switch causes the cell to kill solely itself or itself and neighboring cells through apoptosis or necrosis.

The term “subject” refers to a mammalian subject, preferably a human. A “subject in need thereof” may refer to a subject who has been diagnosed with a disease, or is at an elevated risk of developing a disease. The phrases “subject” and “patient” are used interchangeably herein.

A “therapeutically effective amount” as used herein is an amount that produces a desired effect in a subject for treating a disease. In certain embodiments, the therapeutically effective amount is an amount that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. A therapeutically effective amount for a particular composition will vary based on a variety of factors, including, but not limited, to the characteristics of the therapeutic composition (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers, excipients, and preservatives in the composition, and the route of administration. One skilled in the clinical and pharmacological arts will be able to determine a therapeutically effective amount through routine experimentation, namely by monitoring a subject's response to administration of the host cell, or the pharmaceutical composition containing the same, and adjusting the dosage accordingly. For additional guidance, see, e.g., Remington: The Science and Practice of Pharmacy, 22nd Edition, Pharmaceutical Press, London, 2012, and Goodman & Gilman's The Pharmacological Basis of Therapeutics, 12th Edition, McGraw-Hill, New York, NY, 2011, the entire disclosures of which are incorporated by reference herein.

The term “tolerogenic factor” as used herein includes hypoimmunity factors, complement inhibitors, and other factors that modulate or affect (e.g., reduce) the ability of a cell to be recognized by the immune system of a host or recipient subject upon administration, transplantation, or engraftment. Tolerogenic factors include but are not limited to CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, Serpinb9, CCl21, Mfge8, A20/TNFAIP3, CCL21, CD16 Fc receptor, CD27, CR1, DUX4, H2-M3 (HLA-G), HLA-F, IL15-RF, MANF, IL-39, and B2M-HLA-E.

A “transmembrane region” is a portion of a transmembrane protein that can insert into or span a cell membrane.

The terms “treat,” “treating,” and “treatment” as used herein with regard to cancer refers to alleviating the cancer partially or entirely; preventing the cancer; decreasing the likelihood of occurrence or recurrence of the cancer; slowing the progression or development of the cancer; eliminating, reducing, or slowing the development of one or more symptoms associated with the cancer; or increasing progression-free or overall survival of the cancer. For example, “treating” may refer to preventing or slowing the existing cancer from growing larger; preventing or slowing the formation or metastasis of cancer; and/or slowing the development of certain symptoms of the cancer. In some embodiments, the term “treat,” “treating,” or “treatment” means that the subject has a reduced number or size of cancer cells comparing to a subject without being administered with the treatment. In some embodiments, the term “treat,” “treating,” or “treatment” means that one or more symptoms of the cancer are alleviated in a subject receiving the treatment as disclosed and described herein comparing to a subject who does not receive such treatment.

The term “variable region” or “variable domain” refers to a portion of an antibody heavy or light chain that is involved in antigen binding. Variable domains of antibody heavy (VH) and light (VL) chains each generally comprise four generally conserved framework regions (FRs) and three complementarity determining regions (CDRs). Framework regions separate CDRs, such that CDRs are situated between framework regions.

A “vector” refers to a DNA construct containing a nucleic acid molecule that is operably linked to a suitable control sequence capable of effecting the expression of the nucleic acid molecule in a suitable host. Such control sequences may include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself.

### I. Methods of Manufacture

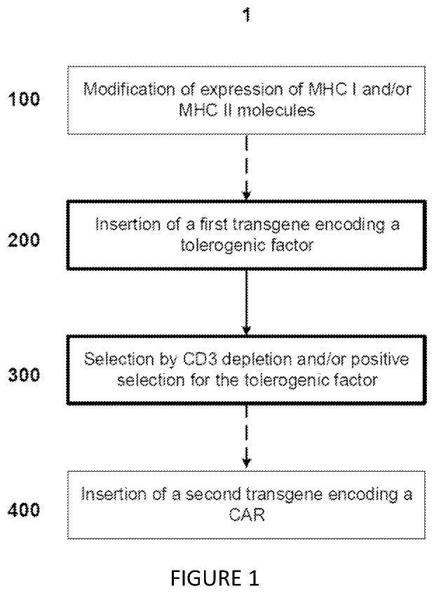

In some aspects, the present technology provides methods for generating a population of T cells, such as immune evasive allogeneic T cells, for cell therapy (FIG. 1). A flow chart of certain embodiments of the methods is shown in FIG. 1 (process 1). In some embodiments, the method comprises (a) inserting a first transgene encoding a tolerogenic factor into an endogenous TCR gene locus (e.g., the TRAC and/or TRBC loci including TRBC1 and/or TRBC2) of the T cells (FIG. 1, step 200), and (b) selecting for T cells that have the transgene inserted by CD3 depletion and/or positive selection for the tolerogenic factor (e.g., selection for expression of the tolerogenic factor) (FIG. 1, step 300). The endogenous TCR gene locus may be a genomic locus within any gene encoding a TCR or a component thereof, including, for example, the TRAC and/or TRBC (including TRBC1 and TRBC2) loci. Inserting a tolerogenic factor at the endogenous TCR gene locus may achieve the dual purposes of reducing or eliminating TCR expression and increasing expression of the tolerogenic factor in the T cells (especially allogenic T cells) in one manufacturing step, so that the resulting T cells can be made immune evasive and not subject to immune rejection when transplanted into a recipient, thereby increasing both the efficiency of the manufacturing process and the effectiveness of cell-based therapies. In some embodiments, the methods further comprise modifying the expression of MHC class I and/or MHC class II molecules in the T cells (FIG. 1, step 100). In some embodiments, methods further comprise inserting a second transgene encoding a CAR to a genomic locus of the T cells (FIG. 1, step 400).

a. Insertion of a First Transgene Encoding a Tolerogenic Factor

### 1. Tolerogenic Factors

In some embodiments, the tolerogenic factor is selected from the group consisting of CD16, CD24, CD35, CD39, CD46, CD47, CD52, CD55, CD59, CD200, CCL22, CTLA4-Ig, Cl inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF, and any combinations, truncations, modifications, or fusions of the above.

In some embodiments, the tolerogenic factor is CD47. CD47 is a leukocyte surface antigen and has a role in cell adhesion and modulation of integrins. It is expressed on the surface of a cell (e.g., a T cell) and signals to circulating macrophages not to phagocytize the cell. Overexpression of CD47 thus can reduce the immunogenicity of the cell when grafted and improve immune protection in allogeneic recipients.

In some embodiments, the CD47 is human CD47, and in some of these embodiments, the human CD47 comprises or consists of an amino acid sequence set forth in SEQ ID NO:1 or SEQ ID NO:2 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:1 or SEQ ID NO:2. In some embodiments, the transgene encoding CD47 comprises a nucleotide sequence corresponding to an mRNA sequence of human CD47. In some embodiments, the transgene encoding CD47 has a nucleotide sequence that is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:3 (coding sequence (CDS) of the nucleotide sequence set forth in NCBI Ref. No. NM_001777.4) or SEQ ID NO:4 (CDS of the nucleotide sequence set forth in NCBI Ref. No. NM_198793.2).

In some embodiments, the transgene encoding CD47 is codon-optimized for expression in a mammalian cell, for example, a human cell. In some embodiments, the codon-optimized transgene encoding CD47 has a nucleotide sequence that is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:5.

TABLE 1
Exemplary sequences of CD47

| SEQ | | |
|---|---|---|
| ID | | |
| NO: | Sequence | Description |
| 1 | MWPLVAALLLGSACCGSAQLLENKTKSVEFTFCND | Amino acid sequence |
| | TVVIPCFVTNMEAQNTTEVYVKWKFKGRDIYTFDG | encoded by CDS of |
| | ALNKSTVPTDFSSAKIEVSQLLKGDASLKMDKSDA | NM_001777.4 |
| | VSHTGNYTCEVTELTREGETIIELKYRVVSWFSPN | |
| | ENILIVIFPIFAILLFWGQFGIKTLKYRSGGMDEK | |
| | TIALLVAGLVITVIVIVGAILFVPGEYSLKNATGL | |
| | GLIVTSTGILILLHYYVFSTAIGLTSFVIAILVIQ | |
| | VIAYILAVVGLSLCIAACIPMHGPLLISGLSILAL | |
| | AQLLGLVYMKFVASNQKTIQPPRKAVEEPLNAFKE | |
| | SKGMMNDE | |
| 2 | MWPLVAALLLGSACCGSAQLLENKTKSVEFTFCND | Amino acid sequence |
| | TVVIPCFVTNMEAQNTTEVYVKWKFKGRDIYTFDG | encoded by CDS of |
| | ALNKSTVPTDFSSAKIEVSQLLKGDASLKMDKSDA | NM_198793.2 |
| | VSHTGNYTCEVTELTREGETIIELKYRVVSWFSPN | |
| | ENILIVIFPIFAILLFWGQFGIKTLKYRSGGMDEK | |
| | TIALLVAGLVITVIVIVGAILFVPGEYSLKNATGL | |
| | GLIVTSTGILILLHYYVFSTAIGLTSFVIAILVIQ | |
| | VIAYILAVVGLSLCIAACIPMHGPLLISGLSILAL | |
| | AQLLGLVYMKFVASNQKTIQPPRNN | |
| 3 | atgtggcccctggtagcggcgctgttgctgggctc | Nucleotide sequence of |
| | ggcgtgctgcggatcagctcagctactatttaata | NM_001777.4 CDS (nts |
| | aaacaaaatctgtagaattcacgttttgtaatgac | 124-1095) |
| | actgtcgtcattccatgctttgttactaatatgga | |
| | ggcacaaaacactactgaagtatacgtaaagtgga | |
| | aatttaaaggaagagatatttacacctttgatgga | |
| | gctctaaacaagtccactgtccccactgactttag | |
| | tagtgcaaaaattgaagtctcacaattactaaaag | |
| | gagatgcctctttgaagatggataagagtgatgct | |
| | gtctcacacacaggaaactacacttgtgaagtaac | |
| | agaattaaccagagaaggtgaaacgatcatcgagc | |
| | taaaatatcgtgttgtttcatggttttctccaaat | |
| | gaaaatattcttattgttattttcccaatttttgc | |
| | tatactcctgttctggggacagtttggtattaaaa | |
| | cacttaaatatagatccggtggtatggatgagaaa | |
| | acaattgctttacttgttgctggactagtgatcac | |
| | tgtcattgtcattgttggagccattcttttcgtcc | |
| | caggtgaatattcattaaagaatgctactggcctt | |
| | ggtttaattgtgacttctacagggatattaatatt | |
| | acttcactactatgtgtttagtacagcgattggat | |
| | taacctccttcgtcattgccatattggttattcag | |
| | gtgatagcctatatcctcgctgtggttggactgag | |
| | tctctgtattgcggcgtgtataccaatgcatggcc | |
| | ctcttctgatttcaggtttgagtatcttagctcta | |
| | gcacaattacttggactagtttatatgaaatttgt | |
| | ggcttccaatcagaagactatacaacctcctagga | |
| | aagctgtagaggaaccccttaatgcattcaaagaa | |
| | tcaaaaggaatgatgaatgatgaataa | |
| 4 | atgtggcccctggtagcggcgctgttgctgggctc | Nucleotide sequence of |
| | ggcgtgctgcggatcagctcagctactatttaata | NM_198793.2 CDS (nts |
| | aaacaaaatctgtagaattcacgttttgtaatgac | 181-1098) |
| | actgtcgtcattccatgctttgttactaatatgga | |
| | ggcacaaaacactactgaagtatacgtaaagtgga | |
| | aatttaaaggaagagatatttacacctttgatgga | |
| | gctctaaacaagtccactgtccccactgactttag | |
| | tagtgcaaaaattgaagtctcacaattactaaaag | |
| | gagatgcctctttgaagatggataagagtgatgct | |
| | gtctcacacacaggaaactacacttgtgaagtaac | |
| | agaattaaccagagaaggtgaaacgatcatcgagc | |
| | taaaatatcgtgttgtttcatggttttctccaaat | |
| | gaaaatattcttattgttattttcccaatttttgc | |
| | tatactcctgttctggggacagtttggtattaaaa | |
| | cacttaaatatagatccggtggtatggatgagaaa | |
| | acaattgctttacttgttgctggactagtgatcac | |
| | tgtcattgtcattgttggagccattcttttcgtcc | |
| | caggtgaatattcattaaagaatgctactggcctt | |
| | ggtttaattgtgacttctacagggatattaatatt | |
| | acttcactactatgtgtttagtacagcgattggat | |
| | taacctccttcgtcattgccatattggttattcag | |
| | gtgatagcctatatcctcgctgtggttggactgag | |
| | tctctgtattgcggcgtgtataccaatgcatggcc | |
| | ctcttctgatttcaggtttgagtatcttagctcta | |
| | gcacaattacttggactagtttatatgaaatttgt | |
| | ggcttccaatcagaagactatacaacctcctagga | |
| | ataactga | |
| 5 | atgtggcccctggtcgccgccctgttgctgggctc | Codon-optimized |
| | ggcatgctgcggatcagctcagctactgtttaata | nucleotide sequence |
| | aaacaaaatctgtagaattcacgttttgtaacgac | encoding CD47 |
| | actgtcgtgatcccatgctttgttactaatatgga | |
| | ggcacaaaacaccactgaagtgtacgtgaagtgga | |
| | aattcaaaggcagagacatttacacctttgacggc | |
| | gccctcaacaagtccaccgtgcccactgactttag | |
| | tagcgcaaaaattgaggtcagccaattactaaaag | |
| | gagatgcctctttgaagatggacaagagcgatgct | |
| | gtcagccacacagggaactacacttgtgaagtaac | |
| | agagttaacccgcgaaggtgaaacgatcatcgagc | |
| | tgaagtatcgagtggtgtcctggttttctccgaac | |
| | gagaatatccttatcgtaattttcccaattttcgc | |
| | tatcctcctgttctggggccagtttggtatcaaga | |
| | cactcaaatatcggtccggtgggatggatgagaag | |
| | acaattgccctgcttgttgctggactcgtgatcac | |
| | cgtcatcgtgattgttggggccatccttttcgtcc | |
| | caggggagtacagcctgaagaatgctacgggcctg | |
| | ggattaattgtgacctctacagggatactcatcct | |
| | gcttcactactatgtgttcagtaccgcgattggac | |
| | tgacctccttcgtcattgccatattggtgattcag | |
| | gtgatagcctacatcctcgccgtggttggcctgag | |
| | tctctgtatcgcggcgtgcatacccatgcatggcc | |
| | ctcttctgatttcagggttgagtatcctcgcacta | |
| | gcacagttgctgggactggtttatatgaaatttgt | |
| | ggcctccaaccagaagactatacagcctcctagga | |
| | aggctgtagaggagcccctgaatgcattcaaggaa | |
| | tcaaaaggcatgatgaatgatgaa | |

In some embodiments, a first transgene encoding a first tolerogenic factor at an insertion site at a TCR gene locus has a reverse sequence orientation (5′ to 3′) relative to the sequence of the TCR gene locus. In some embodiments, a first transgene encoding a first tolerogenic factor at an insertion site at a TCR gene locus comprises a promoter that has a reverse sequence orientation (5′ to 3′) relative to the sequence of the TCR gene locus. In some embodiments, the promoter that has a reverse sequence orientation (5′ to 3′) relative to the sequence of the TCR gene locus drives transcription of a first transgene encoding a first tolerogenic factor in a reverse sequence orientation relative to the TCR gene locus. In some embodiments, a first transgene encoding a first tolerogenic factor at an insertion site at a TCR gene locus comprises (in 5′ to 3′ order relative to the TCR gene locus) a poly-A tail sequence, a reverse orientation transgene sequence, and a reverse orientation promoter sequence. In some embodiments, a TCR gene locus comprises a first transgene encoding a first tolerogenic factor and a second transgene encoding a CAR in a reverse sequence orientation (5′ to 3′) relative to the sequence of the TCR gene locus. In some embodiments, a TCR gene locus comprises a first transgene encoding a first tolerogenic factor in a reverse sequence orientation (5′ to 3′) relative to the sequence of the TCR gene locus and a second transgene encoding a CAR in the forward orientation (i.e., the same orientation) relative to the sequence of the TCR gene locus. In some embodiments, a TCR gene locus comprises a first transgene encoding a first tolerogenic factor and a second transgene encoding a second tolerogenic factor in a reverse sequence orientation (5′ to 3′) relative to the sequence of the TCR gene locus. In some embodiments, a TCR gene locus comprises a first transgene encoding a first tolerogenic factor in a reverse sequence orientation (5′ to 3′) relative to the sequence of the TCR gene locus and a second transgene encoding a second tolerogenic factor in the forward orientation (i.e., the same orientation) relative to the sequence of the TCR gene locus. In some embodiments, a TCR gene locus comprises a first transgene encoding a first tolerogenic factor, a second transgene encoding a second tolerogenic factor, and a third transgene encoding a CAR in a reverse sequence orientation (5′ to 3′) relative to the sequence of the TCR gene locus. In some embodiments, a TCR gene locus comprises a first transgene encoding a first tolerogenic factor and a second transgene encoding a second tolerogenic factor in a reverse sequence orientation (5′ to 3′) relative to the sequence of the TCR gene locus, and a third transgene encoding a CAR in the forward orientation (i.e., the same orientation) relative to the sequence of the TCR gene locus. In some embodiments, a TCR gene locus comprises a first transgene encoding a first tolerogenic factor in a reverse sequence orientation (5′ to 3′) relative to the sequence of the TCR gene locus, a second transgene encoding a second tolerogenic factor in the forward orientation (i.e., the same orientation) relative to the sequence of the TCR gene locus, and a third transgene encoding a CAR in the forward orientation (i.e., the same orientation) relative to the sequence of the TCR gene locus.

### 2. Regulatory Elements

In some embodiments, a transgene comprises a gene and one or more regulatory elements. In some embodiments, expression of the tolerogenic factor may be operably linked to an endogenous promoter at the TCR gene locus (e.g., TRAC, TRBC1, and/or TRBC2). In certain of these embodiments, the first transgene encoding the tolerogenic factor to be inserted need not include an exogenous promoter however, in some embodiments, the transgene may include an exogenous insulator and/or an exogenous enhancer.

Alternatively, in other embodiments, the first transgene encoding a tolerogenic factor may additionally comprise an exogenous promoter to drive expression of the tolerogenic factor in the host cell. In certain of these embodiments, the exogenous promoter may be one that drives constitutive gene expression in mammalian cells. Those frequently used include, for example, elongation factor 1 alpha (EF1α) promoter, cytomegalovirus (CMV) immediate-early promoter (Greenaway et al., Gene 18: 355-360 (1982)), simian vacuolating virus 40 (SV40) early promoter (Fiers et al., Nature 273:113-120 (1978)), spleen focus-forming virus (SFFV) promoter, phosphoglycerate kinase (PGK) promoter (Adra et al., Gene 60(1):65-74 (1987)), human beta actin promoter, polyubiquitin C gene (UBC) promoter, CAG promoter (Nitoshi et al., Gene 108:193-199 (1991)), MND (MPSV LTR, NCR deleted, and d/587 PBS; Challita et al., J. Virol 69(2):748-755 (1995)) promoter, SSFV promoter, and ICOS promoter. An example of a promoter that is capable of expressing a transgene in a mammalian cell (e.g., a T cell) is the EF1α promoter. The native EF1α promoter drives expression of the alpha subunit of the elongation factor-1 complex, which is responsible for the enzymatic delivery of aminoacyl tRNAs to the ribosome. The EF1α promoter has been extensively used in mammalian expression plasmids and has been shown to be effective in driving CAR expression from transgenes cloned into a lentiviral vector. See, e.g., Milone et al., Mol. Ther. 17(8):1453-1464 (2009). For another example, an MND promoter is a synthetic promoter that contains the U3 region of a modified gammaretrovirus-derived MoMuLV LTR with myeloproliferative sarcoma virus enhancer, and this promoter has been shown to be highly and constitutively active in the hematopoietic system and to resist transcriptional silencing. See, e.g., Halene et al., Blood 94(10):3349-3357 (1999).

In some embodiments, the first transgene encoding a tolerogenic factor may comprise additional regulatory elements operatively linked to the tolerogenic factor sequence and/or promoter, including, for example, insulators, enhancers, polyadenylation (poly(A)) tails, and/or ubiquitous chromatin opening elements. As known to a skilled artisan, these regulatory elements may be needed to affect the expression and processing of coding sequences to which they are operatively linked. Regulatory elements used for transgene expression modulation may include appropriate transcription initiation, termination, promoter, and enhancer sequences; efficient RNA processing signals, such as splicing and polyadenylation signals; sequences that stabilize cytoplasmic mRNA; sequences that enhance translation efficiency; sequences that enhance protein stability; and possibly sequences that enhance protein secretion.

In some embodiments, the first transgene encoding a tolerogenic factor may additionally comprise an insulator to modulate the expression of the tolerogenic factor in the host cell. Insulators are DNA elements (usually about 50 nucleotides in length) that can shelter genes from inappropriate regulatory interactions. In some embodiments, insulators insulate genes located in one domain from promiscuous regulation by enhancers or silencers in neighboring domains. Insulators that disrupt communication between an enhancer and its promoter when positioned between the two are called enhancer-blockers, and insulators that are located between a silencer and a promoter and protect the promoter from silencing are called barriers. In some embodiments, insulators that are barriers prevent the advance of nearby condensed chromatin and protect gene expression from positive and negative chromatin effects. Thus, in the design of a transgene, insulators are usually placed upstream of the promoter. Non-limiting examples of insulators include 5′HS5, DMD/ICR, BEAD-1, apoB (−57 kb), apoB (+43 kb), DM1 site 1, DM1 site 2 (from human); BEAD-1, HS2-6, DMR/ICR, SINE (from mouse); SF1, scs/scs′, gypsy, Fab-7, Fab-8, faswab, eve (from fruit fly); HMR tRNAThr, Chal UAS, UASrpg, STAR (from yeast); Lys 5′A, HS4, or 3′HS (from chicken); sns, URI (from sea urchin); and RO (from frog). Other examples of insulators include Mcp, Neighbor of Homie (Nhomie) insulator and Homing insulator at eve (Homie), and Su(Hw)-dependent insulators. In some embodiments, the first transgene encoding a tolerogenic factor may comprise an insulator having a sequence that is at least 80% identical (e.g., at least 80%, at least 85%, at least 90%, at least 95%, at least 96%, at least 97%, at least 98%, at least 99%, or 100% identical) to any of the described insulators.

In some embodiments, the first transgene encoding a tolerogenic factor comprises one copy of an insulator. In some embodiments, the transgene comprises a multimerized insulator. In some embodiments, a transgene comprises two copies of an insulator. In some embodiments, a transgene comprises three copies of an insulator. In some embodiments, a transgene comprises four copies of an insulator. In some embodiments, a transgene comprises five or more copies of an insulator. Insulator effectiveness is influenced by its structure and by the nature of the enhancer, promoter, and genomic context. In some embodiments, the first transgene encoding a tolerogenic factor may comprise two or more heterologous insulators. In some embodiments, the two or more heterologous insulators interact with each other. In some embodiments, the first transgene encoding a tolerogenic factor comprises an insulator and a regulatory protein that binds to the insulator.

In some embodiments, the first transgene encoding a tolerogenic factor may additionally comprise an enhancer to increase expression of the tolerogenic factor in the host cell. Enhancer sequences are regulatory DNA sequences that, when bound by specific proteins called transcription factors, enhance the transcription of an associated gene. Enhancers are regions of DNA, typically 100 to 1000 bp in size, that contain transcription factor-binding sites that stimulate the initiation and elongation of transcription from promoters. In most housekeeping genes, enhancers are located in close proximity to promoters. Some genes feature complex regulatory regions that can consist of dozens of enhancers located at variable distances from the regulated promoter. During transcriptional activation, enhancers are usually located in close proximity to gene promoters. Some promoters described herein already have an enhancer incorporated; for example, the CAG promoter is constructed by combining the CMV early enhancer element, the chicken beta actin gene promoter, and the splice acceptor of the rabbit beta globin gene.

Enhancers may consist of combinations of short, degenerate sites, 6-12 bp in length, that are recognized by DNA-binding transcription factors, which determine enhancer activity. The combination of DNA-binding transcription factors on a given enhancer creates a platform that attracts co-activators and co-repressors that determine the enhancer activity in each specific group of cells. The ability of an enhancer to stimulate transcription depends on the combination of transcription factor sites that positively or negatively affect enhancer activity and the relative concentrations of enhancer-binding transcription factors within the nuclei of a given group of cells. Recently, super-enhancers have been identified, representing a special class of regulatory elements, characterized by large sizes, sometimes reaching tens of thousands of bp, with a high degree of transcription factor and co-activator enrichment. Super-enhancers are often located adjacent to genes known to be critical for cell differentiation. A more detailed study of super-enhancers has shown that they often consist of separate domains that can either function together to enhance the overall activity of each domain or play independent roles during the simultaneous activation of a large number of promoters.

During the activation of transcription, enhancers recruit several key complexes. The p300/CBP and M113/M114/COMPASS complexes have acetyltransferase and methyltransferase activities, respectively. The proteins M113 and M114 both contain a C-terminal SET (suppressor of variegation, enhancer of zeste, trithorax) domain, which is responsible for the monomethylation of lysine 4 of histone H3 (H3K4me1). The complexes formed by M113 and M114 have partially overlapping and insufficiently studied functions in the regulation of enhancer activity. M113 and M114 are also known to be involved in the recruitment of the p300/CBP co-activator, which is responsible for the acetylation of histone H3 at lysine 27 (H3K27ac). H3K27ac and H3K4me1 histone marks are distinctive features of active enhancers and are used to identify enhancers in genomes.

In some embodiments, the first transgene encoding a tolerogenic factor may additionally comprise a poly(A) tail. A poly(A) tail is a long chain of adenine nucleotides that is added to an mRNA molecule during RNA processing to increase the stability of the molecule. Immediately after a gene in a eukaryotic cell is transcribed, the new RNA molecule undergoes several modifications known as RNA processing. These modifications alter both ends of the primary RNA transcript to produce a mature mRNA molecule. The processing of the 3′ end adds a poly-A tail to the RNA molecule. First, the 3′ end of the transcript is cleaved to free a 3′ hydroxyl. Then an enzyme called poly-A polymerase adds a chain of adenine nucleotides to the RNA. This process, called polyadenylation, adds a poly-A tail that is between 100 and 250 residues long. The poly-A tail makes the RNA molecule more stable and prevents its degradation. Additionally, the poly-A tail allows the mature messenger RNA molecule to be exported from the nucleus and translated into a protein by ribosomes in the cytoplasm.

In some embodiments, the first transgene encoding a tolerogenic factor may additionally comprise a ubiquitous chromatin opening element (UCOE). The integration of a transgene into a heterochromatic chromatin environment and the methylation of promoter DNA are major mechanisms that are antagonistic to gene expression, resulting in a variegated pattern of gene expression or silencing. Because stable and high level transgene expression are essential for the efficient and rapid production of clonal cell lines in biomanufacturing as well as for the lifelong expression of a transgene at a therapeutic level in gene therapy, genetic regulatory elements that can prevent gene silencing and maintain high levels of expression for long periods of time are crucial.

Genetic regulatory elements that confer a transcriptionally permissive state can be broadly dichotomized into those that actively function through dominant chromatin remodeling mechanisms and those that function as border or boundary elements to restrict the spread of heterochromatin marks into regions of euchromatin. The latter include insulators, scaffold/matrix attachment regions (S/MARs), and stabilizing anti-repressor (STAR) elements, whilst the former comprise locus control regions (LCRs) and UCOEs. LCRs and UCOEs are defined by their ability to consistently confer site of integration-independent stable transgene expression that is proportional to transgene copy number, even when integrated into heterochromatin. LCRs are tissue-specific regulatory elements that consist of multiple subcomponents characterized by DNase I hypersensitivity and a high density of transcription factor binding sites. In contrast, UCOEs function ubiquitously and neither consist of multiple DNase I hypersensitive sites that are characteristic of LCRs, nor are they required to flank a transgene at both 5′ and 3′ ends in order to exert their function as in the case of insulators and S/MARs. Thus, structurally and functionally UCOEs represent a distinct class of genetic regulatory element. UCOEs have found widespread usage in protein therapeutic biomanufacturing applications as a means to manage costs and resources as well as to reliably expedite the generation of highly expressing recombinant cell clones. In some embodiments, UCOEs provide stable ubiquitous or tissue-specific expression in somatic tissues as well as in adult, embryonic, and induced pluripotent stem cells and their differentiated progeny.

### 3. Site-Directed Genomic Insertion

In some embodiments, the first transgene encoding a tolerogenic factor and/or regulatory elements may be delivered into a host cell for targeted genomic insertion in the form of a vector. The delivery vector can be any type of vector suitable for introduction of nucleotide sequences into a cell, including, for example, plasmids, adenoviral vectors, adeno-associated viral (AAV) vectors, retroviral vectors, lentiviral vectors, phages, and HDR-based donor vectors. The different components may be introduced into a cell together or separately, and may be delivered in a single vector or multiple vectors. The vector may be introduced into a cell by any known method in the field, including, for example, viral transformation, calcium phosphate transfection, lipid-mediated transfection, DEAE-dextran, electroporation, microinjection, nucleoporation, liposomes, nanoparticles, or other methods. Insertion of the first transgene encoding a tolerogenic factor and/or regulatory elements into an endogenous TCR gene locus may be carried out using any of the site-directed insertion methods and/or systems described herein, including, for example, zinc finger nucleases (ZFNs), transcription activator-like effector nucleases (TALENs), meganucleases, transposases, and clustered regularly interspaced short palindromic repeat (CRISPR)/Cas systems. Insertion of the first transgene encoding a tolerogenic factor and/or regulatory elements into an endogenous TCR gene locus may be carried out using a genome-modifying protein described herein, including for example, a CRISPR-associated transposase, prime editing, or Programmable Addition via Site-specific Targeting Elements (PASTE). Insertion of the first transgene encoding a tolerogenic factor and/or regulatory elements into an endogenous TCR gene locus may be carried out using a genome-modifying protein described herein, including for example, TnpB polypeptides. In cases where a homology directed repair (HDR)-based approach as described is used, the transgene is usually flanked by homology arms (i.e., left homology arm (LHA) and right homology arm (RHA)) that are specific to the target site of insertion. The homology arms are specifically designed for the target genomic locus for the fragment to serve as a template for HDR. The length of each homology arm is generally dependent on the size of the insert being introduced, with larger insertions requiring longer homology arms.

B. TCR Depletion, CD3 Depletion, and/or Positive Selection for the Tolerogenic Factor

In some embodiments, the methods described herein for generating a population of T cells, such as immune evasive allogeneic T cells, comprise selecting for cells containing the first transgene encoding a tolerogenic factor integrated into an endogenous TCR gene locus of the T cells, wherein integration of the first transgene into the TCR gene locus reduces or eliminates expression of a functional TCR complex at a surface of the T cells, which in turn prevents CD3 from locating to the cell surface. In some embodiments, the selecting comprises CD3 depletion (FIG. 1, step 300). In some embodiments, the selecting comprises positive selection for the tolerogenic factor (e.g., selection for expression of the tolerogenic factor) (FIG. 1, step 300). In some embodiments, CD3 depletion comprises selecting for T cells that have reduced or eliminated expression of endogenous TCR on a cell surface and therefore have reduced or eliminated CD3 associated with a functional TCR complex on the cell surface. In some embodiments, T cells with reduced or eliminated CD3 expression on the cell surface have reduced or eliminated binding to CD3-binding antibodies and/or other CD3-binding proteins. In some embodiments, T cells with reduced or eliminated CD3 expression on the cell surface do not bind to a column and/or a sorting surface with attached CD3-binding antibodies and/or other CD3-binding proteins. In some embodiments, the population of T cells which fails to bind to the CD3-binding antibodies flows through the column and is collected. This population of T cells may also be referred to as enriched for CD3-negative T cells or enriched for T cells having reduced surface expression of CD3. In some embodiments, the selecting comprises TCR depletion. In some embodiments, TCR depletion comprises selecting for T cells that have reduced or eliminated expression of endogenous TCR on a cell surface and therefore have reduced or eliminated TCR complex on the cell surface. In some embodiments, T cells with reduced or eliminated TCR expression on the cell surface have reduced or eliminated binding to TCR-binding antibodies and/or other TCR-binding proteins. In some embodiments, T cells with reduced or eliminated TCR expression on the cell surface do not bind to a column and/or a sorting surface with attached TCR-binding antibodies and/or other TCR-binding proteins. In some embodiments, the population of T cells which fails to bind to the TCR-binding antibodies flows through the column and is collected. This population of T cells may also be referred to as enriched for TCR-negative T cells or enriched for T cells having reduced surface expression of TCR. In some embodiments, positive selection for the tolerogenic factor (e.g., CD47) comprises selecting for T cells that express the tolerogenic factor on the cell surface, for example, at a higher level than endogenous expression levels of the tolerogenic factor. In some embodiments, positive selection for the tolerogenic factor comprises selecting for T cells that express the tolerogenic factor on the cell surface, for example, at a higher level than endogenous expression levels of the tolerogenic factor if the cell expresses any endogenous tolerogenic factor. In these embodiments, antibodies and/or proteins that bind the tolerogenic factor are selected based on a desired affinity and/or avidity for the tolerogenic factor. For example, antibodies and/or proteins having higher affinities and/or avidities for the tolerogenic factor may be selected over lower affinities and/or avidities for use with cells which express endogenous levels of the tolerogenic factor. In some embodiments, T cells expressing the tolerogenic factor on the cell surface bind to antibodies and/or proteins that bind to the tolerogenic factor. In some embodiments, T cells expressing the tolerogenic factor on the cell surface bind to a column and/or a sorting surface with attached antibodies and/or other proteins binding the tolerogenic factor.

In some embodiments, the methods described herein for generating a population of T cells, such as immune evasive allogeneic T cells, comprises selecting for cells containing the first transgene encoding a tolerogenic factor integrated into an endogenous TCR gene locus of the T cells, wherein integration of the first transgene into the endogenous TCR gene locus reduces or eliminates expression of a functional TCR complex at a surface of the T cells. In some embodiments, the selecting comprises CD3 depletion, wherein the T cells with reduced or eliminated expression of CD3 on the cell surface are sorted by affinity binding, flow cytometry, and/or immunomagnetic selection using CD3-binding antibodies and/or other CD3-binding proteins. In some embodiments, the selecting comprises TCR depletion, wherein the T cells with reduced or eliminated expression of TCR on the cell surface are sorted by affinity binding, flow cytometry, and/or immunomagnetic selection using TCR-binding antibodies and/or other TCR-binding proteins. In some embodiments, the methods described herein for generating T cells, such as immune evasive allogeneic T cells, comprises selecting for cells containing the first transgene encoding a tolerogenic factor using positive selection for the tolerogenic factor. In some embodiments, the positive selection for the tolerogenic factor comprises selecting for T cells that express the tolerogenic factor on the cell surface by affinity binding, flow cytometry, and/or immunomagnetic selection using antibodies and/or other proteins that bind the tolerogenic factor. In some embodiments, the tolerogenic factor is CD47.

Several methods of sorting living cells based on whether and/or how much they express or do not express a specific protein on their cell surface are known to those of skill in the art. For example, fluorescence activated cell sorting (FACS) of live cells separates a population of cells into sub-populations based on fluorescent labeling using a flow cytometer. Cells stained using fluorophore-conjugated antibodies to an antigen or marker of interest, such as CD3, TCR, or CD47, can be separated from one another depending on which fluorophore they have been stained with. For example, a cell expressing one cell marker may be detected using an FITC-conjugated antibody that recognizes the marker, and another cell type expressing a different marker could be detected using a PE-conjugated antibody specific for that marker.

Another example of a cell sorting method is magnetic-activated cell sorting (MACS). MACS is a method for separation of various cell populations depending on their surface antigens, such as CD3, TCR, or CD47. The method uses superparamagnetic nanoparticles and columns. The superparamagnetic nanoparticles are of the order of 100 nm. They are used to tag the targeted cells in order to capture them inside the column. The column is placed between permanent magnets so that when the magnetic particle-cell complex passes through it, the tagged cells can be captured. The column consists of steel wool which increases the magnetic field gradient to maximize separation efficiency when the column is placed between the permanent magnets. The MACS method allows cells to be separated by using magnetic nanoparticles coated with antibodies against a particular surface antigen, such as CD3, TCR, and/or CD47. This causes the cells expressing this antigen to attach to the magnetic nanoparticles. After incubating the beads and cells, the solution is transferred to a column in a strong magnetic field. In this step, the cells attached to the nanoparticles (expressing the antigen) stay on the column, while other cells (not expressing the antigen) flow through. With this method, the cells can be separated positively or negatively with respect to the particular antigen(s). With positive selection, the cells expressing the antigen(s) of interest, which are attached to the magnetic column, are washed out to a separate vessel, after removing the column from the magnetic field. In some embodiments, positive selection methods can be used to distinguish cells expressing endogenous tolerogenic factors from cells expressing tolerogenic factors encoded by transgenes. For example, endogenous expression levels of tolerogenic factors are generally lower than expression levels of tolerogenic factors encoded by transgenes. In these instances, a positive selection method could include contacting the cells with beads conjugated to a first antibody against the tolerogenic factor having a first avidity and/or a first affinity which may bind preferentially to cells expressing both exogenous transgene encoded tolerogenic factors as well as endogenous tolerogenic factor molecules. Any cells expressing mostly the endogenous tolerogenic factor would flow through the column. With negative selection, the antibody used is against surface antigen(s) which are known to be present on cells that are not of interest. After administration of the cells/magnetic nanoparticles solution onto the column the cells expressing these antigens bind to the column and the fraction that goes through is collected, as it contains almost no cells with these undesired antigens.

Another example of a cell sorting method is the Streptamer technology, which allows reversible isolation and staining of antigen-specific T cells. In principle, the T cells are separated by establishing a specific interaction between the T cell of interest and a molecule that is conjugated to a marker, which enables the isolation. The reversibility of this interaction and the fact that it is performed at low temperatures is the reason for the successful isolation and characterization of functional T cells. Because T cells remain phenotypically and functionally indistinguishable from untreated cells, this method offers new strategies in clinical and basic T cell research. The Streptamer staining principle combines the classic method of T cell isolation by MHC-multimers with the Strep-tag/Strep-Tactin technology. The Strep-tag is a short peptide sequence that displays moderate binding affinity for the biotin-binding site of a mutated streptavidin molecule, called Strep-Tactin. For the Streptamer technology, the Strep-Tactin molecules are multimerized, thus creating a platform for binding to strep-tagged proteins. Further, the Strep-Tactin backbone has a fluorescent label to allow flow cytometry analysis. Incubation of MHC-Strep-tag fusion proteins with the Strep-Tactin backbone results in the formation of an MHC-multimer, which is capable for antigen-specific staining of T cells.

Other examples of cell separation using methodological standards that ensure high purity are rapid and label-free separation procedures based on surface marker density. Exemplary procedures involve the use of an anti-surface marker antibody-immobilized cell-rolling column, that can separate cells depending on the surface marker density of the cell surfaces. Various conditions for the cell-rolling column can be optimized including adjustment of the column tilt angle and medium flow rate.

In some embodiments, the T cells generated by methods according to various embodiments of the present technology have at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the T cells in the population having the first transgene encoding a tolerogenic factor (e.g., CD47) inserted into an endogenous TCR gene locus. In some embodiments, have at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the generated T cells have reduced expression of CD3 and/or increased expression of a tolerogenic factor (e.g., CD47) encoded by a transgene. In some embodiments, have at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the generated T cells have reduced expression of TCR and/or increased expression of a tolerogenic factor (e.g., CD47) encoded by a transgene. In any of these embodiments, the remainder T cells in the population do not possess the described selection characteristic(s).

### C. Insertion of a Second Transgene Encoding a CAR

In some embodiments, the methods described herein for generating a population of T cells, such as immune evasive allogeneic T cells, may further comprise inserting a second transgene encoding one or more CARs to a genomic locus of the T cells (FIG. 1, step 400), in order to generate CAR-T cells for use in cell-based therapies against various target antigens. This step of inserting a second transgene encoding one or more CARs may occur before, with, or after the step of inserting a first transgene encoding a tolerogenic factor, although the flow chart of FIG. 1 only shows an embodiment where insertion of the second transgene follows insertion of the first transgene.

### 1. CAR

CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. A CAR may comprise an extracellular binding domain (also referred to as a “binder”) that specifically binds a target antigen, a transmembrane domain, and an intracellular signaling domain. In certain embodiments, the CAR may further comprise one or more additional elements, including one or more signal peptides, one or more extracellular hinge domains, and/or one or more intracellular costimulatory domains. Domains may be directly adjacent to one another, or there may be one or more amino acids linking the domains. The nucleotide sequence encoding a CAR may be derived from a mammalian sequence, for example, a mouse sequence, a primate sequence, a human sequence, or combinations thereof. In the cases where the nucleotide sequence encoding a CAR is non-human, the sequence of the CAR may be humanized. The nucleotide sequence encoding a CAR may also be codon-optimized for expression in a mammalian cell, for example, a human cell. In any of these embodiments, the nucleotide sequence encoding a CAR may be at least 80% identical (e.g., at least 80%, at least 85%, at least 90%, at least 95%, at least 96%, at least 97%, at least 98%, at least 99%, or 100% identical) to any of the nucleotide sequences disclosed herein. The sequence variations may be due to codon-optimalization, humanization, restriction enzyme-based cloning scars, and/or additional amino acid residues linking the functional domains, etc.

In certain embodiments, the CAR may comprise a signal peptide at the N-terminus. Non-limiting examples of signal peptides include CD8a signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 2 below.

TABLE 2
Exemplary sequences of signal peptides

| SEQ ID NO: | Sequence | Description |
|---|---|---|
| 6 | MALPVTALLLP | CD8α signal peptide |
| | LALLLHAARP | |
| 7 | METDTLLLWV | IgK signal peptide |
| | LLLWVPGSTG | |
| 8 | MLLLVTSLLLC | GMCSFR-α (CSF2RA) |
| | ELPHPAFLLIP | signal peptide |

In certain embodiments, the extracellular binding domain of the CAR may comprise one or more antibodies specific to one target antigen or multiple target antigens. The antibody may be an antibody fragment, for example, an scFv, or a single-domain antibody fragment, for example, a VHH. In certain embodiments, the scFv may comprise a heavy chain variable region (V_{H}) and a light chain variable region (V_{L}) of an antibody connected by a linker. The V_{H} and the V_{L} may be connected in either order, i.e., V_{H}-linker-V_{L} or V_{L}-linker-V_{H}. Non-limiting examples of linkers include Whitlow linker, (G₄S)ₙ (n can be a positive integer, e.g., 1, 2, 3, 4, 5, 6, etc.) linker, and variants thereof. In certain embodiments, the antigen may be an antigen that is exclusively or preferentially expressed on tumor cells, or an antigen that is characteristic of an autoimmune or inflammatory disease. Exemplary target antigens include, but are not limited to, CD5, CD19, CD20, CD22, CD23, CD30, CD33, CD70, Kappa, Lambda, B cell maturation agent (BCMA), and G-protein coupled receptor family C group 5 member D (GPRC5D) (associated with leukemias); CS1/SLAMF7, CD38, CD138, GPRC5D, TACI, and BCMA (associated with myelomas); CD123, LeY, NKG2D ligand, and WT1 (associated with other hematological cancers); GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, ROR1, C-Met, CD133, Ep-CAM, GPC3, HPV16-E6, IL13Ra2, MAGEA3, MAGEA4, MART1, NY-ESO-1, VEGFR2, α-Folate receptor, CD24, CD44v7/8, EGP-2, EGP-40, erb-B2, erb-B 2,3,4, FBP, Fetal acethylcholine e receptor, G_{D2}, G_{D3}, HMW-MAA, IL-11Rα, KDR, Lewis Y, L1-cell adhesion molecule, MAGE-A1, Oncofetal antigen (h5T4), and TAG-72 (associated with solid tumors); A*02 (associated with organ transplantation); fibroblast activation protein (FAP)(associated with fibrosis); urokinase-type plasminogen activator receptor (uPAR) (associated with senescence). In certain embodiments, the CAR can be re-engineered as a chimeric autoantibody receptor (CAAR) to selectively deplete autoreactive immune cells. In certain embodiments, CAARs are engineered to target autoantibodies present on immune cells. Exemplary target antigens for CAARs include, but are not limited to, DSG3 (associated with pemphigus volgaris); factor VIII (FVIII)(associated with haemophilia). In any of these embodiments, the extracellular binding domain of the CAR can be codon-optimized for expression in a host cell or have variant sequences to increase functions of the extracellular binding domain.

In certain embodiments, the CAR may comprise a hinge domain, also referred to as a spacer. The terms “hinge” and “spacer” may be used interchangeably in the present disclosure. Non-limiting examples of hinge domains include CD8a hinge domain, CD28 hinge domain, IgG4 hinge domain, IgG4 hinge-CH2-CH3 domain, and variants thereof, the amino acid sequences of which are provided in Table 3 below.

TABLE 3
Exemplary sequences of hinge domains

| SEQ | | |
|---|---|---|
| ID | | |
| NO: | Sequence | Description |
| 9 | TTTPAPRPPTPAPTIASQPLSLRP | CD8α hinge |
| | EACRPAAGGAVHTRGLDFACD | domain |
| 10 | IEVMYPPPYLDNEKSNGTIIHVKG | CD28 hinge |
| | KHLCPSPLFPGPSKP | domain |
| 11 | AAAIEVMYPPPYLDNEKSNGTIIH | CD28 hinge |
| | VKGKHLCPSPLFPGPSKP | domain |
| 12 | ESKYGPPCPPCP | IgG4 hinge |
| | | domain |
| 13 | ESKYGPPCPSCP | IgG4 hinge |
| | | domain |
| 14 | ESKYGPPCPPCPAPEFLGGPSVFL | IgG4 hinge- |
| | FPPKPKDTLMISRTPEVTCVVVDV | CH2-CH3 |
| | SQEDPEVQFNWYVDGVEVHNAKTK | domain |
| | PREEQFNSTYRVVSVLTVLHQDWL | |
| | NGKEYKCKVSNKGLPSSIEKTISK | |
| | AKGQPREPQVYTLPPSQEEMTKNQ | |
| | VSLTCLVKGFYPSDIAVEWESNGQ | |
| | PENNYKTTPPVLDSDGSFFLYSRL | |
| | TVDKSRWQEGNVFSCSVMHEALHN | |
| | HYTQKSLSLSLGK | |

In certain embodiments, the transmembrane domain of the CAR may comprise a transmembrane region of the alpha, beta, or zeta chain of a T cell receptor, CD28, CD3a, CD45, CD4, CD5, CD8, CD9, CD16, CD22, CD33, CD37, CD64, CD80, CD86, CD134, CD137, CD154, or a functional variant thereof, including the human versions of each of these sequences. In other embodiments, the transmembrane domain may comprise a transmembrane region of CD8a, CD803, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B3, or a functional variant thereof, including the human versions of each of these sequences. Table 4 provides the amino acid sequences of a few exemplary transmembrane domains.

TABLE 4
Exemplary sequences of transmembrane domains

| SEQ ID NO: | Sequence | Description |
|---|---|---|
| 15 | IYIWAPLAGTCGV | CD8α transmembrane |
| | LLLSLVITLYC | domain |
| 16 | FWVLVVVGGVLAC | CD28 transmembrane |
| | YSLLVTVAFIIFW | domain |
| | V | |
| 17 | MFWVLVVVGGVLA | CD28 transmembrane |
| | CYSLLVTVAFIIF | domain |
| | WV | |

In certain embodiments, the intracellular signaling domain and/or intracellular costimulatory domain of the CAR may comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3ζ, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain and/or intracellular costimulatory domain comprises one or more signaling domains selected from a CD3ζ domain, an ITAM, a CD28 domain, 4-1BB domain, or a functional variant thereof. Table 5 provides the amino acid sequences of a few exemplary intracellular costimulatory and/or signaling domains. In certain embodiments, as in the case of tisagenlecleucel as described below, the CD3ζ signaling domain of SEQ ID NO:20 may have a mutation, e.g., a glutamine (Q) to lysine (K) mutation, at amino acid position 14 (see SEQ ID NO:21).

TABLE 5
Exemplary sequences of intracellular
costimulatory and/or signaling domains

| SEQ | | |
|---|---|---|
| ID | | |
| NO: | Sequence | Description |
| 18 | KRGRKKLLYIFKQPFMRPVQTTQEED | 4-1BB |
| | GCSCRFPEEEEGGCEL | costimulatory |
| | | domain |
| 19 | RSKRSRLLHSDYMNMTPRRPGPTRKH | CD28 |
| | YQPYAPPRDFAAYRS | costimulatory |
| | | domain |
| 20 | RVKFSRSADAPAYQQGQNQLYNELNL | CD3ζ signaling |
| | GRREEYDVLDKRRGRDPEMGGKPRRK | domain |
| | NPQEGLYNELQKDKMAEAYSEIGMKG | |
| | ERRRGKGHDGLYQGLSTATKDTYDAL | |
| | HMQALPPR | |
| 21 | RVKFSRSADAPAYKQGQNQLYNELNL | CD3 signaling |
| | GRREEYDVLDKRRGRDPEMGGKPRRK | domain (with |
| | NPQEGLYNELQKDKMAEAYSEIGMKG | Q to K mutation |
| | ERRRGKGHDGLYQGLSTATKDTYDAL | at position 14) |
| | HMQALPPR | |

i. CD19 CAR

In some embodiments, the CAR is a CD19 CAR, and in these embodiments, the second transgene comprises a nucleotide sequence encoding a CD19 CAR. In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the signal peptide of the CD19 CAR comprises a CD8a signal peptide. In some embodiments, the CD8a signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34(16-17):1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 6 below. In some embodiments, the CD19-specific scFv comprises or consists of an amino acid sequence set forth in SEQ ID NO: 22, 23, or 28, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 22, 23, or 28. In some embodiments, the CD19-specific scFv may comprise one or more CDRs having amino acid sequences set forth in SEQ ID NOs: 24-26 and 29-31. In some embodiments, the CD19-specific scFv may comprise a light chain with one or more CDRs having amino acid sequences set forth in SEQ ID NOs: 24-26. In some embodiments, the CD19-specific scFv may comprise a heavy chain with one or more CDRs having amino acid sequences set forth in SEQ ID NOs: 29-31. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the V_{H} and the V_{L} portions of the scFv is a Whitlow linker having an amino acid sequence set forth in SEQ ID NO:27. In some embodiments, the Whitlow linker may be replaced by a different linker, for example, a 3×G₄S linker having an amino acid sequence set forth in SEQ ID NO:33, which gives rise to a different FMC63-derived scFv having an amino acid sequence set forth in SEQ ID NO:32. In certain of these embodiments, the CD19-specific scFv comprises or consists of an amino acid sequence set forth in SEQ ID NO:32 or an amino acid sequence that is at least 80% identical (e.g., at least 80%, at least 8500, at least 90%, at least 9500, at least 96%, at least 97%, at least 98%, at least 99%, or 100% identical) to the amino acid sequence set forth in of SEQ ID NO:32.

TABLE 6
Exemplary sequences of anti-CD19 scFv and components

| SEQ ID NO: | Amino Acid Sequence | Description |
|---|---|---|
| 22 | DIQMTQTTSSLSASLGDRVTISCRASQDI | Anti-CD19 FMC63 |
| | SKYLNWYQQKPDGTVKLLIYHTSRLHS | scFv entire sequence, |
| | GVPSRFSGSGSGTDYSLTISNLEQEDIAT | with Whitlow linker |
| | YFCQQGNTLPYTFGGGTKLEITGSTSGS | |
| | GKPGSGEGSTKGEVKLQESGPGLVAPSQ | |
| | SLSVTCTVSGVSLPDYGVSWIRQPPRKG | |
| | LEWLGVIWGSETTYYNSALKSRLTIIKD | |
| | NSKSQVFLKMNSLQTDDTAIYYCAKHY | |
| | YYGGSYAMDYWGQGTSVTVSS | |
| 23 | DIQMTQTTSSLSASLGDRVTISCRASQDI | Anti-CD19 FMC63 |
| | SKYLNWYQQKPDGTVKLLIYHTSRLHS | scFv light chain |
| | GVPSRFSGSGSGTDYSLTISNLEQEDIAT | variable region |
| | YFCQQGNTLPYTFGGGTKLEIT | |
| 24 | QDISKY | Anti-CD19 FMC63 |
| | | scFv light chain CDR1 |
| 25 | HTS | Anti-CD19 FMC63 |
| | | scFv light chain CDR2 |
| 26 | QQGNTLPYT | Anti-CD19 FMC63 |
| | | scFv light chain CDR3 |
| 27 | GSTSGSGKPGSGEGSTKG | Whitlow linker |
| 28 | EVKLQESGPGLVAPSQSLSVTCTVSGVS | Anti-CD19 FMC63 |
| | LPDYGVSWIRQPPRKGLEWLGVIWGSET | scFv heavy chain |
| | TYYNSALKSRLTIIKDNSKSQVFLKMNS | variable region |
| | LQTDDTAIYYCAKHYYYGGSYAMDYW | |
| | GQGTSVTVSS | |
| 29 | GVSLPDYG | Anti-CD19 FMC63 |
| | | scFv heavy chain |
| | | CDR1 |
| 30 | IWGSETT | Anti-CD19 FMC63 |
| | | scFv heavy chain |
| | | CDR2 |
| 31 | AKHYYYGGSYAMDY | Anti-CD19 FMC63 |
| | | scFv heavy chain |
| | | CDR3 |
| 32 | DIQMTQTTSSLSASLGDRVTISCRASQDI | Anti-CD19 FMC63 |
| | SKYLNWYQQKPDGTVKLLIYHTSRLHS | scFv entire sequence, |
| | GVPSRFSGSGSGTDYSLTISNLEQEDIAT | with 3xG₄S linker |
| | YFCQQGNTLPYTFGGGTKLEITGGGGSG | |
| | GGGSGGGGSEVKLQESGPGLVAPSQSLS | |
| | VTCTVSGVSLPDYGVSWIRQPPRKGLE | |
| | WLGVIWGSETTYYNSALKSRLTIIKDNS | |
| | KSQVFLKMNSLQTDDTAIYYCAKHYYY | |
| | GGSYAMDYWGQGTSVTVSS | |
| 33 | GGGGSGGGGSGGGGS | 3xG₄S linker |

In some embodiments, the extracellular binding domain of the CD19 CAR is derived from an antibody specific to CD19, including, for example, SJ25C1 (Bejcek et al., Cancer Res. 55:2346-2351 (1995)), HD37 (Pezutto et al., J. Immunol. 138(9):2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148(10): 2983-2987 (1992)), and CLB-CD19 (De Rie Cell. Immunol. 118:368-381(1989)). In any of these embodiments, the extracellular binding domain of the CD19 CAR can comprise or consist of the V_{H}, the V_{L}, and/or one or more CDRs of any of the antibodies.

In some embodiments, the hinge domain of the CD19 CAR comprises a CD8a hinge domain, for example, a human CD8a hinge domain. In some embodiments, the CD8a hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO: 12 or SEQ ID NO: 13, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 12 or SEQ ID NO: 13. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:14.

In some embodiments, the transmembrane domain of the CD19 CAR comprises a CD8a transmembrane domain, for example, a human CD8a transmembrane domain. In some embodiments, the CD8a transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 15. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO: 18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 18. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO:19 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:19. In some embodiments, the intracellular costimulatory domain of the CD19 CAR comprises a 4-1BB costimulatory domain and a CD28 costimulatory domain as described.

In some embodiments, the intracellular signaling domain of the CD19 CAR comprises a CD3 zeta (ζ) signaling domain. CD3ζ associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In some embodiments, the CD3ζ signaling domain is human. In some embodiments, the CD3ζ signaling domain comprises or consists of an amino acid sequence set forth in SEQ ID NO:20 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:20.

In some embodiments, the second transgene comprises a nucleotide sequence encoding a CD19 CAR, including, for example, a CD19 CAR comprising the CD19-specific scFv having sequences set forth in SEQ ID NO:22 or SEQ ID NO:32, the CD8a hinge domain of SEQ ID NO:9, the CD8a transmembrane domain of SEQ ID NO:15, the 4-1BB costimulatory domain of SEQ ID NO:18, the CD3ζ signaling domain of SEQ ID NO:20, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8a signal peptide) as described.

In some embodiments, the second transgene comprises a nucleotide sequence encoding a CD19 CAR, including, for example, a CD19 CAR comprising the CD19-specific scFv having sequences set forth in SEQ ID NO:22 or SEQ ID NO:32, the IgG4 hinge domain of SEQ ID NO: 12 or SEQ ID NO: 13, the CD28 transmembrane domain of SEQ ID NO: 16, the 4-1BB costimulatory domain of SEQ ID NO:18, the CD3ζ signaling domain of SEQ ID NO:20, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the second transgene comprises a nucleotide sequence encoding a CD19 CAR, including, for example, a CD19 CAR comprising the CD19-specific scFv having sequences set forth in SEQ ID NO:22 or SEQ ID NO:32, the CD28 hinge domain of SEQ ID NO: 10, the CD28 transmembrane domain of SEQ ID NO:16, the CD28 costimulatory domain of SEQ ID NO:19, the CD3ζ signaling domain of SEQ ID NO:20, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the second transgene comprises a nucleotide sequence encoding a CD19 CAR as set forth in SEQ ID NO:34 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:34 (see Table 7). The encoded CD19 CAR has a corresponding amino acid sequence set forth in SEQ ID NO:35 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:35, with the following components: CD8α signal peptide, FMC63 scFv (V_{L}-Whitlow linker-V_{H}), CD8α hinge domain, CD8α transmembrane domain, 4-1BB costimulatory domain, and CD3ζ signaling domain.

In some embodiments, the second transgene comprises a nucleotide sequence encoding a commercially available embodiment of CD19 CAR. Non-limiting examples of commercially available embodiments of CD19 CARs expressed and/or encoded by T cells include tisagenlecleucel, lisocabtagene maraleucel, axicabtagene ciloleucel, and brexucabtagene autoleucel.

In some embodiments, the second transgene comprises a nucleotide sequence encoding tisagenlecleucel or portions thereof. Tisagenlecleucel comprises a CD19 CAR with the following components: CD8α signal peptide, FMC63 scFv (V_{L}-3×G₄S linker-V_{H}), CD8α hinge domain, CD8α transmembrane domain, 4-1BB costimulatory domain, and CD3ζ signaling domain. The nucleotide and amino acid sequence of the CD19 CAR in tisagenlecleucel are provided in Table 7, with annotations of the sequences provided in Table 8.

In some embodiments, the second transgene comprises a nucleotide sequence encoding lisocabtagene maraleucel or portions thereof. Lisocabtagene maraleucel comprises a CD19 CAR with the following components: GMCSFR-α or CSF2RA signal peptide, FMC63 scFv (V_{L}-Whitlow linker-V_{H}), IgG4 hinge domain, CD28 transmembrane domain, 4-1BB costimulatory domain, and CD3ζ signaling domain. The nucleotide and amino acid sequence of the CD19 CAR in lisocabtagene maraleucel are provided in Table 7, with annotations of the sequences provided in Table 9.

In some embodiments, the second transgene comprises a nucleotide sequence encoding axicabtagene ciloleucel or portions thereof. Axicabtagene ciloleucel comprises a CD19 CAR with the following components: GMCSFR-α or CSF2RA signal peptide, FMC63 scFv (V_{L}-Whitlow linker-V_{H}), CD28 hinge domain, CD28 transmembrane domain, CD28 costimulatory domain, and CD3ζ signaling domain. The nucleotide and amino acid sequence of the CD19 CAR in axicabtagene ciloleucel are provided in Table 7, with annotations of the sequences provided in Table 10.

In some embodiments, the second transgene comprises a nucleotide sequence encoding brexucabtagene autoleucel or portions thereof. Brexucabtagene autoleucel comprises a CD19 CAR with the following components: GMCSFR-α signal peptide, FMC63 scFv, CD28 hinge domain, CD28 transmembrane domain, CD28 costimulatory domain, and CD3ζ signaling domain.

In some embodiments, the second transgene comprises a nucleotide sequence encoding a CD19 CAR as set forth in SEQ ID NO: 36, 38, or 40, or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 36, 38, or 40. The encoded CD19 CAR has a corresponding amino acid sequence set forth in SEQ ID NO: 37, 39, or 41, respectively, or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 37, 39, or 41, respectively.

TABLE 7
Exemplary sequences of CD19 CARs

| SEQ | | |
|---|---|---|
| ID | | |
| NO: | Sequence | Description |
| 34 | atggccttaccagtgaccgccttgctcctgccgctggccttgctg | Exemplary |
| | ctccacgccgccaggccggacatccagatgacacagactacatcc | CD19 CAR |
| | tccctgtctgcctctctgggagacagagtcaccatcagttgcagg | nucleotide |
| | gcaagtcaggacattagtaaatatttaaattggtatcagcagaaa | sequence |
| | ccagatggaactgttaaactcctgatctaccatacatcaagatta | |
| | cactcaggagtcccatcaaggttcagtggcagtgggtctggaaca | |
| | gattattctctcaccattagcaacctggagcaagaagatattgcc | |
| | acttacttttgccaacagggtaatacgcttccgtacacgttcgga | |
| | ggggggaccaagctggagatcacaggctccacctctggatccggc | |
| | aagcccggatctggcgagggatccaccaagggcgaggtgaaactg | |
| | caggagtcaggacctggcctggtggcgccctcacagagcctgtcc | |
| | gtcacatgcactgtctcaggggtctcattacccgactatggtgta | |
| | agctggattcgccagcctccacgaaagggtctggagtggctggga | |
| | gtaatatggggtagtgaaaccacatactataattcagctctcaaa | |
| | tccagactgaccatcatcaaggacaactccaagagccaagttttc | |
| | ttaaaaatgaacagtctgcaaactgatgacacagccatttactac | |
| | tgtgccaaacattattactacggtggtagctatgctatggactac | |
| | tggggccaaggaacctcagtcaccgtctcctcaaccacgacgcca | |
| | gcgccgcgaccaccaacaccggcgcccaccatcgcgtcgcagccc | |
| | ctgtccctgcgcccagaggcgtgccggccagcggcggggggcgca | |
| | gtgcacacgagggggctggacttcgcctgtgatatctacatctgg | |
| | gcgcccttggccgggacttgtggggtccttctcctgtcactggtt | |
| | atcaccctttactgcaaacggggcagaaagaaactcctgtatata | |
| | ttcaaacaaccatttatgagaccagtacaaactactcaagaggaa | |
| | gatggctgtagctgccgatttccagaagaagaagaaggaggatgt | |
| | gaactgagagtgaagttcagcaggagcgcagacgcccccgcgtac | |
| | cagcagggccagaaccagctctataacgagctcaatctaggacga | |
| | agagaggagtacgatgttttggacaagagacgtggccgggaccct | |
| | gagatggggggaaagccgagaaggaagaaccctcaggaaggcctg | |
| | tacaatgaactgcagaaagataagatggcggaggcctacagtgag | |
| | attgggatgaaaggcgagcgccggaggggcaaggggcacgatggc | |
| | ctttaccagggtctcagtacagccaccaaggacacctacgacgcc | |
| | cttcacatgcaggccctgccccctcgc | |
| 35 | MALPVTALLLPLALLLHAARPDIQMTQTTSSLSASLGDRVTISCR | Exemplary |
| | ASQDISKYLNWYQQKPDGTVKLLIYHTSRLHSGVPSRFSGSGSGT | CD19 CAR |
| | DYSLTISNLEQEDIATYFCQQGNTLPYTFGGGTKLEITGSTSGSG | amino acid |
| | KPGSGEGSTKGEVKLQESGPGLVAPSQSLSVTCTVSGVSLPDYGV | sequence |
| | SWIRQPPRKGLEWLGVIWGSETTYYNSALKSRLTIIKDNSKSQVF | |
| | LKMNSLQTDDTAIYYCAKHYYYGGSYAMDYWGQGTSVTVSSTTTP | |
| | APRPPTPAPTIASQPLSLRPEACRPAAGGAVHTRGLDFACDIYIW | |
| | APLAGTCGVLLLSLVITLYCKRGRKKLLYIFKQPFMRPVQTTQEE | |
| | DGCSCRFPEEEEGGCELRVKFSRSADAPAYQQGQNQLYNELNLGR | |
| | REEYDVLDKRRGRDPEMGGKPRRKNPQEGLYNELQKDKMAEAYSE | |
| | IGMKGERRRGKGHDGLYQGLSTATKDTYDALHMQALPPR | |
| 36 | atggccttaccagtgaccgccttgctcctgccgctggccttgctg | Tisagenlecleucel |
| | ctccacgccgccaggccggacatccagatgacacagactacatcc | CD19 CAR |
| | tccctgtctgcctctctgggagacagagtcaccatcagttgcagg | nucleotide |
| | gcaagtcaggacattagtaaatatttaaattggtatcagcagaaa | sequence |
| | ccagatggaactgttaaactcctgatctaccatacatcaagatta | |
| | cactcaggagtcccatcaaggttcagtggcagtgggtctggaaca | |
| | gattattctctcaccattagcaacctggagcaagaagatattgcc | |
| | acttacttttgccaacagggtaatacgcttccgtacacgttcgga | |
| | ggggggaccaagctggagatcacaggtggcggtggctcgggcggt | |
| | ggtgggtcgggtggcggcggatctgaggtgaaactgcaggagtca | |
| | ggacctggcctggtggcgccctcacagagcctgtccgtcacatgc | |
| | actgtctcaggggtctcattacccgactatggtgtaagctggatt | |
| | cgccagcctccacgaaagggtctggagtggctgggagtaatatgg | |
| | ggtagtgaaaccacatactataattcagctctcaaatccagactg | |
| | accatcatcaaggacaactccaagagccaagttttcttaaaaatg | |
| | aacagtctgcaaactgatgacacagccatttactactgtgccaaa | |
| | cattattactacggtggtagctatgctatggactactggggccaa | |
| | ggaacctcagtcaccgtctcctcaaccacgacgccagcgccgcga | |
| | ccaccaacaccggcgcccaccatcgcgtcgcagcccctgtccctg | |
| | cgcccagaggcgtgccggccagcggggggggcgcagtgcacacga | |
| | gggggctggacttcgcctgtgatatctacatctgggcgcccttgg | |
| | ccgggacttgtggggtccttctcctgtcactggttatcacccttt | |
| | actgcaaacggggcagaaagaaactcctgtatatattcaaacaac | |
| | catttatgagaccagtacaaactactcaagaggaagatggctgta | |
| | gctgccgatttccagaagaagaagaaggaggatgtgaactgagag | |
| | tgaagttcagcaggagcgcagacgcccccgcgtacaagcagggcc | |
| | agaaccagctctataacgagctcaatctaggacgaagagaggagt | |
| | acgatgttttggacaagagacgtggccgggaccctgagatggggg | |
| | gaaagccgagaaggaagaaccctcaggaaggcctgtacaatgaac | |
| | tgcagaaagataagatggcggaggcctacagtgagattgggatga | |
| | aaggcgagcgccggaggggcaaggggcacgatggcctttaccagg | |
| | gtctcagtacagccaccaaggacacctacgacgcccttcacatgc | |
| | aggccctgccccctcgc | |
| 37 | MALPVTALLLPLALLLHAARPDIQMTQTTSSLSASLGDRVTISCR | Tisagenlecleucel |
| | ASQDISKYLNWYQQKPDGTVKLLIYHTSRLHSGVPSRFSGSGSGT | CD19 CAR |
| | DYSLTISNLEQEDIATYFCQQGNTLPYTFGGGTKLEITGGGGSGG | amino acid |
| | GGSGGGGSEVKLQESGPGLVAPSQSLSVTCTVSGVSLPDYGVSWI | sequence |
| | RQPPRKGLEWLGVIWGSETTYYNSALKSRLTIIKDNSKSQVFLKM | |
| | NSLQTDDTAIYYCAKHYYYGGSYAMDYWGQGTSVTVSSTTTPAPR | |
| | PPTPAPTIASQPLSLRPEACRPAAGGAVHTRGLDFACDIYIWAPL | |
| | AGTCGVLLLSLVITLYCKRGRKKLLYIFKQPFMRPVQTTQEEDGC | |
| | SCRFPEEEEGGCELRVKFSRSADAPAYKQGQNQLYNELNLGRREE | |
| | YDVLDKRRGRDPEMGGKPRRKNPQEGLYNELQKDKMAEAYSEIGM | |
| | KGERRRGKGHDGLYQGLSTATKDTYDALHMQALPPR | |
| 38 | atgctgctgctggtgaccagcctgctgctgtgcgagctgccccac | Lisocabtagene |
| | cccgcctttctgctgatccccgacatccagatgacccagaccacc | maraleucel |
| | tccagcctgagcgccagcctgggcgaccgggtgaccatcagctgc | CD19 CAR |
| | cgggccagccaggacatcagcaagtacctgaactggtatcagcag | nucleotide |
| | aagcccgacggcaccgtcaagctgctgatctaccacaccagccgg | sequence |
| | ctgcacagcggcgtgcccagccggtttagcggcagcggctccggc | |
| | accgactacagcctgaccatctccaacctggaacaggaagatatc | |
| | gccacctacttttgccagcagggcaacacactgccctacaccttt | |
| | ggcggcggaacaaagctggaaatcaccggcagcacctccggcagc | |
| | ggcaagcctggcagcggcgagggcagcaccaagggcgaggtgaag | |
| | ctgcaggaaagcggccctggcctggtggcccccagccagagcctg | |
| | agcgtgacctgcaccgtgagcggcgtgagcctgcccgactacggc | |
| | gtgagctggatccggcagccccccaggaagggcctggaatggctg | |
| | ggcgtgatctggggcagcgagaccacctactacaacagcgccctg | |
| | aagagccggctgaccatcatcaaggacaacagcaagagccaggtg | |
| | ttcctgaagatgaacagcctgcagaccgacgacaccgccatctac | |
| | tactgcgccaagcactactactacggcggcagctacgccatggac | |
| | tactggggccagggcaccagcgtgaccgtgagcagcgaatctaag | |
| | tacggaccgccctgccccccttgccctatgttctgggtgctggtg | |
| | gtggtcggaggcgtgctggcctgctacagcctgctggtcaccgtg | |
| | gccttcatcatcttttgggtgaaacggggcagaaagaaactcctg | |
| | tatatattcaaacaaccatttatgagaccagtacaaactactcaa | |
| | gaggaagatggctgtagctgccgatttccagaagaagaagaagga | |
| | ggatgtgaactgcgggtgaagttcagcagaagcgccgacgcccct | |
| | gcctaccagcagggccagaatcagctgtacaacgagctgaacctg | |
| | ggcagaagggaagagtacgacgtcctggataagcggagaggccgg | |
| | gaccctgagatgggcggcaagcctcggcggaagaacccccaggaa | |
| | ggcctgtataacgaactgcagaaagacaagatggccgaggcctac | |
| | agcgagatcggcatgaagggcgagcggaggcggggcaagggccac | |
| | gacggcctgtatcagggcctgtccaccgccaccaaggatacctac | |
| | gacgccctgcacatgcaggccctgcccccaagg | |
| 39 | MLLLVTSLLLCELPHPAFLLIPDIQMTQTTSSLSASLGDRVTISC | Lisocabtagene |
| | RASQDISKYLNWYQQKPDGTVKLLIYHTSRLHSGVPSRFSGSGSG | maraleucel |
| | TDYSLTISNLEQEDIATYFCQQGNTLPYTFGGGTKLEITGSTSGS | CD19 CAR |
| | GKPGSGEGSTKGEVKLQESGPGLVAPSQSLSVTCTVSGVSLPDYG | amino acid |
| | VSWIRQPPRKGLEWLGVIWGSETTYYNSALKSRLTIIKDNSKSQV | sequence |
| | FLKMNSLQTDDTAIYYCAKHYYYGGSYAMDYWGQGTSVTVSSESK | |
| | YGPPCPPCPMFWVLVVVGGVLACYSLLVTVAFIIFWVKRGRKKLL | |
| | YIFKQPFMRPVQTTQEEDGCSCRFPEEEEGGCELRVKFSRSADAP | |
| | AYQQGQNQLYNELNLGRREEYDVLDKRRGRDPEMGGKPRRKNPQE | |
| | GLYNELQKDKMAEAYSEIGMKGERRRGKGHDGLYQGLSTATKDTY | |
| | DALHMQALPPR | |
| 40 | atgcttctcctggtgacaagccttctgctctgtgagttaccacac | Axicabtagene |
| | ccagcattcctcctgatcccagacatccagatgacacagactaca | ciloleucel CD19 |
| | tcctccctgtctgcctctctgggagacagagtcaccatcagttgc | CAR nucleotide |
| | agggcaagtcaggacattagtaaatatttaaattggtatcagcag | sequence |
| | aaaccagatggaactgttaaactcctgatctaccatacatcaaga | |
| | ttacactcaggagtcccatcaaggttcagtggcagtgggtctgga | |
| | acagattattctctcaccattagcaacctggagcaagaagatatt | |
| | gccacttacttttgccaacagggtaatacgcttccgtacacgttc | |
| | ggaggggggactaagttggaaataacaggctccacctctggatcc | |
| | ggcaagcccggatctggcgagggatccaccaagggcgaggtgaaa | |
| | ctgcaggagtcaggacctggcctggtggcgccctcacagagcctg | |
| | tccgtcacatgcactgtctcaggggtctcattacccgactatggt | |
| | gtaagctggattcgccagcctccacgaaagggtctggagtggctg | |
| | ggagtaatatggggtagtgaaaccacatactataattcagctctc | |
| | aaatccagactgaccatcatcaaggacaactccaagagccaagtt | |
| | ttcttaaaaatgaacagtctgcaaactgatgacacagccatttac | |
| | tactgtgccaaacattattactacggtggtagctatgctatggac | |
| | tactggggtcaaggaacctcagtcaccgtctcctcagcggccgca | |
| | attgaagttatgtatcctcctccttacctagacaatgagaagagc | |
| | aatggaaccattatccatgtgaaagggaaacacctttgtccaagt | |
| | cccctatttcccggaccttctaagcccttttgggtgctggtggtg | |
| | gttgggggagtcctggcttgctatagcttgctagtaacagtggcc | |
| | tttattattttctgggtgaggagtaagaggagcaggctcctgcac | |
| | agtgactacatgaacatgactccccgccgccccgggcccacccgc | |
| | aagcattaccagccctatgccccaccacgcgacttcgcagcctat | |
| | cgctccagagtgaagttcagcaggagcgcagacgcccccgcgtac | |
| | cagcagggccagaaccagctctataacgagctcaatctaggacga | |
| | agagaggagtacgatgttttggacaagagacgtggccgggaccct | |
| | gagatggggggaaagccgagaaggaagaaccctcaggaaggcctg | |
| | tacaatgaactgcagaaagataagatggcggaggcctacagtgag | |
| | attgggatgaaaggcgagcgccggaggggcaaggggcacgatggc | |
| | ctttaccagggtctcagtacagccaccaaggacacctacgacgcc | |
| | cttcacatgcaggccctgccccctcgc | |
| 41 | MLLLVTSLLLCELPHPAFLLIPDIQMTQTTSSLSASLGDRVTISC | Axicabtagene |
| | RASQDISKYLNWYQQKPDGTVKLLIYHTSRLHSGVPSRFSGSGSG | ciloleucel CD19 |
| | TDYSLTISNLEQEDIATYFCQQGNTLPYTFGGGTKLEITGSTSGS | CAR amino acid |
| | GKPGSGEGSTKGEVKLQESGPGLVAPSQSLSVTCTVSGVSLPDYG | sequence |
| | VSWIRQPPRKGLEWLGVIWGSETTYYNSALKSRLTIIKDNSKSQV | |
| | FLKMNSLQTDDTAIYYCAKHYYYGGSYAMDYWGQGTSVTVSSAAA | |
| | IEVMYPPPYLDNEKSNGTIIHVKGKHLCPSPLFPGPSKPFWVLVV | |
| | VGGVLACYSLLVTVAFIIFWVRSKRSRLLHSDYMNMTPRRPGPTR | |
| | KHYQPYAPPRDFAAYRSRVKFSRSADAPAYQQGQNQLYNELNLGR | |
| | REEYDVLDKRRGRDPEMGGKPRRKNPQEGLYNELQKDKMAEAYSE | |
| | IGMKGERRRGKGHDGLYQGLSTATKDTYDALHMQALPPR | |

TABLE 8
Annotation of tisagenlecleucel CD19 CAR sequences

| | Nucleotide | Amino Acid |
|---|---|---|
| | Sequence | Sequence |
| Feature | Position | Position |
| CD8α signal peptide | 1-63 | 1-21 |
| FMC63 scFv | 64-789 | 22-263 |
| (V_{L}-3xG₄S linker-V_{H}) | | |
| CD8α hinge domain | 790-924 | 264-308 |
| CD8α transmembrane domain | 925-996 | 309-332 |
| 4-1BB costimulatory domain | 997-1122 | 333-374 |
| CD3ζ signaling domain | 1123-1458 | 375-486 |

TABLE 9
Annotation of lisocabtagene maraleucel CD19 CAR sequences

| | Nucleotide | Amino Acid |
|---|---|---|
| | Sequence | Sequence |
| Feature | Position | Position |
| GMCSFR-α signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB costimulatory domain | 922-1047 | 308-349 |
| CD3ζ signaling domain | 1048-1383 | 350-461 |

TABLE 10
Annotation of axicabtagene ciloleucel CD19 CAR sequences

| | Nucleotide | Amino Acid |
|---|---|---|
| | Sequence | Sequence |
| Feature | Position | Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 costimulatory domain | 1009-1131 | 337-377 |
| CD3ζ signaling domain | 1132-1467 | 378-489 |

In some embodiments, the second transgene comprises a nucleotide sequence encoding CD19 CAR as set forth in SEQ ID NO: 31, 33, or 35, or at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 31, 33, or 35. The encoded CD19 CAR has a corresponding amino acid sequence set forth in SEQ ID NO: 32, 34, or 36, respectively, is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 32, 34, or 36, respectively.

ii. CD20 CAR

In some embodiments, the CAR is a CD20 CAR, and in these embodiments, the second transgene comprises a nucleotide sequence encoding a CD20 CAR. CD20 is an antigen found on the surface of B cells as early at the pro-B phase and progressively at increasing levels until B cell maturity, as well as on the cells of most B-cell neoplasms. CD20 positive cells are also sometimes found in cases of Hodgkin's disease, myeloma, and thymoma. In some embodiments, the CD20 CAR may comprise a signal peptide, an extracellular binding domain that specifically binds CD20, a hinge domain, a transmembrane domain, an intracellular costimulatory domain, and/or an intracellular signaling domain in tandem.

In some embodiments, the signal peptide of the CD20 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Leu16, IF5, 1.5.3, rituximab, obinutuzumab, ibritumomab, ofatumumab, tositumumab, odronextamab, veltuzumab, ublituximab, and ocrelizumab. In any of these embodiments, the extracellular binding domain of the CD20 CAR can comprise or consist of the V_{H}, the V_{L}, and/or one or more CDRs of any of the antibodies.

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14(12):1025-1033 (2001). In some embodiments, the linker is a 3×G₄S linker. In other embodiments, the linker is a Whitlow linker as described herein. In some embodiments, the amino acid sequences of different portions of the entire Leu16-derived scFv (also referred to as Leu16 scFv) and its different portions are provided in Table 11 below. In some embodiments, the CD20-specific scFv comprises or consists of an amino acid sequence set forth in SEQ ID NO: 42, 43, or 47, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 42, 43, or 47. In some embodiments, the CD20-specific scFv may comprise one or more CDRs having amino acid sequences set forth in SEQ ID NOs: 44-46, 48, and 49. In some embodiments, the CD20-specific scFv may comprise a light chain with one or more CDRs having amino acid sequences set forth in SEQ ID NOs: 44-46. In some embodiments, the CD20-specific scFv may comprise a heavy chain with one or more CDRs having amino acid sequences set forth in SEQ ID NOs: 48-49. In any of these embodiments, the CD20-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD20 CAR comprises or consists of the one or more CDRs as described herein.

TABLE 11
Exemplary sequences of anti-CD20 scFv and components

| SEQ ID NO: | Amino Acid Sequence | Description |
|---|---|---|
| 42 | DIVLTQSPAILSASPGEKVTMTCRASSSVNYM | Anti-CD20 Leu16 scFv |
| | DWYQKKPGSSPKPWIYATSNLASGVPARFSGS | entire sequence, with |
| | GSGTSYSLTISRVEAEDAATYYCQQWSFNPPT | Whitlow linker |
| | FGGGTKLEIKGSTSGSGKPGSGEGSTKGEVQL | |
| | QQSGAELVKPGASVKMSCKASGYTFTSYNMH | |
| | WVKQTPGQGLEWIGAIYPGNGDTSYNQKFKG | |
| | KATLTADKSSSTAYMQLSSLTSEDSADYYCAR | |
| | SNYYGSSYWFFDVWGAGTTVTVSS | |
| 43 | DIVLTQSPAILSASPGEKVTMTCRASSSVNYM | Anti-CD20 Leu16 scFv |
| | DWYQKKPGSSPKPWIYATSNLASGVPARFSGS | light chain variable |
| | GSGTSYSLTISRVEAEDAATYYCQQWSFNPPT | region |
| | FGGGTKLEIK | |
| 44 | RASSSVNYMD | Anti-CD20 Leu16 scFv |
| | | light chain CDR1 |
| 45 | ATSNLAS | Anti-CD20 Leu16 scFv |
| | | light chain CDR2 |
| 46 | QQWSFNPPT | Anti-CD20 Leu16 scFv |
| | | light chain CDR3 |
| 47 | EVQLQQSGAELVKPGASVKMSCKASGYTFTS | Anti-CD20 Leu16 scFv |
| | YNMHWVKQTPGQGLEWIGAIYPGNGDTSYNQ | heavy chain |
| | KFKGKATLTADKSSSTAYMQLSSLTSEDSAD | |
| | YYCARSNYYGSSYWFFDVWGAGTTVTVSS | |
| 48 | SYNMH | Anti-CD20 Leu16 scFv |
| | | heavy chain CDR1 |
| 49 | AIYPGNGDTSYNQKFKG | Anti-CD20 Leu16 scFv |
| | | heavy chain CDR2 |

In some embodiments, the hinge domain of the CD20 CAR comprises a CD8α hinge domain, for example, a human CD8 at hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO: 10 or an amino acid sequence that is at least 80% identical (e.g., at least 80%, at least 85%, at least 90%, at least 95% at least 96%, at least 97%, at least 98%, at least 99% or 100% identical) to the amino acid sequence set forth in of SEQ ID NO: 10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO: 12 or SEQ ID NO: 13, or an amino acid sequence that is at least 80% identical (e.g., at least 80%, at least 85%, at least 90%, at least 95% at least 96%, at least 97% at least 98%, at least 99% or 100% identical) to the amino acid sequence set forth in of SEQ ID NO: 12 or SEQ ID NO: 13. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:14.

In some embodiments, the transmembrane domain of the CD20 CAR comprises a CD8α transmembrane domain, for example, a human CD8α transmembrane domain. In some embodiments, the CD8α transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 15. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16.

In some embodiments, the intracellular costimulatory domain of the CD20 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 18. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:19 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:19.

In some embodiments, the intracellular signaling domain of the CD20 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:20 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:20.

In some embodiments, the second transgene comprises a nucleotide sequence encoding a CD20 CAR, including, for example, a CD20 CAR comprising the CD20-specific scFv having sequences set forth in SEQ ID NO:42, the CD8α hinge domain of SEQ ID NO:9, the CD8α transmembrane domain of SEQ ID NO:15, the 4-1BB costimulatory domain of SEQ ID NO:18, the CD3ζ signaling domain of SEQ ID NO:20, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD20 CAR, including, for example, a CD20 CAR comprising the CD20-specific scFv having sequences set forth in SEQ ID NO:42, the CD28 hinge domain of SEQ ID NO: 10, the CD8α transmembrane domain of SEQ ID NO:15, the 4-1BB costimulatory domain of SEQ ID NO:18, the CD3ζ signaling domain of SEQ ID NO:20, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD20 CAR, including, for example, a CD20 CAR comprising the CD20-specific scFv having sequences set forth in SEQ ID NO:42, the IgG4 hinge domain of SEQ ID NO:12 or SEQ ID NO: 13, the CD8α transmembrane domain of SEQ ID NO:15, the 4-1BB costimulatory domain of SEQ ID NO:18, the CD3ζ signaling domain of SEQ ID NO:20, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD20 CAR, including, for example, a CD20 CAR comprising the CD20-specific scFv having sequences set forth in SEQ ID NO:42, the CD8α hinge domain of SEQ ID NO:9, the CD28 transmembrane domain of SEQ ID NO:16, the 4-1BB costimulatory domain of SEQ ID NO:18, the CD3ζ signaling domain of SEQ ID NO:20, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD20 CAR, including, for example, a CD20 CAR comprising the CD20-specific scFv having sequences set forth in SEQ ID NO:42, the CD28 hinge domain of SEQ ID NO:10, the CD28 transmembrane domain of SEQ ID NO:16, the 4-1BB costimulatory domain of SEQ ID NO:18, the CD3ζ signaling domain of SEQ ID NO:20, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD20 CAR, including, for example, a CD20 CAR comprising the CD20-specific scFv having sequences set forth in SEQ ID NO:42, the IgG4 hinge domain of SEQ ID NO:12 or SEQ ID NO: 13, the CD28 transmembrane domain of SEQ ID NO:16, the 4-1BB costimulatory domain of SEQ ID NO:18, the CD3ζ signaling domain of SEQ ID NO:20, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

iii. CD22 CAR

In some embodiments, the CAR is a CD22 CAR, and in these embodiments, the second transgene comprises a nucleotide sequence encoding a CD22 CAR. CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise a signal peptide, an extracellular binding domain that specifically binds CD22, a hinge domain, a transmembrane domain, an intracellular costimulatory domain, and/or an intracellular signaling domain in tandem.

In some embodiments, the signal peptide of the CD22 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, and pinatuzumab. In any of these embodiments, the extracellular binding domain of the CD22 CAR can comprise or consist of the V_{H}, the V_{L}, and/or one or more CDRs of any of the antibodies.

In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a 3×G₄S linker. In other embodiments, the Whitlow linker may be used instead. In some embodiments, the amino acid sequences of the entire m971-derived scFv (also referred to as m971 scFv) and its different portions are provided in Table 12 below. In some embodiments, the CD22-specific scFv comprises or consists of an amino acid sequence set forth in SEQ ID NO: 50, 51, or 55, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 50, 51, or 55. In some embodiments, the CD22-specific scFv may comprise one or more CDRs having amino acid sequences set forth in SEQ ID NOs: 52-54 and 56-58. In some embodiments, the CD22-specific scFv may comprise a heavy chain with one or more CDRs having amino acid sequences set forth in SEQ ID NOs: 52-54. In some embodiments, the CD22-specific scFv may comprise a light chain with one or more CDRs having amino acid sequences set forth in SEQ ID NOs: 56-58. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 pM). In some embodiments, the scFv derived from m971-L7 comprises the V_{H} and the V_{L} of m971-L7 connected by a 3×G₄S linker. In other embodiments, the Whitlow linker may be used instead. In some embodiments, the amino acid sequences of the entire m971-L7-derived scFv (also referred to as m971-L7 scFv) and its different portions are provided in Table 12 below. In some embodiments, the CD22-specific scFv comprises or consists of an amino acid sequence set forth in SEQ ID NO: 59, 60, or 64, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 59, 60, or 64. In some embodiments, the CD22-specific scFv may comprise one or more CDRs having amino acid sequences set forth in SEQ ID NOs: 61-63 and 65-67. In some embodiments, the CD22-specific scFv may comprise a heavy chain with one or more CDRs having amino acid sequences set forth in SEQ ID NOs: 61-63. In some embodiments, the CD22-specific scFv may comprise a light chain with one or more CDRs having amino acid sequences set forth in SEQ ID NOs: 65-67. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

TABLE 12
Exemplary sequences of anti-CD22 scFv and components

| SEQ ID NO: | Amino Acid Sequence | Description |
|---|---|---|
| 50 | QVQLQQSGPGLVKPSQTLSLTCAISGDSVSSN | Anti-CD22 m971 |
| | SAAWNWIRQSPSRGLEWLGRTYYRSKWYND | scFv entire sequence, |
| | YAVSVKSRITINPDTSKNQFSLQLNSVTPEDT | with 3xG4S linker |
| | AVYYCAREVTGDLEDAFDIWGQGTMVTVSS | |
| | GGGGSGGGGSGGGGSDIQMTQSPSSLSASVG | |
| | DRVTITCRASQTIWSYLNWYQQRPGKAPNLL | |
| | IYAASSLQSGVPSRFSGRGSGTDFTLTISSLQA | |
| | EDFATYYCQQSYSIPQTFGQGTKLEIK | |
| 51 | QVQLQQSGPGLVKPSQTLSLTCAISGDSVSSN | Anti-CD22 m971 |
| | SAAWNWIRQSPSRGLEWLGRTYYRSKWYND | scFv heavy chain |
| | YAVSVKSRITINPDTSKNQFSLQLNSVTPEDT | variable region |
| | AVYYCAREVTGDLEDAFDIWGQGTMVTVSS | |
| 52 | GDSVSSNSAA | Anti-CD22 m971 |
| | | scFv heavy chain |
| | | CDR1 |
| 53 | TYYRSKWYN | Anti-CD22 m971 |
| | | scFv heavy chain |
| | | CDR2 |
| 54 | AREVTGDLEDAFDI | Anti-CD22 m971 |
| | | scFv heavy chain |
| | | CDR3 |
| 55 | DIQMTQSPSSLSASVGDRVTITCRASQTIWSY | Anti-CD22 m971 |
| | LNWYQQRPGKAPNLLIYAASSLQSGVPSRFS | scFv light chain |
| | GRGSGTDFTLTISSLQAEDFATYYCQQSYSIP | |
| | QTFGQGTKLEIK | |
| 56 | QTIWSY | Anti-CD22 m971 |
| | | scFv light chain |
| | | CDR1 |
| 57 | AAS | Anti-CD22 m971 |
| | | scFv light chain |
| | | CDR2 |
| 58 | QQSYSIPQT | Anti-CD22 m971 |
| | | scFv light chain |
| | | CDR3 |
| 59 | QVQLQQSGPGMVKPSQTLSLTCAISGDSVSSN | Anti-CD22 m971-L7 |
| | SVAWNWIRQSPSRGLEWLGRTYYRSTWYND | scFv entire sequence, |
| | YAVSMKSRITINPDTNKNQFSLQLNSVTPEDT | with 3xG4S linker |
| | AVYYCAREVTGDLEDAFDIWGQGTMVTVSS | |
| | GGGGSGGGGSGGGGSDIQMIQSPSSLSASVG | |
| | DRVTITCRASQTIWSYLNWYRQRPGEAPNLLI | |
| | YAASSLQSGVPSRFSGRGSGTDFTLTISSLQAE | |
| | DFATYYCQQSYSIPQTFGQGTKLEIK | |
| 60 | QVQLQQSGPGMVKPSQTLSLTCAISGDSVSSN | Anti-CD22 m971-L7 |
| | SVAWNWIRQSPSRGLEWLGRTYYRSTWYND | scFv heavy chain |
| | YAVSMKSRITINPDTNKNQFSLQLNSVTPEDT | variable region |
| | AVYYCAREVTGDLEDAFDIWGQGTMVTVSS | |
| 61 | GDSVSSNSVA | Anti-CD22 m971-L7 |
| | | scFv heavy chain |
| | | CDR1 |
| 62 | TYYRSTWYN | Anti-CD22 m971-L7 |
| | | scFv heavy chain |
| | | CDR2 |
| 63 | AREVTGDLEDAFDI | Anti-CD22 m971-L7 |
| | | scFv heavy chain |
| | | CDR3 |
| 64 | DIQMIQSPSSLSASVGDRVTITCRASQTIWSYL | Anti-CD22 m971-L7 |
| | NWYRQRPGEAPNLLIYAASSLQSGVPSRFSGR | scFv light chain |
| | GSGTDFTLTISSLQAEDFATYYCQQSYSIPQTF | variable region |
| | GQGTKLEIK | |
| 65 | QTIWSY | Anti-CD22 m971-L7 |
| | | scFv light chain |
| | | CDR1 |
| 66 | AAS | Anti-CD22 m971-L7 |
| | | scFv light chain |
| | | CDR2 |
| 67 | QQSYSIPQT | Anti-CD22 m971-L7 |
| | | scFv light chain |
| | | CDR3 |

In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280(1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO: 12 or SEQ ID NO: 13, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 12 or SEQ ID NO: 13. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:14.

In some embodiments, the transmembrane domain of the CD22 CAR comprises a CD8α transmembrane domain, for example, a human CD8α transmembrane domain. In some embodiments, the CD8α transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 15. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16.

In some embodiments, the intracellular costimulatory domain of the CD22 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 18. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:19 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:19.

In some embodiments, the intracellular signaling domain of the CD22 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:20 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:20.

In some embodiments, the second transgene comprises a nucleotide sequence encoding a CD22 CAR, including, for example, a CD22 CAR comprising the CD22-specific scFv having sequences set forth in SEQ ID NO:50 or SEQ ID NO:59, the CD8α hinge domain of SEQ ID NO:9, the CD8α transmembrane domain of SEQ ID NO:15, the 4-1BB costimulatory domain of SEQ ID NO:18, the CD3ζ signaling domain of SEQ ID NO:20, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD22 CAR, including, for example, a CD22 CAR comprising the CD22-specific scFv having sequences set forth in SEQ ID NO:50 or SEQ ID NO:59, the CD28 hinge domain of SEQ ID NO: 10, the CD8α transmembrane domain of SEQ ID NO:15, the 4-1BB costimulatory domain of SEQ ID NO:18, the CD3ζ signaling domain of SEQ ID NO:20, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD22 CAR, including, for example, a CD22 CAR comprising the CD22-specific scFv having sequences set forth in SEQ ID NO:50 or SEQ ID NO:59, the IgG4 hinge domain of SEQ ID NO: 12 or SEQ ID NO: 13, the CD8α transmembrane domain of SEQ ID NO: 15, the 4-1BB costimulatory domain of SEQ ID NO:18, the CD3ζ signaling domain of SEQ ID NO:20, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD22 CAR, including, for example, a CD22 CAR comprising the CD22-specific scFv having sequences set forth in SEQ ID NO:50 or SEQ ID NO:59, the CD8α hinge domain of SEQ ID NO:9, the CD28 transmembrane domain of SEQ ID NO:16, the 4-1BB costimulatory domain of SEQ ID NO:18, the CD3ζ signaling domain of SEQ ID NO:20, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD22 CAR, including, for example, a CD22 CAR comprising the CD22-specific scFv having sequences set forth in SEQ ID NO:50 or SEQ ID NO:59, the CD28 hinge domain of SEQ ID NO: 10, the CD28 transmembrane domain of SEQ ID NO:16, the 4-1BB costimulatory domain of SEQ ID NO:18, the CD3ζ signaling domain of SEQ ID NO:20, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the second transgene comprises a nucleotide sequence encoding a CD22 CAR, including, for example, a CD22 CAR comprising the CD22-specific scFv having sequences set forth in SEQ ID NO:50 or SEQ ID NO:59, the IgG4 hinge domain of SEQ ID NO: 12 or SEQ ID NO: 13, the CD28 transmembrane domain of SEQ ID NO: 16, the 4-1BB costimulatory domain of SEQ ID NO:18, the CD3ζ signaling domain of SEQ ID NO:20, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

iv. BCMA CAR

In some embodiments, the CAR is a BCMA CAR, and in these embodiments, the second transgene comprises a nucleotide sequence encoding a BCMA CAR. BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR may comprise a signal peptide, an extracellular binding domain that specifically binds BCMA, a hinge domain, a transmembrane domain, an intracellular costimulatory domain, and/or an intracellular signaling domain in tandem.

In some embodiments, the signal peptide of the BCMA CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, erlanatamab, teclistamab, LCAR-B38M, and ciltacabtagene. In any of these embodiments, the extracellular binding domain of the BCMA CAR can comprise or consist of the V_{H}, the V_{L}, and/or one or more CDRs of any of the antibodies.

In some embodiments, the extracellular binding domain of the BCMA CAR comprises an scFv derived from C11D5.3, a murine monoclonal antibody as described in Carpenter et al., Clin. Cancer Res. 19(8):2048-2060 (2013). See also PCT Application Publication No. WO2010/104949. The C11D5.3-derived scFv may comprise the heavy chain variable region (V_{H}) and the light chain variable region (V_{L}) of C11D5.3 connected by the Whitlow linker, the amino acid sequences of which is provided in Table 13 below. In some embodiments, the BCMA-specific extracellular binding domain comprises or consists of an amino acid sequence set forth in SEQ ID NO:68, 69, or 73, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:68, 69, or 73. In some embodiments, the BCMA-specific extracellular binding domain may comprise one or more CDRs having amino acid sequences set forth in SEQ ID NOs: 70-72 and 74-76. In some embodiments, the BCMA-specific extracellular binding domain may comprise a light chain with one or more CDRs having amino acid sequences set forth in SEQ ID NOs: 70-72. In some embodiments, the BCMA-specific extracellular binding domain may comprise a heavy chain with one or more CDRs having amino acid sequences set forth in SEQ ID NOs: 74-76. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from another murine monoclonal antibody, C12A3.2, as described in Carpenter et al., Clin. Cancer Res. 19(8):2048-2060 (2013) and PCT Application Publication No. WO2010/104949, the amino acid sequence of which is also provided in Table 13 below. In some embodiments, the BCMA-specific extracellular binding domain comprises or consists of an amino acid sequence set forth in SEQ ID NO:77, 78, or 82, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:77, 78, or 82. In some embodiments, the BCMA-specific extracellular binding domain may comprise one or more CDRs having amino acid sequences set forth in SEQ ID NOs: 79-81 and 83-85. In some embodiments, the BCMA-specific extracellular binding domain may comprise a light chain with one or more CDRs having amino acid sequences set forth in SEQ ID NOs: 79-81. In some embodiments, the BCMA-specific extracellular binding domain may comprise a heavy chain with one or more CDRs having amino acid sequences set forth in SEQ ID NOs: 83-85. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29(5):585-601 (2018)). See also, PCT Application Publication No. WO2012163805.

In some embodiments, the extracellular binding domain of the BCMA CAR comprises single variable fragments of two heavy chains (VHH) that can bind to two epitopes of BCMA as described in Zhao et al., J. Hematol. Oncol. 11(1):141 (2018), also referred to as LCAR-B38M. See also, PCT Application Publication No. WO2018/028647.

In some embodiments, the extracellular binding domain of the BCMA CAR comprises a fully human heavy-chain variable domain (FHVH) as described in Lam et al., Nat. Commun. 11(1):283 (2020), also referred to as FHVH33. See also, PCT Application Publication No. WO2019/006072. The amino acid sequences of FHVH33 and its CDRs are provided in Table 13 below. In some embodiments, the BCMA-specific extracellular binding domain comprises or consists of an amino acid sequence set forth in SEQ ID NO:86 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:86. In some embodiments, the BCMA-specific extracellular binding domain may comprise one or more CDRs having amino acid sequences set forth in SEQ ID NOs: 87-89. In any of these embodiments, the BCMA-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from CT103A (or CAR0085) as described in U.S. Pat. No. 11,026,975 B2, the amino acid sequence of which is provided in Table 13 below. In some embodiments, the BCMA-specific extracellular binding domain comprises or consists of an amino acid sequence set forth in SEQ ID NO:90, 91, or 95, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 90, 91, or 95. In some embodiments, the BCMA-specific extracellular binding domain may comprise one or more CDRs having amino acid sequences set forth in SEQ ID NOs: 92-94 and 96-98. In some embodiments, the BCMA-specific extracellular binding domain may comprise a light chain with one or more CDRs having amino acid sequences set forth in SEQ ID NOs: 92-94. In some embodiments, the BCMA-specific extracellular binding domain may comprise a heavy chain with one or more CDRs having amino acid sequences set forth in SEQ ID NOs: 96-98. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

Additionally, CARs and binders directed to BCMA have been described in U.S. Application Publication Nos. 2020/0246381 A1 and 2020/0339699 A1, the entire contents of each of which are incorporated by reference herein.

TABLE 13
Exemplary sequences of anti-BCMA binder and components

| SEQ ID | | |
|---|---|---|
| NO: | Amino Acid Sequence | Description |
| 68 | DIVLTQSPASLAMSLGKRATISCRASESVSVIG | Anti-BCMA C11D5.3 |
| | AHLIHWYQQKPGQPPKLLIYLASNLETGVPAR | scFv entire sequence, |
| | FSGSGSGTDFTLTIDPVEEDDVAIYSCLQSRIFP | with Whitlow linker |
| | RTFGGGTKLEIKGSTSGSGKPGSGEGSTKGQIQ | |
| | LVQSGPELKKPGETVKISCKASGYTFTDYSIN | |
| | WVKRAPGKGLKWMGWINTETREPAYAYDER | |
| | GRFAFSLETSASTAYLQINNLKYEDTATYFCA | |
| | LDYSYAMDYWGQGTSVTVSS | |
| 69 | DIVLTQSPASLAMSLGKRATISCRASESVSVIG | Anti-BCMA C11D5.3 |
| | AHLIHWYQQKPGQPPKLLIYLASNLETGVPAR | scFv light chain |
| | FSGSGSGTDFTLTIDPVEEDDVAIYSCLQSRIFP | variable region |
| | RTFGGGTKLEIK | |
| 70 | RASESVSVIGAHLIH | Anti-BCMA C11D5.3 |
| | | scFv light chain |
| | | CDR1 |
| 71 | LASNLET | Anti-BCMA C11D5.3 |
| | | scFv light chain |
| | | CDR2 |
| 72 | LQSRIFPRT | Anti-BCMA C11D5.3 |
| | | scFv light chain |
| | | CDR3 |
| 73 | QIQLVQSGPELKKPGETVKISCKASGYTFTDYS | Anti-BCMA C11D5.3 |
| | INWVKRAPGKGLKWMGWINTETREPAYAYD | scFv heavy chain |
| | FRGRFAFSLETSASTAYLQINNLKYEDTATYFC | variable region |
| | ALDYSYAMDYWGQGTSVTVSS | |
| 74 | DYSIN | Anti-BCMA C11D5.3 |
| | | scFv heavy chain |
| | | CDR1 |
| 75 | WINTETREPAYAYDFRG | Anti-BCMA C11D5.3 |
| | | scFv heavy chain |
| | | CDR2 |
| 76 | DYSYAMDY | Anti-BCMA C11D5.3 |
| | | scFv heavy chain |
| | | CDR3 |
| 77 | DIVLTQSPPSLAMSLGKRATISCRASESVTILGS | Anti-BCMA C12A3.2 |
| | HLIYWYQQKPGQPPTLLIQLASNVQTGVPARF | scFv entire sequence, |
| | SGSGSRTDFTLTIDPVEEDDVAVYYCLQSRTIP | with Whitlow linker |
| | RTFGGGTKLEIKGSTSGSGKPGSGEGSTKGQIQ | |
| | LVQSGPELKKPGETVKISCKASGYTFRHYSMN | |
| | WVKQAPGKGLKWMGRINTESGVPIYADDFKG | |
| | RFAFSVETSASTAYLVINNLKDEDTASYFCSN | |
| | DYLYSLDFWGQGTALTVSS | |
| 78 | DIVLTQSPPSLAMSLGKRATISCRASESVTILGS | Anti-BCMA C12A3.2 |
| | HLIYWYQQKPGQPPTLLIQLASNVQTGVPARF | scFv light chain |
| | SGSGSRTDFTLTIDPVEEDDVAVYYCLQSRTIP | variable region |
| | RTFGGGTKLEIK | |
| 79 | RASESVTILGSHLIY | Anti-BCMA C12A3.2 |
| | | scFv light chain |
| | | CDR1 |
| 80 | LASNVQT | Anti-BCMA C12A3.2 |
| | | scFv light chain |
| | | CDR2 |
| 81 | LQSRTIPRT | Anti-BCMA C12A3.2 |
| | | scFv light chain |
| | | CDR3 |
| 82 | QIQLVQSGPELKKPGETVKISCKASGYTFRHYS | Anti-BCMA C12A3.2 |
| | MNWVKQAPGKGLKWMGRINTESGVPIYADD | scFv heavy chain |
| | FKGRFAFSVETSASTAYLVINNLKDEDTASYF | variable region |
| | CSNDYLYSLDFWGQGTALTVSS | |
| 83 | HYSMN | Anti-BCMA C12A3.2 |
| | | scFv heavy chain |
| | | CDR1 |
| 84 | RINTESGVPIYADDFKG | Anti-BCMA C12A3.2 |
| | | scFv heavy chain |
| | | CDR2 |
| 85 | DYLYSLDF | Anti-BCMA C12A3.2 |
| | | scFv heavy chain |
| | | CDR3 |
| 86 | EVQLLESGGGLVQPGGSLRLSCAASGFTFSSY | Anti-BCMA FHVH33 |
| | AMSWVRQAPGKGLEWVSSISGSGDYIYYADS | entire sequence |
| | VKGRFTISRDISKNTLYLQMNSLRAEDTAVYY | |
| | CAKEGTGANSSLADYRGQGTLVTVSS | |
| 87 | GFTFSSYA | Anti-BCMA FHVH33 |
| | | CDR1 |
| 88 | ISGSGDYI | Anti-BCMA FHVH33 |
| | | CDR2 |
| 89 | AKEGTGANSSLADY | Anti-BCMA FHVH33 |
| | | CDR3 |
| 90 | DIQMTQSPSSLSASVGDRVTITCRASQSISSYLN | Anti-BCMA CT103A |
| | WYQQKPGKAPKLLIYAASSLQSGVPSRFSGSG | scFv entire sequence, |
| | SGTDFTLTISSLQPEDFATYYCQQKYDLLTFGG | with Whitlow linker |
| | GTKVEIKGSTSGSGKPGSGEGSTKGQLQLQES | |
| | GPGLVKPSETLSLTCTVSGGSISSSSYYWGWIR | |
| | QPPGKGLEWIGSISYSGSTYYNPSLKSRVTISV | |
| | DTSKNQFSLKLSSVTAADTAVYYCARDRGDTI | |
| | LDVWGQGTMVTVSS | |
| 91 | DIQMTQSPSSLSASVGDRVTITCRASQSISSYLN | Anti-BCMA CT103A |
| | WYQQKPGKAPKLLIYAASSLQSGVPSRFSGSG | scFv light chain |
| | SGTDFTLTISSLQPEDFATYYCQQKYDLLTFGG | variable region |
| | GTK VEIK | |
| 92 | QSISSY | Anti-BCMA CT103A |
| | | scFv light chain |
| | | CDR1 |
| 93 | AAS | Anti-BCMA CT103A |
| | | scFv light chain |
| | | CDR2 |
| 94 | QQKYDLLT | Anti-BCMA CT103A |
| | | scFv light chain |
| | | CDR3 |
| 95 | QLQLQESGPGLVKPSETLSLTCTVSGGSISSSSY | Anti-BCMA CT103A |
| | YWGWIRQPPGKGLEWIGSISYSGSTYYNPSLK | scFv heavy chain |
| | SRVTISVDTSKNQFSLKLSSVTAADTAVYYCA | variable region |
| | RDRGDTILDVWGQGTMVTVSS | |
| 96 | GGSISSSSYY | Anti-BCMA CT103A |
| | | scFv heavy chain |
| | | CDR1 |
| 97 | ISYSGST | Anti-BCMA CT103A |
| | | scFv heavy chain |
| | | CDR2 |
| 98 | ARDRGDTILDV | Anti-BCMA CT103A |
| | | scFv heavy chain |
| | | CDR3 |

In some embodiments, the hinge domain of the BCMA CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO: 12 or SEQ ID NO: 13, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 12 or SEQ ID NO: 13. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:14.

In some embodiments, the transmembrane domain of the BCMA CAR comprises a CD8α transmembrane domain, for example, a human CD8α transmembrane domain. In some embodiments, the CD8α transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 15. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16.

In some embodiments, the intracellular costimulatory domain of the BCMA CAR comprises a 4-1BB costimulatory domain, for example, a human 4-1BB costimulatory domain. In some embodiments, the 4-1BB costimulatory domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 18. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:19 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:19.

In some embodiments, the intracellular signaling domain of the BCMA CAR comprises a CD3 zeta (ζ) signaling domain, for example, a human CD3ζ signaling domain. In some embodiments, the CD3ζ signaling domain comprises or consists of an amino acid sequence set forth in SEQ ID NO:20 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:20.

In some embodiments, the second transgene comprises a nucleotide sequence encoding a BCMA CAR, including, for example, a BCMA CAR comprising any of the BCMA-specific extracellular binding domains as described, the CD8α hinge domain of SEQ ID NO:9, the CD8α transmembrane domain of SEQ ID NO:15, the 4-1BB costimulatory domain of SEQ ID NO:18, the CD3ζ signaling domain of SEQ ID NO:20, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide (e.g., a CD8α signal peptide) as described.

In some embodiments, the second transgene comprises a nucleotide sequence encoding a BCMA CAR, including, for example, a BCMA CAR comprising any of the BCMA-specific extracellular binding domains as described, the CD8α hinge domain of SEQ ID NO:9, the CD8α transmembrane domain of SEQ ID NO:15, the CD28 costimulatory domain of SEQ ID NO:19, the CD3ζ signaling domain of SEQ ID NO:20, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide as described.

In some embodiments, the second transgene comprises a nucleotide sequence encoding a BCMA CAR as set forth in SEQ ID NO:99 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:99 (see Table 14). The encoded BCMA CAR has a corresponding amino acid sequence set forth in SEQ ID NO:100 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:100, with the following components: CD8α signal peptide, CT103A scFv (V_{L}-Whitlow linker-V_{H}), CD8α hinge domain, CD8α transmembrane domain, 4-1BB costimulatory domain, and CD3ζ signaling domain.

In some embodiments, the second transgene comprises a nucleotide sequence encoding a commercially available embodiment of BCMA CAR, including, for example, idecabtagene vicleucel (ide-cel, also called bb2121). In some embodiments, the second transgene comprises a nucleotide sequence encoding idecabtagene vicleucel or portions thereof. Idecabtagene vicleucel comprises a BCMA CAR with the following components: the BB2121 binder, CD8α hinge domain, CD8α transmembrane domain, 4-1BB costimulatory domain, and CD3ζ signaling domain.

TABLE 14
Exemplary sequences of BCMA CARs

| SEQ | | |
|---|---|---|
| ID | | |
| NO: | Sequence | Description |
| 99 | atggccttaccagtgaccgccttgctcctg | Exemplary BCMA |
| | ccgctggccttgctgctccacgccgccagg | CAR nucleotide |
| | ccggacatccagatgacccagtctccatcc | sequence |
| | tccctgtctgcatctgtaggagacagagtc | |
| | accatcacttgccgggcaagtcagagcatt | |
| | agcagctatttaaattggtatcagcagaaa | |
| | ccagggaaagcccctaagctcctgatctat | |
| | gctgcatccagtttgcaaagtggggtccca | |
| | tcaaggttcagtggcagtggatctgggaca | |
| | gatttcactctcaccatcagcagtctgcaa | |
| | cctgaagattttgcaacttactactgtcag | |
| | caaaaatacgacctcctcacttttggcgga | |
| | gggaccaaggttgagatcaaaggcagcacc | |
| | agcggctccggcaagcctggctctggcgag | |
| | ggcagcacaaagggacagctgcagctgcag | |
| | gagtcgggcccaggactggtgaagccttcg | |
| | gagaccctgtccctcacctgcactgtctct | |
| | ggtggctccatcagcagtagtagttactac | |
| | tggggctggatccgccagcccccagggaag | |
| | gggctggagtggattgggagtatctcctat | |
| | agtgggagcacctactacaacccgtccctc | |
| | aagagtcgagtcaccatatccgtagacacg | |
| | tccaagaaccagttctccctgaagctgagt | |
| | tctgtgaccgccgcagacacggcggtgtac | |
| | tactgcgccagagatcgtggagacaccata | |
| | ctagacgtatggggtcagggtacaatggtc | |
| | accgtcagctcattcgtgcccgtgttcctg | |
| | ccogccaaacctaccaccacccctgcccct | |
| | agacctcocaccccagccccaacaatcgcc | |
| | agccagcctctgtctctgcggcccgaagcc | |
| | tgtagacctgctgccggcggagccgtgcac | |
| | accagaggcctggacttcgcctgcgacatc | |
| | tacatctgggcccctctggccggcacctgt | |
| | ggcgtgctgctgctgagcctggtgatcacc | |
| | ctgtactgcaaccaccggaacaaacggggc | |
| | agaaagaaactcctgtatatattcaaacaa | |
| | ccatttatgagaccagtacaaactactcaa | |
| | gaggaagatggctgtagctgccgatttcca | |
| | gaagaagaagaaggaggatgtgaactgaga | |
| | gtgaagttcagcagatccgccgacgcccct | |
| | gcctaccagcagggacagaaccagctgtac | |
| | aacgagctgaacctgggcagacgggaagag | |
| | tacgacgtgctggacaagcggagaggccgg | |
| | gaccccgagatgggcggaaagcccagacgg | |
| | aagaacccccaggaaggcctgtataacgaa | |
| | ctgcagaaagacaagatggccgaggcctac | |
| | agcgagatcggcatgaagggcgagcggagg | |
| | cgcggcaagggccacgatggcctgtaccag | |
| | ggcctgagcaccgccaccaaggacacctac | |
| | gacgccctgcacatgcaggccctgcccccc | |
| | aga | |
| 100 | MALPVTALLLPLALLLHAARPDIQMTQSPS | Exemplary BCMA |
| | SLSASVGDRVTITCRASQSISSYLNWYQQK | CAR amino acid |
| | PGKAPKLLIYAASSLQSGVPSRFSGSGSGT | sequence |
| | DFTLTISSLQPEDFATYYCQQKYDLLTFGG | |
| | GTKVEIKGSTSGSGKPGSGEGSTKGQLQLQ | |
| | ESGPGLVKPSETLSLTCTVSGGSISSSSYY | |
| | WGWIRQPPGKGLEWIGSISYSGSTYYNPSL | |
| | KSRVTISVDTSKNQFSLKLSSVTAADTAVY | |
| | YCARDRGDTILDVWGQGTMVTVSSFVPVFL | |
| | PAKPTTTPAPRPPTPAPTIASQPLSLRPEA | |
| | CRPAAGGAVHTRGLDFACDIYIWAPLAGTC | |
| | GVLLLSLVITLYCNHRNKRGRKKLLYIFKQ | |
| | PFMRPVQTTQEEDGCSCRFPEEEEGGCELR | |
| | VKFSRSADAPAYQQGQNQLYNELNLGRREE | |
| | YDVLDKRRGRDPEMGGKPRRKNPQEGLYNE | |
| | LQKDKMAEAYSEIGMKGERRRGKGHDGLYQ | |
| | GLSTATKDTYDALHMQALPPR | |

v. Multiple CARs

In some embodiments, the second transgene comprises two or more nucleotide sequences, each encoding a CAR targeting a specific target antigen. In these embodiments, the second transgene encodes two or more different CARs specific to different target antigens (e.g., a CD19 CAR and a CD22 CAR). The two or more CARs may each comprise an extracellular binding domain specific to a specific target antigen, and may comprise the same, or one or more different, non-antigen binding domains. For example, the two or more CARs may comprise different signal peptides, hinge domains, transmembrane domains, costimulatory domains, and/or intracellular signaling domains, in order to minimize the risk of recombination due to sequence similarities. Or, alternatively, the two or more CARs may comprise the same non-antigen binding domains. In the cases where the same non-antigen binding domain(s) and/or backbone are used, it is optional to introduce codon divergence at the nucleotide sequence level to minimize the risk of recombination. As one non-limiting example, the second transgene may comprise a nucleotide sequence encoding a CD19 CAR and a nucleotide sequence encoding a CD22 CAR. The CD19 CAR may comprise one transmembrane domain (e.g., CD28 transmembrane domain) while the CD22 CAR comprises a different transmembrane domain (e.g., CD80a transmembrane domain), or vice versa. As another non-limiting example, the CD19 CAR may comprise one costimulatory domain (e.g., 4-1BB costimulatory domain) while the CD22 CAR comprises a different costimulatory domain (e.g., CD28 costimulatory domain), or vice versa. Or, alternatively, the CD22 CAR and the CD19 CARs may comprise the same non-antigen binding domains but have codon divergence introduced at the nucleotide sequence level to minimize the risk of recombination. In any of these embodiments, the two or more nucleotide sequences of the second transgene may be connected by one or more cleavage sites as described (e.g., a 2A site and/or a furin site), in the form of polycistronic constructs as described herein.

### 2. Regulatory Elements

In some embodiments, the second transgene encoding a CAR may comprise additional regulatory elements operatively linked to the CAR encoding sequence as described, including, for example, promoters, insulators, enhancers, polyadenylation (poly(A)) tails, and/or ubiquitous chromatin opening elements.

### 3. Genomic Insertion

In some embodiments, the second transgene encoding a CAR may be delivered into a host cell in the form of a vector for insertion into the host genome. The insertion may be random (i.e., insertion into a random genomic locus of the host cell) or targeted (i.e., insertion into a specific genomic locus of the host cell), using any of the random or site-directed insertion methods described herein.

In some embodiments, the first transgene encoding a tolerogenic factor and the second transgene encoding a CAR may be introduced into a host for genomic insertion separately. In some embodiments, the first transgene encoding a tolerogenic factor and the second transgene encoding a CAR may be introduced into a host for genomic insertion at the same time, via a single vector or multiple vectors. In cases where the first and the second transgene are delivered into a host cell together in a single vector, the first and the second transgene may be designed as a polycistronic construct as described below.

### 4. Polycistronic Constructs

In some embodiments, the first transgene encoding a tolerogenic factor and the second transgene encoding a CAR, and/or the multiple CAR encoding sequences of the second transgene, may be in the form of polycistronic constructs. Polycistronic constructs have two or more expression cassettes for co-expression of two or more proteins of interest in a host cell. In some embodiments, the polycistronic construct comprises two expression cassettes, i.e., is bicistronic. In some embodiments, the polycistronic construct comprises three expression cassettes, i.e., is tricistronic. In some embodiments, the polycistronic construct comprises four expression cassettes, i.e., is quadcistronic. In some embodiments, the polycistronic construct comprises more than four expression cassettes. In any of these embodiments, each of the expression cassettes comprises a nucleotide sequence encoding a protein of interest (e.g., a tolerogenic or a CAR). In certain embodiments, the two or more genes being expressed are under the control of a single promoter and are separated from one another by one or more cleavage sites to achieve co-expression of the proteins of interest from one transcript. In other embodiments, the two or more genes may be under the control of separate promoters.

In some embodiments, the two or more expression cassettes of the polycistronic construct may be separated by one or more cleavage sites. As the name suggests, a polycistronic construct allows simultaneous expression of two or more separate proteins from one mRNA transcript in a host cell. Cleavage sites can be used in the design of a polycistronic construct to achieve such co-expression of multiple genes.

In some embodiments, the one or more cleavage sites comprise one or more self-cleaving sites. In some embodiments, the self-cleaving site comprises a 2A site. 2A peptides are a class of 18-22 amino acid-long peptides first discovered in picornaviruses and can induce ribosomal skipping during translation of a protein, thus producing equal amounts of multiple genes from the same mRNA transcript. 2A peptides function to “cleave” an mRNA transcript by making the ribosome skip the synthesis of a peptide bond at the C-terminus, between the glycine (G) and proline (P) residues, leading to separation between the end of the 2A sequence and the next peptide downstream. There are four 2A peptides commonly employed in molecular biology, T2A, P2A, E2A, and F2A, the sequences of which are summarized in Table 15. A glycine-serine-glycine (GSG) linker is optionally added to the N-terminal of a 2A peptide to increase cleavage efficiency. The use of “( )” around a sequence in the present disclosure means that the enclosed sequence is optional.

TABLE 15
Sequences of 2A peptides

| SEQ | | |
|---|---|---|
| ID | | |
| NO: | Amino Acid Sequence | 2A Peptide |
| 101 | (GSG) EGRGSLLTCGDVEENPGP | T2A |
| 102 | (GSG) ATNFSLLKQAGDVEENPGP | P2A |
| 103 | (GSG) QCTNYALLKLAGDVESNPGP | E2A |
| 104 | (GSG) VKQTLNFDLLKLAGDVESNPGP | F2A |

In some embodiments, the one or more cleavage sites additionally comprise one or more protease sites. The one or more protease sites can either precede or follow the self-cleavage sites (e.g., 2A sites) in the 5′ to 3′ order. The protease site may be cleaved by a protease after translation of the full transcript or after translation of each expression cassette such that the first expression product is released prior to translation of the next expression cassette. In these embodiments, having a protease site in addition to the 2A site, especially preceding the 2A site in the 5′ to 3′ order, may reduce the number of extra amino acid residues attached to the expressed proteins of interest. In some embodiments, the protease site comprises a furin site, also known as a Paired basic Amino acid Cleaving Enzyme (PACE) site. There are at least three furin cleavage sequences, FC1, FC2, and FC3, the amino acid sequences of which are summarized in Table 16. Similar to the 2A sites, one or more optional glycine-serine-glycine (GSG) sequences can be included for cleavage efficiency.

TABLE 16
Sequences of furin sites

| SEQ ID NO: | Amino Acid Sequence | Furin site |
|---|---|---|
| 105 | RRRR (GSG) | FC1 |
| 106 | RKRR (GSG) | FC2 |
| 107 | RKRR (GSG) TPDPW (GSG) | FC3 |

In some embodiments, the one or more cleavage sites comprise one or more self-cleaving sites, one or more protease sites, and/or any combination thereof. For example, the cleavage site can include a 2A site alone. For another example, the cleavage site can include a FC2 or FC3 site, followed by a 2A site. In these embodiments, the one or more self-cleaving sites may be the same or different. Similarly, the one or more protease sites may be the same or different.

In some embodiments, the polycistronic construct may be in the form of a vector. Any type of vector suitable for introduction of nucleotide sequences into a host cell can be used, including, for example, plasmids, adenoviral vectors, adenoviral-associated vectors, retroviral vectors, lentiviral vectors, phages, and homology-directed repair (HDR)-based donor vectors.

### D. Additional Modifications

In some embodiments, the methods described herein for generating a population of T cells, such as immune evasive allogeneic T cells, may further comprise performing additional modifications of the T cells to further reduce the immunogenicity of these cells, in order to reduce potential graft-versus-host risks after infusion into the recipient or risks of being eliminated by the recipient's innate immune system. In some embodiments, the additional modifications comprise reducing or eliminating the expression of MHC class I (MHC I) and/or MHC class II (MHC II) molecules in the T cells (FIG. 1, step 100). This step of modifying MHC 1 and/or MHC II molecules may occur before, with, or after the step of inserting a first transgene encoding a tolerogenic factor or the step of inserting a second transgene encoding a CAR. The flow chart of FIG. 1 shows an embodiment where the modifying step occurs before insertion of the first transgene and insertion of the second transgene.

MHC I and/or MHC II genes encode cell surface molecules specialized to present antigenic peptides to immune cells. Reduced expression of MHC I and/or MHC II molecules in allogeneic cells may prevent recognition of these cells by the immune cells of the recipient and thus rejection of the graft. The MHC in humans is called human leukocyte antigen (HLA). Class I HLA (corresponding to MHC class I) include the HLA-A, HLA-B, and HLA-C genes, and Class II HLA (corresponding to MHC class II) include the HLA-DR, HLA-DQ, HLA-DP, HLA-DM, and HLA-DO genes.

In some embodiments, the T cells, such as immune evasive allogeneic T cells, may be modified to have reduced expression of MHC I genes by targeting and modulating the P2 microglobulin (B2M) locus. The B2M gene encodes a component of MHC I molecules. In some embodiments, the genetic modification targeting the B2M locus occurs through insertion-deletion (indel) modifications of the B2M locus, for example, by using the CRISPR/Cas system as described. In some embodiments, the genetic modification targeting the B2M locus comprises inserting an exogenous nucleic acid at the B2M locus to disrupt expression of the B2M gene. By modifying (e.g., reducing or eliminating) expression of B2M, surface trafficking of MHC I molecules is blocked, and the cell is thus rendered hypoimmunogenic. In some embodiments, the allogeneic T cells modified to have reduced expression of MHC I genes have a reduced ability to induce an immune response in a recipient subject. In some embodiments, reduced expression of B2M reduces or eliminates expression of one or more of the HLA-A, HLA-B, and HLA-C genes. In some embodiments, the allogeneic T cells have B2M knockout.

In some embodiments, the T cells, such as immune evasive allogeneic T cells, may be modified to have reduced expression of MHC II genes by targeting and modulating the class II transactivator (CIITA) locus. CIITA is a member of the nucleotide binding domain (NBD) leucine-rich repeat (LRR) family of proteins and regulates the transcription of MHC II by associating with the MHC enhanceosome. In some embodiments, the genetic modification targeting the CIITA locus occurs through insertion-deletion (indel) modifications of the CIITA locus, for example, by using the CRISPR/Cas system as described. In some embodiments, the genetic modification targeting the CIITA locus comprises inserting an exogenous nucleic acid at the CIITA locus to disrupt expression of the CIITA gene. In some embodiments, reduced expression of CIITA reduces or eliminates expression of one or more of the HLA-DR, HLA-DQ, HLA-DP, HLA-DM, and HLA-DO genes. In some embodiments, the allogeneic T cells have CIITA knockout.

In some embodiments, the T cells, such as immune evasive allogeneic T cells, have genetic modifications at the B2M and/or CIITA loci, or have B2M and/or CIITA knockout. The B2M and/or CIITA knockout can occur at one allele, or both alleles, of the respective gene locus. In some embodiments, the B2M and/or CIITA loci are modified so that the allogeneic T cells have reduced or no expression of B2M and/or CIITA. In these embodiments, the allogeneic T cells have reduced expression of MHC I and/or MHC II genes (HLA I and/or HLA II in humans) as a result of B2M and/or CIITA deletion or knockout. In some embodiments, reducing expression of one or more MHC class I molecule and/or one or more MHC class II molecule comprises reducing expression of one or more of B2M, TAP I, NLRC5, CIITA, HLA-A, HLA-B, HLA-C, HLA-DP, HLA-DM, HLA-DOA, HLA-DOB, HLA-DQ, HLA-DR, RFX5, RFXANK, RFXAP, NFY-A, NFY-B and/or NFY-C.

In some embodiments, the T cells generated by methods according to various embodiments of the present technology have at least 50%, at least 55%, at least 60%, at least 65%, at least 70%, at least 75%, at least 80%, at least 85%, at least 90%, at least 95%, or at least 100% of the T cells having reduced expression of MHC I and/or MHC II molecules. In some embodiments, the T cells generated by methods according to various embodiments of the present technology have at least 50%, at least 55%, at least 60%, at least 65%, at least 70%, at least 75%, at least 80%, at least 85%, at least 90%, at least 95%, or at least 100% of the T cells having reduced expression of B2M and/or CIITA. In some embodiments, the T cells generated by methods according to various embodiments of the present technology have at least 50%, at least 55%, at least 60%, at least 65%, at least 70%, at least 75%, at least 80%, at least 85%, at least 90%, at least 95%, or at least 100% of the T cells having B2M and/CIITA knockout.

In some embodiments, at least 50%, at least 55%, at least 60%, at least 65%, at least 70%, at least 75%, at least 80%, at least 85%, at least 90%, at least 95%, or at least 100% of the T cells in the population have one or more of: (i) reduced expression of CD3; (ii) increased expression of a tolerogenic factor (e.g., CD47) encoded by a transgene; (iii) reduced expression of MHC I and/or MHC II molecules; (iv) reduced expression of B2M and/or CIITA; and (v) B2M and/CIITA knockout. In any of these embodiments, the remainder T cells in the population (e.g., cells that do not possess the described characteristic(s)) may be a heterogeneous population, and each of the remainder T cells may possess none, one, or more (but not all) of the characteristics.

### II. Gene Editing Systems for Insertion of Transgenes

In some aspects, the first transgene encoding a tolerogenic factor and/or the second transgene encoding a CAR can be integrated into the genome of a host cell (e.g., a T cell) using certain methods and compositions described herein.

### A. Random Insertion

In some embodiments, the first transgene encoding a tolerogenic factor and/or the second transgene encoding a CAR can be inserted into a random genomic locus of a host cell. As known to a person skilled in the art, viral vectors, including, for example, retroviral vectors, lentiviral vectors, adenoviral vectors, and adeno-associated viral vectors, are commonly used to deliver genetic material into host cells and randomly insert the foreign or exogenous gene into the host cell genome to facilitate stable expression and replication of the gene.

### B. Site-Directed Insertion (Knock-In)

In some embodiments, the first transgene encoding a tolerogenic factor and/or the second transgene encoding a CAR can be inserted into a specific genomic locus of the host cell. A number of gene editing methods can be used to insert a transgene into a specific genomic locus of choice. Gene editing is a type of genetic engineering in which a nucleotide sequence may be inserted, deleted, modified, or replaced in the genome of a living organism. In some embodiments, the gene editing technology can include systems involving nucleases, integrases, transposases, and/or recombinases. In some embodiments, the gene editing technology mediates single-strand breaks (SSB). In some embodiments, the gene editing technology mediates double-strand breaks (DSB), including in connection with non-homologous end-joining (NHEJ) or homology-directed repair (HDR). In some embodiments, the gene editing technology can include DNA-based editing or prime-editing. In some embodiments, the gene editing technology can include Programmable Addition via Site-specific Targeting Elements (PASTE). In some embodiments, the gene editing technology can include TnpB polypeptides. Many gene editing techniques generally utilize the innate mechanism for cells to repair double-strand breaks (DSBs) in DNA.

Eukaryotic cells repair DSBs by two primary repair pathways: non-homologous end-joining (NHEJ) and homology-directed repair (HDR). HDR typically occurs during late S phase or G2 phase, when a sister chromatid is available to serve as a repair template. NHEJ is more common and can occur during any phase of the cell cycle, but it is more error prone. In gene editing, NHEJ is generally used to produce insertion/deletion mutations (indels), which can produce targeted loss of function in a target gene by shifting the open reading frame (ORF) and producing alterations in the coding region or an associated regulatory region. HDR, on the other hand, is a preferred pathway for producing targeted knock-ins, knockouts, or insertions of specific mutations in the presence of a repair template with homologous sequences. Several methods are known to a skilled artisan to improve HDR efficiency, including, for example, chemical modulation (e.g., treating cells with inhibitors of key enzymes in the NHEJ pathway); timed delivery of the gene editing system at S and G2 phases of the cell cycle; cell cycle arrest at S and G2 phases; and introduction of repair templates with homology sequences. The methods provided herein may utilize HDR-mediated repair, NHEJ-mediated repair, or a combination thereof.

In some embodiments, the methods provided herein for HDR-mediated insertion utilize a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems.

### 1. ZFNs

ZFNs are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3- to 4-bp DNA sequence. Tandem domains can thus potentially bind to an extended nucleotide sequence that is unique within a cell's genome.

Various zinc fingers of known specificity can be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

### 2. TALENs

TALENs are another example of an artificial nuclease which can be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39:e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease can be produced specific to any desired DNA sequence. Similar to ZFNs, TALENs can be introduced into a cell to generate DSBs at a desired target site in the genome, and so can be used to knock out genes or knock in mutations in similar, HDR-mediated pathways. See Boch, Nature Biotech. (2011) 29:135-136; Boch et al., Science (2009) 326:1509-1512; Moscou et al., Science (2009) 326:3501.

### 3. Meganucleases

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29(18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29(18):3757-3774. Members of the NHN family are defined by motifs containing two pairs of conserved histidines surrounded by asparagine residues. See Chevalier et al., Nucleic Acids Res. (2001) 29(18):3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363(2):283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

### 4. Transposases

Transposases are enzymes that bind to the end of a transposon and catalyze its movement to another part of the genome by a cut and paste mechanism or a replicative transposition mechanism. By linking transposases to other systems such as the CRISPR/Cas system, new gene editing tools can be developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

### 5. CRISPR/Cas

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, III, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. See, e.g., Jinek et al., Science (2012) 337 (6096):816-821; Dang et al., Genome Biology (2015) 16:280; Ran et al., Nature (2015) 520:186-191; Zetsche et al., Cell (2015) 163:759-771; Strecker et al., Nature Comm. (2019) 10:212; Yan et al., Science (2019) 363:88-91. The most widely used Cas9 is a type II Cas protein and is described herein as illustrative. These Cas proteins may be originated from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

While the foregoing description has focused on Cas9 nuclease, it should be appreciated that other RNA-guided nucleases exist which utilize gRNAs that differ in some ways from those described to this point. For instance, Cpf1 (CRISPR from Prevotella and Franciscella 1; also known as Cas12a) is an RNA-guided nuclease that only requires a crRNA and does not need a tracrRNA to function.

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA:tracrRNA complexes, including in certain embodiments via a single gRNA. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing, which are summarized in Table 17 below.

TABLE 17
Exemplary Cas nuclease variants and their PAM sequences

| CRISPR Nuclease | Source Organism | PAM Sequence (5′→3′) |
|---|---|---|
| SpCas9 | Streptococcus pyogenes | ngg or nag |
| SaCas9 | Staphylococcus aureus | ngrrt or ngrrn |
| NmeCas9 | Neisseria meningitidis | nnnngatt |
| CjCas9 | Campylobacter jejuni | nnnnryac |
| StCas9 | Streptococcus thermophilus | nnagaaw |
| TdCas9 | Treponema denticola | naaaac |
| LbCas12a (Cpf1) | Lachnospiraceae bacterium | tttv |
| AsCas 12a (Cpf1) | Acidaminococcus sp. | tttv |
| AacCas12b | Alicyclobacillus acidiphilus | ttn |
| BhCas12b v4 | Bacillus hisashii | attn, tttn, or gttn |

r = a or g; y = c or t; w = a or t; v = a or c or g; n = any base

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HypaSpCas9, HeFSpCas9, and evoSpCas9 high-fidelity variants of SpCas9). For another example, the Cas nuclease may have one or more mutations that alter its PAM specificity.

In some embodiments, CRISPR systems of the present disclosure comprise TnpB polypeptides. In some embodiments, TnpB polypeptides may comprise a Ruv-C-like domain. The RuvC domain may be a split RuvC domain comprising RuvC-I, RuvC-II, and RuvC-III subdomains. In some embodiments, a TnpB may further comprise one or more of a HTH domain, a bridge helix domain and a zinc finger domain. TnpB polypeptides do not comprise an HNH domain. In one exemplary embodiment, a TnpB protein comprises, starting at the N-terminus: a HTH domain, a RuvC-I subdomain, a bridge helix domain, a RuvC-II sub-domain, a zinger finger domain, and a RuvC-III sub-domain. In some embodiments, a RuvC-III sub-domain forms the C-terminus of a TnpB polypeptide. In some embodiments, a TnpB polypeptide is from Epsilonproteobacteria bacterium, Actinoplanes lobatus strain DSM 43150, Actinomadura celluolosilytica strain DSM 45823, Actinomadura namibiensis strain DSM 44197, Alicyclobacillus macrosprangiidus strain DSM 17980, Lipingzhangella halophila strain DSM 102030, or Ktedonobacter recemifer. In some embodiments, a TnpB polypeptide is from Ktedonobacter racemifer, or comprises a conserved RNA region with similarity to the 5′ ITR of K. racemifer TnpB loci. In some embodiments, a TnpB may comprise a Fanzor protein, a TnpB homolog found in eukaryotic genomes. In some embodiments, a CRISPR system comprising a TnpB polypeptide binds a target adjacent motif (TAM) sequence 5′ of a target polynucleotide. In some embodiments, a TAM is a transposon-associated motif. In some embodiments, a TAM sequence comprises TCA. In some embodiments, a TAM sequence comprises TTCAN. In some embodiments, a TAM sequence comprises TTGAT. In some embodiments, a TAM sequence comprises ATAAA.

In certain embodiments, the first and/or the second transgene may function as a DNA repair template to be integrated into the target site through HDR in associated with a gene editing system (e.g., the CRISPR/Cas system) as described. Generally, the transgene to be inserted would comprise at least the expression cassette encoding the protein of interest (e.g., the tolerogenic factor or CAR) and would optionally also include one or more regulatory elements (e.g., promoters, insulators, enhancers). In certain of these embodiments, the transgene to be inserted would be flanked by homologous sequence immediately upstream and downstream of the target, i.e., left homology arm (LHA) and right homology arm (RHA), specifically designed for the target genomic locus to serve as template for HDR. The length of each homology arm is generally dependent on the size of the insert being introduced, with larger insertions requiring longer homology arms.

In some embodiments, target-primed reverse transcription (TPRT) or prime editing may be used to engineer exogenous genes, such as exogenous transgenes encoding a tolerogenic factor (e.g., CD47) into specific loci. In some embodiments, prime editing mediates targeted insertions, deletions, all 12 possible base-to-base conversions, and combinations thereof in human cells without requiring DSBs or donor DNA templates.

Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors search and locate the desired target site to be edited, and encode a replacement strand containing a desired edit which is installed in place of the corresponding target site endogenous DNA strand at the same time. For example, prime editing can be adapted for conducting precision CRISPR/Cas-based genome editing in order to bypass double stranded breaks. In some embodiments, a homologous protein is or encodes for a Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. In some embodiments, a prime editor protein is paired with two prime editing guide RNAs (pegRNAs) that template the synthesis of complementary DNA flaps on opposing strands of genomic DNA, resulting in the replacement of endogenous DNA sequence between the PE-induced nick sites with pegRNA-encoded sequences.

In some embodiments, a gene editing technology is associated with a prime editor that is a reverse transcriptase, or any DNA polymerase known in the art. Thus, in one aspect, a prime editor may comprise Cas9 (or an equivalent napDNAbp) which is programmed to target a DNA sequence by associating it with a specialized guide RNA (i.e., PEgRNA) containing a spacer sequence that anneals to a complementary protospacer in the target DNA. Such methods include any disclosed in Anzalone et al., (doi.org/10.1038/s41586-019-1711-4), or in PCT publication Nos. WO2020191248, WO2021226558, or WO2022067130, which are hereby incorporated in their entirety.

In some embodiments, the base editing technology may be used to introduce single-nucleotide variants (SNVs) into DNA or RNA in living cells. Base editing is a CRISPR-Cas9-based genome editing technology that allows the introduction of point mutations in RNAs or DNAs without generating DSBs. Base editors (BEs) are typically fusions of a Cas (“CRISPR-associated”) domain and a nucleobase modification domain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”)) domains. In some embodiments, base editors may also include proteins or domains that alter cellular DNA repair processes to increase the efficiency and/or stability of the resulting single-nucleotide change. Two major classes of base editors have been developed: cytidine base editors (CBEs) (e.g., BE4) that allow C:G to T:A conversions and adenine base editors (ABEs) (e.g., ABE7.10) that allow A:T to G:C conversions. Base editors are composed by a catalytically dead Cas9 (dCas9) or a nickase Cas9 (nCas9) fused to a deaminase and guided by a sgRNA to the locus of interest. The d/nCas9 recognizes a specific PAM sequence and the DNA unwinds thanks to the complementarity between the sgRNA and the DNA sequence usually located upstream of the PAM (also called protospacer). Then, the opposite DNA strand is accessible to the deaminase that converts the bases located in a specific DNA stretch of the protospacer. Compared to HDR-based strategies, base editing is a promising tool to precisely correct genetic mutations as it avoids gene disruption by NHEJ associated with failed HDR-mediated gene correction. Rat deaminase APOBEC1 (rAPOBEC1) fused to deactivated Cas9 (dCas9) has been used to successfully convert cytidines to thymidines upstream of the PAM of the sgRNA. In some embodiments, this first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U:A base pair, which is then converted to T:A during DNA replication.

In some embodiments, a base editor is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, a base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, a base editor is a fusion protein that comprises a DNA domain of a CRISPR-Cas (e.g., Cas9) having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, a base editor is an adenine-to-thymine or “ATBE” (or thymine-to-adenine or “TABE”) transversion base editor. Exemplary base editor and base editor systems include any as described in patent publication Nos. US20220127622, US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, a gene editing technology is Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Ioannidi et al. (doi.org/10.1101/2021.11.01.466786), PASTE does not generate double stranded breaks, but allows for integration of sequences as large as ˜36 kb. In some embodiments, a serine integrase can be any known in the art. In some embodiments, a serine integrase has sufficient orthogonality such that PASTE can be used for multiplexed gene integration, simultaneously integrating at least two different genes at at least two genomic loci. In some embodiments, PASTE has editing efficiencies comparable to or better than those of homology directed repair or non-homologous end joining based integration, with activity in non-dividing cells and fewer detectable off-target events.

### C. Genomic Loci for Insertion of the First Transgene

In some embodiments, the genomic locus for site-directed insertion of the first transgene encoding a tolerogenic factor is an endogenous TCR gene locus. In some embodiments, the endogenous TCR gene locus is selected from the group consisting of a TRAC locus, a TRBC1 locus, and a TRBC2 locus. The specific site for insertion within a gene locus may be located within any suitable region of the gene, including but not limited to a gene coding region (also known as a coding sequence or “CDS”), an exon, an intron, a sequence spanning a portion of an exon and a portion of an adjacent intron, or a regulatory region (e.g., promoter, enhancer). In some embodiments, the insertion occurs in one allele of the specific genomic locus. In some embodiments, the insertion occurs in both alleles of the specific genomic locus. In either of these embodiments, the orientation of the transgene inserted into the target genomic locus can be either the same or the reverse of the direction of the endogenous gene in that locus.

### 1. TRAC

TCRs recognize foreign antigens which have been processed as small peptides and bound to MHC molecules at the surface of antigen presenting cells (APC). Each TCR is a dimer consisting of one alpha and one beta chain (most common) or one delta and one gamma chain. The genes encoding the TCR alpha chain are clustered on chromosome 14. The TCR alpha chain is formed when one of at least 70 variable (V) genes, which encode the N-terminal antigen recognition domain, rearranges to 1 of 61 joining (J) gene segments to create a functional variable region that is transcribed and spliced to a constant region gene segment encoding the C-terminal portion of the molecule. The beta chain, on the other hand, is generated by recombination of the V, D (diversity), and J segment genes.

The TRAC gene encodes the TCR alpha chain constant region. The human TRAC gene resides on chromosome 14 at 22,547,506-22,552,156, forward strand. The TRAC genomic sequence is set forth in Ensembl ID ENSG00000277734.

### 2. TRBC1 and TRBC2

The TRBC gene encodes the TCR beta chain constant region. TRBC1 and TRBC2 are analogs of the same gene, and T cells mutually exclusively express either TRBC1 and TRBC2. The human TRBC1 gene resides on chromosome 7 at 142,791,694-142,793,368, forward strand, and its genomic sequence is set forth in Ensembl ID ENSG00000211751. The human TRBC2 gene resides on chromosome 7 at 142,801,041-142,802,748, forward strand, and its genomic sequence is set forth in Ensembl ID ENSG00000211772.

### D. Genomic Loci for Insertion of the Second Transgene

In some embodiments, the genomic locus for insertion of the second transgene encoding a CAR can be a random locus (by random insertion) or a specific locus (by site-directed insertion). If a specific locus is desired, it can be the same as or a different locus from that of the first transgene. In some embodiments, the genomic locus for insertion of the second transgene encoding a CAR is a specific locus selected from the group consisting of a TRAC locus, a TRBC1 locus, a TRBC2 locus, a B2M locus, a CIITA locus, and a safe harbor locus. Non-limiting examples of safe harbor loci include, but are not limited to, an AAVS1 (also known as PPP1R12C), ABO, CCR5, CLYBL, CXCR4, F3 (also known as CD142), FUT1, HMGB1, KDM5D, LRP1 (also known as CD91), MICA, MICB, RHD, ROSA26, and SHS231 gene locus. In some embodiments, the genomic locus for insertion of the second transgene encoding a CAR is a specific locus comprising a TRAC locus, a TRBC1 locus, a TRBC2 locus, a B2M locus, a CIITA locus, an AAVS1 (also known as PPP1R12C) locus, an ABO locus, a CCR5 locus, a CLYBL locus, aCXCR4 locus, an F3 (also known as CD142) locus, a FUT1 locus, an HMGB1 locus, a KDM5D locus, an LRP1 (also known as CD91) locus, a MICA locus, an MICB locus, an RHD locus, a ROSA26 locus, or an SHS231 locus. The second transgene can be inserted within any suitable region of any of the described locus, including but not limited to a gene coding region (also known as a coding sequence or “CDS”), an exon, an intron, a sequence spanning a portion of an exon and a portion of an adjacent intron, or a regulatory region (e.g., promoter, enhancer). In some embodiments, the insertion occurs in one allele of the genomic locus. In some embodiments, the insertion occurs in both alleles of the genomic locus. In either of these embodiments, the orientation of the transgene inserted into the genomic locus can be either the same or the reverse of the direction of the original gene in that locus. In some embodiments, the second transgene is inserted with the first transgene such as the first transgene and the second transgene are carried by a polycistronic vector.

E. Guide RNAs (gRNAs) for Site-Directed Insertion

In some embodiments, provided are gRNAs for use in site-directed insertion of a transgene in according to various embodiments provided herein, especially in association with the CRISPR/Cas system. The gRNAs comprise a crRNA sequence, which in turn comprises a complementary region (also called a spacer) that recognizes and binds a complementary target DNA of interest. The length of the spacer or complementary region is generally between 15 and 30 nucleotides, usually about 20 nucleotides in length, although will vary based on the requirements of the specific CRISPR/Cas system. In certain embodiments, the spacer or complementary region is fully complementary to the target DNA sequence. In other embodiments, the spacer is partially complementary to the target DNA sequence, for example at least 80%, 85%, 90%, 95%, 98%, or 99% complementary.

In certain embodiments, the gRNAs provided herein further comprise a tracrRNA sequence, which comprises a scaffold region for binding to a nuclease. The length and/or sequence of the tracrRNA may vary depending on the specific nuclease being used for editing. In certain embodiments, nuclease binding by the gRNA does not require a tracrRNA sequence. In those embodiments where the gRNA comprises a tracrRNA, the crRNA sequence may further comprise a repeat region for hybridization with complementary sequences of the tracrRNA.

In some embodiments, the gRNAs provided herein comprise two or more gRNA molecules, for example, a crRNA and a tracrRNA, as two separate molecules. In other embodiments, the gRNAs are single guide RNAs (sgRNAs), including sgRNAs comprising a crRNA and a tracrRNA on a single RNA molecule. In certain of these embodiments, the crRNA and tracrRNA are linked by an intervening tetraloop.

In some embodiments, one gRNA can be used in association with a site-directed nuclease for targeted editing of a gene locus of interest. In other embodiments, two or more gRNAs targeting the same gene locus of interest can be used in association with a site-directed nuclease.

In some embodiments, exemplary gRNAs (e.g., sgRNAs) for use with various common Cas nucleases that require both a crRNA and tracrRNA, including Cas9 and Cas12b (C2c1), are provided in Table 18. See, e.g., Jinek et al., Science (2012) 337 (6096):816-821; Dang et al., Genome Biology (2015) 16:280; Ran et al., Nature (2015) 520:186-191; Strecker et al., Nature Comm. (2019) 10:212. For each exemplary gRNA, sequences for different portions of the gRNA, including the complementary region or spacer, crRNA repeat region, tetraloop, and tracrRNA, are shown. In some embodiments, the gRNA comprises all or a portion of the nucleotide sequences set forth in SEQ ID NOs: 108-111. In some embodiments, the gRNA comprises all or a portion of the nucleotide sequences set forth in SEQ ID NOs: 112-115. In some embodiments, the gRNA comprises all or a portion of the nucleotide sequences set forth in SEQ ID NOs: 116-119. In some embodiments, the gRNA comprises all or a portion of the nucleotide sequences set forth in SEQ ID NOs: 120-123.

In some embodiments, the gRNA comprises a crRNA repeat region comprising, consisting of, or consisting essentially of the nucleotide sequence set forth in SEQ ID NO: 109, SEQ ID NO: 113, SEQ ID NO: 117, or SEQ ID NO: 122. In some embodiments, the gRNA comprises a tetraloop comprising, consisting of, or consisting essentially of the nucleotide sequence set forth in SEQ ID NO: 110 or SEQ TD NO: 121. In some embodiments, the gRNA comprises a tracrRNA comprising, consisting of, or consisting essentially of the nucleotide sequence set forth in SEQ ID NO: 111, SEQ ID NO: 115, SEQ ID NO: 119, or SEQ ID NO: 120.

TABLE 18
Exemplary gRNA structure and sequence for CRISPR/Cas

| SEQ ID NO: | Sequence (5′→3′) | Description |
|---|---|---|
| 108 | nnnnnnnnnnnnnnnnnnnn | Exemplary spCas9 1 |
| | | Complementary region |
| | | (spacer) |
| 109 | guuuuagagcua | Exemplary spCas9 1 |
| | | crRNA repeat region |
| 110 | gaaa | Exemplary spCas9 1 |
| | | tetraloop |
| 111 | uagcaaguuaaaauaaggcuaguccguuaucaacuugaaaaa | Exemplary spCas9 1 |
| | | tracrRNA |
| 112 | nnnnnnnnnnnnnnnnnnnn | Exemplary spCas9 2 |
| | | Complementary region |
| | | (spacer) |
| 113 | guuusagagcuaugcug | Exemplary spCas9 2 |
| | | crRNA repeat region |
| 114 | gaaa | Exemplary spCas9 2 |
| | | tetraloop |
| 115 | cagcauagcaaguusaaauaaggcuaguccguuaucaacuug | Exemplary spCas9 2 |
| | | tracrRNA |
| 116 | nnnnnnnnnnnnnnnnnnnn | Exemplary saCas9 |
| | | Complementary region |
| | | (spacer) |
| 117 | guuuuaguacucug | Exemplary saCas9 crRNA |
| | | repeat region |
| 118 | gaaa | Exemplary saCas9 |
| | | tetraloop |
| 119 | cagaaucuacuaaaacaaggcaaaaugccguguuuaucucgu | Exemplary saCas9 |
| | | tracrRNA |
| 120 | gucgucuauaggacggcgaggacaacgggaagugccaaugug | Exemplary AkCas12b |
| | cucuuuccaagagcaaacaccccguuggcuucaagaugaccg | tracrRNA |
| | cucg | |
| 121 | aaaa | Exemplary AkCas12b |
| | | tetraloop |
| 122 | cgagcggucugagaaguggcacu | Exemplary AkCas12b |
| | | crRNA repeat region |
| 123 | nnnnnnnnnnnnnnnnnnnn | Exemplary AkCas12b |
| | | Complementary region |
| | | (spacer) |

s = c or g; n = any base

In some embodiments, the gRNA comprises a complementary region specific to a target gene locus of interest, for example, the TRAC locus, the TRBC1 locus, the TRBC2 locus, B2M locus, the CIITA locus, or a safe harbor locus selected from the group consisting of an AAVS1, ABO, CCR5, CLYBL, CXCR4, F3, FUT1, HMGB1, KDM5D, LRP1, MICA, MICB, RHD, ROSA26, and SHS231 gene locus. The complementary region may bind a sequence in any region of the target gene locus, including for example, a CDS, an exon, an intron, a sequence spanning a portion of an exon and a portion of an adjacent intron, or a regulatory region (e.g., promoter, enhancer). Where the target sequence is a CDS, exon, intron, or sequence spanning portions of an exon and intron, the CDS, exon, intron, or exon/intron boundary may be defined according to any splice variant of the target gene. In some embodiments, the genomic locus targeted by the gRNA is located within 4000 bp, within 3500 bp, within 3000 bp, within 2500 bp, within 2000 bp, within 1500 bp, within 1000 bp, or within 500 bp of any of the loci or regions thereof as described. Further provided herein are compositions comprising one or more gRNAs provided herein and a Cas protein or a nucleotide sequence encoding a Cas protein. In certain of these embodiments, the one or more gRNAs and a nucleotide sequence encoding a Cas protein are comprised within a vector, for example, a viral vector.

In some embodiments, provided are methods of identifying new loci and/or gRNA sequences for use in the site-directed genomic insertion approaches as described. For example, for CRISPR/Cas systems, when an existing gRNA for a particular locus (e.g., within an endogenous TCR gene locus) is known, an “inch worming” approach can be used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus can be between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide can be designed according to the sequence of that locus for use in site-directed insertion of transgenes. Although the CRISPR/Cas system is described as illustrative, any gene editing approaches as described can be used in this method of identifying new loci, including those using ZFNs, TALENs, meganucleases, and transposases.

In some embodiments, the activity, stability, and/or other characteristics of gRNAs can be altered through the incorporation of chemical and/or sequential modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not being bound by a particular theory, it is believed that certain modified gRNAs described herein can exhibit a reduced innate immune response when introduced into a population of cells, particularly the cells of the present technology. As used herein, the term “innate immune response” includes a cellular response to exogenous nucleic acids, including single stranded nucleic acids, generally of viral or bacterial origin, which involves the induction of cytokine expression and release, particularly the interferons, and cell death. Other common chemical modifications of gRNAs to improve stabilities, increase nuclease resistance, and/or reduce immune response include 2′-O-methyl modification, 2′-fluoro modification, 2′-O-methyl phosphorothioate linkage modification, and 2′-O-methyl 3′ thioPACE modification.

One common 3′ end modification is the addition of a poly(A) tract comprising one or more (and typically 5-200) adenine (A) residues. The poly(A) tract can be contained in the nucleic acid sequence encoding the gRNA or can be added to the gRNA during chemical synthesis, or following in vitro transcription using a polyadenosine polymerase (e.g., E. coli poly(A) polymerase). In vivo, poly(A) tracts can be added to sequences transcribed from DNA vectors through the use of polyadenylation signals. Examples of such signals are provided in Maeder. Other suitable gRNA modifications include, without limitations, those described in U.S. Patent Application No. US 2017/0073674 A1 and International Publication No. WO 2017/165862 A1, the entire contents of each of which are incorporated by reference herein.

In some embodiments, a tool for designing a gRNA as disclosed herein comprises: Benchling, Broad Institute GPP, CasOFFinder, CHOPCHOP, CRISPick, CRISPOR, Deskgen, E-CRISP, Geneious, Guides, Horizon Discovery, IDT, Off-Spotter, Synthego, or TrueDesign (ThermoFisher). One of ordinary skill in the art would understand that a tool that predicts both activity and specificity (e.g., to limit off-target modification) would be useful for designing a gRNA in certain instances as disclosed herein.

F. Delivery of Gene Editing Systems into a Host Cell

In some embodiments, provided are compositions comprising one or more components of a gene editing system described herein, including one or more gRNAs, a site-directed nuclease (e.g., a Cas nuclease) or a nucleotide sequence encoding a site-directed nuclease protein, and a transgene for targeted insertion. In some embodiments, the compositions are formulated for delivery into a cell.

In some embodiments, components of a gene editing system provided herein, including one or more gRNAs, a site-directed nuclease (e.g., a Cas nuclease) or a nucleotide sequence encoding a site-directed nuclease protein, and a transgene (e.g., the first transgene encoding a tolerogenic factor and/or the second transgene encoding a CAR) for targeted insertion, may be delivered into a cell in the form of a delivery vector. The delivery vector can be any type of vector suitable for introduction of nucleotide sequences into a cell, including, for example, plasmids, adenoviral vectors, adeno-associated viral (AAV) vectors, retroviral vectors, lentiviral vectors, phages, and HDR-based donor vectors. The different components may be introduced into a cell together or separately, and may be delivered in a single vector or multiple vectors.

In some embodiments, the delivery vector may be introduced into a cell by any known method in the field, including, for example, viral transformation, calcium phosphate transfection, lipid-mediated transfection, DEAE-dextran, electroporation, microinjection, nucleoporation, liposomes, nanoparticles, or other methods.

In some embodiments, the present technology provides compositions comprising a delivery vector according to various embodiments disclosed herein. In some embodiments, the compositions may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient may be a liquid or solid filler, diluent, excipient, solvent, or encapsulating material, or some combination thereof. Each component of the carrier or excipient must be “pharmaceutically acceptable,” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. Suitable excipients include water, saline, dextrose, glycerol, or the like and combinations thereof. In some embodiments, compositions comprising cells as disclosed herein further comprise a suitable infusion media.

In some embodiments, provided are cells or compositions thereof comprising one or more components of a gene editing system described herein, including one or more gRNAs, a site-directed nuclease (e.g., a Cas nuclease) or a nucleotide sequence encoding a site-directed nuclease protein, and a transgene for targeted insertion.

### III. Cells and Compositions Thereof

### A. Hypoimmunogenic Cells

In some embodiments, the present disclosure is directed to pluripotent stem cells (e.g., pluripotent stem cells and induced pluripotent stem cells (iPSCs)), differentiated cells derived from such pluripotent stem cells (such as, but not limited to, T cells and NK cells), and primary cells (such as, but not limited to, primary T cells and primary NK cells). In some embodiments, the pluripotent stem cells, differentiated cells derived therefrom, such as T cells and NK cells, and primary cells such as primary T cells and primary NK cells, are engineered for reduced expression or lack of expression of MHC class I and/or MHC class II human leukocyte antigens, and in some instances, for reduced expression or lack of expression of a T-cell receptor (TCR) complex. In some embodiments, the hypoimmune (HIP) T cells and primary T cells overexpress CD47 and a chimeric antigen receptor (CAR) in addition to reduced expression or lack of expression of MHC class I and/or MHC class II human leukocyte antigens, and have reduced expression or lack expression of a T-cell receptor (TCR) complex. In some embodiments, the CAR comprises an antigen binding domain that binds to any one selected from the group consisting of CD19, CD22, CD38, CD123, CD138, and BCMA. In some embodiments, the CAR is a CD19-specific CAR. In some embodiments, the CAR is a CD22-specific CAR. In some embodiments, the CAR is a CD38-specific CAR. In some embodiments, the CAR is a CD123-specific CAR. In some embodiments, the CAR is a CD138-specific CAR. In some instances, the CAR is a BCMA-specific CAR. In some embodiments, the CAR is a bispecific CAR. In some embodiments, the bispecific CAR is a CD19/CD22-bispecific CAR. In some embodiments, the bispecific CAR is a BCMA/CD38-bispecific CAR. In some embodiments, the cells described express a CD 19-specific CAR and a different CAR, such as, but not limited to a CD22-specific CAR, a CD38-specific CAR, a CD123-specific CAR, a CD138-specific CAR, and a BCMA-specific CAR. In some embodiments, the cells described express a CD22-specific CAR and a different CAR, such as, but not limited to a CD19-specific CAR, a CD38-specific CAR, a CD123-specific CAR, a CD138-specific CAR, and a BCMA-specific CAR. In some embodiments, the cells described express a CD38-specific CAR and a different CAR, such as, but not limited to a CD22-specific CAR, a CD18-specific CAR, a CD123-specific CAR, a CD138-specific CAR, and a BCMA-specific CAR. In some embodiments, the cells described express a CD123-specific CAR and a different CAR, such as, but not limited to a CD22-specific CAR, a CD38-specific CAR, a CD19-specific CAR, a CD138-specific CAR, and a BCMA-specific CAR. In some embodiments, the cells described express a CD138-specific CAR and a different CAR, such as, but not limited to a CD22-specific CAR, a CD38-specific CAR, a CD123-specific CAR, a CD19-specific CAR, and a BCMA-specific CAR. In some embodiments, the cells described express a BCMA-specific CAR and a different CAR, such as, but not limited to a CD22-specific CAR, a CD38-specific CAR, a CD123-specific CAR, a CD138-specific CAR, and a CD19-specific CAR. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, engineered and/or hypoimmune (HIP) T cells and primary T cells overexpress CD47 and a chimeric antigen receptor (CAR), and include a genomic modification of the B2M gene. In some embodiments, engineered and/or hypoimmune (HIP) T cells and primary T cells overexpress CD47 and include a genomic modification of the CIITA gene. In some embodiments, engineered and/or hypoimmune (HIP) T cells and primary T cells overexpress CD47 and a CAR, and include a genomic modification of the TRAC gene. In some embodiments, engineered and/or hypoimmune (HIP) T cells and primary T cells overexpress CD47 and a CAR, and include a genomic modification of the TRB gene. In some embodiments, engineered and/or hypoimmune (HIP) T cells and primary T cells overexpress CD47 and a CAR, and include one or more genomic modifications selected from the group consisting of the B2M, CIIT A, TRAC, and TRB genes. In some embodiments, engineered and/or hypoimmune (HIP) T cells and primary T cells overexpress CD47 and a CAR, and include genomic modifications of the B2M, CIITA, TRAC, and TRB genes. In some embodiments, the cells are B2M^{-/-}, CIITA^{-/-,} TRAC^{-/-,} CD47tg cells that also express CARs. In some embodiments, engineered and/or hypoimmune (HIP) T cells are produced by differentiating induced pluripotent stem cells such as engineered and/or hypoimmunogenic induced pluripotent stem cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, the engineered and/or hypoimmune (HIP) T cells and primary T cells are B2M^{-/-}, CIITA^{-/-}, TRB^{-/-,} CD47tg cells that also express CARs. In some embodiments, the cells are B2M^{-/-}, CIITA^{-/-}, TRAC^{-/-}, TRB^{-/-}, CD47tg cells that also express CARs. In certain embodiments, the cells are B2M^{indel/indel}, CIITA^{indel/indel}, TRAC^{indel/indel}, CD47tg cells that also express CARs. In certain embodiments, the cells are B2M^{indel/indel}, CIITA^{indel/indel}, TRB^{indel/indel}, CD47tg cells that also express CARs. In certain embodiments, the cells are B₂M^{indel/indel}, CIITA^{indel/indel}, TRAC^{indel/indel}, TRB^{indel/indel}, CD47tg cells that also express CARs. In some embodiments, the engineered or modified cells described are pluripotent stem cells, induced pluripotent stem cells, NK cells differentiated from such pluripotent stem cells and induced pluripotent stem cells, T cells differentiated from such pluripotent stem cells and induced pluripotent stem cells, or primary T cells. Non-limiting examples of primary T cells include CD3+ T cells, CD4+ T cells, CDS+ T cells, naive T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tern) cells, effector memory T (Tern) cells, effector memory T cells express CD45RA (TEMRA cells), tissue-resident memory (Trm) cells, virtual memory T cells, innate memory T cells, memory stem cell (Tse), yo T cells, and any other subtype of T cells. In some embodiments, the primary T cells are selected from a group that includes cytotoxic T-cells, helper T-cells, memory T-cells, regulatory T-cells, tumor infiltrating lymphocytes, and combinations thereof. Non-limiting examples of NK cells and primary NK cells include immature NK cells and mature NK cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, the primary T cells are from a pool of primary T cells from one or more donor subjects that are different than the recipient subject (e.g., the patient administered the cells). The primary T cells can be obtained from 1, 2, 3, 4, 5, 6, 7, 8, 9, 10, 20, 50, 100 or more donor subjects and pooled together. The primary T cells can be obtained from 1 or more, 2 or more, 3 or more, 4 or more, 5 or more, 6 or more, 7 or more, 8 or more, 9 or more, 10, or more 20 or more, 50 or more, or 100 or more donor subjects and pooled together. In some embodiments, the primary T cells are harvested from one or a plurality of individuals, and in some instances, the primary T cells or the pool of primary T cells are cultured in vitro. In some embodiments, the primary T cells or the pool of primary T cells are engineered to exogenously express CD47 and cultured in vitro.

In certain embodiments, the primary T cells or the pool of primary T cells are engineered to express a chimeric antigen receptor (CAR). The CAR can be any known to those skilled in the art. Useful CARs include those that bind an antigen selected from a group that includes CD19, CD20, CD22, CD38, CD123, CD138, and BCMA. In some cases, the CAR is the same or equivalent to those used in FDA-approved CAR-T cell therapies such as, but not limited to, those used in tisagenlecleucel and axicabtagene ciloleucel, or others under investigation in clinical trials.

In some embodiments, the primary T cells or the pool of primary T cells are engineered to exhibit reduced expression of an endogenous T cell receptor compared to unmodified primary T cells. In certain embodiments, the primary T cells or the pool of primary T cells are engineered to exhibit reduced expression of CTLA-4, PD-1, or both CTLA-4 and PD-1, as compared to unmodified primary T cells. Methods of genetically modifying a cell including a T cell are described in detail, for example, in WO2020/018620 and WO2016/183041, the disclosures of which are herein incorporated by reference in their entireties, including the tables, appendices, sequence listing and figures.

In some embodiments, the CAR-T cells comprise a CAR selected from a group including: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments, the CAR-T cells comprise a CAR comprising an antigen binding domain, a transmembrane, and one or more signaling domains. In some embodiments, the CAR also comprises a linker. In some embodiments, the CAR comprises a CD 19 antigen binding domain. In some embodiments, the CAR comprises a CD28 or a CD8α transmembrane domain. In some embodiments, the CAR comprises a CD8α signal peptide. In some embodiments, the CAR comprises a Whitlow linker GSTSGSGKPGSGEGSTKG (SEQ ID NO: 126). In some embodiments, the antigen binding domain of the CAR is selected from a group including, but not limited to, (a) an antigen binding domain targets an antigen characteristic of a neoplastic cell; (b) an antigen binding domain that targets an antigen characteristic of a T cell; (c) an antigen binding domain targets an antigen characteristic of an autoimmune or inflammatory disorder; (d) an antigen binding domain that targets an antigen characteristic of senescent cells; (e) an antigen binding domain that targets an antigen characteristic of an infectious disease; and (f) an antigen binding domain that binds to a cell surface antigen of a cell.

In some embodiments, the CAR further comprises one or more linkers. The format of an scFv is generally two variable domains linked by a flexible peptide sequence, or a “linker,” either in the orientation VH-linker-VL or VL-linker-VH. Any suitable linker known to those in the art in view of the specification can be used in the CARs. Examples of suitable linkers include, but are not limited to, a GS based linker sequence, and a Whitlow linker GSTSGSGKPGSGEGSTKG (SEQ ID NO: 126). In some embodiments, the linker is a GS or a gly-ser linker. Exemplary gly-ser polypeptide linkers comprise the amino acid sequence Ser(Gly₄Ser)ₙ, as well as (Gly₄Ser)ₙ and/or (Gly₄Sen)ₙ. In some embodiments, n=1. In some embodiments, n=2. In some embodiments, n=3, i.e., Ser(Gly₄Ser)₃. In some embodiments, n=4, i.e., Ser(Gly₄Ser)₄. In some embodiments, n=5. In some embodiments, n=6. In some embodiments, n=7. In some embodiments, n=8. In some embodiments, n=9. In some embodiments, n=10. Another exemplary gly-ser polypeptide linker comprises the amino acid sequence Ser(Gly₄Ser)ₙ. In some embodiments, n=1. In some embodiments, n=2. In some embodiments, n=3. In another embodiment, n=4. In some embodiments, n=5. In some embodiments, n=6. Another exemplary gly-ser polypeptide linker comprises (Gly₄Ser)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ. In some embodiments, n=1. In some embodiments, n=2. In some embodiments, n=3. In some embodiments, n=4. In another embodiment, n=5. In yet another embodiment, n=6. Another exemplary gly-ser polypeptide linker comprises (Gly₄Sen)ₙ. In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ. In some embodiments, n=1. In some embodiments, n=2. In some embodiments, n=3. In some embodiments, n=4. In another embodiment, n=5. In yet another embodiment, n=6.

In some embodiments, the antigen binding domain is selected from a group that includes an antibody, an antigen-binding portion or fragment thereof, an scFv, and a Fab. In some embodiments, the antigen binding domain binds to CD19, CD20, CD22, CD38, CD123, CD138, or BCMA. In some embodiments, the antigen binding domain is an anti-CD19 scFv such as but not limited to FMC63.

In some embodiments, the transmembrane domain comprises one selected from a group that includes a transmembrane region of TCRα, TCRβ, TCRζ, CD3ε, CD3γ, CD35, CD3ζ, CD4, CDS, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRI γ, VEGFR2, FAS, FGFR2B, and functional variant thereof.

In some embodiments, the signaling domain(s) of the CAR comprises a costimulatory domain(s). For instance, a signaling domain can contain a costimulatory domain. Or, a signaling domain can contain one or more costimulatory domains. In certain embodiments, the signaling domain comprises a costimulatory domain. In other embodiments, the signaling domains comprise costimulatory domains. In some cases, when the CAR comprises two or more costimulatory domains, two costimulatory domains are not the same. In some embodiments, the costimulatory domains comprise two costimulatory domains that are not the same. In some embodiments, the costimulatory domain enhances cytokine production, CAR-T cell proliferation, and/or CAR-T cell persistence during T cell activation. In some embodiments, the costimulatory domains enhance cytokine production, CAR-T cell proliferation, and/or CAR-T cell persistence during T cell activation.

As described herein, a fourth generation CAR can contain an antigen binding domain, a transmembrane domain, three or four signaling domains, and a domain which upon successful signaling of the CAR induces expression of a cytokine gene. In some instances, the cytokine gene is an endogenous or exogenous cytokine gene of the hypoimmunogenic cells. In some cases, the cytokine gene encodes a pro-inflammatory cytokine. In some embodiments, the pro-inflammatory cytokine is selected from a group that includes IL-1, IL-2, IL-9, IL-12, IL-18, TNF, IFN-gamma, and a functional fragment thereof. In some embodiments, the domain which upon successful signaling of the CAR induces expression of the cytokine gene comprises a transcription factor or functional domain or fragment thereof.

In some embodiments, the CAR comprises a CD3 zeta (CD3ζ) domain or an immunoreceptor tyrosine-based activation motif (IT AM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In other embodiments, the CAR comprises (i) a CD3 zeta domain, or an immunoreceptor tyrosine-based activation motif (IT AM), or functional variant thereof; (ii) a CD28 domain or functional variant thereof; and (iii) a 4-1BB domain, or a CD134 domain, or functional variant thereof. In certain embodiments, the CAR comprises (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene. In some embodiments, the CAR comprises a (i) an anti-CD19 scFv; (ii) a CD8α hinge and transmembrane domain or functional variant thereof; (iii) a 4-1BB costimulatory domain or functional variant thereof; and (iv) a CD31; signaling domain or functional variant thereof.

Methods for introducing a CAR construct or producing a CAR-T cells are well known to those skilled in the art. Detailed descriptions are found, for example, in Vormittag et al., Curr Opin Biotechnol, 2018, 53, 162-181; and Eyquem et al., Nature, 2017, 543, 113-117.

In some embodiments, the cells derived from primary T cells comprise reduced expression of an endogenous T cell receptor, for example by disruption of an endogenous T cell receptor gene (e.g., T cell receptor alpha constant region (TRAC) or T cell receptor beta constant region (TRB)). In some embodiments, an exogenous nucleic acid encoding a polypeptide as disclosed herein (e.g., a chimeric antigen receptor, CD47, or another tolerogenic factor disclosed herein) is inserted at the disrupted T cell receptor gene. In some embodiments, an exogenous nucleic acid encoding a polypeptide is inserted at a TRAC or a TRB gene locus.

In some embodiments, the cells derived from primary T cells comprise reduced expression of cytotoxic T-lymphocyte-associated protein 4 (CTLA4) and/or programmed cell death (PD1). Methods of reducing or eliminating expression of CTLA4, PD1 and both CTLA4 and PD1 can include any recognized by those skilled in the art, such as but not limited to, genetic modification technologies that utilize rare-cutting endonucleases and RNA silencing or RNA interference technologies. Non-limiting examples of a rare-cutting endonuclease include any Cas protein, T ALEN, zinc finger nuclease, meganuclease, and/or homing endonuclease. In some embodiments, an exogenous nucleic acid encoding a polypeptide as disclosed herein (e.g., a chimeric antigen receptor, CD47, or another tolerogenic factor disclosed herein) is inserted at a CTLA4 and/or PD1 gene locus. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

In some embodiments, a CD47 transgene is inserted into a pre-selected locus of the cell. In some embodiments, a CD47 transgene is inserted into a random locus of the cell. In some embodiments, a trans gene encoding a CAR is inserted into a pre-selected locus of the cell. In some embodiments, a transgene encoding a CAR is inserted into a random locus of the cell. In certain embodiments, a CD47 transgene and a transgene encoding a CAR are inserted into a pre-selected locus of the cell. In some embodiments, a trans gene encoding a CAR is inserted into a random or pre-selected locus of the cell, including a safe harbor locus, via viral vector transduction/integration. In some embodiments, a CD47 transgene and a transgene encoding a CAR are inserted into a random or pre-selected locus of the cell, including a safe harbor locus, via viral vector transduction/integration. In some embodiments, the vector is a self-inactivating lentiviral vector pseudotyped with a vesicular stomatitis VSVG envelope. In some embodiments, the transgene encoding a CAR is inserted into at least one allele of the cell using viral transduction. In some embodiments, the exogenous polynucleotide is inserted into at least one allele of the cell using a lentivirus based viral vector. The random and/or pre-selected locus can be a safe harbor or target locus. Non-limiting examples of a safe harbor locus include, but are not limited to, a CCR5 gene locus, a PPP1R12C (also known as AAVS1) gene locus, and a CLYBL gene locus, a Rosa gene locus (e.g., ROSA26 gene locus). Non-limiting examples of a target locus include, but are not limited to, a CXCR4 gene locus, an albumin gene locus, a SHS231 gene locus, an F3 gene locus (also known as CD142), a MICA gene locus, a MICB gene locus, a LRP1 gene locus (also known as a CD91 gene locus), a HMGB1 gene locus, an ABO gene locus, ad RHD gene locus, a FUT1 locus, and a KDM5D gene locus. The CD47 transgene can be inserted in Introns 1 or 2 for PPP1R12C (i.e., AAVS1) or CCR5. The CD47 transgene can be inserted in Exons 1 or 2 or 3 for CCR5. The CD47 transgene can be inserted in intron 2 for CLYBL. The CD47 transgene can be inserted in a 500 bp window in Ch-4:58,976,613 (i.e., SHS231). The CD47 trans gene can be insert in any suitable region of the aforementioned safe harbor or target loci that allows for expression of the exogenous polynucleotide, including, for example, an intron, an exon or a coding sequence region in a safe harbor or target locus. In some embodiments, the pre-selected locus is selected from the group consisting of the B2M locus, the CIITA locus, the TRAC locus, and the TRB locus. In some embodiments, the preselected locus is the B2Mlocus. In some embodiments, the pre-selected locus is the CIITA locus. In some embodiments, the pre-selected locus is the TRAC locus. In some embodiments, the pre-selected locus is the TRB locus. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

In some embodiments, a CD47 transgene and a transgene encoding a CAR are inserted into the same locus. In some embodiments, a CD47 transgene and a transgene encoding a CAR are inserted into different loci. In many instances, a CD47 transgene is inserted into a safe harbor or target locus. In many instances, a transgene encoding a CAR is inserted into a safe harbor or target locus. In some instances, a CD47 transgene is inserted into a B2M locus. In some instances, a trans gene encoding a CAR is inserted into a B2M locus. In certain instances, a CD47 transgene is inserted into a CIITA locus. In certain instances, a transgene encoding a CAR is inserted into a CIITA locus. In particular instances, a CD47 transgene is inserted into a TRAC locus. In particular instances, a transgene encoding a CAR is inserted into a TRAC locus. In many other instances, a CD47 transgene is inserted into a TRB locus. In many other instances, a trans gene encoding a CAR is inserted into a TRB locus. In some embodiments, a CD47 transgene and a transgene encoding a CAR are inserted into a safe harbor or target locus (e.g., a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus.

In certain embodiments, a CD47 transgene and a transgene encoding a CAR are inserted into a safe harbor or target locus. In certain embodiments, a CD47 transgene and a trans gene encoding a CAR are controlled by a single promoter and are inserted into a safe harbor or target locus. In certain embodiments, a CD47 transgene and a transgene encoding a CAR are controlled by their own promoters and are inserted into a safe harbor or target locus. In certain embodiments, a CD47 transgene and a transgene encoding a CAR are inserted into a TRAC locus. In certain embodiments, a CD47 transgene and a transgene encoding a CAR are controlled by a single promoter and are inserted into a TRAC locus. In certain embodiments, a CD4 7 transgene and a trans gene encoding a CAR are controlled by their own promoters and are inserted into a TRAC locus. In some embodiments, a CD47 transgene and a transgene encoding a CAR are inserted into a TRB locus. In some embodiments, a CD47 transgene and a transgene encoding a CAR are controlled by a single promoter and are inserted into a TRB locus. In some embodiments, a CD47 transgene and a transgene encoding a CAR are controlled by their own promoters and are inserted into a TRB locus. In other embodiments, a CD47 transgene and a transgene encoding a CAR are inserted into a B2Mlocus. In other embodiments, a CD47 transgene and a transgene encoding a CAR are controlled by a single promoter and are inserted into a B2M locus. In other embodiments, a CD47 transgene and a transgene encoding a CAR are controlled by their own promoters and are inserted into a B2M locus. In various embodiments, a CD47 transgene and a transgene encoding a CAR are inserted into a CIITA locus. In various embodiments, a CD47 transgene and a transgene encoding a CAR are controlled by a single promoter and are inserted into a CIITA locus. In various embodiments, a CD47 transgene and a transgene encoding a CAR are controlled by their own promoters and are inserted into a CIITA locus. In some instances, the promoter controlling expression of any transgene described is a constitutive promoter. In other instances, the promoter for any transgene described is an inducible promoter. In some embodiments, the promoter is an EF1 a promoter. In some embodiments, the promoter is CAG promoter. In some embodiments, a CD47 transgene and a transgene encoding a CAR are both controlled by a constitutive promoter. In some embodiments, a CD47 transgene and a transgene encoding a CAR are both controlled by an inducible promoter. In some embodiments, a CD47 transgene is controlled by a constitutive promoter and a transgene encoding a CAR is controlled by an inducible promoter. In some embodiments, a CD47 transgene is controlled by an inducible promoter and a transgene encoding a CAR is controlled by a constitutive promoter. In various embodiments, a CD47 transgene is controlled by an EF1α promoter and a transgene encoding a CAR is controlled by an EF1α promoter. In some embodiments, a CD47 transgene is controlled by a CAG promoter and a transgene encoding a CAR is controlled by a CAG promoter. In some embodiments, a CD47 transgene is controlled by a CAG promoter and a transgene encoding a CAR is controlled by an EF1α promoter. In some embodiments, a CD47 transgene is controlled by an EF1α promoter and a transgene encoding a CAR is controlled by a CAG promoter. In some embodiments, expression of both a CD47 transgene and a transgene encoding a CAR is controlled by a single EF1α promoter. In some embodiments, expression of both a CD47 transgene and a transgene encoding a CAR is controlled by a single CAG promoter.

In another embodiment, the present disclosure disclosed herein is directed to pluripotent stem cells, (e.g., pluripotent stem cells and induced pluripotent stem cells (iPSCs)), differentiated cells derived from such pluripotent stem cells (e.g., hypoimmune (HIP) T cells), and primary T cells that overexpress CD47 (such as exogenously express CD47 proteins), have reduced expression or lack expression of MHC class I and/or MHC class II human leukocyte antigens, and have reduced expression or lack expression of a T-cell receptor (TCR) complex. In some embodiments, the hypoimmune (HIP) T cells and primary T cells overexpress CD47 (such as exogenously express CD47 proteins), have reduced expression or lack expression of MHC class I and/or MHC class II human leukocyte antigens, and have reduced expression or lack expression of a T-cell receptor (TCR) complex.

In some embodiments, pluripotent stem cells, (e.g., pluripotent stem cells and induced pluripotent stem cells (iPSCs)), differentiated cells derived from such pluripotent stem cells (e.g., hypoimmune (HIP) T cells), and primary T cells overexpress CD47 and include a genomic modification of the B2M gene. In some embodiments, pluripotent stem cells, differentiated cell derived from such pluripotent stem cells and primary T cells overexpress CD47 and include a genomic modification of the CIITA gene. In some embodiments, pluripotent stem cells, T cells differentiated from such pluripotent stem cells and primary T cells overexpress CD47 and include a genomic modification of the TRAC gene. In some embodiments, pluripotent stem cells, T cells differentiated from such pluripotent stem cells and primary T cells overexpress CD47 and include a genomic modification of the TRB gene. In some embodiments, pluripotent stem cells, T cells differentiated from such pluripotent stem cells and primary T cells overexpress CD47 and include one or more genomic modifications selected from the group consisting of the B2M, CIITA, TRAC and TRB genes. In some embodiments, pluripotent stem cells, T cells differentiated from such pluripotent stem cells and primary T cells overexpress CD47 and include genomic modifications of the B2M, CIITA and TRAC genes. In some embodiments, pluripotent stem cells, T cells differentiated from such pluripotent stem cells and primary T cells overexpress CD47 and include genomic modifications of the B2M, CIITA and TRB genes. In some embodiments, pluripotent stem cells, T cells differentiated from such pluripotent stem cells and primary T cells overexpress CD47 and include genomic modifications of the B2M, CIITA, TRAC and TRB genes. In certain embodiments, the pluripotent stem cells, differentiated cell derived from such pluripotent stem cells and primary T cells are B2M^{-/-}, CIITA^{-/-}, TRAC^{-/-}, CD47tg cells. In certain embodiments, the cells are B2M^{-/-}, CIITA^{-/-}, TRB^{-/-}, CD47tg cells. In certain embodiments, the cells are B2M^{-/-}, CIITA^{-/-}, TRAC^{-/-}, TRB^{-/-}, CD47tg cells. In some embodiments, the cells are B2M^{indel/indel}, CIITA^{indel/indel}, TRAC^{indel/indel}, CD47tg cells. In some embodiments, the cells are B2M^{indel/indel}, CIITA^{indel/indel}, TRB^{indel/indel}, CD47tg cells. In some embodiments, the cells are B2M^{indel/indel}, CIITA^{indel/indel}, TRAC^{indel/indel}, TRB^{indel/indel}, CD47tg cells. In some embodiments, the engineered or modified cells described are pluripotent stem cells, T cells differentiated from such pluripotent stem cells or primary T cells. Non-limiting examples of primary T cells include CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, a CD47 transgene is inserted into a pre-selected locus of the cell. The pre-selected locus can be a safe harbor or target locus. Non-limiting examples of a safe harbor or target locus includes a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus. In some embodiments, the pre-selected locus is the TRAC locus. In some embodiments, a CD47 transgene is inserted into a safe harbor or target locus (e.g., a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus. In certain embodiments, a CD47 transgene is inserted into the B2M locus. In certain embodiments, a CD47 transgene is inserted into the B2M locus. In certain embodiments, a CD47 transgene is inserted into the TRAC locus. In certain embodiments, a CD47 transgene is inserted into the TRB locus. In some embodiments, the CD47 transgene is inserted into a pre-selected locus of the cell, including a safe harbor locus, via viral vector transduction/integration. In some embodiments, the vector is a self-inactivating lentiviral vector pseudotyped with a vesicular stomatitis VSV-G envelope. In some embodiments, the CD47 transgene is inserted into at least one allele of the cell using viral transduction. In some embodiments, the exogenous polynucleotide is inserted into at least one allele of the cell using a lentivirus based viral vector.

In some instances, expression of a CD47 transgene is controlled by a constitutive promoter. In other instances, expression of a CD47 transgene is controlled by an inducible promoter. In some embodiments, the promoter is an EF1alpha (EF1α) promoter. In some embodiments, the promoter a CAG promoter.

In yet another embodiment, the present disclosure disclosed herein is directed to pluripotent stem cells, (e.g., pluripotent stem cells and induced pluripotent stem cells (iPSCs)), T cells derived from such pluripotent stem cells (e.g., hypoimmune (HIP) T cells), and primary T cells that have reduced expression or lack expression of MHC class I and/or MHC class II human leukocyte antigens and have reduced expression or lack expression of a T-cell receptor (TCR) complex. In some embodiments, the cells have reduced or lack expression of MHC class I antigens, MHC class II antigens, and TCR complexes.

In some embodiments, pluripotent stem cells (e.g., iPSCs), differentiated cells derived from such (e.g., T cells differentiated from such), and primary T cells include a genomic modification of the B2M gene. In some embodiments, pluripotent stem cells (e.g., iPSCs), differentiated cells derived from such (e.g., T cells differentiated from such), and primary T cells include a genomic modification of the CIITA gene. In some embodiments, pluripotent stem cells (e.g., iPSCs), T cells differentiated from such, and primary T cells include a genomic modification of the TRAC gene. In some embodiments, pluripotent stem cells (e.g., iPSCs), T cells differentiated from such, and primary T cells include a genomic modification of the TRB gene. In some embodiments, pluripotent stem cells (e.g., iPSCs), T cells differentiated from such, and primary T cells include one or more genomic modifications selected from the group consisting of the B2M, CIITA and TRAC genes. In some embodiments, pluripotent stem cells (e.g., iPSCs), T cells differentiated from such, and primary T cells include one or more genomic modifications selected from the group consisting of the B2M, CIITA and TRB genes. In some embodiments, pluripotent stem cells (e.g., iPSCs), T cells differentiated from such, and primary T cells include one or more genomic modifications selected from the group consisting of the B2M, CIITA, TRAC and TRB genes. In certain embodiments, the cells including iPSCs, T cells differentiated from such, and primary T cells are B2M^{-/-}, CIITA^{-/-}, TRAC^{-/-} cells. In certain embodiments, the cells including iPSCs, T cells differentiated from such, and primary T cells are B2M^{-/-}, CIITA^{-/-}, TRB^{-/-} cells. In some embodiments, the cells including iPSCs, T cells differentiated from such, and primary T cells are B2M^{indel/indel}, CIITA^{indel/indel}, TRAC^{indel/indel} cells. In some embodiments, the cells including iPSCs, T cells differentiated from such, and primary T cells are B2M^{indel/indel}, CIITA^{indel/indel}, TRB^{indel/indel} cells. In some embodiments, the cells including iPSCs, T cells differentiated from such, and primary T cells are B2M^{indel/indel}, CIITA^{indel/indel}, TRAC^{indel/indel}, TRB^{indel/indel} cells. In some embodiments, the modified cells described are pluripotent stem cells, induced pluripotent stem cells, T cells differentiated from such pluripotent stem cells and induced pluripotent stem cells, or primary T cells. Non-limiting examples of primary T cells include CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector I (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

Cells of the present disclosure exhibit reduced or lack expression of MHC class I antigens, MHC class II antigens, and/or TCR complexes. Reduction of MHC I and/or MHC II expression can be accomplished, for example, by one or more of the following: (1) targeting the polymorphic HLA alleles (HLA-A, HLA-B, HLA-C) and MHC-II genes directly; (2) removal of B2M, which will prevent surface trafficking of all MIC-I molecules; (3) removal of CIITA, which will prevent surface trafficking of all MHC-II molecules; and/or (4) deletion of components of the MHC enhanceosomes, such as LRC5, RFX5, RFXANK, RFXAP, IRF1, NF-Y (including NFY-A, NFY-B, NFY-C), and CIITA that are critical for HLA expression.

In some embodiments, HLA expression is interfered with by targeting individual HLAs (e.g., knocking out, knocking down, or reducing expression of HLA-A, HLA-B, HLA-C, HLA-DP, HLA-DQ, and/or HLA-DR), targeting transcriptional regulators of HLA expression (e.g., knocking out, knocking down, or reducing expression of NLRC5, CIITA, RFX5, RFXAP, RFXANK, NFY-A, NFY-B, NFY-C and/or IRF-1), blocking surface trafficking of MHC class I molecules (e.g., knocking out, knocking down, or reducing expression of B2M and/or TAP1), and/or targeting with HLA-Razor (see, e.g., WO2016183041).

In some embodiments, the cells disclosed herein including, but not limited to, pluripotent stem cells, induced pluripotent stem cells, differentiated cells derived from such stem cells, and primary T cells do not express one or more human leukocyte antigens (e.g., HLA-A, HLA-B, HLA-C, HLA-DP, HLA-DQ, and/or HLA-DR) corresponding to MHC-I and/or MHC-II and are thus characterized as being hypoimmunogenic. For example, in certain embodiments, the pluripotent stem cells and induced pluripotent stem cells disclosed have been modified such that the stem cell or a differentiated stem cell prepared therefrom do not express or exhibit reduced expression of one or more of the following MHC-I molecules: HLA-A, HLA-B and HLA-C. In some embodiments, one or more of HLA-A, HLA-B and HLA-C may be “knocked-out” of a cell. A cell that has a knocked-out HLA-A gene, HLA-B gene, and/or HLA-C gene may exhibit reduced or eliminated expression of each knocked-out gene.

In some embodiments, guide RNAs, shRNAs, siRNAs, or miRNAs that allow simultaneous deletion of all MHC class I alleles by targeting a conserved region in the HLA genes are identified as HLA Razors. In some embodiments, the gRNAs are part of a CRISPR system. In alternative embodiments, the gRNAs are part of a TALEN system. In some embodiments, an HLA Razor targeting an identified conserved region in HLAs is described in WO2016183041. In some embodiments, multiple HLA Razors targeting identified conserved regions are utilized. It is generally understood that any guide, siRNA, shRNA, or miRNA molecule that targets a conserved region in HLAs can act as an HLA Razor.

Methods provided are useful for inactivation or ablation of MHC class I expression and/or MHC class II expression in cells such as but not limited to pluripotent stem cells, differentiated cells, and primary T cells. In some embodiments, genome editing technologies utilizing rare-cutting endonucleases (e.g., the CRISPR/Cas, TALEN, zinc finger nuclease, meganuclease, and homing endonuclease systems) are also used to reduce or eliminate expression of genes involved in an immune response (e.g., by deleting genomic DNA of genes involved in an immune response or by insertions of genomic DNA into such genes, such that gene expression is impacted) in cells. In certain embodiments, genome editing technologies or other gene modulation technologies are used to insert tolerance-inducing factors in human cells, rendering them and the differentiated cells prepared therefrom hypoimmunogenic cells. As such, the hypoimmunogenic cells have reduced or eliminated expression of MHC I and MHC II expression. In some embodiments, the cells are nonimmunogenic (e.g., do not induce an innate and/or an adaptive immune response) in a recipient subject.

In some embodiments, the cell includes a modification to increase expression of CD47 and one or more factors selected from the group consisting of DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, CD16, CD52, H2-M3, CD16 Fc receptor, IL15-RF, H2-M3(HLA-G), B2M-HLA-E, A20/TNFAIP3, CR1, HLA-F, MANF, and/or Serpinb9.

In some embodiments, the cell comprises a genomic modification of one or more target polynucleotide sequences that regulate the expression of either MHC class I molecules, MHC class II molecules, or MHC class I and MHC class II molecules. In some embodiments, a genetic editing system is used to modify one or more target polynucleotide sequences. In some embodiments, the targeted polynucleotide sequence is one or more selected from the group including B2M, CIITA, and NLRC5. In some embodiments, the cell comprises a genetic editing modification to the B2M gene. In some embodiments, the cell comprises a genetic editing modification to the CIITA gene. In some embodiments, the cell comprises a genetic editing modification to the NLRC5 gene. In some embodiments, the cell comprises genetic editing modifications to the B2M and CIITA genes. In some embodiments, the cell comprises genetic editing modifications to the B2M and NLRC5 genes. In some embodiments, the cell comprises genetic editing modifications to the CIITA and NLRC5 genes. In numerous embodiments, the cell comprises genetic editing modifications to the B2M, CIITA and NLRC5 genes. In certain embodiments, the genome of the cell has been altered to reduce or delete critical components of HLA expression. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, the present disclosure provides a cell (e.g., stem cell, induced pluripotent stem cell, differentiated cell such as a primary NK cell, CAR-NK cell, primary T cell or CAR-T cell) or population thereof comprising a genome in which a gene has been edited to delete a contiguous stretch of genomic DNA, thereby reducing or eliminating surface expression of MHC class I molecules in the cell or population thereof. In certain embodiments, the present disclosure provides a cell (e.g., stem cell, induced pluripotent stem cell, differentiated cell such as a primary NK cell, CAR-NK cell, primary T cell or CAR-T cell) or population thereof comprising a genome in which a gene has been edited to delete a contiguous stretch of genomic DNA, thereby reducing or eliminating surface expression of MHC class II molecules in the cell or population thereof. In numerous embodiments, the present disclosure provides a cell (e.g., stem cell, induced pluripotent stem cell, differentiated cell, hematopoietic stem cell, primary T cell or CAR-T cell) or population thereof comprising a genome in which one or more genes has been edited to delete a contiguous stretch of genomic DNA, thereby reducing or eliminating surface expression of MHC class I and II molecules in the cell or population thereof.

In certain embodiments, the expression of MHC I molecules and/or MHC II molecules is modulated by targeting and deleting a contiguous stretch of genomic DNA, thereby reducing or eliminating expression of a target gene selected from the group consisting of B2M, CIITA, and NLRC5. In some embodiments, described herein are genetically edited cells (e.g., modified human cells) comprising exogenous CD47 proteins and inactivated or modified CIITA gene sequences, and in some instances, additional gene modifications that inactivate or modify B2M gene sequences. In some embodiments, described herein are genetically edited cells comprising exogenous CD47 proteins and inactivated or modified CIITA gene sequences, and in some instances, additional gene modifications that inactivate or modify NLRC5 gene sequences. In some embodiments, described herein are genetically edited cells comprising exogenous CD47 proteins and inactivated or modified B2M gene sequences, and in some instances, additional gene modifications that inactivate or modify NLRC5 gene sequences. In some embodiments, described herein are genetically edited cells comprising exogenous CD47 proteins and inactivated or modified B2M gene sequences, and in some instances, additional gene modifications that inactivate or modify CIITA gene sequences and NLRC5 gene sequences.

Provided herein are cells exhibiting a modification of one or more targeted polynucleotide sequences that regulates the expression of any one of the following: (a) MHC I antigens, (b) MHC II antigens, (c) TCR complexes, (d) both MHC I and II antigens, and (e) MHC I and II antigens and TCR complexes. In certain embodiments, the modification includes increasing expression of CD47. In some embodiments, the cells include an exogenous or recombinant CD47 polypeptide. In certain embodiments, the modification includes expression of a chimeric antigen receptor. In some embodiments, the cells comprise an exogenous or recombinant chimeric antigen receptor polypeptide.

In some embodiments, the cell includes a genomic modification of one or more targeted polynucleotide sequences that regulates the expression of MHC I antigens, MHC II antigens and/or TCR complexes. In some embodiments, a genetic editing system is used to modify one or more targeted polynucleotide sequences. In some embodiments, the polynucleotide sequence targets one or more genes selected from the group consisting of B2M, CIITA, TRAC, and TRB. In certain embodiments, the genome of a T cell (e.g., a T cell differentiated from hypoimmunogenic iPSCs and a primary T cell) has been altered to reduce or delete critical components of HLA and TCR expression, e.g., HLA-A antigen, HLA-B antigen, HLA-C antigen, HLA-DP antigen, HLA-DQ antigen, HLA-DR antigens, TCR-alpha and TCR-beta.

In some embodiments, the present disclosure provides a cell or population thereof comprising a genome in which a gene has been edited to delete a contiguous stretch of genomic DNA, thereby reducing or eliminating surface expression of MHC class I molecules in the cell or population thereof. In certain embodiments, the present disclosure provides a cell or population thereof comprising a genome in which a gene has been edited to delete a contiguous stretch of genomic DNA, thereby reducing or eliminating surface expression of MHC class II molecules in the cell or population thereof. In certain embodiments, the present disclosure provides a cell or population thereof comprising a genome in which a gene has been edited to delete a contiguous stretch of genomic DNA, thereby reducing or eliminating surface expression of TCR molecules in the cell or population thereof. In numerous embodiments, the present disclosure provides a cell or population thereof comprising a genome in which one or more genes has been edited to delete a contiguous stretch of genomic DNA, thereby reducing or eliminating surface expression of MHC class I and II molecules and TCR complex molecules in the cell or population thereof.

In some embodiments, the cells and methods described herein include genomically editing human cells to cleave CIITA gene sequences as well as editing the genome of such cells to alter one or more additional target polynucleotide sequences such as, but not limited to, B2M TRAC, and TRB. In some embodiments, the cells and methods described herein include genomically editing human cells to cleave B2M gene sequences as well as editing the genome of such cells to alter one or more additional target polynucleotide sequences such as, but not limited to, CIITA, TRAC, and TRB. In some embodiments, the cells and methods described herein include genomically editing human cells to cleave TRAC gene sequences as well as editing the genome of such cells to alter one or more additional target polynucleotide sequences such as, but not limited to, B2M, CIITA, and TRB. In some embodiments, the cells and methods described herein include genomically editing human cells to cleave TRB gene sequences as well as editing the genome of such cells to alter one or more additional target polynucleotide sequences such as, but not limited to, B2M, CIITA, and TRAC.

Provided herein are hypoimmunogenic stem cells comprising reduced expression of HLA-A, HLA-B, HLA-C, HLA-DP, HLA-DQ, HLA-DR, B2M, CIITA, TCR-alpha, and TCR-beta relative to a wild-type stem cell, the hypoimmunogenic stem cell further comprising a set of exogenous polynucleotides comprising a first exogenous polynucleotide encoding CD47 and a second exogenous polynucleotide encoding a chimeric antigen receptor (CAR), wherein the first and/or second exogenous polynucleotides are inserted into a specific locus of at least one allele of the cell. Also provided herein are hypoimmunogenic primary T cells including any subtype of primary T cells comprising reduced expression of HLA-A, HLA-B, HLA-C, HLA-DP, HLA-DQ, HLA-DR, B2M, CIITA, TCR-alpha, and TCR-beta relative to a wild-type primary T cell, the hypoimmunogenic stem cell further comprising a set of exogenous polynucleotides comprising a first exogenous polynucleotide encoding CD47 and a second exogenous polynucleotide encoding a chimeric antigen receptor (CAR), wherein the first and/or second exogenous polynucleotides are inserted into a specific locus of at least one allele of the cell. Further provided herein are hypoimmunogenic T cells differentiated from hypoimmunogenic induced pluripotent stem cells comprising reduced expression of HLA-A, HLA-B, HLA-C, HLA-DP, HLA-DQ, HLA-DR, B2M, CIITA, TCR-alpha, and TCR-beta relative to a wild-type primary T cell, the hypoimmunogenic stem cell further comprising a set of exogenous polynucleotides comprising a first exogenous polynucleotide encoding CD47 and a second exogenous polynucleotide encoding a chimeric antigen receptor (CAR), wherein the first and/or second exogenous polynucleotides are inserted into a specific locus of at least one allele of the cell.

In some embodiments, the population of engineered cells described evades NK cell mediated cytotoxicity upon administration to a recipient patient. In some embodiments, the population of engineered cells evades NK cell mediated cytotoxicity by one or more subpopulations of NK cells. In some embodiments, the population of engineered is protected from cell lysis by NK cells, including immature and/or mature NK cells upon administration to a recipient patient. In some embodiments, the population of engineered cells evades macrophage engulfment upon administration to a recipient patient. In some embodiments, the population of engineered cells does not induce an innate and/or an adaptive immune response to the cell upon administration to a recipient patient.

In some embodiments, the cells described herein comprise a safety switch. The term “safety switch” used herein refers to a system for controlling the expression of a gene or protein of interest that, when downregulated or upregulated, leads to clearance or death of the cell, e.g., through recognition by the host's immune system. A safety switch can be designed to be triggered by an exogenous molecule in case of an adverse clinical event. A safety switch can be engineered by regulating the expression on the DNA, RNA and protein levels. A safety switch includes a protein or molecule that allows for the control of cellular activity in response to an adverse event. In one embodiment, the safety switch is a “kill switch” that is expressed in an inactive state and is fatal to a cell expressing the safety switch upon activation of the switch by a selective, externally provided agent. In one embodiment, the safety switch gene is cis-acting in relation to the gene of interest in a construct. Activation of the safety switch causes the cell to kill solely itself or itself and neighboring cells through apoptosis or necrosis. In some embodiments, the cells described herein, e.g., stem cells, induced pluripotent stem cells, hematopoietic stem cells, primary cells, or differentiated cell, including, but not limited to, T cells, CAR-T cells, NK cells, and/or CAR-NK cells, comprise a safety switch.

In some embodiments, the safety switch comprises a therapeutic agent that inhibits or blocks the interaction of CD47 and SIRPα. In some aspects, the CD47-SIRPα blockade agent is an agent that neutralizes, blocks, antagonizes, or interferes with the cell surface expression of CD47, SIRPα, or both. In some embodiments, the CD47-SIRPα blockade agent inhibits or blocks the interaction of CD47, SIRPα or both. In some embodiments, a CD47-SIRPα blockade agent (e.g., a CD47-SIRPα blocking, inhibiting, reducing, antagonizing, neutralizing, or interfering agent) comprises an agent selected from a group that includes an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof.

In some embodiments, the cells described herein comprise a “suicide gene” (or “suicide switch”). The suicide gene can cause the death of the hypoimmunogenic cells should they grow and divide in an undesired manner. The suicide gene ablation approach includes a suicide gene in a gene transfer vector encoding a protein that results in cell killing only when activated by a specific compound. A suicide gene can encode an enzyme that selectively converts a nontoxic compound into highly toxic metabolites. In some embodiments, the cells described herein, e.g., stem cells, induced pluripotent stem cells, hematopoietic stem cells, primary cells, or differentiated cell, including, but not limited to, T cells, CAR-T cells, NK cells, and/or CAR-NK cells, comprise a suicide gene.

In some embodiments, the population of engineered cells described elicits a reduced level of immune activation or no immune activation upon administration to a recipient subject. In some embodiments, the cells elicit a reduced level of systemic TH1 activation or no systemic TH1 activation in a recipient subject. In some embodiments, the cells elicit a reduced level of immune activation of peripheral blood mononuclear cells (PBMCs) or no immune activation of PBMCs in a recipient subject. In some embodiments, the cells elicit a reduced level of donor-specific IgG antibodies or no donor specific IgG antibodies against the cells upon administration to a recipient subject. In some embodiments, the cells elicit a reduced level of IgM and IgG antibody production or no IgM and IgG antibody production against the cells in a recipient subject. In some embodiments, the cells elicit a reduced level of cytotoxic T cell killing of the cells upon administration to a recipient subject.

### B. CIITA

In some embodiments, the technologies disclosed herein modulate (e.g., reduces or eliminates) the expression of MHC II genes by targeting and modulating (e.g., reducing or eliminating) Class II transactivator (CIITA) expression. In some embodiments, the modulation occurs using a CRISPR/Cas system. CIITA is a member of the LR or nucleotide binding domain (NBD) leucine-rich repeat (LRR) family of proteins and regulates the transcription of MHC II by associating with the MHC enhanceosome.

In some embodiments, the target polynucleotide sequence of the present disclosure is a variant of CIITA. In some embodiments, the target polynucleotide sequence is a homolog of CIITA. In some embodiments, the target polynucleotide sequence is an ortholog of CIITA.

In some embodiments, reduced or eliminated expression of CIITA reduces or eliminates expression of one or more of the following MHC class II are HLA-DP, HLA-DM, HLA-DOA, HLA-DOB, HLA-DQ, and HLA-DR.

In some embodiments, the cells described herein comprise gene modifications at the gene locus encoding the CIITA protein. In other words, the cells comprise a genetic modification at the CIITA locus. In some instances, the nucleotide sequence encoding the CIITA protein is set forth in RefSeq. No. NM_000246.4 and NCBI Genbank No. U18259. In some instances, the CIITA gene locus is described in NCBI Gene ID No. 4261. In certain cases, the amino acid sequence of CIITA is depicted as NCBI GenBank No. AAA88861.1. Additional descriptions of the CIITA protein and gene locus can be found in Uniprot No. P33076, HGNC Ref No. 7067, and OMIM Ref. No. 600005.

In some embodiments, the hypoimmunogenic cells outlined herein comprise a genetic modification targeting the CIITA gene. In some embodiments, the genetic modification targeting the CIITA gene by the rare-cutting endonuclease comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOs:5184-36352 of Table 12 of WO2016183041, which is herein incorporated by reference. In some embodiments, the cell has a reduced ability to induce an innate and/or an adaptive immune response in a recipient subject. In some embodiments, an exogenous nucleic acid encoding a polypeptide as disclosed herein (e.g., a chimeric antigen receptor, CD47, or another tolerogenic factor disclosed herein) is inserted at the CIITA gene.

Assays to test whether the CIITA gene has been inactivated are known and described herein. In some embodiments, the resulting genetic modification of the CIITA gene by PCR and the reduction of HLA-II expression can be assays by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating genetic modification. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

C. B2M In some embodiments, the technologies disclosed herein modulate (e.g., reduce or eliminate) the expression of MHC-I genes by targeting and modulating (e.g., reducing or eliminating) expression of the accessory chain B2M. In some embodiments, the modulation occurs using a CRISPR/Cas system. By modulating (e.g., reducing or deleting) expression of B2M, surface trafficking of MHC-I molecules is blocked and the cell rendered hypoimmunogenic. In some embodiments, the cell has a reduced ability to induce an innate and/or an adaptive immune response in a recipient subject.

In some embodiments, the target polynucleotide sequence of the present disclosure is a variant of B2M. In some embodiments, the target polynucleotide sequence is a homolog of B2M. In some embodiments, the target polynucleotide sequence is an ortholog of B2M.

In some embodiments, decreased or eliminated expression of B2M reduces or eliminates expression of one or more of the following MHC I molecules: HLA-A, HLA-B, and HLA-C.

In some embodiments, the cells described herein comprise gene modifications at the gene locus encoding the B2M protein. In other words, the cells comprise a genetic modification at the B2M locus. In some instances, the nucleotide sequence encoding the B2M protein is set forth in RefSeq. No. NM_004048.4 and Genbank No. AB021288.1. In some instances, the B2M gene locus is described in NCBI Gene ID No. 567. In certain cases, the amino acid sequence of B2M is depicted as NCBI GenBank No. BAA35182.1. Additional descriptions of the B2M protein and gene locus can be found in Uniprot No. P61769, HGNC Ref. No. 914, and OMIM Ref. No. 109700.

In some embodiments, the hypoimmunogenic cells outlined herein comprise a genetic modification targeting the B2M gene. In some embodiments, the genetic modification targeting the B2M gene by the rare-cutting endonuclease comprises a Cas protein or a polynucleotide encoding a Cas protein, and at least one guide ribonucleic acid sequence for specifically targeting the B2M gene. In some embodiments, the at least one guide ribonucleic acid sequence for specifically targeting the B2M gene is selected from the group consisting of SEQ ID NOS:81240-85644 of Table 15 of WO2016183041, which is herein incorporated by reference. In some embodiments, an exogenous nucleic acid encoding a polypeptide as disclosed herein (e.g., a chimeric antigen receptor, CD47, or another tolerogenic factor disclosed herein) is inserted at the B2M gene. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

Assays to test whether the B2M gene has been inactivated are known and described herein. In some embodiments, the resulting genetic modification of the B2M gene by PCR and the reduction of HLA-I expression can be assays by FACS analysis. In another embodiment, B2M protein expression is detected using a Western blot of cells lysates probed with antibodies to the B2M protein. In another embodiment, reverse transcriptase polymerase chain reactions (RT-PCR) are used to confirm the presence of the inactivating genetic modification.

### D. NLRC5

In many embodiments, the technologies disclosed herein modulate (e.g., reduce or eliminate) the expression of MHC-I genes by targeting and modulating (e.g., reducing or eliminating) expression of the NLR family, CARD domain containing 5/NOD27/CLR16.1 (NLRC5). In some embodiments, the modulation occurs using a CRISPR/Cas system. NLRC5 is a critical regulator of MHC-I-mediated immune responses and, similar to CIITA, NLRC5 is highly inducible by IFN-γ and can translocate into the nucleus. NLRC5 activates the promoters of MHC-I genes and induces the transcription of MHC-I as well as related genes involved in MHC-I antigen presentation.

In some embodiments, the target polynucleotide sequence is a variant of NLRC5. In some embodiments, the target polynucleotide sequence is a homolog of NLRC5. In some embodiments, the target polynucleotide sequence is an ortholog of NLRC5.

In some embodiments, decreased or eliminated expression of NLRC5 reduces or eliminates expression of one or more of the following MHC I molecules—HLA-A, HLA-B, and HLA-C.

In some embodiments, the cells outlined herein comprise a genetic modification targeting the NLRC5 gene. In some embodiments, the genetic modification targeting the NLRC5 gene by the rare-cutting endonuclease comprises a Cas protein or a polynucleotide encoding a Cas protein, and at least one guide ribonucleic acid sequence for specifically targeting the NLRC5 gene. In some embodiments, the at least one guide ribonucleic acid sequence for specifically targeting the NLRC5 gene is selected from the group consisting of SEQ ID NOS:36353-81239 of Appendix 3 or Table 14 of WO2016183041, the disclosure is incorporated by reference in its entirety.

Assays to test whether the NLRC5 gene has been inactivated are known and described herein. In some embodiments, the resulting genetic modification of the NLRC5 gene by PCR and the reduction of HLA-I expression can be assays by FACS analysis. In another embodiment, NLRC5 protein expression is detected using a Western blot of cells lysates probed with antibodies to the NLRC5 protein. In another embodiment, reverse transcriptase polymerase chain reactions (RT-PCR) are used to confirm the presence of the inactivating genetic modification.

### E. TRAC

In many embodiments, the technologies disclosed herein modulate (e.g., reduce or eliminate) the expression of TCR genes including the TRAC gene by targeting and modulating (e.g., reducing or eliminating) expression of the constant region of the T cell receptor alpha chain. In some embodiments, the modulation occurs using a CRISPR/Cas system. By modulating (e.g., reducing or deleting) expression of TRAC, surface trafficking of TCR molecules is blocked. In some embodiments, the cell also has a reduced ability to induce an innate and/or an adaptive immune response in a recipient subject.

In some embodiments, the target polynucleotide sequence of the present disclosure is a variant of TRAC. In some embodiments, the target polynucleotide sequence is a homolog of TRAC. In some embodiments, the target polynucleotide sequence is an ortholog of TRAC.

In some embodiments, decreased or eliminated expression of TRAC reduces or eliminates TCR surface expression.

In some embodiments, the cells, such as, but not limited to, pluripotent stem cells, induced pluripotent stem cells, T cells differentiated from induced pluripotent stem cells, primary T cells, and cells derived from primary T cells comprise gene modifications at the gene locus encoding the TRAC protein. In other words, the cells comprise a genetic modification at the TRAC locus. In some instances, the nucleotide sequence encoding the TRAC protein is set forth in Genbank No. X02592.1. In some instances, the TRAC gene locus is described in RefSeq. No. NG_001332.3 and NCBI Gene ID No. 28755. In certain cases, the amino acid sequence of TRAC is depicted as Uniprot No. P01848. Additional descriptions of the TRAC protein and gene locus can be found in Uniprot No. P01848, HGNC Ref No. 12029, and OMIM Ref No. 186880.

In some embodiments, the hypoimmunogenic cells outlined herein comprise a genetic modification targeting the TRAC gene. In some embodiments, the genetic modification targeting the TRAC gene by the rare-cutting endonuclease comprises a Cas protein or a polynucleotide encoding a Cas protein, and at least one guide ribonucleic acid sequence for specifically targeting the TRAC gene. In some embodiments, the at least one guide ribonucleic acid sequence for specifically targeting the TRAC gene is selected from the group consisting of SEQ ID NOS:532-609 and 9102-9797 of US20160348073, which is herein incorporated by reference.

Assays to test whether the TRAC gene has been inactivated are known and described herein. In some embodiments, the resulting genetic modification of the TRAC gene by PCR and the reduction of TCR expression can be assays by FACS analysis. In another embodiment, TRAC protein expression is detected using a Western blot of cells lysates probed with antibodies to the TRAC protein. In another embodiment, reverse transcriptase polymerase chain reactions (RT-PCR) are used to confirm the presence of the inactivating genetic modification.

### F. TRB

In many embodiments, the technologies disclosed herein modulate (e.g., reduce or eliminate) the expression of TCR genes including the gene encoding T cell antigen receptor, beta chain (e.g., the TRB, TRBC, or TCRB gene) by targeting and modulating (e.g., reducing or eliminating) expression of the constant region of the T cell receptor beta chain. In some embodiments, the modulation occurs using a CRISPR/Cas system. By modulating (e.g., reducing or deleting) expression of TRB, surface trafficking of TCR molecules is blocked. In some embodiments, the cell also has a reduced ability to induce an innate and/or an adaptive immune response in a recipient subject.

In some embodiments, the target polynucleotide sequence of the present disclosure is a variant of TRB. In some embodiments, the target polynucleotide sequence is a homolog of TRB. In some embodiments, the target polynucleotide sequence is an ortholog of TRB.

In some embodiments, decreased or eliminated expression of TRB reduces or eliminates TCR surface expression.

In some embodiments, the cells, such as, but not limited to, pluripotent stem cells, induced pluripotent stem cells, T cells differentiated from induced pluripotent stem cells, primary T cells, and cells derived from primary T cells comprise gene modifications at the gene locus encoding the TRB protein. In other words, the cells comprise a genetic modification at the TRB gene locus. In some instances, the nucleotide sequence encoding the TRB protein is set forth in UniProt No. PODSE2. In some instances, the TRB gene locus is described in RefSeq. No. NG_001333.2 and NCBI Gene ID No. 6957. In certain cases, the amino acid sequence of TRB is depicted as Uniprot No. P01848. Additional descriptions of the TRB protein and gene locus can be found in GenBank No. L36092.2, Uniprot No. PODSE2, and HGNC Ref No. 12155.

In some embodiments, the hypoimmunogenic cells outlined herein comprise a genetic modification targeting the TRB gene. In some embodiments, the genetic modification targeting the TRB gene by the rare-cutting endonuclease comprises a Cas protein or a polynucleotide encoding a Cas protein, and at least one guide ribonucleic acid sequence for specifically targeting the TRB gene. In some embodiments, the at least one guide ribonucleic acid sequence for specifically targeting the TRB gene is selected from the group consisting of SEQ ID NOS:610-765 and 9798-10532 of US20160348073, which is herein incorporated by reference.

Assays to test whether the TRB gene has been inactivated are known and described herein. In some embodiments, the resulting genetic modification of the TRB gene by PCR and the reduction of TCR expression can be assays by FACS analysis. In another embodiment, TRB protein expression is detected using a Western blot of cells lysates probed with antibodies to the TRB protein. In another embodiment, reverse transcriptase polymerase chain reactions (RT-PCR) are used to confirm the presence of the inactivating genetic modification.

### G. CD142

In many embodiments, the technologies disclosed herein modulate (e.g., reduce or eliminate) the expression of CD142, which is also known as tissue factor, factor III, and F3. In some embodiments, the modulation occurs using a gene editing system (e.g., CRISPR/Cas).

In some embodiments, the target polynucleotide sequence is CD142 or a variant of CD142. In some embodiments, the target polynucleotide sequence is a homolog of CD142. In some embodiments, the target polynucleotide sequence is an ortholog of CD142.

In some embodiments, the cells outlined herein comprise a genetic modification targeting the CD142 gene. In some embodiments, the genetic modification targeting the CD142 gene by the rare-cutting endonuclease comprises a Cas protein or a polynucleotide encoding a Cas protein, and at least one guide ribonucleic acid (gRNA) sequence for specifically targeting the CD142 gene. Useful methods for identifying gRNA sequences to target CD142 are described below.

Assays to test whether the CD142 gene has been inactivated are known and described herein. In some embodiments, the resulting genetic modification of the CD142 gene by PCR and the reduction of CD142 expression can be assays by FACS analysis. In another embodiment, CD142 protein expression is detected using a Western blot of cells lysates probed with antibodies to the CD142 protein. In another embodiment, reverse transcriptase polymerase chain reactions (RT-PCR) are used to confirm the presence of the inactivating genetic modification.

Useful genomic, polynucleotide and polypeptide information about the human CD142 are provided in, for example, the GeneCard Identifier GC01M094530, HGNC No. 3541, NCBI Gene ID 2152, NCBI RefSeq Nos. NM_001178096.1, NM_001993.4, NP_001171567.1, and NP_001984.1, UniProt No. P13726, and the like.

### H. CTLA-4

In some embodiments, the target polynucleotide sequence is CTLA-4 or a variant of CTLA-4. In some embodiments, the target polynucleotide sequence is a homolog of CTLA-4. In some embodiments, the target polynucleotide sequence is an ortholog of CTLA-4.

In some embodiments, the cells outlined herein comprise a genetic modification targeting the CTLA-4 gene. In certain embodiments, primary T cells comprise a genetic modification targeting the CTLA-4 gene. The genetic modification can reduce expression of CTLA-4 polynucleotides and CTLA-4 polypeptides in T cells includes primary T cells and CAR-T cells. In some embodiments, the genetic modification targeting the CTLA-4 gene by the rare-cutting endonuclease comprises a Cas protein or a polynucleotide encoding a Cas protein, and at least one guide ribonucleic acid (gRNA) sequence for specifically targeting the CTLA-4 gene. Useful methods for identifying gRNA sequences to target CTLA-4 are described below.

Assays to test whether the CTLA-4 gene has been inactivated are known and described herein. In some embodiments, the resulting genetic modification of the CTLA-4 gene by PCR and the reduction of CTLA-4 expression can be assays by FACS analysis. In another embodiment, CTLA-4 protein expression is detected using a Western blot of cells lysates probed with antibodies to the CTLA-4 protein. In another embodiment, reverse transcriptase polymerase chain reactions (RT-PCR) are used to confirm the presence of the inactivating genetic modification.

Useful genomic, polynucleotide and polypeptide information about the human CTLA-4 are provided in, for example, the GeneCard Identifier GC02P203867, HGNC No. 2505, NCBI Gene ID 1493, NCBI RefSeq Nos. NM_005214.4, NM 001037631.2, NP_001032720.1 and NP_005205.2, UniProt No. P16410, and the like.

### I. PD-1

In some embodiments, the target polynucleotide sequence is PD-1 or a variant of PD-1. In some embodiments, the target polynucleotide sequence is a homolog of PD-1. In some embodiments, the target polynucleotide sequence is an ortholog of PD-1.

In some embodiments, the cells outlined herein comprise a genetic modification targeting the gene encoding the programmed cell death protein 1 (PD-1) protein or the PDCD1 gene. In certain embodiments, primary T cells comprise a genetic modification targeting the PDCD1 gene. The genetic modification can reduce expression of PD-1 polynucleotides and PD-1 polypeptides in T cells includes primary T cells and CAR-T cells. In some embodiments, the genetic modification targeting the PDCD1 gene by the rare-cutting endonuclease comprises a Cas protein or a polynucleotide encoding a Cas protein, and at least one guide ribonucleic acid (gRNA) sequence for specifically targeting the PDCD1 gene. Useful methods for identifying gRNA sequences to target PD-1 are described below.

Assays to test whether the PDCD1 gene has been inactivated are known and described herein. In some embodiments, the resulting genetic modification of the PDCD1 gene by PCR and the reduction of PD-1 expression can be assays by FACS analysis. In another embodiment, PD-1 protein expression is detected using a Western blot of cells lysates probed with antibodies to the PD-1 protein. In another embodiment, reverse transcriptase polymerase chain reactions (RT-PCR) are used to confirm the presence of the inactivating genetic modification.

Useful genomic, polynucleotide and polypeptide information about human PD-1 including the PDCD1 gene are provided in, for example, the GeneCard Identifier GC02M241849, HGNC No. 8760, NCBI Gene ID 5133, Uniprot No. Q15116, and NCBI RefSeq Nos. NM_005018.2 and NP_005009.2.

### J. CD47

In some embodiments, the present disclosure provides a cell or population thereof that has been modified to express the tolerogenic factor (e.g., immunomodulatory polypeptide) CD47. In some embodiments, the present disclosure provides a method for altering a cell genome to express CD47. In some embodiments, the stem cell expresses exogenous CD47. In some instances, the cell expresses an expression vector comprising a nucleotide sequence encoding a human CD47 polypeptide. In some embodiments, the cell is genetically modified to comprise an integrated exogenous polynucleotide encoding CD47 using homology-directed repair. In some instances, the cell expresses a nucleotide sequence encoding a human CD47 polypeptide such that the nucleotide sequence is inserted into at least one allele of a safe harbor or target locus. In some instances, the cell expresses a nucleotide sequence encoding a human CD47 polypeptide wherein the nucleotide sequence is inserted into at least one allele of an AAVS1 locus. In some instances, the cell expresses a nucleotide sequence encoding a human CD47 polypeptide wherein the nucleotide sequence is inserted into at least one allele of a CCR5 locus. In some instances, the cell expresses a nucleotide sequence encoding a human CD47 polypeptide wherein the nucleotide sequence is inserted into at least one allele of a safe harbor or target gene locus, such as, but not limited to, a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus. In some instances, the cell expresses a nucleotide sequence encoding a human CD47 polypeptide wherein the nucleotide sequence is inserted into at least one allele of a TRAC locus.

CD47 is a leukocyte surface antigen and has a role in cell adhesion and modulation of integrins. It is expressed on the surface of a cell and signals to circulating macrophages not to eat the cell.

In some embodiments, the cell outlined herein comprises a nucleotide sequence encoding a CD47 polypeptide has at least 95% sequence identity (e.g., 95%, 96%, 97%, 98%, 99%, or more) to an amino acid sequence as set forth in NCBI Ref Sequence Nos. NP_001768.1 and NP_942088.1. In some embodiments, the cell outlined herein comprises a nucleotide sequence encoding a CD47 polypeptide having an amino acid sequence as set forth in NCBI Ref Sequence Nos. NP_001768.1 and NP_942088.1. In some embodiments, the cell comprises a nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises a nucleotide sequence for CD47 as set forth in NCBI Ref. Sequence Nos. NM_001777.3 and NM_198793.2. In some embodiments, the nucleotide sequence encoding a CD47 polynucleotide is a codon optimized sequence. In some embodiments, the nucleotide sequence encoding a CD47 polynucleotide is a human codon optimized sequence.

In some embodiments, the cell comprises a CD47 polypeptide having at least 95% sequence identity (e.g., 95%, 96%, 97%, 98%, 99%, or more) to an amino acid sequence as set forth in NCBI Ref Sequence Nos. NP_001768.1 and NP_942088.1. In some embodiments, the cell outlined herein comprises a CD47 polypeptide having an amino acid sequence as set forth in NCBI Ref. Sequence Nos. NP_001768.1 and NP_942088.1.

Exemplary amino acid sequences of human CD47 with a signal sequence and without a signal sequence are provided in Table 1.

In some embodiments, the cell comprises a CD47 polypeptide having at least 95% sequence identity (e.g., 95%, 96%, 97%, 98%, 99%, or more) to the amino acid sequence of SEQ ID NO: 1. In some embodiments, the cell comprises a CD47 polypeptide having the amino acid sequence of SEQ ID NO:1. In some embodiments, the cell comprises a CD47 polypeptide having at least 95% sequence identity (e.g., 95%, 96%, 97%, 98%, 99%, or more) to the amino acid sequence of SEQ ID NO:2. In some embodiments, the cell comprises a CD47 polypeptide having the amino acid sequence of SEQ ID NO:2.

In some embodiments, the cell comprises a nucleotide sequence encoding a CD47 polypeptide having at least 95% sequence identity (e.g., 95%, 96%, 97%, 98%, 99%, or more) to the amino acid sequence of SEQ ID NO: 1. In some embodiments, the cell comprises a nucleotide sequence encoding a CD47 polypeptide having the amino acid sequence of SEQ ID NO:1. In some embodiments, the cell comprises a nucleotide sequence encoding a CD47 polypeptide having at least 95% sequence identity (e.g., 95%, 96%, 97%, 98%, 99%, or more) to the amino acid sequence of SEQ ID NO:2. In some embodiments, the cell comprises a nucleotide sequence encoding a CD47 polypeptide having the amino acid sequence of SEQ ID NO:2. In some embodiments, the nucleotide sequence is codon optimized for expression in a particular cell.

In some embodiments, a suitable gene editing system (e.g., CRISPR/Cas system or any of the gene editing systems described herein) is used to facilitate the insertion of a polynucleotide encoding CD47, into a genomic locus of the hypoimmunogenic cell. In some cases, the polynucleotide encoding CD47 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding CD47 is inserted into a B2M gene locus, a CIITA gene locus, a TRAC gene locus, or a TRB gene locus. In certain embodiments, the polynucleotide encoding CD47 is operably linked to a promoter.

In some embodiments, the polynucleotide encoding CD47 is inserted into at least one allele of the T cell using viral transduction. In some embodiments, the polynucleotide encoding CD47 is inserted into at least one allele of the T cell using a lentivirus based viral vector. In some embodiments, the lentivirus based viral vector is a pseudotyped, self-inactivating lentiviral vector that carries the polynucleotide encoding CD47. In some embodiments, the lentivirus based viral vector is a self-inactivating lentiviral vector pseudotyped with a vesicular stomatitis VSV-G envelope, and which carries the polynucleotide encoding CD47.

In another embodiment, CD47 protein expression is detected using a Western blot of cell lysates probed with antibodies against the CD47 protein. In another embodiment, reverse transcriptase polymerase chain reactions (RT-PCR) are used to confirm the presence of the exogenous CD47 mRNA.

### K. CD24

In some embodiments, the present disclosure provides a cell or population thereof that has been modified to express the tolerogenic factor (e.g., immunomodulatory polypeptide) CD24. In some embodiments, the present disclosure provides a method for altering a cell genome to express CD24. In some embodiments, the stem cell expresses exogenous CD24. In some instances, the cell expresses an expression vector comprising a nucleotide sequence encoding a human CD24 polypeptide. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

CD24 which is also referred to as a heat stable antigen or small-cell lung cancer cluster 4 antigen is a glycosylated glycosylphosphatidylinositol-anchored surface protein (Pirruccello et al., J Immunol, 1986, 136, 3779-3784; Chen et al., Glycobiology, 2017, 57, 800-806). It binds to Siglec-10 on innate immune cells. Recently it has been shown that CD24 via Siglec-10 acts as an innate immune checkpoint (Barkal et al., Nature, 2019, 572, 392-396).

In some embodiments, the cell outlined herein comprises a nucleotide sequence encoding a CD24 polypeptide has at least 95% sequence identity (e.g., 95%, 96%, 97%, 98%, 99%, or more) to an amino acid sequence set forth in NCBI Ref Nos. NP_001278666.1, NP_001278667.1, NP_001278668.1, and NP_037362.1. In some embodiments, the cell outlined herein comprises a nucleotide sequence encoding a CD24 polypeptide having an amino acid sequence set forth in NCBI Ref. Nos. NP_001278666.1, NP_001278667.1, NP_001278668.1, and NP_037362.1.

In some embodiments, the cell comprises a nucleotide sequence having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref Nos. NM_00129737.1, NM_00129738.1, NM_001291739.1, and NM_013230.3. In some embodiments, the cell comprises a nucleotide sequence as set forth in NCBI Ref. Nos. NM_00129737.1, NM_00129738.1, NM_001291739.1, and NM_013230.3.

In some embodiments, a suitable gene editing system (e.g., CRISPR/Cas system or any of the gene editing systems described herein) is used to facilitate the insertion of a polynucleotide encoding CD24, into a genomic locus of the hypoimmunogenic cell. In some cases, the polynucleotide encoding CD24 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding CD24 is inserted into a B2M gene locus, a CIITA gene locus, a TRAC gene locus, or a TRB gene locus. In some embodiments, the polynucleotide encoding CD24 is inserted into any one of the gene loci depicted in Table 15 provided herein. In certain embodiments, the polynucleotide encoding CD24 is operably linked to a promoter.

In another embodiment, CD24 protein expression is detected using a Western blot of cells lysates probed with antibodies against the CD24 protein. In another embodiment, reverse transcriptase polymerase chain reactions (RT-PCR) are used to confirm the presence of the exogenous CD24 mRNA.

In some embodiments, a suitable gene editing system (e.g., CRISPR/Cas system or any of the gene editing systems described herein) is used to facilitate the insertion of a polynucleotide encoding CD24, into a genomic locus of the hypoimmunogenic cell. In some cases, the polynucleotide encoding CD24 is inserted into a safe harbor or target locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus. In some embodiments, the polynucleotide encoding CD24 is inserted into a B2M gene locus, a CIITA gene locus, a TRAC gene locus, or a TRB gene locus. In some embodiments, the polynucleotide encoding CD24 is inserted into any one of the gene loci depicted in Table 15 provided herein. In certain embodiments, the polynucleotide encoding CD24 is operably linked to a promoter.

### L. DUX4

In some embodiments, the present disclosure provides a cell (e.g., stem cell, induced pluripotent stem cell, differentiated cell, hematopoietic stem cell, primary T cell or CAR-T cell) or population thereof comprising a genome modified to increase expression of a tolerogenic or immunosuppressive factor such as DUX4. In some embodiments, the present disclosure provides a method for altering a cell's genome to provide increased expression of DUX4, including through a exogenous polynucleotide. In some embodiments, the disclosure provides a cell or population thereof comprising exogenously expressed DUX4 proteins. In some embodiments, increased expression of DUX4 suppresses, reduces or eliminates expression of one or more of the following MHC I molecules —HLA-A, HLA-B, and HLA-C. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

DUX4 is a transcription factor that is active in embryonic tissues and induced pluripotent stem cells, and is silent in normal, healthy somatic tissues (Feng et al., 2015, ELife4; De Iaco et al., 2017, Nat Genet, 49, 941-945; Hendrickson et al., 2017, Nat Genet, 49, 925-934; Snider et al., 2010, PLoS Genet, e1001181; Whiddon et al., 2017, Nat Genet). DUX4 expression acts to block IFN-gamma mediated induction of major histocompatibility complex (MHC) class I gene expression (e.g., expression of B2M, HLA-A, HLA-B, and HLA-C). DUX4 expression has been implicated in suppressed antigen presentation by MHC class I (Chew et al., Developmental Cell, 2019, 50, 1-14). DUX4 functions as a transcription factor in the cleavage-stage gene expression (transcriptional) program. Its target genes include, but are not limited to, coding genes, noncoding genes, and repetitive elements.

There are at least two isoforms of DUX4, with the longest isoform comprising the DUX4 C-terminal transcription activation domain. The isoforms are produced by alternative splicing. See, e.g., Geng et al., 2012, Dev Cell, 22, 38-51; Snider et al., 2010, PLoS Genet, e1001181. Active isoforms for DUX4 comprise its N-terminal DNA-binding domains and its C-terminal activation domain. See, e.g., Choi et al., 2016, Nucleic Acid Res, 44, 5161-5173.

It has been shown that reducing the number of CpG motifs of DUX4 decreases silencing of a DUX4 transgene (Jagannathan et al., Human Molecular Genetics, 2016, 25(20):4419-4431). The nucleic acid sequence provided in Jagannathan et al., supra represents a codon altered sequence of DUX4 comprising one or more base substitutions to reduce the total number of CpG sites while preserving the DUX4 protein sequence. The nucleic acid sequence is commercially available from Addgene, Catalog No. 99281.

In many embodiments, at least one or more polynucleotides may be utilized to facilitate the exogenous expression of DUX4 by a cell, e.g., a stem cell, induced pluripotent stem cell, differentiated cell, hematopoietic stem cell, primary T cell or CAR-T cell.

In some embodiments, a suitable gene editing system (e.g., CRISPR/Cas system or any of the gene editing systems described herein) is used to facilitate the insertion of a polynucleotide encoding DUX4, into a genomic locus of the hypoimmunogenic cell. In some cases, the polynucleotide encoding DUX4 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding DUX4 is inserted into a B2M gene locus, a CIITA gene locus, a TRAC gene locus, or a TRB gene locus. In some embodiments, the polynucleotide encoding DUX4 is inserted into any one of the gene loci depicted in Table 15 provided herein. In certain embodiments, the polynucleotide encoding DUX4 is operably linked to a promoter.

In some embodiments, the polynucleotide encoding DUX4 is inserted into at least one allele of the T cell using viral transduction. In some embodiments, the polynucleotide encoding DUX4 is inserted into at least one allele of the T cell using a lentivirus based viral vector. In some embodiments, the lentivirus based viral vector is a pseudotyped, self-inactivating lentiviral vector that carries the polynucleotide encoding DUX4. In some embodiments, the lentivirus based viral vector is a self-inactivating lentiviral vector pseudotyped with a vesicular stomatitis VSV-G envelope, and which carries the polynucleotide encoding DUX4.

In some embodiments, the polynucleotide sequence encoding DUX4 comprises a polynucleotide sequence comprising a codon altered nucleotide sequence of DUX4 comprising one or more base substitutions to reduce the total number of CpG sites while preserving the DUX4 protein sequence. In some embodiments, the polynucleotide sequence encoding DUX4 comprising one or more base substitutions to reduce the total number of CpG sites has at least 85% (e.g., 85%, 86%, 87%, 88%, 89%, 90%, 91%, 92%, 93%, 94%, 95%, 96%, 97%, 98%, 99% or 100%) sequence identity to SEQ ID NO:1 of PCT/US2020/44635, filed Jul. 31, 2020. In some embodiments, the polynucleotide sequence encoding DUX4 is SEQ ID NO:1 of PCT/US2020/44635.

In some embodiments, the polynucleotide sequence encoding DUX4 is a nucleotide sequence encoding a polypeptide sequence having at least 95% (e.g., 95%, 96%, 97%, 98%, 99% or 100%) sequence identity to a sequence selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO: 9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29, as provided in PCT/US2020/44635. In some embodiments, the polynucleotide sequence encoding DUX4 is a nucleotide sequence encoding a polypeptide sequence is selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO: 9, SEQ ID NO: 10, SEQ ID NO:11, SEQ ID NO:12, SEQ ID NO:13, SEQ ID NO:14, SEQ ID NO:15, SEQ ID NO: 16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29. Amino acid sequences set forth as SEQ ID NOS:2-29 are shown in FIG. 1A-1G of PCT/US2020/44635.

In some instances, the DUX4 polypeptide comprises an amino acid sequence having at least 95% sequence identity to the sequence set forth in GenBank Accession No. ACN62209.1 or an amino acid sequence set forth in GenBank Accession No. ACN62209.1. In some instances, the DUX4 polypeptide comprises an amino acid sequence having at least 95% sequence identity to the sequence set forth in NCBI RefSeq No. NP_001280727.1 or an amino acid sequence set forth in NCBI RefSeq No. NP_001280727.1. In some instances, the DUX4 polypeptide comprises an amino acid sequence having at least 95% sequence identity to the sequence set forth in GenBank Accession No. ACP30489.1 or an amino acid sequence set forth in GenBank Accession No. ACP30489.1. In some instances, the DUX4 polypeptide comprises an amino acid sequence having at least 95% sequence identity to the sequence set forth in UniProt No. POCJ85.1 or an amino acid sequence set forth in UniProt No. POCJ85.1. In some instances, the DUX4 polypeptide comprises an amino acid sequence having at least 95% sequence identity to the sequence set forth in GenBank Accession No. AUA60622.1 or an amino acid sequence set forth in GenBank Accession No. AUA60622.1. In some instances, the DUX4 polypeptide comprises an amino acid sequence having at least 95% sequence identity to the sequence set forth in GenBank Accession No. ADK24683.1 or an amino acid sequence set forth in GenBank Accession No. ADK24683.1. In some instances, the DUX4 polypeptide comprises an amino acid sequence having at least 95% sequence identity to the sequence set forth in GenBank Accession No. ACN62210.1 or an amino acid sequence set forth in GenBank Accession No. ACN62210.1. In some instances, the DUX4 polypeptide comprises an amino acid sequence having at least 95% sequence identity to the sequence set forth in GenBank Accession No. ADK24706.1 or an amino acid sequence set forth in GenBank Accession No. ADK24706.1. In some instances, the DUX4 polypeptide comprises an amino acid sequence having at least 95% sequence identity to the sequence set forth in GenBank Accession No. ADK24685.1 or an amino acid sequence set forth in GenBank Accession No. ADK24685.1. In some instances, the DUX4 polypeptide comprises an amino acid sequence having at least 95% sequence identity to the sequence set forth in GenBank Accession No. ACP30488.1 or an amino acid sequence set forth in GenBank Accession No. ACP30488.1. In some instances, the DUX4 polypeptide comprises an amino acid sequence having at least 95% sequence identity to the sequence set forth in GenBank Accession No. ADK24687.1 or an amino acid sequence set forth in GenBank Accession No. ADK24687.1. In some instances, the DUX4 polypeptide comprises an amino acid sequence having at least 95% sequence identity to the sequence set forth in GenBank Accession No. ACP30487.1 or an amino acid sequence set forth in GenBank Accession No. ACP30487.1. In some instances, the DUX4 polypeptide comprises an amino acid sequence having at least 95% sequence identity to the sequence set forth in GenBank Accession No. ADK24717.1 or an amino acid sequence set forth in GenBank Accession No. ADK24717.1. In some instances, the DUX4 polypeptide comprises an amino acid sequence having at least 95% sequence identity to the sequence set forth in GenBank Accession No. ADK24690.1 or an amino acid sequence set forth in GenBank Accession No. ADK24690.1. In some instances, the DUX4 polypeptide comprises an amino acid sequence having at least 95% sequence identity to the sequence set forth in GenBank Accession No. ADK24689.1 or an amino acid sequence set forth in GenBank Accession No. ADK24689.1. In some instances, the DUX4 polypeptide comprises an amino acid sequence having at least 95% sequence identity to the sequence set forth in GenBank Accession No. ADK24692.1 or an amino acid sequence set forth in GenBank Accession No. ADK24692.1. In some instances, the DUX4 polypeptide comprises an amino acid sequence having at least 95% sequence identity to the sequence set forth in GenBank Accession No. ADK24693.1 or an amino acid sequence of set forth in GenBank Accession No. ADK24693.1. In some instances, the DUX4 polypeptide comprises an amino acid sequence having at least 95% sequence identity to the sequence set forth in GenBank Accession No. ADK24712.1 or an amino acid sequence set forth in GenBank Accession No. ADK24712.1. In some instances, the DUX4 polypeptide comprises an amino acid sequence having at least 95% sequence identity to the sequence set forth in GenBank Accession No. ADK24691.1 or an amino acid sequence set forth in GenBank Accession No. ADK24691.1. In some instances, the DUX4 polypeptide comprises an amino acid sequence having at least 95% sequence identity to the sequence set forth in UniProt No. POCJ87.1 or an amino acid sequence of set forth in UniProt No. POCJ87.1. In some instances, the DUX4 polypeptide comprises an amino acid sequence having at least 95% sequence identity to the sequence set forth in GenBank Accession No. ADK24714.1 or an amino acid sequence set forth in GenBank Accession No. ADK24714.1. In some instances, the DUX4 polypeptide comprises an amino acid sequence having at least 95% sequence identity to the sequence set forth in GenBank Accession No. ADK24684.1 or an amino acid sequence of set forth in GenBank Accession No. ADK24684.1. In some instances, the DUX4 polypeptide comprises an amino acid sequence having at least 95% sequence identity to the sequence set forth in GenBank Accession No. ADK24695.1 or an amino acid sequence set forth in GenBank Accession No. ADK24695.1. In some instances, the DUX4 polypeptide comprises an amino acid sequence having at least 95% sequence identity to the sequence set forth in GenBank Accession No. ADK24699.1 or an amino acid sequence set forth in GenBank Accession No. ADK24699.1. In some instances, the DUX4 polypeptide comprises an amino acid sequence having at least 95% sequence identity to the sequence set forth in NCBI RefSeq No. NP_001768.1 or an amino acid sequence set forth in NCBI RefSeq No. NP_001768. In some instances, the DUX4 polypeptide comprises an amino acid sequence having at least 95% sequence identity to the sequence set forth in NCBI RefSeq No. NP_942088.1 or an amino acid sequence set forth in NCBI RefSeq No. NP_942088.1. In some instances, the DUX4 polypeptide comprises an amino acid sequence having at least 95% sequence identity to SEQ ID NO:28 provided in PCT/US2020/44635 or an amino acid sequence of SEQ ID NO:28 provided in PCT/US2020/44635. In some instances, the DUX4 polypeptide comprises an amino acid sequence having at least 95% sequence identity to SEQ ID NO:29 provided in PCT/US2020/44635 or an amino acid sequence of SEQ ID NO:29 provided in PCT/US2020/44635.

In other embodiments, expression of tolerogenic factors is facilitated using an expression vector. In some embodiments, the expression vector comprises a polynucleotide sequence encoding DUX4 is a codon altered sequence comprising one or more base substitutions to reduce the total number of CpG sites while preserving the DUX4 protein sequence. In some cases, the codon altered sequence of DUX4 comprises SEQ ID NO:1 of PCT/US2020/44635. In some cases, the codon altered sequence of DUX4 is SEQ ID NO:1 of PCT/US2020/44635. In other embodiments, the expression vector comprises a polynucleotide sequence encoding DUX4 comprising SEQ ID NO:1 of PCT/US2020/44635. In some embodiments, the expression vector comprises a polynucleotide sequence encoding a DUX4 polypeptide sequence having at least 95% sequence identity to a sequence selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO: 13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29 of PCT/US2020/44635. In some embodiments, the expression vector comprises a polynucleotide sequence encoding a DUX4 polypeptide sequence selected from a group including SEQ ID NO:2, SEQ ID NO:3, SEQ ID NO:4, SEQ ID NO:5, SEQ ID NO:6, SEQ ID NO:7, SEQ ID NO: 8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO: 19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29 of PCT/US2020/44635.

An increase of DUX4 expression can be assayed using known techniques, such as Western blots, ELISA assays, FACS assays, immunoassays, and the like.

### M. Additional Tolerogenic Factors

In many embodiments, one or more tolerogenic factors can be inserted or reinserted into genome-edited cells to create immune-privileged universal donor cells, such as universal donor stem cells, universal donor T cells, or universal donor cells. In certain embodiments, the hypoimmunogenic cells disclosed herein have been further modified to express one or more tolerogenic factors. Exemplary tolerogenic factors include, without limitation, one or more of CD47, DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39 FasL, CCL21, CCL22, Mfge8, CD16, CD52, H2-M3, CD16 Fc receptor, IL15-RF, H2-M3(HLA-G), B2M-HLA-E, A20/TNFAIP3, CR1, HLA-F, and MANF, and Serpinb9. In some embodiments, the tolerogenic factors are selected from the group consisting of CD200, HLA-G, HLA-E, HLA-C, HLA-E heavy chain, PD-L1, IDO1, CTLA4-Ig, ILL-10, IL-35, FasL, Serpinb9, CCL21, CCL22, and Mfge8. In some embodiments, the tolerogenic factors are selected from the group consisting of DUX4, HLA-C, HLA-E, HLA-F, HLA-G, PD-L1, CTLA-4-Ig, C1-inhibitor, and IL-35. In some embodiments, the tolerogenic factors are selected from the group consisting of HLA-C, HLA-E, HLA-F, HLA-G, PD-L1, CTLA-4-Ig, C1-inhibitor, and IL-35. In some embodiments, the tolerogenic factors are selected from a group including CD47, DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39 FasL, CCL21, CCL22, Mfge8, CD16, CD52, H2-M3, CD16 Fc receptor, IL15-RF, H2-M3(HLA-G), B2M-HLA-E, A20/TNFAIP3, CR1, HLA-F, and MANF, and Serpinb9.

In some embodiments, the polynucleotide encoding the one or more tolerogenic factors is inserted into at least one allele of the T cell using viral transduction. In some embodiments, the polynucleotide encoding the one or more tolerogenic factors is inserted into at least one allele of the T cell using a lentivirus based viral vector. In some embodiments, the lentivirus based viral vector is a pseudotyped, self-inactivating lentiviral vector that carries the polynucleotide encoding the one or more tolerogenic factors. In some embodiments, the lentivirus based viral vector is a self-inactivating lentiviral vector pseudotyped with a vesicular stomatitis VSV-G envelope, and which carries the polynucleotide encoding the one or more tolerogenic factors.

Useful genomic, polynucleotide and polypeptide information about human CD27 (which is also known as CD27L receptor, Tumor Necrosis Factor Receptor Superfamily Member 7, TNFSF7, T Cell Activation Antigen 5152, Tp55, and T14) are provided in, for example, the GeneCard Identifier GC12P008144, HGNC No. 11922, NCBI Gene ID 939, Uniprot No. P26842, and NCBI RefSeq Nos. NM_001242.4 and NP_001233.1.

Useful genomic, polynucleotide and polypeptide information about human CD46 are provided in, for example, the GeneCard Identifier GC01P207752, HGNC No. 6953, NCBI Gene ID 4179, Uniprot No. P15529, and NCBI RefSeq Nos. NM_002389.4, NM_153826.3, NM_172350.2, NM_172351.2, NM_172352.2 NP_758860.1, NM 172353.2, NM_172359.2, NM_172361.2, NP_002380.3, NP 722548.1, NP 758860.1, NP_758861.1, NP_758862.1, NP_758863.1, NP_758869.1, and NP_758871.1.

Useful genomic, polynucleotide and polypeptide information about human CD55 (also known as complement decay-accelerating factor) are provided in, for example, the GeneCard Identifier GC01P207321, HGNC No. 2665, NCBI Gene ID 1604, Uniprot No. P08174, and NCBI RefSeq Nos. NM_000574.4, NM_001114752.2, NM_001300903.1, NM 001300904.1, NP_000565.1, NP_001108224.1, NP_001287832.1, and NP_001287833.1.

Useful genomic, polynucleotide and polypeptide information about human CD59 are provided in, for example, the GeneCard Identifier GC11M033704, HGNC No. 1689, NCBI Gene ID 966, Uniprot No. P13987, and NCBI RefSeq Nos. NP_000602.1, NM_000611.5, NP_001120695.1, NM_001127223.1, NP_001120697.1, NM 001127225.1, NP_001120698.1, NM 001127226.1, NP_001120699.1, NM_001127227.1, NP_976074.1, NM_203329.2, NP_976075.1, NM_203330.2, NP_976076.1, and NM_203331.2.

Useful genomic, polynucleotide and polypeptide information about human CD200 are provided in, for example, the GeneCard Identifier GC03P112332, HGNC No. 7203, NCBI Gene ID 4345, Uniprot No. P41217, and NCBI RefSeq Nos. NP_001004196.2, NM_001004196.3, NP_001305757.1, NM_001318828.1, NP 005935.4, NM_005944.6, XP_005247539.1, and XM_005247482.2.

Useful genomic, polynucleotide and polypeptide information about human HLA-C are provided in, for example, the GeneCard Identifier GC06M031272, HGNC No. 4933, NCBI Gene ID 3107, Uniprot No. P10321, and NCBI RefSeq Nos. NP_002108.4 and NM_002117.5.

Useful genomic, polynucleotide and polypeptide information about human HLA-E are provided in, for example, the GeneCard Identifier GC06P047281, HGNC No. 4962, NCBI Gene ID 3133, Uniprot No. P13747, and NCBI RefSeq Nos. NP_005507.3 and NM_005516.5.

Useful genomic, polynucleotide and polypeptide information about human HLA-G are provided in, for example, the GeneCard Identifier GC06P047256, HGNC No. 4964, NCBI Gene ID 3135, Uniprot No. P17693, and NCBI RefSeq Nos. NP_002118.1 and NM_002127.5.

Useful genomic, polynucleotide and polypeptide information about human PD-L1 or CD274 are provided in, for example, the GeneCard Identifier GC09P005450, HGNC No. 17635, NCBI Gene ID 29126, Uniprot No. Q9NZQ7, and NCBI RefSeq Nos. NP_001254635.1, NM_001267706.1, NP_054862.1, and NM_014143.3.

Useful genomic, polynucleotide and polypeptide information about human IDO1 are provided in, for example, the GeneCard Identifier GC08P039891, HGNC No. 6059, NCBI Gene ID 3620, Uniprot No. P14902, and NCBI RefSeq Nos. NP_002155.1 and NM_002164.5.

Useful genomic, polynucleotide and polypeptide information about human IL-10 are provided in, for example, the GeneCard Identifier GC01M206767, HGNC No. 5962, NCBI Gene ID 3586, Uniprot No. P22301, and NCBI RefSeq Nos. NP_000563.1 and NM_000572.2.

Useful genomic, polynucleotide and polypeptide information about human Fas ligand (which is known as FasL, FASLG, CD178, TNFSF6, and the like) are provided in, for example, the GeneCard Identifier GC01P172628, HGNC No. 11936, NCBI Gene ID 356, Uniprot No. P48023, and NCBI RefSeq Nos. NP_000630.1, NM_000639.2, NP_001289675.1, and NM_001302746.1.

Useful genomic, polynucleotide and polypeptide information about human CCL21 are provided in, for example, the GeneCard Identifier GC09M034709, HGNC No. 10620, NCBI Gene ID 6366, Uniprot No. 000585, and NCBI RefSeq Nos. NP_002980.1 and NM_002989.3.

Useful genomic, polynucleotide and polypeptide information about human CCL22 are provided in, for example, the GeneCard Identifier GC16P057359, HGNC No. 10621, NCBI Gene ID 6367, Uniprot No. 000626, and NCBI RefSeq Nos. NP_002981.2, NM 002990.4, XP_016879020.1, and XM_017023531.1.

Useful genomic, polynucleotide and polypeptide information about human Mfge8 are provided in, for example, the GeneCard Identifier GC15M088898, HGNC No. 7036, NCBI Gene ID 4240, Uniprot No. Q08431, and NCBI RefSeq Nos. NP_001108086.1, NM_001114614.2, NP_001297248.1, NM_001310319.1, NP_001297249.1, NM_001310320.1, NP_001297250.1, NM_001310321.1, NP_005919.2, and NM_005928.3.

Useful genomic, polynucleotide and polypeptide information about human SerpinB9 are provided in, for example, the GeneCard Identifier GC06M002887, HGNC No. 8955, NCBI Gene ID 5272, Uniprot No. P50453, and NCBI RefSeq Nos. NP_004146.1, NM_004155.5, XP_005249241.1, and XM_005249184.4.

Methods for modulating expression of genes and factors (proteins) include genome editing technologies, RNA or protein expression technologies, and the like. For all of these technologies, well known recombinant techniques are used, to generate recombinant nucleic acids as outlined herein.

In some embodiments, the cells (e.g., stem cell, induced pluripotent stem cell, differentiated cell, hematopoietic stem cell, primary T cell or CAR-T cell) possess genetic modifications that inactivate the B2M and CIITA genes and express a plurality of exogenous polypeptides selected from the group including CD47 and DUX4, CD47 and CD24, CD47 and CD27, CD47 and CD35, CD47 and CD46, CD47 and CD55, CD47 and CD59, CD47 and CD200, CD47 and HLA-C, CD47 and HLA-E, CD47 and HLA-E heavy chain, CD47 and HLA-G, CD47 and PD-L1, CD47 and IDO1, CD47 and CTLA4-Ig, CD47 and C1-Inhibitor, CD47 and IL-10, CD47 and IL-35, CD47 and IL-39, CD47 and FasL, CD47 and CCL21, CD47 and CCL22, CD47 and Mfge8, CD47 and CD16, CD47 and CD52, CD47 and CD16 Fc receptor, CD47 and IL15-RF, CD47 and H2-M3((HLA-G), CD47 and B2M-HLA-E, CD47 and A20/TNFAIP3, CD47 and CR1, CD47 and HLA-F, CD47 and MANF, and CD47 and Serpinb9, and any combination thereof. In some instances, such cells also possess a genetic modification that inactivates the CD142 gene.

In some instances, a gene editing system such as the CRISPR/Cas system is used to facilitate the insertion of tolerogenic factors, such as the tolerogenic factors into a safe harbor or target locus, such as the AAVS1 locus, to actively inhibit immune rejection. In some instances, the tolerogenic factors are inserted into a safe harbor or target locus using an expression vector. In some embodiments, the safe harbor or target locus is an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus.

In some embodiments, expression of a target gene (e.g., DUX4, CD47, or another tolerogenic factor gene) is increased by expression of fusion protein or a protein complex containing (1) a site-specific binding domain specific for the endogenous target gene (e.g., DUX4, CD47, or another tolerogenic factor gene) and (2) a transcriptional activator.

In some embodiments, the regulatory factor is comprised of a site specific DNA-binding nucleic acid molecule, such as a guide RNA (gRNA). In some embodiments, the method is achieved by site specific DNA-binding targeted proteins, such as zinc finger proteins (ZFP) or fusion proteins containing ZFP, which are also known as zinc finger nucleases (ZFNs). In some embodiments, the method is achieved by a genome-modifying protein described herein, including for example, a CRISPR-associated transposase, prime editing, or Programmable Addition via Site-specific Targeting Elements (PASTE). In some embodiments, the method is achieved by a genome-modifying protein described herein, including for example, TnpB polypeptides.

In some embodiments, the regulatory factor comprises a site-specific binding domain, such as using a DNA binding protein or DNA-binding nucleic acid, which specifically binds to or hybridizes to the gene at a targeted region. In some embodiments, the provided polynucleotides or polypeptides are coupled to or complexed with a site-specific nuclease, such as a modified nuclease. For example, in some embodiments, the administration is effected using a fusion comprising a DNA-targeting protein of a modified nuclease, such as a meganuclease or an RNA-guided nuclease such as a clustered regularly interspersed short palindromic nucleic acid (CRISPR)-Cas system, such as CRISPR-Cas9 system. In some embodiments, the nuclease is modified to lack nuclease activity. In some embodiments, the modified nuclease is a catalytically dead dCas9.

In some embodiments, the site specific binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIlI, I-CreI, I-TevI, I-TevII and I-TevIII. See also U.S. Pat. Nos. 5,420,032; 6,833,252; Belfort et al., (1997) Nucleic Acids Res. 25:3379-3388; Dujon et al., (1989) Gene 82:115-118; Perler et al, (1994) Nucleic Acids Res. 22, 1125-1127; Jasin (1996) Trends Genet. 12:224-228; Gimble et al., (1996) J. Mol. Biol. 263:163-180; Argast et al, (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al, (2002) Molec. Cell 10:895-905; Epinat et al, (2003) Nucleic Acids Res. 31:2952-2962; Ashworth et al, (2006) Nature 441:656-659; Paques et al, (2007) Current Gene Therapy 7:49-66; U.S. Patent Publication No. 2007/0117128.

Zinc finger, TALE, and CRISPR system binding domains can be “engineered” to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the site-specific binding domain comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion.

Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed.

In some embodiments, the site-specific binding domain comprises a naturally occurring or engineered (non-naturally occurring) transcription activator-like protein (TAL) DNA binding domain, such as in a transcription activator-like protein effector (TALE) protein, See, e.g., U.S. Patent Publication No. 20110301073, incorporated by reference in its entirety herein.

In some embodiments, the site-specific binding domain is derived from the CRISPR/Cas system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system, or a “targeting sequence”), and/or other sequences and transcripts from a CRISPR locus.

In general, a guide sequence includes a targeting domain comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain of the gRNA is complementary, e.g., at least 80, 85, 90, 95, 98 or 99% complementary, e.g., fully complementary, to the target sequence on the target nucleic acid.

In some embodiments, the target site is upstream of a transcription initiation site of the target gene. In some embodiments, the target site is adjacent to a transcription initiation site of the gene. In some embodiments, the target site is adjacent to an RNA polymerase pause site downstream of a transcription initiation site of the gene.

In some embodiments, the targeting domain is configured to target the promoter region of the target gene to promote transcription initiation, binding of one or more transcription enhancers or activators, and/or RNA polymerase. One or more gRNA can be used to target the promoter region of the gene. In some embodiments, one or more regions of the gene can be targeted. In certain aspects, the target sites are within 600 base pairs on either side of a transcription start site (TSS) of the gene.

It is within the level of a skilled artisan to design or identify a gRNA sequence that is or comprises a sequence targeting a gene, including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see e.g., genescript.com/gRNA-database.html; see also, Sanjana et al. (2014) Nat. Methods, 11:783-4; www.e-crisp.org/E-CRISP/; crispr.mit.edu/). In some embodiments, the gRNA sequence is or comprises a sequence with minimal off-target binding to a non-target gene.

In some embodiments, the regulatory factor further comprises a functional domain, e.g., a transcriptional activator.

In some embodiments, the transcriptional activator is or contains one or more regulatory elements, such as one or more transcriptional control elements of a target gene, whereby a site-specific domain as provided above is recognized to drive expression of such gene. In some embodiments, the transcriptional activator drives expression of the target gene. In some cases, the transcriptional activator, can be or contain all or a portion of an heterologous transactivation domain. For example, in some embodiments, the transcriptional activator is selected from Herpes simplex-derived transactivation domain, Dnmt3a methyltransferase domain, p65, VP16, and VP64.

In some embodiments, the regulatory factor is a zinc finger transcription factor (ZF-TF). In some embodiments, the regulatory factor is VP64-p65-Rta (VPR).

In certain embodiments, the regulatory factor further comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g., kinases, acetylases and deacetylases); and DNA modifying enzymes (e.g., methyltransferases such as members of the DNMT family (e.g., DNMT1, DNMT3A, DNMT3B, DNMT3L, etc., topoisomerases, helicases, ligases, kinases, phosphatases, polymerases, endonucleases) and their associated factors and modifiers. See, e.g., U.S. Publication No. 2013/0253040, incorporated by reference in its entirety herein.

Suitable domains for achieving activation include the HSV VP 16 activation domain (see, e.g., Hagmann et al, J. Virol. 71, 5952-5962 (197)) nuclear hormone receptors (see, e.g., Torchia et al., Curr. Opin. Cell. Biol. 10:373-383 (1998)); the p65 subunit of nuclear factor kappa B (Bitko & Bank, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, Spl, AP-2, and CTF1 (Seipel etal, EMBOJ. 11, 4961-4968 (1992) as well as p300, CBP, PCAF, SRC1 PvALF, AtHD2A and ERF-2. See, for example, Robyr et al, (2000) Mol. Endocrinol. 14:329-347; Collingwood et al, (1999) J. Mol. Endocrinol 23:255-275; Leo et al, (2000) Gene 245:1-11; Manteuffel-Cymborowska (1999) Acta Biochim. Pol. 46:77-89; McKenna et al, (1999) J. Steroid Biochem. Mol. Biol. 69:3-12; Malik et al, (2000) Trends Biochem. Sci. 25:277-283; and Lemon et al, (1999) Curr. Opin. Genet. Dev. 9:499-504. Additional exemplary activation domains include, but are not limited to, OsGAI, HALF-1, C1, AP1, ARF-5, -6, -1, and -8, CPRF1, CPRF4, MYC-RP/GP, and TRAB1, See, for example, Ogawa et al, (2000) Gene 245:21-29; Okanami et al, (1996) Genes Cells 1:87-99; Goff et al, (1991) Genes Dev. 5:298-309; Cho et al, (1999) Plant Mol Biol 40:419-429; Ulmason et al, (1999) Proc. Natl. Acad. Sci. USA 96:5844-5849; Sprenger-Haussels et al, (2000) Plant J. 22:1-8; Gong et al, (1999) Plant Mol. Biol. 41:33-44; and Hobo et al., (1999) Proc. Natl. Acad. Sci. USA 96:15,348-15,353.

Exemplary repression domains that can be used to make genetic repressors include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B, DNMT3L, etc.), Rb, and MeCP2. See, for example, Bird et al, (1999) Cell 99:451-454; Tyler et al, (1999) Cell 99:443-446; Knoepfler et al, (1999) Cell 99:447-450; and Robertson et al, (2000) Nature Genet. 25:338-342. Additional exemplary repression domains include, but are not limited to, ROM2 and AtHD2A. See, for example, Chem et al, (1996) Plant Cell 8:305-321; and Wu et al, (2000) Plant J. 22:19-27.

In some instances, the domain is involved in epigenetic regulation of a chromosome. In some embodiments, the domain is a histone acetyltransferase (HAT), e.g., type-A, nuclear localized such as MYST family members MOZ, Ybf2/Sas3, MOF, and Tip60, GNAT family members Gcn5 or pCAF, the p300 family members CBP, p300 or Rttl09 (Bemdsen and Denu (2008) Curr Opin Struct Biol 18(6):682-689). In other instances the domain is a histone deacetylase (HD AC) such as the class I (HDAC-1, 2, 3, and 8), class II (HDAC IIA (HDAC-4, 5, 7 and 9), HD AC IIB (HDAC 6 and 10)), class IV (HDAC-11), class III (also known as sirtuins (SIRTs); SIRT1-7) (see Mottamal et al., (2015) Molecules 20(3):3898-3941). Another domain that is used in some embodiments is a histone phosphorylase or kinase, where examples include MSK1, MSK2, ATR, ATM, DNA-PK, Bubl, VprBP, IKK-a, PKCpi, Dik/Zip, JAK2, PKC5, WSTF and CK2. In some embodiments, a methylation domain is used and may be chosen from groups such as Ezh2, PRMT1/6, PRMT5/7, PRMT 2/6, CARM1, set7/9, MLL, ALL-1, Suv 39h, G9a, SETDB1, Ezh2, Set2, Dotl, PRMT 1/6, PRMT 5/7, PR-Set7 and Suv4-20h, Domains involved in sumoylation and biotinylation (Lys9, 13, 4, 18 and 12) may also be used in some embodiments (review see Kousarides (2007) Cell 128:693-705).

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, IL) Catalogue. Methods and compositions for making fusions between a minor groove binder and a polypeptide have been described. Mapp et al, (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein.

In some embodiments, the present disclosure provides a cell (e.g., a primary T cell and a hypoimmunogenic stem cell and derivative thereof) or population thereof comprising a genome in which the cell genome has been modified to express CD47. In some embodiments, the present disclosure provides a method for altering a cell genome to express CD47. In certain embodiments, at least one ribonucleic acid or at least one pair of ribonucleic acids may be utilized to facilitate the insertion of CD47 into a cell line. In certain embodiments, the at least one ribonucleic acid or the at least one pair of ribonucleic acids is selected from the group consisting of SEQ ID NOS:200784-231885 of Table 29 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-C. In some embodiments, the present disclosure provides a method for altering a cell genome to express HLA-C. In certain embodiments, at least one ribonucleic acid or at least one pair of ribonucleic acids may be utilized to facilitate the insertion of HLA-C into a cell line. In certain embodiments, the at least one ribonucleic acid or the at least one pair of ribonucleic acids is selected from the group consisting of SEQ ID NOS:3278-5183 of Table 10 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-E. In some embodiments, the present disclosure provides a method for altering a cell genome to express HLA-E. In certain embodiments, at least one ribonucleic acid or at least one pair of ribonucleic acids may be utilized to facilitate the insertion of HLA-E into a cell line. In certain embodiments, the at least one ribonucleic acid or the at least one pair of ribonucleic acids is selected from the group consisting of SEQ ID NOS:189859-193183 of Table 19 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-F. In some embodiments, the present disclosure provides a method for altering a cell genome to express HLA-F. In certain embodiments, at least one ribonucleic acid or at least one pair of ribonucleic acids may be utilized to facilitate the insertion of HLA-F into a cell line. In certain embodiments, the at least one ribonucleic acid or the at least one pair of ribonucleic acids is selected from the group consisting of SEQ ID NOS: 688808-399754 of Table 45 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-G. In some embodiments, the present disclosure provides a method for altering a cell genome to express HLA-G. In certain embodiments, at least one ribonucleic acid or at least one pair of ribonucleic acids may be utilized to facilitate the insertion of HLA-G into a stem cell line. In certain embodiments, the at least one ribonucleic acid or the at least one pair of ribonucleic acids is selected from the group consisting of SEQ ID NOS:188372-189858 of Table 18 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express PD-L1. In some embodiments, the present disclosure provides a method for altering a cell genome to express PD-L1. In certain embodiments, at least one ribonucleic acid or at least one pair of ribonucleic acids may be utilized to facilitate the insertion of PD-L1 into a stem cell line. In certain embodiments, the at least one ribonucleic acid or the at least one pair of ribonucleic acids is selected from the group consisting of SEQ ID NOS:193184-200783 of Table 21 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express CTLA4-Ig. In some embodiments, the present disclosure provides a method for altering a cell genome to express CTLA4-Ig. In certain embodiments, at least one ribonucleic acid or at least one pair of ribonucleic acids may be utilized to facilitate the insertion of CTLA4-Ig into a stem cell line. In certain embodiments, the at least one ribonucleic acid or the at least one pair of ribonucleic acids is selected from any one disclosed in WO2016183041, including the sequence listing.

In some embodiments, the present disclosure provides a cell (e.g., a primary T cell and a hypoimmunogenic stem cell and derivative thereof) or population thereof comprising a genome in which the cell genome has been modified to express C1-inhibitor. In some embodiments, the present disclosure provides a method for altering a cell genome to express C1-inhibitor. In certain embodiments, at least one ribonucleic acid or at least one pair of ribonucleic acids may be utilized to facilitate the insertion of C1-inhibitor into a stem cell line. In certain embodiments, the at least one ribonucleic acid or the at least one pair of ribonucleic acids is selected from any one disclosed in WO2016183041, including the sequence listing.

In some embodiments, the present disclosure provides a cell (e.g., a primary T cell and a hypoimmunogenic stem cell and derivative thereof) or population thereof comprising a genome in which the cell genome has been modified to express IL-35. In some embodiments, the present disclosure provides a method for altering a cell genome to express IL-35. In certain embodiments, at least one ribonucleic acid or at least one pair of ribonucleic acids may be utilized to facilitate the insertion of IL-35 into a stem cell line. In certain embodiments, the at least one ribonucleic acid or the at least one pair of ribonucleic acids is selected from any one disclosed in WO2016183041, including the sequence listing.

In some embodiments, the tolerogenic factors are expressed in a cell using an expression vector. In some embodiments, the tolerogenic factors are introduced to the cell using a viral expression vector that mediates integration of the tolerogenic factor sequence into the genome of the cell. For example, the expression vector for expressing CD47 in a cell comprises a polynucleotide sequence encoding CD47. The expression vector can be an inducible expression vector. The expression vector can be a viral vector, such as but not limited to, a lentiviral vector. In some embodiments, the tolerogenic factors are introduced into the cells using fusogen-mediated delivery or a transposase system selected from the group consisting of conditional or inducible transposases, conditional or inducible PiggyBac transposons, conditional or inducible Sleeping Beauty (SB 11) transposons, conditional or inducible MosI transposons, and conditional or inducible Tol2 transposons.

In some embodiments, the present disclosure provides a cell (e.g., a primary T cell and a hypoimmunogenic stem cell and derivative thereof) or population thereof comprising a genome in which the cell genome has been modified to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, and IDO1. In some embodiments, the present disclosure provides a method for altering a cell genome to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, and IDO1. In certain embodiments, at least one ribonucleic acid or at least one pair of ribonucleic acids may be utilized to facilitate the insertion of the selected polypeptide into a stem cell line. In certain embodiments, the at least one ribonucleic acid or the at least one pair of ribonucleic acids is selected from any one disclosed in Appendices 1-47 and the sequence listing of WO2016183041, the disclosure is incorporated herein by references.

In some embodiments, a suitable gene editing system (e.g., CRISPR/Cas system or any of the gene editing systems described herein) is used to facilitate the insertion of a polynucleotide encoding a tolerogenic factor, into a genomic locus of the hypoimmunogenic cell. In some cases, the polynucleotide encoding the tolerogenic factor is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding the tolerogenic factor is inserted into a B2M gene locus, a CIITA gene locus, a TRAC gene locus, or a TRB gene locus. In some embodiments, the polynucleotide encoding the tolerogenic factor is inserted into any one of the gene loci depicted in Table 15 provided herein. In certain embodiments, the polynucleotide encoding the tolerogenic factor is operably linked to a promoter.

In some embodiments, the cells are engineered to expresses an increased amount of one or more of CD47, DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, CD16, CD52, H2-M3, CD16 Fc receptor, IL15-RF, H2-M3(HLA-G), B2M-HLA-E, A20/TNFAIP3, CR1, HLA-F, MANF, and/or Serpinb9 relative to a cell of the same cell type that does not comprise the modifications.

### N. Characteristics of Hypoimmunogenic Cells

In some embodiments, the population of hypoimmunogenic stem cells retains pluripotency as compared to a control stem cell (e.g., a wild-type stem cell or immunogenic stem cell). In some embodiments, the population of hypoimmunogenic stem cells retains differentiation potential as compared to a control stem cell (e.g., a wild-type stem cell or immunogenic stem cell).

In some embodiments, the administered population of hypoimmunogenic cells such as hypoimmunogenic CAR-T cells elicits a decreased or lower level of immune activation in the subject or patient. In some instances, the level of immune activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation produced by the administration of immunogenic cells. In some embodiments, the administered population of hypoimmunogenic cells fails to elicit immune activation in the subject or patient.

In some embodiments, the administered population of hypoimmunogenic cells such as hypoimmunogenic CAR-T cells elicits a decreased or lower level of T cell response in the subject or patient. In some instances, the level of T cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of T cell response produced by the administration of immunogenic cells. In some embodiments, the administered population of hypoimmunogenic cells fails to elicit a T cell response to the cells in the subject or patient.

In some embodiments, the administered population of hypoimmunogenic cells such as hypoimmunogenic CAR-T cells elicits a decreased or lower level of NK cell response in the subject or patient. In some instances, the level of NK cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of NK cell response produced by the administration of immunogenic cells. In some embodiments, the administered population of hypoimmunogenic cells fails to elicit an NK cell response to the cells in the subject or patient.

In some embodiments, the administered population of hypoimmunogenic cells such as hypoimmunogenic CAR-T cells elicits a decreased or lower level of macrophage engulfment in the subject or patient. In some instances, the level of NK cell response elicited by the cells is at least 5%, 10%, 15%, 20%, 25%, 30%, 35%, 40%, 45% 50%, 55%, 60%, 65%, 70%, 75%, 80%, 85%, 90%, 91%, 92%, 93%, 94%, 95%, 96%, 97%, 98%, or 99% lower compared to the level of macrophage engulfment produced by the administration of immunogenic cells. In some embodiments, the administered population of hypoimmunogenic cells fails to elicit macrophage engulfment of the cells in the subject or patient.

In some embodiments, the administered population of hypoimmunogenic cells such as hypoimmunogenic CAR-T cells elicits a decreased or lower level of systemic TH1 activation in the subject or patient. In some instances, the level of systemic TH1 activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of systemic TH1 activation produced by the administration of immunogenic cells. In some embodiments, the administered population of hypoimmunogenic cells fails to elicit systemic TH1 activation in the subject or patient.

In some embodiments, the administered population of hypoimmunogenic cells such as hypoimmunogenic CAR-T cells elicits a decreased or lower level of NK cell killing in the subject or patient. In some instances, the level of NK cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of NK cell killing produced by the administration of immunogenic cells. In some embodiments, the administered population of hypoimmunogenic cells fails to elicit NK cell killing in the subject or patient.

In some embodiments, the administered population of hypoimmunogenic cells such as hypoimmunogenic CAR-T cells elicits a decreased or lower level of immune activation of peripheral blood mononuclear cells (PBMCs) in the subject or patient. In some instances, the level of immune activation of PBMCs elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation of PBMCs produced by the administration of immunogenic cells. In some embodiments, the administered population of hypoimmunogenic cells fails to elicit immune activation of PBMCs in the subject or patient.

In some embodiments, the administered population of hypoimmunogenic cells such as hypoimmunogenic CAR-T cells elicits a decreased or lower level of donor-specific IgG antibodies in the subject or patient. In some instances, the level of donor-specific IgG antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgG antibodies produced by the administration of immunogenic cells. In some embodiments, the administered population of hypoimmunogenic cells fails to elicit donor-specific IgG antibodies in the subject or patient.

In some embodiments, the administered population of hypoimmunogenic cells such as hypoimmunogenic CAR-T cells elicits a decreased or lower level of donor-specific IgM antibodies in the subject or patient. In some instances, the level of donor-specific IgM antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgM antibodies produced by the administration of immunogenic cells. In some embodiments, the administered population of hypoimmunogenic cells fails to elicit donor-specific IgM antibodies in the subject or patient.

In some embodiments, the administered population of hypoimmunogenic cells such as hypoimmunogenic CAR-T cells elicits a decreased or lower level of IgM and IgG antibody production in the subject or patient. In some instances, the level of IgM and IgG antibody production elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of IgM and IgG antibody production produced by the administration of immunogenic cells. In some embodiments, the administered population of hypoimmunogenic cells fails to elicit IgM and IgG antibody production in the subject or patient.

In some embodiments, the administered population of hypoimmunogenic cells such as hypoimmunogenic CAR-T cells elicits a decreased or lower level of cytotoxic T cell killing in the subject or patient. In some instances, the level of cytotoxic T cell killing elicited by the cells is at least 5%, 10%, 15%, 20%, 25%, 30%, 35%, 40%, 45%, 50%, 55%, 60%6, 65%, 70%, 75%, 80%, 85%0, 90%, 91%, 92%, 93%, 94%, 95%, 96%, 97%, 98%, or 99% lower compared to the level of cytotoxic T cell killing produced by the administration of immunogenic cells. In some embodiments, the administered population of hypoimmunogenic cells fails to elicit cytotoxic T cell killing in the subject or patient.

In some embodiments, the administered population of hypoimmunogenic cells such as hypoimmunogenic CAR-T cells elicits a decreased or lower level of complement-dependent cytotoxicity (CDC) in the subject or patient. In some instances, the level of CDC elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of CDC produced by the administration of immunogenic cells. In some embodiments, the administered population of hypoimmunogenic cells fails to elicit CDC in the subject or patient.

O. Therapeutic Cells from Primary T Cells

Provided herein are hypoimmunogenic cells including, but not limited to, primary T cells that evade immune recognition. In some embodiments, the hypoimmunogenic cells are produced (e.g., generated, cultured, or derived) from T cells such as primary T cells. In some instances, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, the pool of primary T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of T cells do not include cells from the patient. In some embodiments, one or more of the donor subjects from which the pool of T cells is obtained are different from the patient.

In some embodiments, the hypoimmunogenic cells do not activate an innate and/or an adaptive immune response in the patient (e.g., recipient upon administration). Provided are methods of treating a disorder by administering a population of hypoimmunogenic cells to a subject (e.g., recipient) or patient in need thereof. In some embodiments, the hypoimmunogenic cells described herein comprise T cells engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells comprise reduced expression of an endogenous T cell receptor.

In some embodiments, the present disclosure is directed to hypoimmunogenic primary T cells that overexpress CD47 and CARs, and have reduced expression or lack expression of MHC class I and/or MHC class II human leukocyte antigens and have reduced expression or lack expression of TCR complex molecules. The cells outlined herein overexpress CD47 and CARs and evade immune recognition. In some embodiments, the primary T cells display reduced levels or activity of MHC class I antigens, MHC class II antigens, and/or TCR complex molecules. In certain embodiments, primary T cells overexpress CD47 and CARs and harbor a genomic modification in the B2M gene. In some embodiments, T cells overexpress CD47 and CARs and harbor a genomic modification in the CIITA gene. In some embodiments, primary T cells overexpress CD47 and CARs and harbor a genomic modification in the TRAC gene. In some embodiments, primary T cells overexpress CD47 and CARs and harbor a genomic modification in the TRB gene. In some embodiments, T cells overexpress CD47 and CARs and harbor genomic modifications in one or more of the following genes: the B2M, CIITA, TRAC and TRB genes.

Exemplary T cells of the present disclosure are selected from the group consisting of cytotoxic T cells, helper T cells, memory T cells, central memory T cells, effector memory T cells, effector memory RA T cells, regulatory T cells, tissue infiltrating lymphocytes, and combinations thereof. In certain embodiments, the T cells express CCR7, CD27, CD28, and CD45RA. In some embodiments, the central T cells express CCR7, CD27, CD28, and CD45RO. In other embodiments, the effector memory T cells express PD-1, CD27, CD28, and CD45RO. In other embodiments, the effector memory RA T cells express PD-1, CD57, and CD45RA.

In some embodiments, the T cell is a modified (e.g., an engineered) T cell. In some cases, the modified T cell comprise a modification causing the cell to express at least one chimeric antigen receptor that specifically binds to an antigen or epitope of interest expressed on the surface of at least one of a damaged cell, a dysplastic cell, an infected cell, an immunogenic cell, an inflamed cell, a malignant cell, a metaplastic cell, a mutant cell, and combinations thereof. In other cases, the modified T cell comprise a modification causing the cell to express at least one protein that modulates a biological effect of interest in an adjacent cell, tissue, or organ when the cell is in proximity to the adjacent cell, tissue, or organ. Useful modifications to primary T cells are described in detail in US2016/0348073 and WO2020/018620, the disclosures of which are incorporated herein in their entireties.

In some embodiments, the hypoimmunogenic cells described herein comprise T cells that are engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells include reduced expression of an endogenous T cell receptor. In some embodiments, the T cells described herein such as the engineered or modified T cells include reduced expression of cytotoxic T-lymphocyte-associated protein 4 (CTLA-4). In other embodiments, the T cells described herein such as the engineered or modified T cells include reduced expression of programmed cell death (PD-1). In certain embodiments, the T cells described herein such as the engineered or modified T cells include reduced expression of CTLA-4 and PD-1. Methods of reducing or eliminating expression of CTLA-4, PD-1 and both CTLA-4 and PD-1 can include any recognized by those skilled in the art, such as but not limited to, genetic modification technologies that utilize rare-cutting endonucleases and RNA silencing or RNA interference technologies. Non-limiting examples of a rare-cutting endonuclease include any Cas protein, TALEN, zinc finger nuclease, meganuclease, and homing endonuclease. In some embodiments, an exogenous nucleic acid encoding a polypeptide as disclosed herein (e.g., a chimeric antigen receptor, CD47, or another tolerogenic factor disclosed herein) is inserted at a CTLA-4 and/or PD-1 gene locus. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

In some embodiments, the T cells described herein such as the engineered or modified T cells include enhanced expression of PD-L1.

In some embodiments, the hypoimmunogenic T cell includes a polynucleotide encoding a CAR, wherein the polynucleotide is inserted in a genomic locus. In some embodiments, the polynucleotide encoding the CAR is randomly integrated into the genome of the cell. In some embodiments, the polynucleotide encoding the CAR is randomly integrated into the genome of the cell via viral vector transduction. In some embodiments, the polynucleotide encoding the CAR is randomly integrated into the genome of the cell via lentiviral vector transduction. In some embodiments, the polynucleotide is inserted into a safe harbor or target locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus. In some embodiments, the polynucleotide is inserted in a B2M, CIITA, TRAC, TRB, PD-1 or CTLA-4 gene.

In some embodiments, the hypoimmunogenic T cell includes a polynucleotide encoding a CAR that is expressed in a cell using an expression vector. In some embodiments, the CAR is introduced to the cell using a viral expression vector that mediates integration of the CAR sequence into the genome of the cell. For example, the expression vector for expressing the CAR in a cell comprises a polynucleotide sequence encoding the CAR. The expression vector can be an inducible expression vector. The expression vector can be a viral vector, such as but not limited to, a lentiviral vector.

Hypoimmunogenic T cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

P. Therapeutic Cells Differentiated from Hypoimmunogenic Pluripotent Stem Cells

Provided herein are hypoimmunogenic cells including, cells derived from pluripotent stem cells, that evade immune recognition. In some embodiments, the cells do not activate an innate and/or an adaptive immune response in the patient or subject (e.g., recipient upon administration). Provided are methods of treating a disorder comprising repeat dosing of a population of hypoimmunogenic cells to a recipient subject in need thereof.

In some embodiments, the pluripotent stem cell and any cell differentiated from such a pluripotent stem cell is modified to exhibit reduced expression of MHC class I human leukocyte antigens. In other embodiments, the pluripotent stem cell and any cell differentiated from such a pluripotent stem cell is modified to exhibit reduced expression of MHC class II human leukocyte antigens. In certain embodiments, the pluripotent stem cell and any cell differentiated from such a pluripotent stem cell is modified to exhibit reduced expression of TCR complexes. In some embodiments, the pluripotent stem cell and any cell differentiated from such a pluripotent stem cell is modified to exhibit reduced expression of MHC class I and II human leukocyte antigens. In some embodiments, the pluripotent stem cell and any cell differentiated from such a pluripotent stem cell is modified to exhibit reduced expression of MHC class I and II human leukocyte antigens and TCR complexes.

In some embodiments, the pluripotent stem cell and any cell differentiated from such a pluripotent stem cell is modified to exhibit reduced expression of MHC class I and/or II human leukocyte antigens and exhibit increased CD47 expression. In some instances, the cell overexpresses CD47 by harboring one or more CD47 transgenes. In some embodiments, the pluripotent stem cell and any cell differentiated from such a pluripotent stem cell is modified to exhibit reduced expression of MHC class I and II human leukocyte antigens and exhibit increased CD47 expression. In some embodiments, the pluripotent stem cell and any cell differentiated from such a pluripotent stem cell is modified to exhibit reduced expression of MHC class I and II human leukocyte antigens and TCR complexes and exhibit increased CD47 expression.

In some embodiments, the pluripotent stem cell and any cell differentiated from such a pluripotent stem cell is modified to exhibit reduced expression of MHC class I and/or II human leukocyte antigens, to exhibit increased CD47 expression, and to exogenously express a chimeric antigen receptor. In some instances, the cell overexpresses CD47 polypeptides by harboring one or more CD47 transgenes. In some instances, the cell overexpresses CAR polypeptides by harboring one or more CAR transgenes. In some embodiments, the pluripotent stem cell and any cell differentiated from such a pluripotent stem cell is modified to exhibit reduced expression of MHC class I and II human leukocyte antigens, exhibit increased CD47 expression, and to exogenously express a chimeric antigen receptor. In some embodiments, the pluripotent stem cell and any cell differentiated from such a pluripotent stem cell is modified to exhibit reduced expression of MHC class I and II human leukocyte antigens and TCR complexes, to exhibit increased CD47 expression, and to exogenously express a chimeric antigen receptor.

Such pluripotent stem cells are hypoimmunogenic stem cells. Such differentiated cells are hypoimmunogenic cells.

Any of the pluripotent stem cells described herein can be differentiated into any cells of an organism and tissue. In some embodiments, the cells exhibit reduced expression of MHC class I and/or II human leukocyte antigens and reduced expression of TCR complexes. In some instances, expression of MHC class I and/or II human leukocyte antigens is reduced compared to unmodified or wild-type cell of the same cell type. In some instances, expression of TCR complexes is reduced compared to unmodified or wild-type cell of the same cell type. In some embodiments, the cells exhibit increased CD47 expression. In some instances, expression of CD47 is increased in cells encompassed by the present disclosure as compared to unmodified or wild-type cells of the same cell type. In some embodiments, the cells exhibit exogenous CAR expression. Methods for reducing levels of MHC class I and/or II human leukocyte antigens and TCR complexes and increasing the expression of CD47 and CARs are described herein.

In some embodiments, the cells used in the methods described herein evade immune recognition and responses when administered to a patient (e.g., recipient subject). The cells can evade killing by immune cells in vitro and in vivo. In some embodiments, the cells evade killing by macrophages and NK cells. In some embodiments, the cells are ignored by immune cells or a subject's immune system. In other words, the cells administered in accordance with the methods described herein are not detectable by immune cells of the immune system. In some embodiments, the cells are cloaked and therefore avoid immune rejection.

Methods of determining whether a pluripotent stem cell and any cell differentiated from such a pluripotent stem cell evades immune recognition include, but are not limited to, IFN-7 Elispot assays, microglia killing assays, cell engraftment animal models, cytokine release assays, ELISAs, killing assays using bioluminescence imaging or chromium release assay or a real-time, quantitative microelectronic biosensor system for cell analysis (xCELLigence® RTCA system, Agilent), mixed-lymphocyte reactions, immunofluorescence analysis, etc.

Therapeutic cells outlined herein are useful to treat a disorder such as, but not limited to, a cancer, a genetic disorder, a chronic infectious disease, an autoimmune disorder, a neurological disorder, and the like.

1. T Lymphocytes Differentiated from Hypoimmunogenic Pluripotent Cells

Provided herein, T lymphocytes (T cells, including primary T cells) are derived from the HIP cells described herein (e.g., hypoimmunogenic iPSCs). Methods for generating T cells, including CAR-T cells, from pluripotent stem cells (e.g., iPSCs) are described, for example, in Iriguchi et al., Nature Communications 12, 430 (2021); Themeli et al., Cell Stem Cell, 16(4):357-366 (2015); Themeli et al., Nature Biotechnology 31:928-933 (2013).

T lymphocyte derived hypoimmunogenic cells include, but are not limited to, primary T cells that evade immune recognition. In some embodiments, the hypoimmunogenic cells are produced (e.g., generated, cultured, or derived) from T cells such as primary T cells. In some instances, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, the pool of primary T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of T cells does not include cells from the patient. In some embodiments, one or more of the donor subjects from which the pool of T cells is obtained are different from the patient.

In some embodiments, the hypoimmunogenic cells do not activate an immune response in the patient (e.g., recipient upon administration). Provided are methods of treating a disorder by administering a population of hypoimmunogenic cells to a subject (e.g., recipient) or patient in need thereof. In some embodiments, the hypoimmunogenic cells described herein comprise T cells engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells comprise reduced expression of an endogenous T cell receptor.

In some embodiments, the HIP-derived T cell includes a chimeric antigen receptor (CAR). Any suitable CAR can be included in the hyHIP-derived T cell, including the CARs described herein. In some embodiments, the hypoimmunogenic induced pluripotent stem cell-derived T cell includes a polynucleotide encoding a CAR, wherein the polynucleotide is inserted in a genomic locus. In some embodiments, the polynucleotide is inserted into a safe harbor or target locus. In some embodiments, the polynucleotide is inserted in a B2M, CIITA, TRAC, TRB, PD-1 or CTLA-4 gene. Any suitable method can be used to insert the CAR into the genomic locus of the hypoimmunogenic cell including the gene editing methods described herein (e.g., a CRISPR/Cas system).

HIP-derived T cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

2. NK Cells Derived from Hypoimmunogenic Pluripotent Cells

Provided herein, natural killer (NK) cells are derived from the HIP cells described herein (e.g., hypoimmunogenic iPSCs).

NK cells (also defined as ‘large granular lymphocytes’) represent a cell lineage differentiated from the common lymphoid progenitor (which also gives rise to B lymphocytes and T lymphocytes). Unlike T-cells, NK cells do not naturally comprise CD3 at the plasma membrane. Importantly, NK cells do not express a TCR and typically also lack other antigen-specific cell surface receptors (as well as TCRs and CD3, they also do not express immunoglobulin B-cell receptors, and instead typically express CD16 and CD56). NK cell cytotoxic activity does not require sensitization but is enhanced by activation with a variety of cytokines including IL-2. NK cells are generally thought to lack appropriate or complete signaling pathways necessary for antigen-receptor-mediated signaling, and thus are not thought to be capable of antigen receptor-dependent signaling, activation and expansion. NK cells are cytotoxic, and balance activating and inhibitory receptor signaling to modulate their cytotoxic activity. For instance, NK cells expressing CD16 may bind to the Fc domain of antibodies bound to an infected cell, resulting in NK cell activation. By contrast, activity is reduced against cells expressing high levels of MHC class I proteins. On contact with a target cell NK cells release proteins such as perforn, and enzymes such as proteases (granzymes). Perforin can form pores in the cell membrane of a target cell, inducing apoptosis or cell lysis.

There are a number of techniques that can be used to generate NK cells, including CAR-NK-cells, from pluripotent stem cells (e.g., iPSC); see, for example, Zhu et al., Methods Mol Biol. 2019; 2048:107-119; Knorr et al., Stem Cells Transl Med. 2013 2(4):274-83. doi: 10.5966/sctm.2012-0084; Zeng et al., Stem Cell Reports. 2017 Dec. 12; 9(6):1796-1812; Ni et al., Methods Mol Biol. 2013; 1029:33-41; Bernareggi et al., Exp Hematol. 2019 71:13-23; Shankar et al., Stem Cell Res Ther. 2020; 11(1):234, all of which are incorporated herein by reference in their entirety and specifically for the methodologies and reagents for differentiation. Differentiation can be assayed as is known in the art, generally by evaluating the presence of NK cell associated and/or specific markers, including, but not limited to, CD56, KIRs, CD16, NKp44, NKp46, NKG2D, TRAIL, CD122, CD27, CD244, NK1.1, NKG2A/C, NCR1, Ly49, CD49b, CD11b, KLRG1, CD43, CD62L, and/or CD226.

In some embodiments, the hypoimmunogenic pluripotent cells are differentiated into hepatocytes to address loss of the hepatocyte functioning or cirrhosis of the liver. There are a number of techniques that can be used to differentiate HIP cells into hepatocytes; see for example, Pettinato et al., doi: 10.1038/spre32888, Snykers et al., Methods Mol Biol., 2011 698:305-314, Si-Tayeb et al., Hepatology, 2010, 51:297-305 and Asgari et al., Stem Cell Rev., 2013, 9(4):493-504, all of which are incorporated herein by reference in their entirety and specifically for the methodologies and reagents for differentiation. Differentiation can be assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release, and glycogen storage.

In some embodiments, the NK cells do not activate an innate and/or an adaptive immune response in the patient (e.g., recipient upon administration). Provided are methods of treating a disorder by administering a population of NK cells to a subject (e.g., recipient) or patient in need thereof. In some embodiments, the NK cells described herein comprise NK cells engineered (e.g., are modified) to express a chimeric antigen receptor including but not limited to a chimeric antigen receptor described herein. Any suitable CAR can be included in the NK cells, including the CARs described herein. In some embodiments, the NK cell includes a polynucleotide encoding a CAR, wherein the polynucleotide is inserted in a genomic locus. In some embodiments, the polynucleotide is inserted into a safe harbor or a target locus. In some embodiments, the polynucleotide is inserted in a B2M, CIITA, PD1 or CTLA4 gene. Any suitable method can be used to insert the CAR into the genomic locus of the NK cell including the gene editing methods described herein (e.g., a CRISPR/Cas system).

### Q. Assays for Hypoimmunogenicity Phenotypes and Retention of Pluripotency

Once the hypoimmunogenic cells have been generated, they may be assayed for their hypoimmunogenicity and/or retention of pluripotency as is described in WO2016183041 and WO2018132783.

In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g., teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell responses can be assessed by Elispot, ELISA, FACS, PCR, or mass cytometry (CYTOF). B cell responses or antibody responses are assessed using FACS or Luminex. Additionally, or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of hypoimmunogenic cells is determined using an allogenic humanized immunodeficient mouse model. In some instances, the hypoimmunogenic pluripotent stem cells are transplanted into an allogenic humanized NSG-SGM3 mouse and assayed for cell rejection, cell survival, and teratoma formation. In some instances, grafted hypoimmunogenic pluripotent stem cells or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

Similarly, the retention of pluripotency is tested in a number of ways. In some embodiments, pluripotency is assayed by the expression of certain pluripotency-specific factors as generally described herein and shown in FIG. 29 of WO2018132783. Additionally or alternatively, the pluripotent cells are differentiated into one or more cell types as an indication of pluripotency.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A, HLA-B, and HLA-C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the MHC II function (HLA II when the cells are derived from human cells) in the pluripotent cells or their derivatives can be measured using techniques known in the art such as Western blotting using antibodies to the protein, FACS techniques, RT-PCR techniques, etc.

In addition, the cells can be tested to confirm that the HLA II complex is not expressed on the cell surface. Again, this assay is done as is known in the art (See FIG. 21 of WO2018132783, for example) and generally is done using either Western Blots or FACS analysis based on commercial antibodies that bind to human HLA Class II HLA-DR, DP and most DQ antigens.

In addition to the reduction of HLA I and II (or MHC I and II), the hypoimmunogenic cells of the technology have a reduced susceptibility to macrophage phagocytosis and NK cell killing. The resulting hypoimmunogenic cells “escape” the immune macrophage and innate pathways due to reduction or lack of the TCR complex and the expression of one or more CD47 transgenes.

In some aspects, the present technology provides T cells, such as immune evasive allogeneic T cells, that are derived from or generated by methods according to various embodiments disclosed herein. In some embodiments, the generated T cells are suitable for use in adoptive cell therapy, as they have been made to be immune evasive (e.g., by inserting a tolerogenic factor into an endogenous TCR gene locus and/or by modifying the MHC I and/or MHC II genes as described) and to express one or more CARs.

In some embodiments, the T cell is a naïve T cell, a helper T cell (CD4+), a cytotoxic T cell (CD8+), a regulatory T cell (Treg), a central memory T cell (T_{CM}), an effector memory T cell (T_{EM}), a stem cell memory T cell (T_{SCM}), or any combination thereof. More specifically, the T cell can be naïve (not exposed to antigen; increased expression of CD62L, CCR7, CD28, CD3, CD127, and CD45RA, and decreased expression of CD45RO as compared to T_{CM}), memory T cells (antigen-experienced and long-lived), or effector cells (antigen-experienced, cytotoxic). Memory T cells can be further divided into subsets of T_{CM} (increased expression of CD62L, CCR7, CD28, CD127, CD45RO, and CD95, and decreased expression of CD54RA as compared to naïve T cells) and T_{EM} (decreased expression of CD62L, CCR7, CD28, CD45RA, and increased expression of CD127 as compared to naïve T cells or T_{CM}). Effector T cells refer to antigen-experienced CD8+ cytotoxic T cells that has decreased expression of CD62L, CCR7, CD28, and are positive for granzyme and perform as compared to T_{CM}. Helper T cells are CD4+ cells that influence the activity of other immune cells by releasing cytokines. CD4+ T cells can activate or suppress an adaptive immune response, and which of those two functions is induced will depend on the presence of other cells and signals. T cells can be collected using known techniques, and the various subpopulations or combinations thereof can be enriched or depleted by known techniques, such as by affinity binding to antibodies, flow cytometry, or immunomagnetic selection.

In some embodiments, the T cell is an autologous cell, i.e., obtained from the subject who will receive the T cell after modification. In some embodiments, the T cell is an allogeneic T cell, i.e., obtained from someone other than the subject who will receive the T cell after modification. In either of these embodiments, the T cells can be primary T cells obtained from a number of sources, including peripheral blood mononuclear cells, bone marrow, lymph node tissue, cord blood, thymus tissue, tissue from a site of infection, ascites, pleural effusion, spleen tissue, and tumors. In other embodiments, especially in the case of allogeneic T cells, the T cells can be derived or differentiated from embryonic stem cells (ESCs) or induced pluripotent cells (iPSCs).

In some aspects, the present technology provides pharmaceutical compositions comprising a T cell according to various embodiments disclosed herein.

In some embodiments, the compositions can have various formulations, for example, injectable formulations, lyophilized formulations, liquid formulations, oral formulations, etc., depending on the suitable routes of administration.

In some embodiments, the compositions can be co-formulated in the same dosage unit or can be individually formulated in separate dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e., 1 to 10, 1 to 8, 1 to 6, 1 to 4, or 1 to 2) of times per day, or as many times as needed to elicit a therapeutic response.

In some embodiments, a single dosage unit includes at least about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells.

### IV. Methods of Treatment

In some aspects, the present technology provides methods for treating and/or preventing a disease in a subject in need thereof using T cells, such as immune evasive allogeneic T cells, derived from or generated by methods according to various embodiments disclosed herein. The method entails administering to the subject a therapeutically effective amount of the T cell, or a pharmaceutical composition containing the same.

The T cell can be an autologous cell, i.e., obtained from the subject who will receive the T cell after modification. Alternatively, the T cell can be an allogeneic T cell, i.e., obtained from someone other than the subject who will receive the T cell after modification. In either of these embodiments, the T cells can be primary T cells obtained from a number of sources, including peripheral blood mononuclear cells, bone marrow, lymph node tissue, cord blood, thymus tissue, tissue from a site of infection, ascites, pleural effusion, spleen tissue, and tumors. In other embodiments, especially in the case of allogeneic T cells, the T cells can be derived from ESCs or iPSCs.

In some embodiments, the T cell is a naïve T cell, a helper T cell (CD4+), a cytotoxic T cell (CD8+), a regulatory T cell (Treg), a central memory T cell (TCM), an effector memory T cell (TEM), a stem cell memory T cell (TSCM), or any combination thereof. In some embodiments, the T cell expresses a tolerogenic factor (e.g., CD47, HLA-E, HLA-G, PD-L1, CTLA-4) and/or a CAR (e.g., CD19 CAR, CD22 CAR, BCMA CAR). In these embodiments, the T cell recognizes and initiates an immune response to a target cell expressing the antigen the CAR is designed to target (e.g., CD19, CD22, BCMA), and the T cell possesses hypoimmunity in an allogeneic recipient due to expression of the tolerogenic factor.

In some embodiments, the disease is cancer, for example, one associated with CD19, CD22, or BCMA expression, i.e., the cancer cell expresses CD19, CD22, or BCMA. In these embodiments, the method comprises contacting the cancer cell with a T cell generated by methods of the present technology and expressing the corresponding CAR, such that the CAR is activated in response to the antigen expressed on the cancer cell and subsequently initiates killing of the cancer cell.

In some embodiments, the cancer is a hematologic malignancy. Non-limiting examples of hematologic malignancies include myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

In some embodiments, a cancer is solid malignancy. Non-limiting examples of hematologic malignancies comprise: breast cancer, ovarian cancer, colon cancer, prostate cancer, epithelial cancer, renal-cell carcinoma, pancreatic adenocarcinoma, cervical carcinoma, colorectal cancer, glioblastoma, rhabdomyosarcoma, neuroblastoma, melanoma, Ewing sarcoma, osteosarcoma, mesothelioma and adenocarcinoma.

In some embodiments, the disease is an autoimmune disease, including, for example, lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

In some embodiments, the disease is diabetes mellitus, including, for example, Type I diabetes, Type II diabetes, prediabetes, and gestational diabetes.

In some embodiments, the disease is a neurological disease, including, for example, catalepsy, epilepsy, encephalitis, meningitis, migraine, Huntington's, Alzheimer's, Parkinson's, Pelizaeus-Merzbacher disease, and multiple sclerosis.

Provided herein are compositions suitable for use in a subject, including therapeutic compositions and cell therapy compositions. Provided herein are pharmaceutical compositions comprising a population of engineered cells as described herein and a pharmaceutically acceptable additive, carrier, diluent or excipient. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); salts such as sodium chloride; and/or non-ionic surfactants such as polysorbates (TWEEN™), poloxamers (PLURONICS™) or polyethylene glycol (PEG). In some embodiments, the pharmaceutical composition includes a pharmaceutically acceptable buffer (e.g., neutral buffer saline or phosphate buffered saline). In some embodiments, the pharmaceutically acceptable additive, carrier, diluent or excipient comprises one or more of Plasma-Lyte A®, dextrose, dextran, sodium chloride, human serum albumin (HSA), dimethylsulfoxide (DMSO), or a combination thereof. In some embodiments, the composition further comprises a pharmaceutically acceptable buffer. In some embodiments, the pharmaceutically acceptable buffer is neutral buffer saline or phosphate buffered saline.

In some embodiments, the T cell, or a pharmaceutical composition containing the same, according to the present technology may be administered in a manner appropriate to the disease, condition, or disorder to be treated as determined by persons skilled in the medical art. In any of the above embodiments, the T cell, or a pharmaceutical composition containing the same, can be administered intravenously, intraperitoneally, intratumorally, into the bone marrow, into a lymph node, or into the cerebrospinal fluid, so as to encounter the target antigen or cells. An appropriate dose, suitable duration, and frequency of administration of the compositions will be determined by such factors as a condition of the patient; size, type, and severity of the disease, condition, or disorder; the undesired type or level or activity of the tagged cells, the particular form of the active ingredient; and the method of administration.

In some embodiments, the amount of the T cells in a pharmaceutical composition is typically greater than 10² cells, for example, about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰ cells, or more.

In some embodiments, the methods comprise administering to the subject the T cell, or a pharmaceutical composition containing the same, once a day, twice a day, three times a day, or four times a day for a period of about 3 days, about 5 days, about 7 days, about 10 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 1.25 years, about 1.5 years, about 1.75 years, about 2 years, about 2.25 years, about 2.5 years, about 2.75 years, about 3 years, about 3.25 years, about 3.5 years, about 3.75 years, about 4 years, about 4.25 years, about 4.5 years, about 4.75 years, about 5 years, or more than about 5 years. In some embodiments, the host cells or the pharmaceutical composition containing the same can be administered every day, every other day, every third day, weekly, biweekly (i.e., every other week), every third week, monthly, every other month, or every third month.

In some embodiments, the T cell, or a pharmaceutical composition containing the same, may be administered over a pre-determined time period. Alternatively, the T cell, or a pharmaceutical composition containing the same, may be administered until a particular therapeutic benchmark is reached. In some embodiments, the methods provided herein include a step of evaluating one or more therapeutic benchmarks in a biological sample, such as, but not limited to, the level of a cancer biomarker, to determine whether to continue administration of the host cell, or the pharmaceutical composition containing the same.

In some embodiments, the method further entails administering one or more other cancer therapies such as surgery, immunotherapy, radiotherapy, and/or chemotherapy to the subject, sequentially or simultaneously.

In some embodiments, the methods further comprise administering the subject a pharmaceutically effective amount of one or more additional therapeutic agents to obtain improved or synergistic therapeutic effects. In some embodiments, the one or more additional therapeutic agents are selected from the group consisting of an immunotherapy agent, a chemotherapy agent, and a biologic agent. In some embodiments, the subject was administered the one or more additional therapeutic agents before administration of the T cell, or a pharmaceutical composition containing the same. In some embodiments, the subject is co-administered the one or more additional therapeutic agents and the T cell, or a pharmaceutical composition containing the same. In some embodiments, the subject was administered the one or more additional therapeutic agents after administration of the T cell, or a pharmaceutical composition containing the same.

As one of ordinary skill in the art would understand, the one or more additional therapeutic agents and the T cell, or a pharmaceutical composition containing the same, can be administered to a subject in need thereof one or more times at the same or different doses, depending on the diagnosis and prognosis of the subject. One skilled in the art would be able to combine one or more of these therapies in different orders to achieve the desired therapeutic results. In some embodiments, the combinational therapy achieves improved or synergistic effects in comparison to any of the treatments administered alone.

### EXAMPLES

The following examples are provided so as to describe to the skilled artisan how to make and use methods and compositions described herein; and are not intended to limit the scope of the present disclosure. Unless indicated otherwise, temperature is indicated in Celsius and pressure is at or near atmospheric.

In general, the following examples illustrate transgene knock-in efficiency and transgene expression at a TRAC locus.

### Example 1—Genetic Engineering

This Example provides an exemplary method for inserting a transgene encoding a tolerogenic factor at a TCR gene locus. Specifically, this Example 1 demonstrates two exemplary insertion strategies for introducing a CD47 coding region into a human TRAC gene locus. In addition to inserting CD47, both exemplary strategies also knock-out TRAC gene expression.

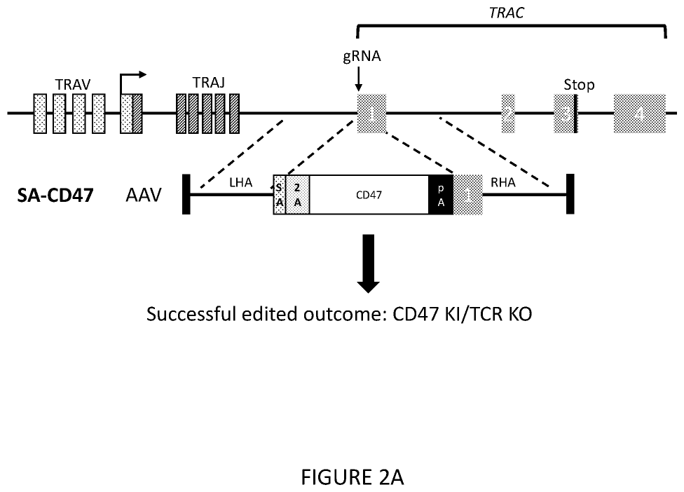

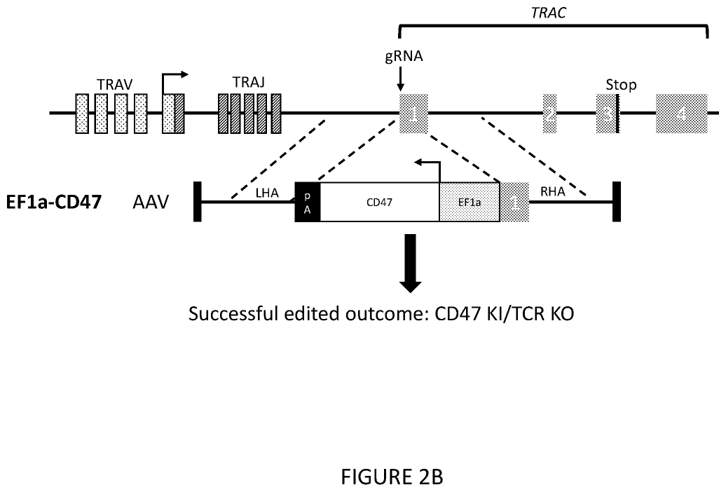

Two knock-in approaches were devised to express an exemplary transgene, CD47, in a human TRAC locus. FIG. 2A illustrates an approach using the SA-CD47 transgene. The SA-CD47 transgene was an AAV construct, which was flanked on each end by an AAV inverted terminal repeat (ITR). From 5′ to 3′, the SA-CD47 transgene further included a left homology arm (LHA), a splice acceptor, a 2A site, a human CD47 coding region, a poly-A tail site, and a right homology arm.

To introduce the SA-CD47 construct into the TRAC locus, CD8+ T cells were first stimulated with a-CD3/CD28/IL-2. Next, hTRAC-gRNA and Cas9 mRNA were introduced into the CD8+ T cells via nucleofection. Later, the transgene cassette (described above) was introduced via AAV6 transduction. Upon integration of SA-CD47 into the TRAC locus, the resulting engineered locus included, from 5′ to 3′: a plurality of T-cell receptor alpha variable (TRAV) genes (including the associated endogenous promoter), a plurality of T-cell receptor alpha joining (TRAJ) genes, a splice acceptor, a 2A site, a human CD47 coding region, a poly-A tail site, a TRAC exon 1 or a portion thereof, and the remaining TRAC exons (e.g., exons 2-4). Driven by the TRAV promoter, RNA was then transcribed from the engineered locus and the desired CD47 protein was expressed.

Flow cytometry analysis was performed to confirm that the resulting cells demonstrated the desired cell surface expression profile and cells were harvested for NGS analysis.

FIG. 2B illustrates a second approach, which used an exogenous promoter to drive transgene expression (e.g., EF1a). The EF1a-CD47 transgene was also an AAV construct, which is flanked on each end by an AAV inverted terminal repeat (ITR). From 5′ to 3′, the EF1a-CD47 transgene further included an LHA, a poly-A tail site, a human CD47 coding region, an EF1a promoter, and a right homology arm.

The EF1a-CD47 construct was introduced into CD8+ T cells as described above in this Example. Upon integration of EF1a-CD47 into the TRAC locus, the resulting engineered locus included, from 5′ to 3′: a plurality of TRAV genes, a plurality of TRAJ genes, a poly-A tail site, a human CD47 coding region, an EF1a promoter, a TRAC exon 1 or a portion thereof, and the remaining TRAC exons (e.g., exons 2-4). Driven by the EF1a promoter, RNA is then transcribed from the engineered locus and the desired CD47 protein is expressed.

Flow cytometry analysis is performed to confirm the desired cell surface expression profile and cells are harvested for next-generation sequencing (NGS) analysis.

### Example 2—gRNA Editing Efficiency

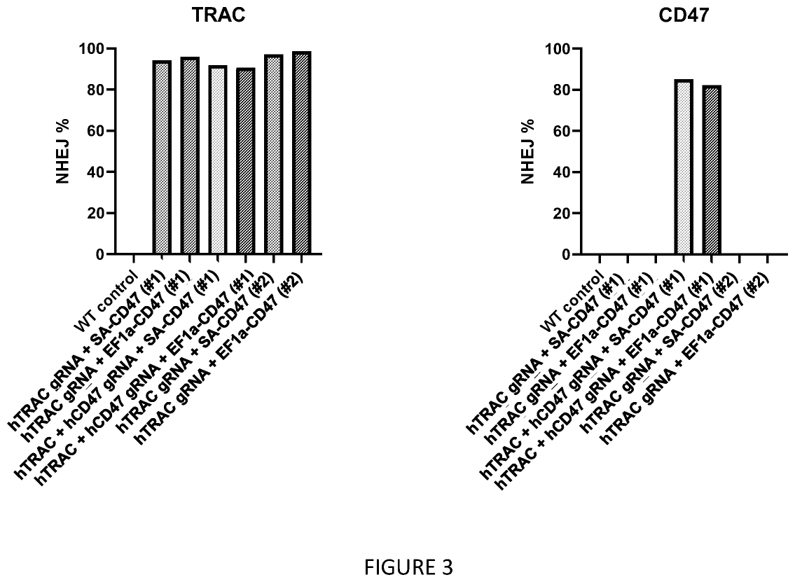

As shown in FIG. 3, PCR was used to assess: 1) the efficiency with which the endogenous TRAC gene or endogenous TRAC gene and endogenous CD47 gene could be knocked out. Exemplary hTRAC gRNA comprising a nucleic acid sequence of TCAGGGTTCTGGATATCTGT (SEQ ID NO: 124), and exemplary hCD47 gRNA comprising a nucleic acid sequence of TTTGGAGAAAACCATGAAAC (SEQ ID NO: 125) were used.

FIG. 3 illustrates that all groups demonstrated high levels of NHEJ of TRAC relative to the wild-type (WT) control. However, only the groups that included hCD47 gRNA demonstrated high levels of NHEJ of CD47 relative to the control.

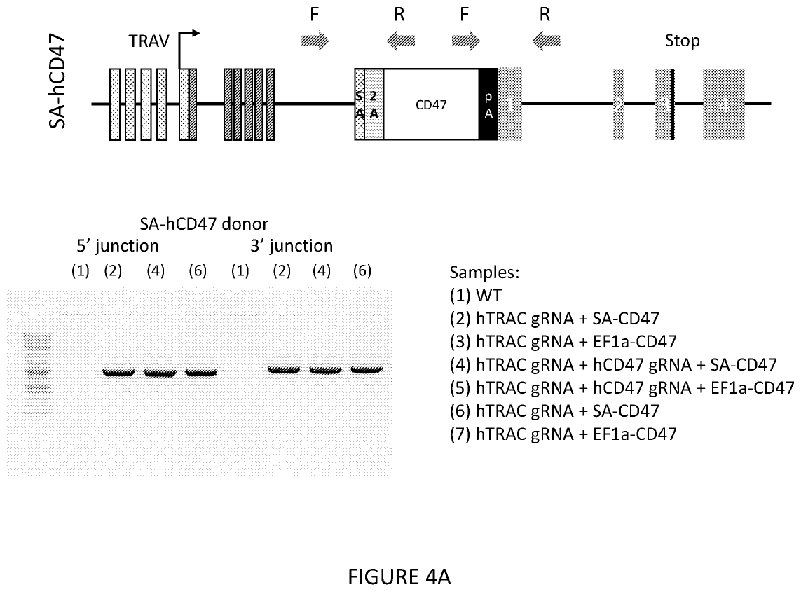

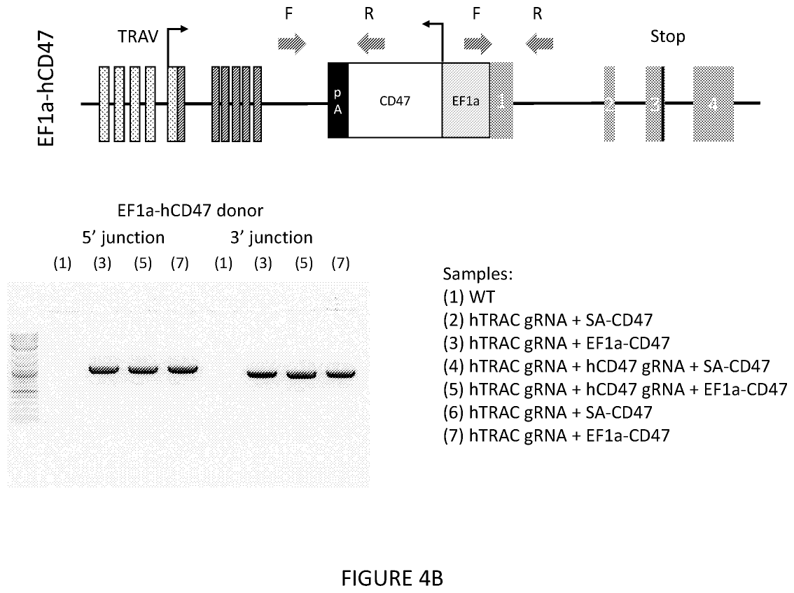

Junction PCR across the insertion site of exemplary CD47 transgenes (see FIGS. 4A and 4B) was used to confirm insertion of the transgene at the target (TRAC) locus.

### Example 3—Generation of Primary CD8+ T Cells with CRIPSR/Cas9 Targeted Integration of CD47 at TRAC Locus

Flow cytometry analysis was performed on primary CD8+ cells to determine TRAC knockout and transgene (CD47) expression. TRAC knockout was assessed by determining levels of CD3 cell surface expression. As TCR levels on a cell surface decrease, levels of CD3 are also expected to decrease. As such, CD3 cell surface levels can be used as a proxy for cell surface TCR expression.

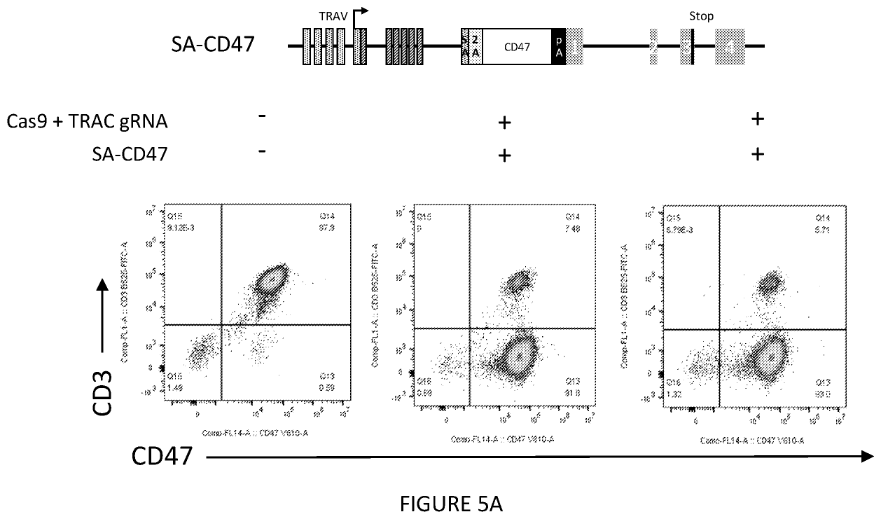

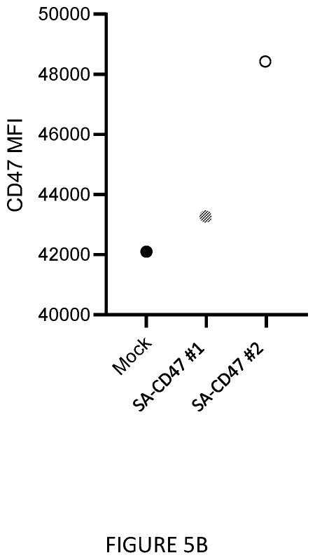

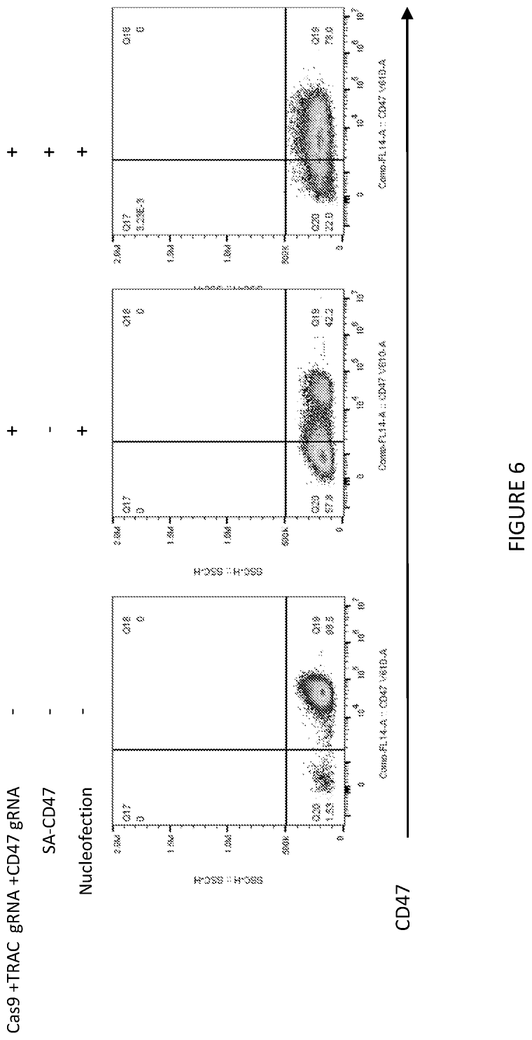

As shown in FIG. 5A, introduction of Cas9 and hTRAC gRNA lead to a decrease in CD3 expression (indicating knock-down of TRAC). Meanwhile, FIG. 5B demonstrates that introduction of SA-CD47 increased CD47 expression. Additionally, wild-type T cells exhibit high expression of CD47. Therefore, in order to assess transgene derived CD47 activity, CD47 expression was evaluated in an endogenous CD47 knock-down background. As shown in FIG. 6, wild-type cells (left graph) expressed CD47. Introduction of Cas9 with TRAC gRNA and CD47 gRNA lead to a reduction in the expression of CD47 (middle graph), which was recovered when the SA-CD47 transgene was introduced into the cells (right graph). These results demonstrate that CD47 expression from a transgene at a TCR gene locus can be successfully achieved.

### Certain Embodiments

Embodiment 1. A method of generating a population of T cells for cell therapy, comprising: (a) inserting a first transgene encoding a tolerogenic factor into an endogenous TCR gene locus of the T cells, and (b) selecting for T cells that have the first transgene inserted by CD3 depletion.

Embodiment 2. The method of embodiment 1, wherein the method further comprises (c) selecting for T cells that have the first transgene inserted by selection for expression of the tolerogenic factor.

Embodiment 3. The method of embodiment 2, wherein the selection for expression of the tolerogenic factor of step (c) is by affinity binding, flow cytometry, and/or immunomagnetic selection using antibodies and/or proteins that bind the tolerogenic factor.

Embodiment 4. The method of any one of the preceding embodiments, wherein the tolerogenic factor is selected from the group consisting of CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, Serpinb9, CCl21, Mfge8, DUX4, B2M-HLA-E, CD27, IL-39, CD16 Fc Receptor, IL15-RF, H2-M3 (HLA-G), A20/TNFAIP3, CR1, HLA-F, and MANF.

Embodiment 5. The method of any one of the preceding embodiments, wherein the tolerogenic factor is CD47.

Embodiment 6. The method of any one of the preceding embodiments, wherein the CD47 is human CD47.

Embodiment 7. The method of any one of the preceding embodiments, wherein the endogenous TCR gene locus is selected from the group consisting of a TRAC locus, a TRBC1 locus, and a TRBC2 locus.

Embodiment 8. The method of any one of the preceding embodiments, wherein the population of T cells has at least 50%, at least 55%, at least 60%, at least 65%, at least 70%, at least 75%, at least 80%, at least 85%, at least 90%, at least 95%, or at least 100% of the T cells having the first transgene inserted into the endogenous TCR gene locus.

Embodiment 9. A method of generating a population of T cells for cell therapy, comprising: (a) inserting a first transgene encoding CD47 into an endogenous TCR gene locus of the T cells, and (b) selecting for T cells that have the first transgene inserted by CD3 depletion, wherein the endogenous TCR gene locus is selected from the group consisting of a TRAC locus, a TRBC1 locus, and a TRBC2 locus.

Embodiment 10. The method of embodiment 9, wherein the method further comprises (c) selecting for T cells that have the first transgene inserted by selection for expression of CD47.

Embodiment 11. The method of embodiment 10, wherein the selection for expression of CD47 of step (c) is by affinity binding, flow cytometry, and/or immunomagnetic selection using antibodies and/or proteins that bind CD47.

Embodiment 12. The method of any one of the preceding embodiments, wherein the population of T cells has at least 50%, at least 55%, at least 60%, at least 65%, at least 70%, at least 75%, at least 80%, at least 85%, at least 90%, at least 95%, or at least 100% of the T cells having the first transgene encoding CD47 inserted into the endogenous TCR gene locus.

Embodiment 13. A method of generating a population of T cells for cell therapy, comprising: (a) inserting a first transgene encoding CD47 into an endogenous TCR gene locus of the T cells, (b) reducing expression of major histocompatibility complex (MHC) class I (MHC I) molecules and/or MHC class II (MHC II) molecules, and (c) selecting for T cells that have the first transgene inserted by CD3 depletion, wherein the endogenous TCR gene locus is selected from the group consisting of a TRAC locus, a TRBC1 locus, and a TRBC2 locus.

Embodiment 14. The method of embodiment 13, wherein the reduction in expression of MHC I molecules is by modulation of the B2M locus, and/or wherein the reduction in expression of MHC II molecules is by modulation of the CIITA locus.

Embodiment 15. A method of generating a population of T cells for cell therapy, comprising: (a) inserting a first transgene encoding CD47 into an endogenous TCR gene locus of the T cells, (b) reducing expression of B2M and/or CIITA, and (c) selecting for T cells that have the first transgene inserted by CD3 depletion, wherein the endogenous TCR gene locus is selected from the group consisting of a TRAC locus, a TRBC1 locus, and a TRBC2 locus.

Embodiment 16. The method of embodiment 15, wherein the reduction in expression of B2M and/or CIITA is by B2M and/or CIITA knockout.

Embodiment 17. The method of embodiment 16, wherein the B2M and/or CIITA knockout occur in both alleles.

Embodiment 18. The method of any one of the preceding embodiments, wherein step (a) occurs before, together with, or after step (b).

Embodiment 19. The method of any one of the preceding embodiments, wherein the method further comprises (d) selecting for T cells that have the first transgene inserted by selection for expression of CD47.

Embodiment 20. The method of embodiment 19, wherein the selection for expression of CD47 of step (c) is by affinity binding, flow cytometry, and/or immunomagnetic selection using antibodies and/or proteins that bind CD47.

Embodiment 21. The method of any one of the preceding embodiments, wherein the population of T cells has at least 50%, at least 55%, at least 60%, at least 65%, at least 70%, at least 75%, at least 80%, at least 85%, at least 90%, at least 95%, or at least 100% of the T cells having the first transgene encoding CD47 inserted into the endogenous TCR gene locus.

Embodiment 22. A method of generating a population of T cells having at least 50% of the T cells with a CD47 transgene inserted into an endogenous TCR gene locus for cell therapy, comprising: (a) inserting the CD47 transgene into the endogenous TCR gene locus of the T cells, (b) optionally, reducing expression of MHC class I and/or MHC class II molecules, and (c) selecting for T cells that have the CD47 transgene inserted by CD3 depletion, wherein the endogenous TCR gene locus is selected from the group consisting of a TRAC locus, a TRBC1 locus, and a TRBC2 locus.

Embodiment 23. The method of embodiment 22, wherein the population of T cells has at least 55%, at least 60%, at least 65%, at least 70%, at least 75%, at least 80%, at least 85%, at least 90%, at least 95%, or at least 100% of the T cells with the CD47 transgene inserted into the endogenous TCR gene locus.

Embodiment 24. The method of embodiment 22 or 23, wherein step (a) occurs before, together with, or after step (b).

Embodiment 25. The method of any one of embodiments 22-24, wherein the method further comprises (d) selecting for T cells that have the CD47 transgene inserted by selection for expression of CD47.

Embodiment 26. The method of any one of the preceding embodiments, wherein the selection for expression of CD47 of step (c) is by affinity binding, flow cytometry, and/or immunomagnetic selection using antibodies and/or proteins that bind CD47.

Embodiment 27. The method of any one of the preceding embodiments, wherein the CD3 depletion is by affinity binding, flow cytometry, and/or immunomagnetic selection using CD3-binding antibodies and/or CD3-binding proteins.

Embodiment 28. A method of generating a population of T cells for cell therapy, comprising: (a) inserting a first transgene encoding a tolerogenic factor into an endogenous TCR gene locus of the T cells, and (b) selecting for T cells that have the first transgene inserted by selection for expression of the tolerogenic factor.

Embodiment 29. The method of embodiment 28, wherein the selection for expression of the tolerogenic factor of step (b) is by affinity binding, flow cytometry, and/or immunomagnetic selection using antibodies and/or proteins that bind the tolerogenic factor.

Embodiment 30. The method of embodiment 28 or 29, wherein the tolerogenic factor is selected from the group consisting of CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, Serpinb9, CCl21, Mfge8, DUX4, B2M-HLA-E, CD27, IL-39, CD16 Fc Receptor, IL15-RF, H2-M3 (HLA-G), A20/TNFAIP3, CR1, HLA-F, and MANF.

Embodiment 31. The method of any one of the preceding embodiments, wherein the tolerogenic factor is CD47.

Embodiment 32. The method of any one of the preceding embodiments, wherein the CD47 is human CD47.

Embodiment 33. The method of any one of the preceding embodiments, wherein the endogenous TCR gene locus is selected from the group consisting of a TRAC locus, a TRBC1 locus, and a TRBC2 locus.

Embodiment 34. The method of any one of the preceding embodiments, wherein the population of T cells has at least 50%, at least 55%, at least 60%, at least 65%, at least 70%, at least 75%, at least 80%, at least 85%, at least 90%, at least 95%, or at least 100% of the T cells having the first transgene inserted into the endogenous TCR gene locus.

Embodiment 35. A method of generating a population of T cells for cell therapy, comprising: (a) inserting a first transgene encoding CD47 into an endogenous TCR gene locus of the T cells, and (b) selecting for T cells that have the first transgene inserted by selection for expression of CD47, wherein the endogenous TCR gene locus is selected from the group consisting of a TRAC locus, a TRBC1 locus, and a TRBC2 locus.

Embodiment 36. The method of embodiment 35, wherein the selection for expression of CD47 of step (b) is by affinity binding, flow cytometry, and/or immunomagnetic selection using antibodies and/or proteins that bind CD47.

Embodiment 37. The method of any one of the preceding embodiments, wherein the population of T cells has at least 50%, at least 55%, at least 60%, at least 65%, at least 70%, at least 75%, at least 80%, at least 85%, at least 90%, at least 95%, or at least 100% of the T cells having the first transgene encoding CD47 inserted into the endogenous TCR gene locus.

Embodiment 38. A method of generating a population of T cells for cell therapy, comprising: (a) inserting a first transgene encoding CD47 into an endogenous TCR gene locus of the T cells, (b) reducing expression of MHC I molecules and/or MHC II molecules, and (c) selecting for T cells that have the first transgene inserted by selection for expression of CD47, wherein the endogenous TCR gene locus is selected from the group consisting of a TRAC locus, a TRBC1 locus, and a TRBC2 locus.

Embodiment 39. The method of embodiment 38, wherein the reduction in expression of MHC I molecules is by modulation of the B2M locus, and/or wherein the reduction in expression of MHC II molecules is by modulation of the CIITA locus.

Embodiment 40. A method of generating a population of T cells for cell therapy, comprising: (a) inserting a first transgene encoding CD47 into an endogenous TCR gene locus of the T cells, (b) reducing expression of B2M and/or CIITA, and (c) selecting for T cells that have the first transgene inserted by selection for expression of CD47, wherein the endogenous TCR gene locus is selected from the group consisting of a TRAC locus, a TRBC1 locus, and a TRBC2 locus.

Embodiment 41. The method of any one of the preceding embodiments, wherein the reduction in expression of B2M and/or CIITA is by B2M and/or CIITA knockout.

Embodiment 42. The method of any one of the preceding embodiments, wherein the B2M and/or CIITA knockout occur in both alleles.

Embodiment 43. The method of any one of the preceding embodiments, wherein step (a) occurs before, together with, or after step (b).

Embodiment 44. The method of any one of the preceding embodiments, wherein the selection for expression of CD47 of step (c) is by affinity binding, flow cytometry, and/or immunomagnetic selection using antibodies and/or proteins that bind CD47.

Embodiment 45. The method of any one of the preceding embodiments, wherein the population of T cells has at least 50%, at least 55%, at least 60%, at least 65%, at least 70%, at least 75%, at least 80%, at least 85%, at least 90%, at least 95%, or at least 100% of the T cells having the first transgene encoding CD47 inserted into the endogenous TCR gene locus.

Embodiment 46. A method of generating a population of T cells having at least 50% of the T cells with a CD47 transgene inserted into an endogenous TCR gene locus for cell therapy, comprising: (a) inserting the CD47 transgene into the endogenous TCR gene locus of the T cells, (b) optionally, reducing expression of MHC class I and/or MHC class II molecules, and (c) selecting for T cells that have the CD47 transgene inserted by selection for expression of CD47, wherein the endogenous TCR gene locus is selected from the group consisting of a TRAC locus, a TRBC1 locus, and a TRBC2 locus.

Embodiment 47. The method of embodiment 46, wherein the population of T cells has at least 55%, at least 60%, at least 65%, at least 70%, at least 75%, at least 80%, at least 85%, at least 90%, at least 95%, or at least 100% of the T cells with the CD47 transgene inserted into the endogenous TCR gene locus.

Embodiment 48. The method of any one of the preceding embodiments, wherein step (a) occurs before, together with, or after step (b).

Embodiment 49. The method of any one of the preceding embodiments, wherein the selection for expression of CD47 of step (c) is by affinity binding, flow cytometry, and/or immunomagnetic selection using antibodies and/or proteins that bind CD47.

Embodiment 50. The method of any one of the preceding embodiments, wherein the inserting of step (a) comprises using a genome-modifying protein.

Embodiment 50a. The method of embodiment 50, wherein the genome-modifying protein comprises a CRISPR-associated transposase, prime editing, or Programmable Addition via Site-specific Targeting Elements (PASTE).

Embodiment 50b. The method of embodiment 50, wherein the genome-modifying protein comprises a TnpB polypeptide.

Embodiment 50c. The method of any one of embodiments 1-49, wherein the inserting of step (a) is by homology-directed repair (HDR)-mediated insertion using a site-directed nuclease.

Embodiment 51. The method of embodiment 50c, wherein the site-directed nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

Embodiment 52. The method of any one of the preceding embodiments, wherein the CD47 comprises an amino acid sequence that is at least 80%, at least 85%, at least 90%, at least 95%, or at least 100% identical to the amino acid sequence set forth in SEQ ID NO:1 or SEQ ID NO:2.

Embodiment 53. The method of embodiment 52, wherein the first transgene encoding CD47 or the CD47 transgene comprises a nucleotide sequence that is at least 80%, at least 85%, at least 90%, at least 95%, or at least 100% identical to the nucleotide sequence set forth in SEQ ID NO:3 or SEQ ID NO:4.

Embodiment 54. The method of any one of the preceding embodiments, wherein the nucleotide sequence is codon-optimized.

Embodiment 55. The method of any one of the preceding embodiments, wherein the nucleotide sequence is at least 80%, at least 85%, at least 90%, at least 95%, or at least 100% identical to the nucleotide sequence set forth in SEQ ID NO:5.

Embodiment 56. The method of any one of the preceding embodiments, wherein the first transgene or the CD47 transgene further comprises a promoter, an insulator, an enhance, a polyadenylation (poly(A)) tail, and/or a ubiquitous chromatin opening element.

Embodiment 57. The method of embodiment 56, wherein the promoter is a constitutive promoter.

Embodiment 58. The method of embodiment 57, wherein the constitutive promoter is an EF1α, CMV, SV40, PGK, UBC CAG, MND, SSFV, or ICOS promoter.

Embodiment 59. The method of any one of the preceding embodiments, further comprising inserting a second transgene encoding a CAR to a genomic locus of the T cell.

Embodiment 60. The method of embodiment 59, wherein the CAR comprises a CD19 CAR.

Embodiment 61. The method of embodiment 60, wherein the CD19 CAR comprises a signal peptide, an extracellular binding domain specific to CD19, a hinge domain, a transmembrane domain, an intracellular costimulatory domain, and/or an intracellular signaling domain.

Embodiment 62. The method of embodiment 61, wherein the signal peptide comprises a CD8α signal peptide, an IgK signal peptide, or a GMCSFR-α signal peptide.

Embodiment 63. The method of embodiment 61, wherein the extracellular binding domain specific to CD19 comprises an scFv.

Embodiment 64. The method of embodiment 63, wherein the scFv comprises the light chain variable region (V_{L}) and the heavy chain variable region (V_{H}) of FMC63.

Embodiment 65. The method of embodiment 63, wherein the scFv comprises one or more complementarity determining regions (CDRs) having amino acid sequences set forth in SEQ ID NOs: 24-26 and 29-31.

Embodiment 66. The method of embodiment 63, wherein the scFv comprises a light chain with one or more CDRs having amino acid sequences set forth in SEQ ID NOs: 24-26.

Embodiment 67. The method of embodiment 63, wherein the scFv comprises a heavy chain with one or more CDRs having amino acid sequences set forth in SEQ ID NOs: 29-31.

Embodiment 68. The method of embodiment 61, wherein the hinge domain comprises a CD8α hinge domain, a CD28 hinge domain, an IgG4 hinge domain, or an IgG4 hinge-CH2-CH3 domain.

Embodiment 69. The method of embodiment 61, wherein the transmembrane comprises a CD8α transmembrane domain or a CD28 transmembrane domain.

Embodiment 70. The method of embodiment 61, wherein the intracellular costimulatory domain comprises a 4-1BB costimulatory domain or a CD28 costimulatory domain.

Embodiment 71. The method of embodiment 61, wherein the intracellular signaling domain comprises a CD3 zeta (ζ) signaling domain.

Embodiment 72. The method of embodiment 60, wherein the CD19 CAR comprises an amino acid sequence set forth in SEQ ID NO: 35, 37, 39, or 41, or an amino acid sequence that is at least 80%, at least 85%, at least 90%, at least 95%, or at least 100% identical to the amino acid sequence set forth in SEQ ID NO: 35, 37, 39, or 41.

Embodiment 73. The method of embodiment 60, wherein the second transgene comprises a nucleotide sequence set forth in SEQ ID NO: 34, 36, 38, or 40, or a nucleotide sequence that is at least 80%, at least 85%, at least 90%, at least 95%, or at least 100% identical to the nucleotide sequence set forth in SEQ ID NO: 35, 37, 39, or 41.

Embodiment 74. The method of embodiment 59, wherein the CAR comprises a CD20 CAR.

Embodiment 75. The method of embodiment 74, wherein the CD20 CAR comprises a signal peptide, an extracellular binding domain specific to CD20, a hinge domain, a transmembrane domain, an intracellular costimulatory domain, and/or an intracellular signaling domain.

Embodiment 76. The method of embodiment 75, wherein the signal peptide comprises a CD8α signal peptide, an IgK signal peptide, or a GMCSFR-α signal peptide.

Embodiment 77. The method of embodiment 75, wherein the extracellular binding domain specific to CD20 comprises an scFv.

Embodiment 78. The method of embodiment 77, wherein the scFv comprises the V_{L} and the V_{H} of Leu16.

Embodiment 79. The method of embodiment 77, wherein the scFv comprises one or more CDRs having amino acid sequences set forth in SEQ ID NOs: 44-46 and 48-49.

Embodiment 80. The method of embodiment 77, wherein the scFv comprises a light chain with one or more CDRs having amino acid sequences set forth in SEQ ID NOs: 44-46.

Embodiment 81. The method of embodiment 77, wherein the scFv comprises a heavy chain with one or more CDRs having amino acid sequences set forth in SEQ ID NOs: 48-49.

Embodiment 82. The method of embodiment 75, wherein the hinge domain comprises a CD8α hinge domain, a CD28 hinge domain, an IgG4 hinge domain, or an IgG4 hinge-CH2-CH3 domain.

Embodiment 83. The method of embodiment 75, wherein the transmembrane comprises a CD8α transmembrane domain or a CD28 transmembrane domain.

Embodiment 84. The method of embodiment 75, wherein the intracellular costimulatory domain comprises a 4-1BB costimulatory domain or a CD28 costimulatory domain.

Embodiment 85. The method of embodiment 75, wherein the intracellular signaling domain comprises a CD3 zeta (ζ) signaling domain.

Embodiment 86. The method of embodiment 59, wherein the CAR comprises a CD22 CAR.

Embodiment 87. The method of embodiment 86, wherein the CD22 CAR comprises a signal peptide, an extracellular binding domain specific to CD22, a hinge domain, a transmembrane domain, an intracellular costimulatory domain, and/or an intracellular signaling domain.

Embodiment 88. The method of embodiment 87, wherein the signal peptide comprises a CD8α signal peptide, an IgK signal peptide, or a GMCSFR-α signal peptide.

Embodiment 89. The method of embodiment 87, wherein the extracellular binding domain specific to CD22 comprises an scFv.

Embodiment 90. The method of embodiment 89, wherein the scFv comprises the V_{H} and the V_{L} of m971.

Embodiment 91. The method of embodiment 89, wherein the scFv comprises one or more CDRs having amino acid sequences set forth in SEQ ID NOs: 52-54 and 56-58.

Embodiment 92. The method of embodiment 89, wherein the scFv comprises a heavy chain with one or more CDRs having amino acid sequences set forth in SEQ ID NOs: 52-54.

Embodiment 93. The method of embodiment 89, wherein the scFv comprises a light chain with one or more CDRs having amino acid sequences set forth in SEQ ID NOs: 56-58.

Embodiment 94. The method of embodiment 89, wherein the scFv comprises the V_{H} and the V_{L} of m971-L7.

Embodiment 95. The method of embodiment 89, wherein the scFv comprises one or more CDRs having amino acid sequences set forth in SEQ ID NOs: 61-63 and 65-67.

Embodiment 96. The method of embodiment 89, wherein the scFv comprises a heavy chain with one or more CDRs having amino acid sequences set forth in SEQ ID NOs: 61-63.

Embodiment 97. The method of embodiment 89, wherein the scFv comprises a light chain with one or more CDRs having amino acid sequences set forth in SEQ ID NOs: 65-67.

Embodiment 98. The method of embodiment 87, wherein the hinge domain comprises a CD8α hinge domain, a CD28 hinge domain, an IgG4 hinge domain, or an IgG4 hinge-CH2-CH3 domain.

Embodiment 99. The method of embodiment 87, wherein the transmembrane comprises a CD8α transmembrane domain or a CD28 transmembrane domain.

Embodiment 100. The method of embodiment 87, wherein the intracellular costimulatory domain comprises a 4-1BB costimulatory domain or a CD28 costimulatory domain.

Embodiment 101. The method of embodiment 87, wherein the intracellular signaling domain comprises a CD3 zeta (Q signaling domain.

Embodiment 102. The method of embodiment 59, wherein the CAR comprises a BCMA CAR.

Embodiment 103. The method of embodiment 102, wherein the BCMA CAR comprises a signal peptide, an extracellular binding domain specific to BCMA, a hinge domain, a transmembrane domain, an intracellular costimulatory domain, and/or an intracellular signaling domain.

Embodiment 104. The method of embodiment 103 wherein the signal peptide comprises a CD8α signal peptide, an IgK signal peptide, or a GMCSFR-α signal peptide.

Embodiment 105. The method of embodiment 103, wherein the extracellular binding domain specific to BCMA comprises an scFv.

Embodiment 106. The method of embodiment 105, wherein the scFv comprises the V_{L} and the V_{H} of C11D5.3.

Embodiment 107. The method of embodiment 105, wherein the scFv comprises one or more CDRs having amino acid sequences set forth in SEQ ID NOs: 70-72 and 74-76.

Embodiment 108. The method of embodiment 105, wherein the scFv comprises a light chain with one or more CDRs having amino acid sequences set forth in SEQ ID NOs: 70-72.

Embodiment 109. The method of embodiment 105, wherein the scFv comprises a heavy chain with one or more CDRs having amino acid sequences set forth in SEQ ID NOs: 74-76.

Embodiment 110. The method of embodiment 105, wherein the scFv comprises the V_{L} and the V_{H} of C12A3.2.

Embodiment 111. The method of embodiment 105, wherein the scFv comprises one or more CDRs having amino acid sequences set forth in SEQ ID NOs: 79-81 and 83-85.

Embodiment 112. The method of embodiment 105, wherein the scFv comprises a light chain with one or more CDRs having amino acid sequences set forth in SEQ ID NOs: 79-81.

Embodiment 113. The method of embodiment 105, wherein the scFv comprises a heavy chain with one or more CDRs having amino acid sequences set forth in SEQ ID NOs: 83-85.

Embodiment 114. The method of embodiment 105, wherein the scFv comprises the V_{L} and the V_{H} of CT103A scFv.

Embodiment 115. The method of embodiment 105, wherein the scFv comprises one or more CDRs having amino acid sequences set forth in SEQ ID NOs: 92-94 and 96-98.

Embodiment 116. The method of embodiment 105, wherein the scFv comprises a light chain with one or more CDRs having amino acid sequences set forth in SEQ ID NOs: 92-94.

Embodiment 117. The method of embodiment 105, wherein the scFv comprises a heavy chain with one or more CDRs having amino acid sequences set forth in SEQ ID NOs: 96-98.

Embodiment 118. The method of embodiment 103, wherein the extracellular binding domain specific to BCMA comprises a fully human heavy-chain variable domain (FHVH).

Embodiment 119. The method of embodiment 118, wherein the FHVH comprises FHVH33.

Embodiment 120. The method of embodiment 119, wherein the FHVH comprises one or more CDRs having amino acid sequences set forth in SEQ ID NOs: 87-89.

Embodiment 121. The method of embodiment 103, wherein the hinge domain comprises a CD8α hinge domain, a CD28 hinge domain, an IgG4 hinge domain, or an IgG4 hinge-CH2-CH3 domain.

Embodiment 122. The method of embodiment 103, wherein the transmembrane comprises a CD8α transmembrane domain or a CD28 transmembrane domain.

Embodiment 123. The method of embodiment 103, wherein the intracellular costimulatory domain comprises a 4-1BB costimulatory domain or a CD28 costimulatory domain.

Embodiment 124. The method of embodiment 103, wherein the intracellular signaling domain comprises a CD3 zeta (Q signaling domain.

Embodiment 125. The method of embodiment 102, wherein the BCMA CAR comprises an amino acid sequence set forth in SEQ ID NO: 100, or an amino acid sequence that is at least 80%, at least 85%, at least 90%, at least 95%, or at least 100% identical to the amino acid sequence set forth in SEQ ID NO: 100.

Embodiment 126. The method of embodiment 102, wherein the second transgene comprises a nucleotide sequence set forth in SEQ ID NO:99, or a nucleotide sequence that is at least 80%, at least 85%, at least 90%, at least 95%, or at least 100% identical to the nucleotide sequence set forth in SEQ ID NO:99.

Embodiment 127. The method of embodiment 59, wherein the CAR comprises a CD19 CAR and a CD22 CAR connected by one or more cleavage sites.

Embodiment 128. The method of embodiment 59, wherein the first transgene and the second transgene are in the form of a polycistronic construct connected by one or more cleavage sites.

Embodiment 129. The method of embodiment 127 or 128, wherein the one or more cleavage sites comprise a self-cleaving site.

Embodiment 130. The method of embodiment 129, wherein the self-cleaving site comprises a 2A site.

Embodiment 131. The method of embodiment 130, wherein the 2A site comprises a T2A, P2A, E2A, or F2A site.

Embodiment 132. The method of any one of embodiments 127-131, wherein the one or more cleavage sites further comprise a protease site.

Embodiment 133. The method of embodiment 132, wherein the protease site comprises a furin site.

Embodiment 134. The method of embodiment 133, wherein the furin site comprises an FC1, FC2, or FC3 site.

Embodiment 135. The method of embodiment 132, wherein the protease site precedes the 2A site in the 5′ to 3′ order.

Embodiment 136. The method of embodiment 59, wherein the second transgene is inserted into a random genomic locus of the T cells.

Embodiment 137. The method of embodiment 59, wherein the second transgene is inserted into a specific genomic locus of the T cells.

Embodiment 138. The method of embodiment 137, wherein the insertion comprises using a genome-modifying protein.

Embodiment 138a. The method of embodiment 138, wherein the genome-modifying protein comprises a CRISPR-associated transposase, prime editing, or Programmable Addition via Site-specific Targeting Elements (PASTE).

Embodiment 138b. The method of embodiment 138, wherein the genome-modifying protein comprises a TnpB polypeptide.

Embodiment 138c. The method of embodiment 137, wherein the insertion is by HDR-mediated insertion using a site-directed nuclease.

Embodiment 139. The method of embodiment 138c, wherein the site-directed nuclease is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

Embodiment 140. The method of embodiment 137, wherein the specific genomic locus is selected from the group consisting of a TRAC locus, a TRBC1 locus, a TRBC2 locus, a B2M locus, a CIITA locus, and a safe harbor locus.

Embodiment 141. The method of embodiment 140, wherein the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus.

Embodiment 142. A population of T cells generated by the method of any one of embodiments 1-141.

Embodiment 143. A population of T cells wherein at least 50% of the T cells each have (a) reduced expression of MHC I and/or MHC II molecules, and (b) increased expression of a tolerogenic factor encoded by a transgene.

Embodiment 144. A population of T cells wherein at least 50% of the T cells each have (a) reduced expression of MHC I and/or MHC II molecules, and (b) increased expression of CD47 encoded by a transgene.

Embodiment 145. A population of T cells wherein at least 50% of the T cells each have (a) reduced expression of B2M and/or CIITA, and (b) increased expression of CD47 encoded by a transgene.

Embodiment 146. A population of T cells wherein at least 50% of the T cells each have (a) B2M and/or CIITA knocked-out, and (b) increased expression of CD47 encoded by a transgene.

Embodiment 147. The population of T cells of any one of embodiments 143-146, wherein at least 55%, at least 60%, at least 65%, at least 70%, at least 75%, at least 80%, at least 85%, at least 90%, at least 95%, or at least 100% of the T cells each have (a) and (b).

Embodiment 148. A population of T cells wherein at least 50% of the T cells each have (a) reduced expression of B2M, (b) reduced expression of CIITA, and (c) increased expression of CD47 encoded by a transgene.

Embodiment 149. The population of T cells of embodiment 148, wherein at least 55%, at least 60%, at least 65%, at least 70%, at least 75%, at least 80%, at least 85%, at least 90%, at least 95%, or at least 100% of the T cells each have (a) and (b).

Embodiment 150. The population of T cells of embodiment 148 or 149, wherein at least 55%, at least 60%, at least 65%, at least 70%, at least 75%, at least 80%, at least 85%, at least 90%, at least 95%, or at least 100% of the T cells each have (a), (b), and (c).

Embodiment 151. The population of T cells of any one of embodiments 148-150, wherein each of the remainder T cells in the population has one of (i)-(vii):
(i) reduced expression of MHC I molecules,
(ii) reduced expression of MHC II molecules,
(iii) increased expression of CD47,
(iv) reduced expression of MHC I molecules and MHC II molecules,
(v) reduced expression of MHC I molecules and increased expression of CD47,
(vi) reduced expression of MHC II molecules and increased expression of CD47, or
(vii) endogenous expression of MHC I molecules, MHC II molecules, and CD47.

Embodiment 152. The population of T cells of any one of embodiments 143-151, wherein the CD47 transgene is inserted into an endogenous TCR gene locus.

Embodiment 153. The population of T cells of embodiment 152, wherein the endogenous TCR gene locus is selected from the group consisting of a TRAC locus, a TRBC1 locus, and a TRBC2 locus.

Embodiment 154. The population of T cells of any one of embodiments 142-153, wherein the T cells are allogeneic T cells.

Embodiment 155. The population of T cells of embodiment 154, wherein the allogeneic T cells are primary T cells.

Embodiment 156. The population of T cells of embodiment 154, wherein the allogeneic T cells are differentiated from an embryonic stem cell (ESC) or an induced pluripotent stem cell (iPSC).

Embodiment 157. A pharmaceutical composition comprising the population of T cells of any one of embodiments 142 to 156.

Embodiment 158. A method of treating a disease in a subject in need thereof, comprising administering the subject the population of T cells of any one of embodiments 142 to 156, or the pharmaceutical composition of embodiment 157.

Embodiment 159. The method of embodiment 158, wherein the disease is cancer.

Embodiment 160. The method of embodiment 159, wherein the cancer is associated with CD19, CD22, and/or BCMA expression.

Embodiment 161. The method of embodiment 159, wherein the cancer is a hematologic malignancy.

Embodiment 162. The method of embodiment 161, wherein the hematologic malignancy is selected from the group consisting of myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

Embodiment 163. The method of embodiment 158, wherein the disease is an autoimmune disease.

Embodiment 164. The method of embodiment 163, wherein the autoimmune disease is selected from the group consisting of lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

Embodiment 165. The method of embodiment 158, wherein the disease is diabetes mellitus.

Embodiment 166. The method of embodiment 165, wherein the diabetes is selected from the group consisting Type I diabetes, Type II diabetes, prediabetes, and gestational diabetes.

Embodiment 167. The method of embodiment 158, wherein the disease is a neurological disease.

Embodiment 168. The method of embodiment 167, wherein the neurological disease is selected from the group consisting of catalepsy, epilepsy, encephalitis, meningitis, migraine, Huntington's, Alzheimer's, Parkinson's, Pelizaeus-Merzbacher disease, and multiple sclerosis.

Embodiment 169. An engineered cell comprising, in its genome, a first transgene encoding a tolerogenic factor,
wherein the first transgene is located at a first insertion site at a T-cell receptor (TCR) gene locus.

Embodiment 169a. The engineered cell of embodiment 169, wherein the first insertion site is in exon 1.

Embodiment 170. The engineered cell of embodiment 169 or 169a, wherein the engineered cell is a human cell.

Embodiment 171. The engineered cell of embodiment 169 or 170, wherein the engineered cell is a T-cell, an islet cell, a cardiomyocyte, a hepatocyte, or a stem cell.

Embodiment 172. The engineered cell of any one of embodiments 169-171, wherein the engineered cell is a T-cell.

Embodiment 173. The engineered cell of any one of embodiments 169-172, wherein the engineered cell is a human T-cell.

Embodiment 174. The engineered cell of any one of embodiments 169-173, wherein the engineered cell is an allogeneic T-cell.

Embodiment 174a. The engineered cell of any one of embodiments 169-173, wherein the engineered cell is an autologous T-cell.

Embodiment 175. The engineered cell of embodiment 174, wherein the allogeneic T-cell is a primary T-cell.

Embodiment 176. The engineered cell of embodiment 175, wherein the allogeneic T-cell has been differentiated from an embryonic stem cell (ESC) or an induced pluripotent stem cell (iPSC).

Embodiment 177. The engineered cell of any one of embodiments 169-176, wherein the tolerogenic factor is or comprises: CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, Serpinb9, CCl21, Mfge8, DUX4, B2M-HLA-E, CD27, IL-39, CD16 Fc Receptor, IL15-RF, H2-M3 (HLA-G), A20/TNFAIP3, CR1, HLA-F, or MANF.

Embodiment 178. The engineered cell of any one of embodiments 169-177, wherein the tolerogenic factor is or comprises CD47.

Embodiment 179. The engineered cell of any one of embodiments 169-178, wherein the tolerogenic factor is or comprises human CD47.

Embodiment 180. The engineered cell of embodiment 178 or 179, wherein the CD47 comprises an amino acid sequence at least 80%, at least 85%, at least 90%, at least 95%, at least 99%, or 100% identical to an amino acid sequence set forth in SEQ ID NO:1 or SEQ ID NO:2.

Embodiment 181. The engineered cell of embodiment 178 or 179, wherein the first transgene encodes CD47 and comprises a nucleotide sequence at least 80%, at least 85%, at least 90%, at least 95%, at least 99%, or 100% identical to a nucleotide sequence set forth in SEQ ID NO:3 or SEQ ID NO:4.

Embodiment 182. The engineered cell of embodiment 169-181, wherein the nucleotide sequence of the first transgene is codon-optimized.

Embodiment 183. The engineered cell of embodiment 182, wherein the first transgene is at least 80%, at least 85%, at least 90%, at least 95%, at least 99%, or 100% identical to a nucleotide sequence set forth in SEQ ID NO:5.

Embodiment 184. The engineered cell of any one of embodiments 169-183, wherein the engineered cell further comprises a transgene encoding a that does not encode: a chimeric antigen receptor (CAR), chimeric co-stimulatory receptor (CCR), cytokine, dominant negative, microenvironment modulator, antibody, biosensor, chimeric receptor ligand (CLR), chimeric immune receptor ligand (CIRL), soluble receptor, solute transporter, enzyme, ribozyme, genetic circuit, epigenetic modifier, transcriptional activator or repressor, or non-coding RNA in its genome.

Embodiment 184a. The engineered cell of any one of embodiments 169-184, wherein the engineered cell further comprises a transgene encoding a that does not encode: 4-IBB, OX40, ICOS, CD70, IL2, IL12, IL15, IL18, inhibitory chimeric antigen receptor (iCAR) (e.g., extracellular scFv domain fused to an intracellular signaling domain derived from inhibitory T-cell receptors (e.g., PD-1 or CTL4)), a secretable soluble cytokine receptor (e.g., for TGFBeta, IL10), a secretable soluble T-cell inhibitory receptor (e.g., derived from PD-1, CTLA4, LAG3, or TIM-3), heparanase, Herpes Virus Entry Mediator (HVEM) (TNFRSF14), antibody against a T-cell inhibitory ligand (e.g., PD-1L, CD80, CD86, Galectin-9, or Fas ligand), toll-like receptor (TLR), Calcium-sensing Calmodulin (CaM) calmodulin-binding peptide, IL4R, IL10R, PD1, CTL4, TIM-3, LAG3, a glucose transporter (e.g., Glut1 or Glut3), PKM2, a pathogen-specific ribozyme, a viral-specific ribozyme, a HIF1a-dependent transcription unit, a TALE-VP64-dependent transcription unit, a cell-surface CD19-specific scFV-NFAT fusion protein, a chimeric programmable sequence-specific DNA binding domain fused to p300 acetyltransferase domain (a histone H3 acetylase), a chimeric programmable sequence-specific DNA binding domain fused to KRAB repressor domain, Foxp3, NFAT, HIF-1alpha, a DNA binding domain (e.g., TAL, zinc-finger, CRISPR/deactivatedCas9) and a transactivation domain (e.g., VP16, VP64, p65, Rta, or combinations of them), fusion proteins comprising a DNA binding domain (e.g., TAL, zinc-finger, CRISPR/deactivatedCas9) and a repressor domain (e.g., KRAB), microRNA (miRNA), small interfering RNA (siRNA), anti-sense RNA targeting e.g., target inhibitory receptor gene messenger RNAs such as messenger RNAs of PD1, TIM-3, LAG3, CTLA-4.

Embodiment 185. The engineered cell of any one of embodiments 169-183, wherein the TCR gene locus is or comprises an endogenous TCR gene locus.

Embodiment 186. The engineered cell of any one of embodiments 169-185, wherein the TCR gene locus is or comprises: a TRAC locus, a TRBC1 locus, or a TRBC2 locus.

Embodiment 187. The engineered cell of any one of embodiments 169-186, wherein the engineered cell does not express a functional endogenous TCR.

Embodiment 188. The engineered cell of any one of embodiments 169-187, wherein the engineered cell does not express an endogenous TCR on its cell surface.

Embodiment 189. The engineered cell of any one of embodiments 169-188, wherein the engineered cell does not express a gene product from a TRAC locus, a TRBC1 locus, a TRBC2 locus, or a combination thereof.

Embodiment 190. The engineered cell of any one of embodiments 169-189, wherein the first insertion site is in an exon.

Embodiment 191. The engineered cell of any one of embodiments 169-189, wherein the first insertion site is in an intron.

Embodiment 192. The engineered cell of any one of embodiments 169-189, wherein the first insertion site is between an intron and an exon.

Embodiment 193. The engineered cell of any one of embodiments 169-189, wherein the first insertion site is in a regulatory region.

Embodiment 194. The engineered cell of any one of embodiments 169-193, wherein the engineered cell comprises a genome-modifying protein.

Embodiment 194a. The engineered cell of embodiment 194, wherein the genome-modifying protein comprises a CRISPR-associated transposase, prime editing, or Programmable Addition via Site-specific Targeting Elements (PASTE).

Embodiment 194b. The engineered cell of embodiment 194, wherein the genome-modifying protein comprises a TnpB polypeptide.

Embodiment 194c. The engineered cell of any one of embodiments 169-194, wherein the engineered cell comprises a site-directed nuclease.

Embodiment 195. The engineered cell of any one of embodiments 194-194c, wherein the genome-modifying protein or site-directed nuclease is capable of cleaving the engineered cell's genome at or adjacent to the first insertion site.

Embodiment 196. The engineered cell of any one of embodiments 194-195, wherein the genome-modifying protein or site-directed nuclease is capable of single strand DNA cleavage.

Embodiment 197. The engineered cell of any one of embodiments 194-195, wherein the genome-modifying protein or site-directed nuclease is capable of double strand DNA cleavage.

Embodiment 198. The engineered cell of any one of embodiments 194-197, wherein the site-directed nuclease is a Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f(C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, zinc finger nuclease (ZFN), transcription activator-like effector nuclease (TALEN), meganuclease, or CRISPR-associated transposase.

Embodiment 199. The engineered cell of any one of embodiments 194-198, wherein the site-directed nuclease is a Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f(C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7 or CRISPR-associated transposase.

Embodiment 199a. The engineered cell of any one of embodiments 194-199, wherein the first insertion site is 25 nucleotides or less from a target adjacent motif (TAM) sequence, wherein the TAM sequence is tea, ttcan, ttgat, or ataaa, and wherein:
(i) n=a, c, t, or g.

Embodiment 199b. The engineered cell of embodiment of embodiments 199a, wherein the engineered cell comprises TnpB and the TAM is tea.

Embodiment 199c. The engineered cell of embodiment of embodiments 199a, wherein the engineered cell comprises TnpB and the TAM is ttcan, wherein
(i) n=a, c, t, or g.

Embodiment 199d. The engineered cell of embodiment of embodiments 199a, wherein the engineered cell comprises TnpB and the TAM is ttgat.

Embodiment 199e. The engineered cell of embodiment of embodiments 199a, wherein the engineered cell comprises TnpB and the TAM is ataaa.

Embodiment 200. The engineered cell of any one of embodiments 169-199, further comprising a guide RNA (gRNA), wherein the gRNA comprises a complementary region that is complementary to a target nucleic acid sequence at the TCR gene locus.

Embodiment 201. The engineered cell of embodiment 200, wherein the target nucleic acid sequence comprises the first insertion site.

Embodiment 202. The engineered cell of any one of embodiments 169-201, wherein the first insertion site is 25 nucleotides or less from a protospacer adjacent motif (PAM) sequence, wherein the PAM sequence is ngg, nag, ngrrt, ngrrn, nnnngatt, nnnnryac, nnagaaw, naaaac, tttv, ttn, attn, tttn, or gttn, and wherein:
(i) r=a or g,
(ii) y=c or t,
(iii) w=a or t,
(iv) v=a or c or g,
(v) n=a, c, t, or g.

Embodiment 203. The engineered cell of embodiments 202, wherein the engineered cell comprises SpCas9 and the PAM is ngg or nag, wherein n=a, c, t, or g.

Embodiment 204. The engineered cell of embodiments 202, wherein the engineered cell comprises SaCas9 and the PAM is ngrrt or ngrrn, wherein
(i) r=a or g, and
(ii) n=a, c, t, or g.

Embodiment 205. The engineered cell of embodiment 202, wherein the engineered cell comprises NmeCas9 and the PAM is nnnngatt, wherein n=a, c, t, or g.

Embodiment 206. The engineered cell of embodiment 202, wherein the engineered cell comprises CjCas9 and the PAM is nnnnryac, wherein:
(i) r=a or g,
(ii) y=c or t, and
(iii) n=a, c, t, or g.

Embodiment 207. The engineered cell of embodiment 202, wherein the engineered cell comprises StCas9 and the PAM is nnagaaw, wherein:
(i) w=a or t, and
(ii) n=a, c, t, or g.

Embodiment 208. The engineered cell of embodiment 202, wherein the engineered cell comprises TdCas9 and the PAM is naaaac, wherein n=a, c, t, or g.

Embodiment 209. The engineered cell of embodiment 202, wherein the engineered cell comprises LbCas12a and the PAM is tttv, wherein v=a or c or g.

Embodiment 210. The engineered cell of embodiment 202, wherein the engineered cell comprises AsCas12a and the PAM is tttv, wherein v=a or c or g.

Embodiment 211. The engineered cell of embodiment 202, wherein the engineered cell comprises AacCas12b and the PAM is ttn, wherein n=a, c, t, or g.

Embodiment 212. The engineered cell of embodiment 202, wherein the engineered cell comprises BhCas12b and the PAM is attn, tttn, or gttn, wherein n=a, c, t, or g.

Embodiment 213. The engineered cell of any one of embodiments 169-212, wherein the first insertion site is 25 nucleotides or less from a zinc finger binding sequence.

Embodiment 214. The engineered cell of any one of embodiments 194-198, wherein the site-directed nuclease is a ZFN.

Embodiment 215. The engineered cell of any one of embodiments 169-214, wherein the first insertion site is 25 nucleotides or less from a transcription activator-like effectors (TALE) binding sequence.

Embodiment 216. The engineered cell of embodiment 215, wherein the site-directed nuclease is a TALEN.

Embodiment 217. The engineered cell of any one of embodiments 169-216, wherein the first transgene comprises a promoter, an insulator, an enhancer, a polyadenylation (poly(A)) tail, and/or a ubiquitous chromatin opening element.

Embodiment 218. The engineered cell of embodiment 217, wherein the first transgene comprises a promoter and the promoter is a constitutive promoter.

Embodiment 218a. The engineered cell of embodiment 217 or 218, wherein the promoter is an endogenous promoter.

Embodiment 218b. The engineered cell of embodiment 217 or 218, wherein the promoter is an exogenous promoter.

Embodiment 219. The engineered cell of embodiment 218, wherein the constitutive promoter is an EF1α, EF1α short, CMV, SV40, PGK, UBC CAG, MND, SSFV, or ICOS promoter.

Embodiment 220. The engineered cell of any one of embodiments 169-219, wherein the engineered cell further comprises a second transgene encoding a tolerogenic factor in its genome.

Embodiment 221. The engineered cell of embodiment 193, wherein the second transgene encodes the same tolerogenic factor as the first transgene.

Embodiment 222. The engineered cell of embodiment 194, wherein the second transgene encodes a different tolerogenic factor than the first transgene.

Embodiment 223. The engineered cell of any one of embodiments 169-222, wherein the engineered cell has reduced expression of one or more major histocompatibility complex (MHC) class I (MHC I) molecules, one or more MHC class II molecules, or both on its cell surface as compared to a comparable unmodified human cell.

Embodiment 224. The engineered cell of any one of embodiments 169-223, wherein the engineered cell does not express major histocompatibility complex (MHC) class I (MHC I) molecules, MHC class II molecules, or both on its cell surface.

Embodiment 225. The engineered cell of any one of embodiments 169-224, wherein the engineered cell further comprises a modification at a B2M locus.

Embodiment 226. The engineered cell of embodiment 225, wherein the modification at a B2M locus comprises a knock-out of the B2M locus.

Embodiment 227. The engineered cell of embodiment 225 or 226, wherein the engineered cell is homozygous for the modification at a B2M locus.

Embodiment 227. The engineered cell of embodiment 225 or 226, wherein the engineered cell is heterozygous for the modification at a B2M locus.

Embodiment 228. The engineered cell of any one of embodiments 169-227, wherein the engineered cell further comprises a modification at a CIITA locus.

Embodiment 229. The engineered cell of embodiment 228, wherein the modification at a CIITA locus comprises a knock-out of the CIITA locus.

Embodiment 230. The engineered cell of embodiment 228 or 229, wherein the engineered cell is homozygous for the modification at a CIITA locus.

Embodiment 230a. The engineered cell of embodiment 228 or 229, wherein the engineered cell is heterozygous for the modification at a CIITA locus.

Embodiment 231. The engineered cell of any one of embodiments 169-230, wherein the engineered cell further comprises a transgene encoding a chimeric antigen receptor (CAR) in its genome.

Embodiment 231a. The engineered cell of embodiment 231, wherein the CAR comprises a CD5, CD19, CD20, CD22, CD23, CD30, CD33, CD70, Kappa, Lambda, B cell maturation agent (BCMA), G-protein coupled receptor family C group 5 member D (GPRC5D), CS1/SLAMF7, CD38, CD138, GPRC5D, TACI, BCMA, CD123, LeY, NKG2D ligand, WT1, GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, ROR1, C-Met, CD133, Ep-CAM, GPC3, HPV16-E6, IL13Ra2, MAGEA3, MAGEA4, MART1, NY-ESO-1, VEGFR2, α-Folate receptor, CD24, CD44v7/8, EGP-2, EGP-40, erb-B2, erb-B 2,3,4, FBP, Fetal acethylcholine e receptor, GD2, GD3, HMW-MAA, IL-11Rα, KDR, Lewis Y, L1-cell adhesion molecule, MAGE-A1, Oncofetal antigen (h5T4), TAG-72, A*02, fibroblast activation protein (FAP), and/or urokinase-type plasminogen activator receptor (uPAR) CAR.

Embodiment 232. The engineered cell of embodiment 231, wherein the CAR comprises a CD19 CAR.

Embodiment 233. The engineered cell of embodiment 232, wherein the CD19 CAR comprises a signal peptide, an extracellular binding domain specific to CD19, a hinge domain, a transmembrane domain, an intracellular costimulatory domain, and/or an intracellular signaling domain.

Embodiment 234. The engineered cell of embodiment 231, wherein the CAR comprises a CD22 CAR.

Embodiment 235. The engineered cell of embodiment 234, wherein the CD22 CAR comprises a signal peptide, an extracellular binding domain specific to CD22, a hinge domain, a transmembrane domain, an intracellular costimulatory domain, and/or an intracellular signaling domain.

Embodiment 236. The engineered cell of embodiment 231, wherein the CAR comprises a BCMA CAR.

Embodiment 237. The engineered cell of embodiment 236, wherein the BCMA CAR comprises a signal peptide, an extracellular binding domain specific to BCMA, a hinge domain, a transmembrane domain, an intracellular costimulatory domain, and/or an intracellular signaling domain.

Embodiment 238. The engineered cell of embodiment 231, wherein the CAR comprises a CD19 CAR and a CD22 CAR connected by one or more cleavage sites.

Embodiment 238a. The engineered cell of any one of embodiments 231-238, wherein the CAR comprises a CD19 CAR and a CD20 CAR.

Embodiment 238b. The engineered cell of any one of embodiments 231-238, wherein the CAR comprises a CD20 CAR and a CD22 CAR.

Embodiment 238c. The engineered cell of any one of embodiments 231-238, wherein the CAR comprises a CD19 CAR, a CD20 CAR, and a CD22 CAR.

Embodiment 239. The engineered cell of embodiment 231, wherein the first transgene and the transgene encoding the CAR are in the form of a polycistronic construct connected by one or more cleavage sites.

Embodiment 240. The engineered cell of embodiment 238 or 239, wherein the one or more cleavage sites comprise a self-cleaving site.

Embodiment 241. The engineered cell of any one of embodiments 238-239, wherein the one or more cleavage sites comprise a protease site.

Embodiment 242. The engineered cell of embodiment 241, wherein the protease site precedes the 2A site in the 5′ to 3′ order.

Embodiment 243. The engineered cell of any one of embodiments 231-242, wherein the transgene encoding a CAR is located at a random genomic locus.

Embodiment 244. The engineered cell of any one of embodiments 231-242, wherein the transgene encoding a CAR is located at a TRAC locus, a TRBC1 locus, a TRBC2 locus, a B2M locus, a CIITA locus, or a safe harbor locus.

Embodiment 245. The engineered cell of embodiment 244, wherein the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus.

Embodiment 246. The engineered cell of embodiment 169, wherein the engineered cell is a T-cell, the tolerogenic factor is CD47, the TCR gene locus is a TRAC locus, and the first insertion site is in an exon.

Embodiment 247. A population of cells comprising one or more engineered cells of any one of embodiments 169-246.

Embodiment 248. A population of engineered cells according to any one of embodiment 169-247.

Embodiment 249. The population of cells according to embodiment 247, wherein the cells are T-cells, and wherein at least 50% of the T-cells each have (a) reduced cell surface expression of MHC I and/or MHC II molecules, and (b) increased expression of a tolerogenic factor encoded by the first transgene.

Embodiment 250. The population of cells according to embodiment 249, wherein the tolerogenic factor is CD47.

Embodiment 251. The population of cells according to embodiment 247, wherein the cells are T-cells, and wherein at least 50% of the T-cells each have (a) reduced expression of B2M and/or CIITA, and (b) increased expression of CD47 encoded by a transgene.

Embodiment 252. The population of cells according to embodiment 247, wherein the cells are T-cells, and wherein at least 50% of the T-cells each have (a) B2M and/or CIITA knocked-out, and (b) increased expression of CD47 encoded by a transgene.

Embodiment 253. The population of cells of any one of embodiments 249-252, wherein at least 55%, at least 60%, at least 65%, at least 70%, at least 75%, at least 80%, at least 85%, at least 90%, at least 95%, or at least 100% of the T-cells each have (a) and (b).

Embodiment 254. The population of cells according to embodiment 247, wherein the cells are T-cells, and wherein at least 50% of the T-cells each have (a) reduced expression of B2M, (b) reduced expression of CIITA, and (c) increased expression of CD47 encoded by a transgene.

Embodiment 255. The population of cells of embodiment 254, wherein at least 55%, at least 60%, at least 65%, at least 70%, at least 75%, at least 80%, at least 85%, at least 90%, at least 95%, or at least 100% of the T-cells each have (a) and (b).

Embodiment 256. The population of cells of embodiment 254 or 255, wherein at least 55%, at least 60%, at least 65%, at least 70%, at least 75%, at least 80%, at least 85%, at least 90%, at least 95%, or at least 100% of the T-cells each have (a), (b), and (c).

Embodiment 257. A composition comprising an engineered cell according to any one of embodiments 1-57 or a population of cells according to any of claims 247-256.

Embodiment 258. A pharmaceutical composition comprising (i) an engineered cell according to any one of claims 169-246 or a population of cells according to any of claims 247-256, and (ii) a pharmaceutically acceptable excipient.

Embodiment 259. A method comprising administering to a subject an engineered cell according to any one of embodiments 169-246, a population of cells according to any of embodiments 247-256, a composition according to embodiment 257, or a pharmaceutical composition according to embodiment 258.

Embodiment 260. The method of embodiment 259, wherein the method is a method of treating disease in a subject in need thereof.

Embodiment 261. An engineered cell according to any one of embodiments 169-246 for use in treating a disease in a subject in need thereof.

Embodiment 262. A population of cells according to any of embodiments 247-256 for use in treating a disease in a subject in need thereof.

Embodiment 263. A composition according to embodiment 257 for use in treating a disease in a subject in need thereof.

Embodiment 264. A pharmaceutical composition of embodiment 258 for use in treating a disease in a subject in need thereof.

Embodiment 265. Use of an engineered cell according to any one of embodiments 169-246, a population of cells according to any of embodiments 247-256, a composition according to embodiment 257, or a pharmaceutical composition according to embodiment 258 for use in treating a disease in a subject in need thereof.

Embodiment 266. Use of an engineered cell according to any one of embodiments 169-246, a population of cells according to any of embodiments 247-256, a composition according to embodiment 257, or a pharmaceutical composition according to embodiment 258 in the manufacture of a medicament for the treatment of a disease.

Embodiment 267. The method of embodiment 259 or 260, the engineered cell of embodiment 261, the population of cells of embodiment 262, the composition of embodiment 263, the pharmaceutical composition of embodiment 264, or the use of embodiment 265 or 266, wherein the disease is cancer.

Embodiment 268. The method of embodiment 259 or 260, the engineered cell of embodiment 261, the population of cells of embodiment 262, the composition of embodiment 263, the pharmaceutical composition of embodiment 264, or the use of embodiment 265 or 266, wherein the cancer is associated with CD19, CD22, and/or BCMA expression.

Embodiment 269. The method of embodiment 259 or 260, the engineered cell of embodiment 261, the population of cells of embodiment 262, the composition of embodiment 263, the pharmaceutical composition of embodiment 264, or the use of embodiment 265 or 266, wherein the cancer is a hematologic malignancy.

Embodiment 270. The method of embodiment 259 or 260, the engineered cell of embodiment 261, the population of cells of embodiment 262, the composition of embodiment 263, the pharmaceutical composition of embodiment 264, or the use of embodiment 265 or 266, wherein the hematologic malignancy is selected from the group consisting of myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

Embodiment 271. The method of embodiment 259 or 260, the engineered cell of embodiment 261, the population of cells of embodiment 262, the composition of embodiment 263, the pharmaceutical composition of embodiment 264, or the use of embodiment 265 or 266, wherein the disease is an autoimmune disease.

Embodiment 272. The method of embodiment 259 or 260, the engineered cell of embodiment 261, the population of cells of embodiment 262, the composition of embodiment 263, the pharmaceutical composition of embodiment 264, or the use of embodiment 265 or 266, wherein the autoimmune disease is selected from the group consisting of lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

Embodiment 273. The method of embodiment 259 or 260, the engineered cell of embodiment 261, the population of cells of embodiment 262, the composition of embodiment 263, the pharmaceutical composition of embodiment 264, or the use of embodiment 265 or 266, wherein the disease is diabetes mellitus.

Embodiment 274. The method of embodiment 259 or 260, the engineered cell of embodiment 261, the population of cells of embodiment 262, the composition of embodiment 263, the pharmaceutical composition of embodiment 264, or the use of embodiment 265 or 266, wherein the diabetes is selected from the group consisting Type I diabetes, Type II diabetes, prediabetes, and gestational diabetes.

Embodiment 275. The method of embodiment 259 or 260, the engineered cell of embodiment 261, the population of cells of embodiment 262, the composition of embodiment 263, the pharmaceutical composition of embodiment 264, or the use of embodiment 265 or 266, wherein the disease is a neurological disease.

Embodiment 276. The method of embodiment 259 or 260, the engineered cell of embodiment 261, the population of cells of embodiment 262, the composition of embodiment 263, the pharmaceutical composition of embodiment 264, or the use of embodiment 265 or 266, wherein the neurological disease is selected from the group consisting of catalepsy, epilepsy, encephalitis, meningitis, migraine, Huntington's, Alzheimer's, Parkinson's, Pelizaeus-Merzbacher disease, and multiple sclerosis.

Embodiment 277. A method of generating a genetically engineered cell, the method comprising: inserting a first transgene encoding a tolerogenic factor at a first insertion site at a TCR gene locus.

Embodiment 278. The method of embodiment 277, wherein the step of inserting comprises using a genome-modifying protein.

Embodiment 278a. The method of embodiment 278, wherein the genome-modifying protein comprises a CRISPR-associated transposase, prime editing, or Programmable Addition via Site-specific Targeting Elements (PASTE).

Embodiment 278b. The method of embodiment 278, wherein the genome-modifying protein comprises a TnpB polypeptide.

Embodiment 278c. The method of embodiment 277, wherein the step of inserting comprises homology-directed repair (HDR)-mediated insertion using a site-directed nuclease.

Embodiment 279. The method of embodiment 278c, wherein the site-directed nuclease is selected from the group consisting of: Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

Embodiment 280. The method of any one of embodiments 277-279, wherein the step of inserting comprises homology-directed repair (HDR)-mediated insertion using a guide RNA (gRNA) and a CRISPR-associated (Cas) nuclease.

Embodiment 281. The method of embodiment 280, wherein the gRNA comprises a complementary region,
wherein the complementary region comprises a nucleic acid sequence that is complementary to a target nucleic acid sequence within the TCR gene locus, and wherein the target nucleic acid sequence comprises the first insertion site.

Embodiment 281a. The method of any one of embodiments 277-281, wherein the first insertion site is 25 nucleotides or less from a target adjacent motif (TAM) sequence, wherein the TAM sequence is tca, ttcan, ttgat, or ataaa, and wherein:
(i) n=a, c, t, or g.

Embodiment 281b. The method of embodiment of embodiments 281a, wherein the engineered cell comprises TnpB and the TAM is tca.

Embodiment 281c. The method of embodiment of embodiments 281a, wherein the engineered cell comprises TnpB and the TAM is ttcan, wherein
(i) n=a, c, t, or g.

Embodiment 281d. The method of embodiment of embodiments 281a, wherein the engineered cell comprises TnpB and the TAM is ttgat.

Embodiment 281e. The method of embodiment of embodiments 281a, wherein the engineered cell comprises TnpB and the TAM is ataaa.

Embodiment 282. The method of embodiment 281, wherein the first insertion site is 25 nucleotides or less from a protospacer adjacent motif (PAM) sequence, wherein the PAM sequence is ngg, nag, ngrrt, ngrrn, nnnngatt, nnnnryac, nnagaaw, naaaac, tttv, ttn, attn, tttn, or gttn, and wherein:
(i) r=a or g,
(ii) y=c or t,
(iii) w=a or t,
(iv) v=a or c or g,
(v) n=a, c, t, or g.

Embodiment 283. The method of embodiment 282, wherein the step of inserting comprises homology-directed repair (HDR)-mediated insertion using SpCas9 and the PAM is ngg or nag, wherein n=a, c, t, or g.

Embodiment 284. The method of embodiment 282, wherein the step of inserting comprises homology-directed repair (HDR)-mediated insertion using SaCas9 and the PAM is ngrrt or ngrrn, wherein
(i) r=a or g, and
(ii) n=a, c, t, or g.

Embodiment 285. The method of embodiment 282, wherein the step of inserting comprises homology-directed repair (HDR)-mediated insertion using NmeCas9 and the PAM is nnnngatt, wherein n=a, c, t, or g.

Embodiment 286. The method of embodiment 282, wherein the step of inserting comprises homology-directed repair (HDR)-mediated insertion using CjCas9 and the PAM is nnnnryac, wherein:
(i) r=a or g,
(ii) y=c or t, and
(iii) n=a, c, t, or g.

Embodiment 287. The method of embodiment 282, wherein the step of inserting comprises homology-directed repair (HDR)-mediated insertion using StCas9 and the PAM is nnagaaw wherein:
(i) w=a or t, and
(ii) n=a, c, t, or g.

Embodiment 288. The method of embodiment 282, wherein the step of inserting comprises homology-directed repair (HDR)-mediated insertion using TdCas9 and the PAM is naaaac, wherein n=a, c, t, or g.

Embodiment 289. The method of embodiment 282, wherein the step of inserting comprises homology-directed repair (HDR)-mediated insertion using LbCas12a and the PAM is tttv, wherein v=a or c or g.

Embodiment 290. The method of embodiment 282, wherein the step of inserting comprises homology-directed repair (HDR)-mediated insertion using AsCas12a and the PAM is tttv, wherein v=a or c or g.

Embodiment 291. The method of embodiment 282, wherein the step of inserting comprises homology-directed repair (HDR)-mediated insertion using AacCas12b and the PAM is ttn, wherein n=a, c, t, or g.

Embodiment 292. The method of embodiment 282, wherein the step of inserting comprises homology-directed repair (HDR)-mediated insertion using BhCas12b and the PAM is attn., tttn, or gttn, wherein n=a, c, t, org.

Embodiment 293. The method of any one of embodiments 277-292, wherein the first insertion site is in an exon.

Embodiment 294. The method of any one of embodiments 277-292, wherein the first insertion site is in an intron.

Embodiment 295. The method of any one of embodiments 277-292, wherein the first insertion site is between an intron and an exon.

Embodiment 296. The method of any one of embodiments 277-292, wherein the first insertion site is in a regulatory region.

Embodiment 297. The method of any one of embodiments 277-296, wherein the engineered cell is a human cell.

Embodiment 298. The method of any one of embodiments 277-297, wherein the engineered cell is a T-cell, an islet cell, a cardiomyocyte, a hepatocyte, or a stem cell.

Embodiment 299. The method of any one of embodiments 277-298, wherein the engineered cell is a T-cell.

Embodiment 300. The method of any one of embodiments 277-299, wherein the engineered cell is a human T-cell.

Embodiment 301. The method of any one of embodiments 297-300, wherein the engineered cell is an allogeneic T-cell.

Embodiment 302. The method of embodiment 301, wherein the allogeneic T-cell is a primary T-cell.

Embodiment 303. The method of embodiment 301, wherein the allogeneic T-cell has been differentiated from an embryonic stem cell (ESC) or an induced pluripotent stem cell (iPSC).

Embodiment 304. The method of any one of embodiments 277-303, wherein the tolerogenic factor is or comprises: CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, Serpinb9, CCl21, Mfge8, DUX4, B2M-HLA-E, CD27, IL-39, CD16 Fe Receptor, IL15-RF, H2-M3 (HLA-G), A20/TNFAIP3, CR1, HLA-F, or MANF.

Embodiment 305. The method of any one of embodiments 277-304, wherein the tolerogenic factor is or comprises CD47.

Embodiment 306. The method of embodiments 277-305, wherein the tolerogenic factor is or comprises human CD47.

Embodiment 307. The method of embodiment 305 or 306, wherein the CD47 comprises an amino acid sequence at least 80%, at least 85%, at least 90%, at least 95%, at least 99%, or 100% identical to an amino acid sequence set forth in SEQ ID NO:1 or SEQ ID NO:2.

Embodiment 308. The method of any one of embodiments 277-307, wherein the first transgene encodes CD47 and comprises a nucleotide sequence at least 80%, at least 85%, at least 90%, at least 95%, at least 99%, or 100% identical to a nucleotide sequence set forth in SEQ ID NO:3 or SEQ ID NO:4.

Embodiment 309. The method of embodiment 277-308, wherein the nucleotide sequence of the first transgene is codon-optimized.

Embodiment 310. The method of embodiment 309, wherein the first transgene is at least 80%, at least 85%, at least 90%, at least 95%, at least 99%, or 100% identical to a nucleotide sequence set forth in SEQ ID NO:5.

Embodiment 311. The method of any one of embodiments 277-310, wherein the engineered cell further comprises a transgene that does not encode: a chimeric antigen receptor (CAR), chimeric co-stimulatory receptor (CCR), cytokine, dominant negative, microenvironment modulator, antibody, biosensor, chimeric receptor ligand (CLR), chimeric immune receptor ligand (CIRL), soluble receptor, solute transporter, enzyme, ribozyme, genetic circuit, epigenetic modifier, transcriptional activator or repressor, or non-coding RNA in its genome.

Embodiment 311a. The method of any one of embodiments 277-311, wherein the engineered cell further comprises a transgene encoding a that does not encode: 4-IBB, OX40, ICOS, CD70, IL2, IL12, IL15, IL18, inhibitory chimeric antigen receptor (iCAR) (e.g., extracellular scFv domain fused to an intracellular signaling domain derived from inhibitory T-cell receptors (e.g., PD-1 or CTL4)), a secretable soluble cytokine receptor (e.g., for TGFBeta, IL10), a secretable soluble T-cell inhibitory receptor (e.g., derived from PD-1, CTLA4, LAG3, or TIM-3), heparanase, Herpes Virus Entry Mediator (HVEM) (TNFRSF14), antibody against a T-cell inhibitory ligand (e.g., PD-1L, CD80, CD86, Galectin-9, or Fas ligand), toll-like receptor (TLR), Calcium-sensing Calmodulin (CaM) calmodulin-binding peptide, IL4R, IL10R, PD1, CTL4, TIM-3, LAG3, a glucose transporter (e.g., Glut1 or Glut3), PKM2, a pathogen-specific ribozyme, a viral-specific ribozyme, a HIF1a-dependent transcription unit, a TALE-VP64-dependent transcription unit, a cell-surface CD19-specific scFV-NFAT fusion protein, a chimeric programmable sequence-specific DNA binding domain fused to p300 acetyltransferase domain (a histone H3 acetylase), a chimeric programmable sequence-specific DNA binding domain fused to KRAB repressor domain, Foxp3, NFAT, HIF-1alpha, a DNA binding domain (e.g., TAL, zinc-finger, CRISPR/deactivatedCas9) and a transactivation domain (e.g., VP16, VP64, p65, Rta, or combinations of them), fusion proteins comprising a DNA binding domain (e.g., TAL, zinc-finger, CRISPR/deactivatedCas9) and a repressor domain (e.g., KRAB), microRNA (miRNA), small interfering RNA (siRNA), anti-sense RNA targeting e.g., target inhibitory receptor gene messenger RNAs such as messenger RNAs of PD1, TIM-3, LAG3, CTLA-4.

Embodiment 312. The method of any one of embodiments 277-311, wherein the TCR gene locus is or comprises an endogenous TCR gene locus.

Embodiment 313. The method of any one of embodiments 277-312, wherein the TCR gene locus is or comprises: a TRAC locus, a TRBC1 locus, or a TRBC2 locus.

Embodiment 314. The method of any one of embodiments 277-313, wherein the engineered cell does not express a functional endogenous TCR.

Embodiment 315. The method of any one of embodiments 277-314, wherein the insertion of the first transgene encoding a tolerogenic factor at a first insertion site at the TCR gene locus prevents expression of a functional TCR.

Embodiment 316. A method of generating a population of genetically engineered cells, the method comprising: inserting a first transgene encoding a tolerogenic factor at a first insertion site at a TCR gene locus in the genome of the cells.

Embodiment 317. The method of embodiment 316, wherein the step of inserting comprises using a genome-modifying protein.

Embodiment 317a. The method of embodiment 317, wherein the genome-modifying protein comprises a CRISPR-associated transposase, prime editing, or Programmable Addition via Site-specific Targeting Elements (PASTE).

Embodiment 317b. The method of embodiment 317, wherein the genome-modifying protein comprises a TnpB polypeptide.

Embodiment 317c. The method of embodiment 316, wherein the step of inserting comprises homology-directed repair (HDR)-mediated insertion using a site-directed nuclease.

Embodiment 318. The method of embodiment 317c, wherein the site-directed nuclease is selected from the group consisting of: Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

Embodiment 319. The method of any one of embodiments 316-318, wherein the step of inserting comprises homology-directed repair (HDR)-mediated insertion using a guide RNA (gRNA) and a CRISPR-associated (Cas) nuclease.

Embodiment 320. The method of embodiment 319, wherein the gRNA comprises a complementary region,
wherein the complementary region comprises a nucleic acid sequence that is complementary to a target nucleic acid sequence within the TCR gene locus, and wherein the target nucleic acid sequence comprises the first insertion site.

Embodiment 320a. The method of any one of embodiments 316-320, wherein the first insertion site is 25 nucleotides or less from a target adjacent motif (TAM) sequence, wherein the TAM sequence is tca, ttcan, ttgat, or ataaa, and wherein:
(i) n=a, c, t, or g.

Embodiment 320b. The method of embodiment of embodiments 320a, wherein the engineered cell comprises TnpB and the TAM is tca.

Embodiment 320c. The method of embodiment of embodiments 320a, wherein the engineered cell comprises TnpB and the TAM is ttcan, wherein
(i) n=a, c, t, or g.

Embodiment 320d. The method of embodiment of embodiments 320a, wherein the engineered cell comprises TnpB and the TAM is ttgat.

Embodiment 320e. The method of embodiment of embodiments 320a, wherein the engineered cell comprises TnpB and the TAM is ataaa.

Embodiment 321. The method of any one of embodiments 316-320, wherein the first insertion site is 25 nucleotides or less from a protospacer adjacent motif (PAM) sequence, wherein the PAM sequence is ngg, nag, ngrrt, ngrrn, nnnngatt, nnnnryac, nnagaaw, naaaac, tttv, ttn, attn, tttn, or gttn, and wherein:
(i) r=a or g,
(ii) y=c or t,
(iii) w=a or t,
(iv) v=a or c or g,
(v) n=a, c, t, or g.

Embodiment 322. The method of embodiment 321, wherein the step of inserting comprises homology-directed repair (HDR)-mediated insertion using SpCas9 and the PAM is ngg or nag, wherein n=a, c, t, or g.

Embodiment 323. The method of embodiment 321, wherein the step of inserting comprises homology-directed repair (HDR)-mediated insertion using SaCas9 and the PAM is ngrrt or ngrrn, wherein
(i) r=a or g, and
(ii) n=a, c, t, or g.

Embodiment 324. The method of embodiment 321, wherein the step of inserting comprises homology-directed repair (HDR)-mediated insertion using NmeCas9 and the PAM is nnnngatt, wherein n=a, c, t, or g.

Embodiment 325. The method of embodiment 321, wherein the step of inserting comprises homology-directed repair (HDR)-mediated insertion using CjCas9 and the PAM is nnnnryac, wherein:
(i) r=a or g,
(ii) y=c or t, and
(iii) n=a, c, t, or g.

Embodiment 326. The method of embodiment 321, wherein the step of inserting comprises homology-directed repair (HDR)-mediated insertion using StCas9 and the PAM is nnagaaw wherein:
(i) w=a or t, and
(ii) n=a, c, t, or g.

Embodiment 327. The method of embodiment 321, wherein the step of inserting comprises homology-directed repair (HDR)-mediated insertion using TdCas9 and the PAM is naaaac, wherein n=a, c, t, or g.

Embodiment 328. The method of embodiment 321, wherein the step of inserting comprises homology-directed repair (HDR)-mediated insertion using LbCas12a and the PAM is tttv, wherein v=a or c or g.

Embodiment 329. The method of embodiment 321, wherein the step of inserting comprises homology-directed repair (HDR)-mediated insertion using AsCas12a and the PAM is tttv, wherein v=a or c or g.

Embodiment 330. The method of embodiment 321, wherein the step of inserting comprises homology-directed repair (HDR)-mediated insertion using AacCas12b and the PAM is ttn, wherein n=a, c, t, or g.

Embodiment 331. The method of embodiment 321, wherein the step of inserting comprises homology-directed repair (HDR)-mediated insertion using BhCas12b and the PAM is attn., tttn, or gttn, wherein n=a, c, t, or g.

Embodiment 332. The method of any one of embodiments 316-331, wherein the first insertion site is in an exon.

Embodiment 333. The method of any one of embodiments 316-331, wherein the first insertion site is in an intron.

Embodiment 334. The method of any one of embodiments 316-331, wherein the first insertion site is between an intron and an exon.

Embodiment 335. The method of any one of embodiments 316-331, wherein the first insertion site is in a regulatory region.

Embodiment 336. The method of any one of embodiments 316-335, wherein the insertion of the first transgene encoding a tolerogenic factor at a first insertion site at a T-cell receptor (TCR) gene locus prevents expression of a functional TCR.

Embodiment 337. The method of any one of embodiments 316-336, wherein the population of genetically engineered cells are human cells.

Embodiment 338. The method of any one of embodiments 316-337, wherein the population of genetically engineered cells are T-cells, islet cells, cardiomyocytes, hepatocytes, or stem cells.

Embodiment 339. The method of any one of embodiments 316-338, wherein the population of genetically engineered cells are T-cells.

Embodiment 340. The method of any one of embodiments 316-339, wherein the population of genetically engineered cells are human T-cells.

Embodiment 341. The method of any one of embodiments 316-340, wherein the population of genetically engineered cells are allogeneic T-cells.

Embodiment 342. The method of embodiment 341, wherein the allogeneic T-cells are primary T-cells.

Embodiment 343. The method of embodiment 341, wherein the allogeneic T-cells have been differentiated from embryonic stem cells (ESCs) or induced pluripotent stem cells (iPSCs).

Embodiment 344. The method of any one of embodiments 316-343, wherein the tolerogenic factor is or comprises: CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, Serpinb9, CCl21, Mfge8, DUX4, B2M-HLA-E, CD27, IL-39, CD16 Fe Receptor, IL15-RF, H2-M3 (HLA-G), A20/TNFAIP3, CR1, HLA-F, or MANF.

Embodiment 345. The method of any one of embodiments 316-343, wherein the tolerogenic factor is or comprises CD47.

Embodiment 346. The method of embodiments 316-345, wherein the tolerogenic factor is or comprises human CD47.

Embodiment 347. The method of embodiment 345 or 346, wherein the CD47 comprises an amino acid sequence at least 80%, at least 85%, at least 90%, at least 95%, at least 99%, or 100% identical to an amino acid sequence set forth in SEQ ID NO:1 or SEQ ID NO:2.

Embodiment 348. The method of any one of embodiments 316-347, wherein the first transgene encodes CD47 and comprises a nucleotide sequence at least 80%, at least 85%, at least 90%, at least 95%, at least 99%, or 100% identical to a nucleotide sequence set forth in SEQ ID NO:3 or SEQ ID NO:4.

Embodiment 349. The method of embodiment 316-348, wherein the nucleotide sequence of the first transgene is codon-optimized.

Embodiment 350. The method of embodiment 349, wherein the first transgene is at least 80%, at least 85%, at least 90%, at least 95%, at least 99%, or 100% identical to a nucleotide sequence set forth in SEQ ID NO:5.

Embodiment 351. The method of any one of embodiments 316-350, wherein the engineered cell further comprises a transgene that does not encode: a chimeric antigen receptor (CAR), chimeric co-stimulatory receptor (CCR), cytokine, dominant negative, microenvironment modulator, antibody, biosensor, chimeric receptor ligand (CLR), chimeric immune receptor ligand (CIRL), soluble receptor, solute transporter, enzyme, ribozyme, genetic circuit, epigenetic modifier, transcriptional activator or repressor, or non-coding RNA in its genome.

Embodiment 351a. The method any one of embodiments 316-351, wherein the engineered cell further comprises a transgene encoding a that does not encode: 4-IBB, OX40, ICOS, CD70, IL2, IL12, IL15, IL18, inhibitory chimeric antigen receptor (iCAR) (e.g., extracellular scFv domain fused to an intracellular signaling domain derived from inhibitory T-cell receptors (e.g., PD-1 or CTL4)), a secretable soluble cytokine receptor (e.g., for TGFBeta, IL10), a secretable soluble T-cell inhibitory receptor (e.g., derived from PD-1, CTLA4, LAG3, or TIM-3), heparanase, Herpes Virus Entry Mediator (HVEM) (TNFRSF14), antibody against a T-cell inhibitory ligand (e.g., PD-IL, CD80, CD86, Galectin-9, or Fas ligand), toll-like receptor (TLR), Calcium-sensing Calmodulin (CaM) calmodulin-binding peptide, IL4R, IL10R, PD1, CTL4, TIM-3, LAG3, a glucose transporter (e.g., Glut1 or Glut3), PKM2, a pathogen-specific ribozyme, a viral-specific ribozyme, a HIF1a-dependent transcription unit, a TALE-VP64-dependent transcription unit, a cell-surface CD19-specific scFV-NFAT fusion protein, a chimeric programmable sequence-specific DNA binding domain fused to p300 acetyltransferase domain (a histone H3 acetylase), a chimeric programmable sequence-specific DNA binding domain fused to KRAB repressor domain, Foxp3, NFAT, HIF-1alpha, a DNA binding domain (e.g., TAL, zinc-finger, CRISPR/deactivatedCas9) and a transactivation domain (e.g., VP16, VP64, p65, Rta, or combinations of them), fusion proteins comprising a DNA binding domain (e.g., TAL, zinc-finger, CRISPR/deactivatedCas9) and a repressor domain (e.g., KRAB), microRNA (miRNA), small interfering RNA (siRNA), anti-sense RNA targeting e.g., target inhibitory receptor gene messenger RNAs such as messenger RNAs of PD1, TIM-3, LAG3, CTLA-4.

Embodiment 352. The method of any one of embodiments 316-351, wherein the TCR gene locus is or comprises an endogenous TCR gene locus.

Embodiment 353. The method of any one of embodiments 316-352, wherein the TCR gene locus is or comprises: a TRAC locus, a TRBC1 locus, or a TRBC2 locus.

Embodiment 354. The method of any one of embodiments 316-353, wherein the population of engineered cells do not express a functional endogenous TCR.

Embodiment 355. The method of any one of embodiments 316-354, wherein the insertion of the first transgene encoding a tolerogenic factor at a first insertion site at a T-cell receptor (TCR) gene locus prevents expression of a functional TCR.

Embodiment 356. The method of any one of embodiments 316-355, wherein the method further comprises reducing expression of major histocompatibility complex (MHC) class I (MHC I) molecules and/or MHC class II (MHC II) molecules in one or more cells in the population.

Embodiment 357. The method of embodiment 356, wherein reducing expression of MHC I molecules comprises modulation of a B2M locus of the one or more cells in the population.

Embodiment 358. The method of embodiment 356, wherein reducing expression of MHC II molecules comprises modulation of a CIITA locus of the one or more cells in the population.

Embodiment 359. The method of embodiment 357 or 358, wherein modulation of the B2M locus comprises knock-out of the B2M locus and/or modulation of the CIITA locus comprises knock-out of the CIITA locus.

Embodiment 360. The method of embodiment 359, wherein knock-out of the B2M locus and/or knock-out of the CIITA locus occurs at: both B2M alleles, both CIITA alleles, or combinations thereof.

Embodiment 361. The method of any one of embodiments 356-360, wherein the step of inserting the first transgene occurs before, after, or together with, the step of reducing expression of MHC I molecules and/or MHC II molecules.

Embodiment 362. The method of any one of embodiments 356-361, wherein the genetically engineered cells are T-cells, and wherein, after the steps of inserting the first transgene and reducing expression of MHC I and/or MHC II molecules, at least 50% of the T-cells each have (a) reduced cell surface expression of MHC I and/or MHC II molecules, and (b) increased expression of a tolerogenic factor encoded by the first transgene.

Embodiment 363. The population of cells according to embodiment 362, wherein the tolerogenic factor is CD47.

Embodiment 364. The method of any one of embodiments 357-361, wherein the genetically engineered cells are T-cells, and wherein after the steps of inserting the first transgene and modulating the B2M locus and/or the CIITA locus, at least 50% of the T-cells each have (a) B2M and/or CIITA knocked-out, and (b) increased expression of CD47 encoded by a transgene.

Embodiment 365. The method of embodiments 362-364, wherein at least 55%, at least 60%, at least 65%, at least 70%, at least 75%, at least 80%, at least 85%, at least 90%, at least 95%, or at least 100% of the T-cells each have (a) and (b).

Embodiment 366. The method of any one of embodiments 357-361, wherein the genetically engineered cells are T-cells and the tolerogenic factor is CD47, and wherein after the steps of inserting the first transgene and modulating the B2M locus and/or the CIITA locus, at least 50% of the T-cells each have (a) reduced expression of B2M, (b) reduced expression of CIITA, and (c) increased expression of CD47 encoded by a transgene.

Embodiment 367. The population of cells of embodiment 366, wherein at least 55%, at least 60%, at least 65%, at least 70%, at least 75%, at least 80%, at least 85%, at least 90%, at least 95%, or at least 100% of the T-cells each have (a) and (b).

Embodiment 368. The method of embodiment 366 or 367, wherein at least 55%, at least 60%, at least 65%, at least 70%, at least 75%, at least 80%, at least 85%, at least 90%, at least 95%, or at least 100% of the T-cells each have (a), (b), and (c).

Embodiment 369. The method of any one of embodiments 316-368, wherein the method further comprises selecting the one or more genetically engineered cells from the population of genetically engineered cells.

Embodiment 370. The method of embodiment 369, wherein the step of selecting comprises selecting for one or more genetically engineered cells based on a level of tolerogenic factor expressed on the cell surface.

Embodiment 371. The method of embodiment 370, wherein selecting for one or more genetically engineered cells based on a level of tolerogenic factor expressed on the cell surface comprises: affinity binding, flow cytometry, and/or immunomagnetic selection using antibodies and/or proteins that bind the tolerogenic factor.

Embodiment 372. The method of any one of embodiments 369-371, wherein the step of selecting comprises selecting for one or more genetically engineered cells based on a level of CD3 expressed on the cell surface.

Embodiment 373. The method of embodiment 372, wherein selecting for one or more genetically engineered cells based on a level of CD3 expressed on the cell surface comprises affinity binding, flow cytometry, and/or immunomagnetic selection using CD3-binding antibodies and/or CD3-binding proteins.

Embodiment 374. The method of any one of embodiments 369-373, wherein the step of selecting comprises selecting for one or more genetically engineered cells based on a level of MHC I and/or MHC II molecules expressed on the cell surface.

Embodiment 375. The method of embodiment 372, wherein selecting for one or more genetically engineered cells based on a level of MHC I and/or MHC II molecules expressed on the cell surface comprises affinity binding, flow cytometry, and/or immunomagnetic selection using CD3-binding antibodies and/or CD3-binding proteins.

Embodiment 376. The method of any one of embodiments 316-375, further comprising enriching for one or more genetically engineered cells that express higher levels of CD47 on their cell surface as compared to comparable unmodified cells.

Embodiment 377. The method of any one of embodiments 316-376, further comprising enriching for one or more genetically engineered cells that express lower levels of MHC I and/or MHC II on their cell surface as compared to comparable unmodified cells.

Embodiment 378. The method of any one of embodiments 316-377, further comprising enriching for one or more genetically engineered cells that express lower levels of CD3 on their cell surface as compared to comparable unmodified cells.

Embodiment 379. The method of any one of embodiments 316-377, further comprising enriching for one or more genetically engineered cells that express lower levels of B2M and/or CIITA as compared to comparable unmodified cells.

Embodiment 380. A method of identifying a site for inserting a first transgene at a TCR gene locus, comprising the steps of:
(a) identifying a PAM sequence in (i) the TCR gene locus, (ii) the 100 bp upstream of the 5′ end of TCR gene locus, or (iii) the 100 bp downstream of the 3′ end of TCR gene locus, and
(b) generating a gRNA comprising a complementary region, wherein the complementary region comprises a nucleic acid sequence that is complementary to a target nucleic acid sequence within the TCR gene locus, wherein the target nucleic acid sequence comprises the first insertion site, and wherein the first insertion site is 25 nucleotides or less from a protospacer adjacent motif (PAM) sequence.

Embodiment 381. A method of producing a composition comprising genetically engineered cells, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprise a transgene encoding a first tolerogenic factor at an insertion site at a T-cell receptor (TCR) gene locus, and
wherein the level of the one or more markers on the cell surface comprise a level of CD3.

Embodiment 382. A method of selecting engineered cells suitable for use in a therapeutic product, the method comprising:
selecting one or more genetically engineered cells from a population of cells based on a level of one or more markers on the cell surface of the one or more genetically engineered cells, and
preparing the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a T-cell receptor (TCR) gene locus, and
wherein the level of the one or more markers on the cell surface comprise a level of CD3.

Embodiment 383. A method of treating a disease in a subject with a composition comprising genetically engineered cells, the method comprising:
selecting one or more genetically engineered cells from a population of cells based on a level of one or more markers on the cell surface of the one or more genetically engineered cells,
formulating the composition comprising the selected one or more genetically engineered cells for treating a disease in a subject, and
administering the formulated composition to a subject,
wherein the one or more genetically engineered cells comprise one or more genetic modifications, and the one or more genetic modifications comprises a transgene encoding a first tolerogenic factor at an insertion site at a T-cell receptor (TCR) gene locus, and
wherein the level of the one or more markers on the cell surface comprise a level of CD3.

Embodiment 384. A method of producing a composition comprising engineered cells with increased purity, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprise a transgene encoding a first tolerogenic factor at an insertion site at a T-cell receptor (TCR) gene locus,
wherein the level of the one or more markers on the cell surface comprise a level of CD3, and
wherein at least 30% of the genetically engineered cells in the formulated composition comprise the transgene encoding the first tolerogenic factor at the insertion site at the TCR gene locus.

Embodiment 385. A method of producing a composition comprising genetically engineered cells with enhanced efficacy, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a T-cell receptor (TCR) gene locus,
wherein the level of the one or more markers on the cell surface comprise a level of CD3, and
wherein the composition with enhanced efficacy is more effective than a composition comprising cells that do not comprise the one or more genetic modifications.

Embodiment 386. A method of producing a composition with reduced host immune response, the method comprising:

selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprise a transgene encoding a first tolerogenic factor at an insertion site at a T-cell receptor (TCR) gene locus,
wherein the level of the one or more markers on the cell surface comprises a level of CD3 on the cell surface of the one or more genetically engineered cells, and
wherein the composition with reduced host immune response elicits a reduced host immune response compared to a composition comprising comparable cells that do not comprise the one or more genetic modifications.

Embodiment 387. A method of formulating a composition with reduced immunogenicity, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a T-cell receptor (TCR) gene locus,
wherein the level of one or more markers on the cell surface comprises a level of CD3, and
wherein the composition with reduced immunogenicity elicits a reduced host immune response compared to a composition comprising comparable cells that do not comprise the one or more genetic modifications.

Embodiment 388. A method of producing a composition comprising genetically engineered cells with reduced immunogenicity, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a T-cell receptor (TCR) gene locus, and
wherein the level of the one or more markers on the cell surface comprise a level of CD3, and
wherein the composition with reduced immunogenicity elicits a reduced host immune response compared to a composition comprising comparable cells that do not comprise the one or more genetic modifications.

Embodiment 389. The method of any one of embodiments 386-388, wherein the host immune response is an immune response of the subject against the one or more genetically engineered cells.

Embodiment 390. The method of embodiment 389, wherein the reduced host immune response comprises reduced donor-specific antibodies in the subject.

Embodiment 391. The method of embodiments 386-389, wherein the reduced host immune response comprises reduced IgM or IgG antibodies in the subject.

Embodiment 392. The method of embodiments 386-389, wherein the reduced host immune response comprises reduced complement-dependent cytotoxicity (CDC) in the subject.

Embodiment 393. The method of embodiments 386-389, wherein the reduced host immune response comprises reduced TH1 activation in the subject.

Embodiment 394. The method of embodiments 386-389, wherein the reduced host immune response comprises reduced NK cell killing in the subject.

Embodiment 395. The method of embodiments 386-389, wherein the reduced host immune response comprises reduced killing by whole PBMCs in the subject.

Embodiment 396. A method of producing a composition comprising genetically engineered cells with a reduced graft versus host response, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprises a transgene encoding a first tolerogenic factor at an insertion site at a T-cell receptor (TCR) gene locus, and
optionally wherein the level of the one or more markers on the cell surface comprise a level of CD3, and
wherein the one or more genetically engineered cells of the composition with a reduced graft versus host response have a reduced immune response against cells of the subject as compared to a composition comprising comparable cells that do not comprise the one or more genetic modifications.

Embodiment 397. A method of producing a composition comprising genetically engineered cells, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, optionally wherein the level of the one or more markers on the cell surface comprise a level of CD3.

Embodiment 398. The method of any embodiments 381-397, wherein the one or more genetic modifications comprises an inserted transgene encoding a first tolerogenic factor.

Embodiment 399. The method of any embodiments 381-398, wherein the method comprises inserting a transgene encoding a first tolerogenic factor into an insertion site in the genome of one or more cells in the population.

Embodiment 400. The method of any embodiments 381-399, wherein the transgene encoding the first tolerogenic factor is inserted at an insertion site at a T-cell receptor (TCR) gene locus.

Embodiment 401. The method of any of embodiments 381-400, wherein the step of inserting comprises homology-directed repair (HDR)-mediated insertion using a genome-modifying protein.

Embodiment 402. The method of any of embodiments 381-401, wherein the step of inserting using a genome modifying protein comprises insertion by a CRISPR-associated transposase, prime editing, a TnpB polypeptide, or Programmable Addition via Site-specific Targeting Elements (PASTE).

Embodiment 403. The method of any of embodiments 381-402, wherein the step of inserting using a genome modifying protein comprises insertion by a site-directed nuclease.

Embodiment 404. The method of any of embodiments 381-403, wherein the site-directed nuclease is selected from a zinc finger nuclease (ZFN), a TAL-effector nuclease (TALEN), or a CRISPR-Cas combination, optionally wherein the Cas is selected from a Cas9 or a Cas12.

Embodiment 405. The method of any of embodiments 381-404, wherein the site-directed nuclease is selected from the group consisting of: Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, and a TnpB polypeptide.

Embodiment 406. The method of any embodiments 381-405, wherein the step of inserting comprises homology-directed repair (HDR)-mediated insertion using a guide RNA (gRNA) and a CRISPR-associated (Cas) nuclease.

Embodiment 407. The method of any embodiments 381-406, wherein the gRNA comprises a complementary region, wherein the complementary region comprises a nucleic acid sequence that is complementary to a target nucleic acid sequence within the TCR gene locus, and wherein the target nucleic acid sequence comprises the insertion site.

Embodiment 408. The method of any embodiments 381-407, wherein the insertion site is 25 nucleotides or less from a protospacer adjacent motif (PAM) sequence, wherein the PAM sequence is ngg, nag, ngrrt, ngrrn, nnnngatt, nnnnryac, nnagaaw, naaaac, tttv, ttn, attn, tttn, or gttn, and wherein:
(i) r=a or g,
(ii) y=c or t,
(iii) w=a or t,
(iv) v=a or c or g,
(v) n=a, c, t, or g.

Embodiment 409. The method of any embodiments 381-408, wherein the step of inserting comprises homology-directed repair (HDR)-mediated insertion using SpCas9 and the PAM is ngg or nag, wherein n=a, c, t, org.

Embodiment 410. The method of any embodiments 381-409, wherein the step of inserting comprises homology-directed repair (HDR)-mediated insertion using SaCas9 and the PAM is ngrrt or ngrrn, wherein:
(i) r=a or g, and
(ii) n=a, c, t, or g.

Embodiment 411. The method of any embodiments 381-410, wherein the step of inserting comprises homology-directed repair (HDR)-mediated insertion using NmeCas9 and the PAM is nnnngatt, wherein n=a, c, t, or g.

Embodiment 412. The method of any embodiments 381-411, wherein the step of inserting comprises homology-directed repair (HDR)-mediated insertion using CjCas9 and the PAM is nnnnryac, wherein:
(i) r=a or g,
(ii) y=c or t, and
(iii) n=a, c, t, or g.

Embodiment 413. The method of any embodiments 381-412, wherein the step of inserting comprises homology-directed repair (HDR)-mediated insertion using StCas9 and the PAM is nnagaaw wherein:
(i) w=a or t, and
(ii) n=a, c, t, or g.

Embodiment 414. The method of any embodiments 381-413, wherein the step of inserting comprises homology-directed repair (HDR)-mediated insertion using TdCas9 and the PAM is naaaac, wherein n=a, c, t, or g.

Embodiment 415. The method of any embodiments 381-414, wherein the step of inserting comprises homology-directed repair (HDR)-mediated insertion using LbCas12a and the PAM is tttv, wherein v=a or c or g.

Embodiment 416. The method of any embodiments 381-415, wherein the step of inserting comprises homology-directed repair (HDR)-mediated insertion using AsCas12a and the PAM is tttv, wherein v=a or c or g.

Embodiment 417. The method of any embodiments 381-416, wherein the step of inserting comprises homology-directed repair (HDR)-mediated insertion using AacCas12b and the PAM is ttn, wherein n=a, c, t, or g.

Embodiment 418. The method of any embodiments 381-417, wherein the step of inserting comprises homology-directed repair (HDR)-mediated insertion using BhCas12b and the PAM is attn., tttn, or gttn, wherein n=a, c, t, or g.

Embodiment 419. The method of any embodiments 381-418, wherein homology-directed repair (HDR)-mediated insertion using a site-directed nuclease is performed with an HDR efficiency equal to or greater than HDR insertion using lentivirus.

Embodiment 420. The method of any embodiments 381-419, wherein the step of inserting comprises homology-directed repair (HDR)-mediated insertion using ZFN.

Embodiment 421. The method of any embodiments 381-420, wherein the first insertion site is 25 nucleotides or less from a zinc finger binding sequence.

Embodiment 422. The method of any embodiments 381-421, wherein the step of inserting comprises homology-directed repair (HDR)-mediated insertion using TALEN.

Embodiment 423. The method of any embodiments 381-422, wherein the first insertion site is 25 nucleotides or less from a transcription activator-like effectors (TALE) binding sequence.

Embodiment 424. The method of any embodiments 381-423, wherein the step of inserting comprises homology-directed repair (HDR)-mediated insertion using a guide RNA (gRNA) and a TnpB polypeptide.

Embodiment 425. The method of any embodiments 381-424, wherein the gRNA comprises a complementary region, wherein the complementary region comprises a nucleic acid sequence that is complementary to a target nucleic acid sequence within the TCR gene locus, and wherein the target nucleic acid sequence comprises the insertion site.

Embodiment 426. The method of any embodiments 381-425, wherein the insertion site is 25 nucleotides or less from a target adjacent motif (TAM) sequence, wherein the TAM sequence is tca, ttcan, ttgatn or ataaa, and wherein:
(i) n=a, c, t, or g.

Embodiment 427. The method of any embodiments 381-426, wherein the step of inserting comprises homology-directed repair (HDR)-mediated insertion using TnpB polypeptide and the TAM is tca.

Embodiment 428. The method of any embodiments 381-427, wherein the step of inserting comprises homology-directed repair (HDR)-mediated insertion using TnpB polypeptide and the TAM is ttcan, wherein n=a, c, t, or g.

Embodiment 429. The method of any embodiments 381-428, wherein the step of inserting comprises homology-directed repair (HDR)-mediated insertion using TnpB polypeptide and the TAM is ttgatn, wherein n=a, c, t, or g.

Embodiment 430. The method of any embodiments 381-429, wherein the step of inserting comprises homology-directed repair (HDR)-mediated insertion using TnpB polypeptide and the TAM is ataaa.

Embodiment 431. The method of any embodiments 381-430, wherein the insertion site is in an exon.

Embodiment 432. The method of any embodiments 381-431, wherein the insertion site is in an intron.

Embodiment 433. The method of any embodiments 381-432, wherein the insertion site is between an intron and an exon.

Embodiment 434. The method of any embodiments 381-433 and 381-433, wherein the insertion site is in a regulatory region.

Embodiment 435. The method of any embodiments 381-434, wherein the transgene encoding the first tolerogenic factor at an insertion site at a T-cell receptor (TCR) gene locus reduces expression of a functional TCR.

Embodiment 436. The method of any embodiments 381-435, wherein the transgene encoding the first tolerogenic factor at an insertion site at a T-cell receptor (TCR) gene locus disrupts expression of a functional TCR.

Embodiment 437. The method of any embodiments 381-436, wherein the transgene encoding the first tolerogenic factor has a reverse orientation (5′ to 3′) relative to the TCR locus.

Embodiment 438. The method of any embodiments 381-437, wherein the TCR locus is an endogenous TCR locus.

Embodiment 439. The method of any embodiments 381-438, wherein the TCR locus is or comprises: a TRAC locus, a TRBC1 locus, or a TRBC2 locus.

Embodiment 440. The method of any of claims 381-439, wherein the TCR locus is or comprises a TRAC locus.

Embodiment 441. The method of any embodiments 381-440, wherein the insertion site is within exon 1 at the TRAC locus.

Embodiment 442. The method of any embodiments 381-441, wherein the step of inserting comprises using an hTRAC gRNA comprising the nucleic acid sequence TCAGGGTTCTGGATATCTGT (SEQ ID NO: 124).

Embodiment 443. The method of any embodiments 381-442, wherein the level of one or more markers on the cell surface comprises a level of the first tolerogenic factor on the cell surface of the one or more genetically engineered cells.

Embodiment 444. The method of any embodiments 381-443, wherein the method comprises detecting a level of the first tolerogenic factor on the cell surface of the one or more genetically engineered cells.

Embodiment 445. The method of any embodiments 381-444, wherein the one or more genetically engineered cells are selected if the first tolerogenic factor is detected on the cell surface of the one or more genetically engineered cells.

Embodiment 446. The method of any embodiments 381-445, wherein the first tolerogenic factor is or comprises A20/TNFAIP3, B2M-HLA-E, C1-Inhibitor, CCL21, CCL22, CD16, CD16 Fc receptor, CD24, CD27, CD35, CD39, CD46, CD47, CD52, CD55, CD59, CD200, CR1, CTLA4-Ig, DUX4, FasL, H2-M3 (HLA-G), HLA-C, HLA-E, HLA-E heavy chain, HLA-F, HLA-G, IDO1, IL-10, IL15-RF, IL-35, IL-39, MANF, Mfge8, PD-L1, or Serpinb9.

Embodiment 447. The method of any embodiments 381-446, wherein the first tolerogenic factor is or comprises CD47.

Embodiment 448. The method of any embodiments 381-447, wherein the first tolerogenic factor is or comprises human CD47.

Embodiment 449. The method of any embodiments 381-448, wherein the CD47 comprises an amino acid sequence at least 80% identical to an amino acid sequence set forth in SEQ ID NO:1 or SEQ ID NO:2.

Embodiment 450. The method of any embodiments 381-449, wherein the transgene encoding the first tolerogenic factor is a transgene that encodes CD47 and the transgene comprises a nucleotide sequence at least 80% identical to a nucleotide sequence set forth in SEQ ID NO:3 or SEQ ID NO:4.

Embodiment 451. The method of any embodiments 381-450, wherein the transgene encoding a first tolerogenic factor is a transgene that encodes CD47 and the nucleotide sequence of the transgene is codon-optimized.

Embodiment 452. The method of any embodiments 381-451, wherein the transgene is at least 80% identical to a nucleotide sequence set forth in SEQ ID NO:5.

Embodiment 453. The method of any embodiments 381-452, wherein the method comprises detecting a level of CD3 on the cell surface of the one or more genetically engineered cells.

Embodiment 454. The method of any embodiments 381-453, wherein the one or more genetically engineered cells are selected if CD3 is not present at a detectable level on the cell surface of the one or more genetically engineered cells.

Embodiment 455. The method of any embodiments 381-454, wherein the level of one or more markers on the cell surface comprises a level of TCR on the cell surface of the one or more genetically engineered cells.

Embodiment 456. The method of any embodiments 381-455, wherein the method comprises detecting a level of TCR on the cell surface of the one or more genetically engineered cells.

Embodiment 457. The method of any embodiments 381-456, wherein the one or more genetically engineered cells are selected if TCR is not present at a detectable level on the cell surface of the one or more genetically engineered cells.

Embodiment 458. The method of any embodiments 381-457, wherein the one or more genetic modifications comprise a modification at a B2M locus, a TAP I locus, a NLRC5 locus, a CIITA locus, an HLA-A locus, an HLA-B locus, an HLA-C locus, an HLA-DP locus, an HLA-DM locus, an HLA-DOA locus, an HLA-DOB locus, an HLA-DQ locus, an HLA-DR locus, a RFX5 locus, a RFXANK locus, a RFXAP locus, an NFY-A locus, an NFY-B locus, an NFY-C locus, or a combination thereof.

Embodiment 459. The method of any embodiments 381-458, wherein the one or more genetic modifications comprise a modification at an HLA-A locus, an HLA-B locus, an HLA-C locus, or a combination thereof.

Embodiment 460. The method of any embodiments 381-459, wherein the modification at the HLA-A locus, the HLA-B locus, the HLA-C locus, or a combination thereof is a heterozygous modification.

Embodiment 461. The method of any embodiments 381-460, wherein the modification at the HLA-A locus, the HLA-B locus, the HLA-C locus, or a combination thereof is a homozygous modification.

Embodiment 462. The method of any embodiments 381-461, wherein the method comprises modifying an HLA-A locus, an HLA-B locus, an HLA-C locus, or a combination thereof.

Embodiment 463. The method of any embodiments 381-462, wherein the modification at the HLA-A locus, the HLA-B locus, the HLA-C locus, or a combination thereof comprises a knock-out of the HLA-A locus, the HLA-B locus, the HLA-C locus, or a combination thereof.

Embodiment 464. The method of any embodiments 381-463, wherein the method comprises knocking out an HLA-A locus, an HLA-B locus, an HLA-C locus, or a combination thereof.

Embodiment 465. The method of any embodiments 381-464, wherein the one or more genetic modifications comprise a modification at an HLA-DM locus, an HLA-DO locus, an HLA-DP locus, an HLA-DQ locus, an HLA-DR locus, or a combination thereof.

Embodiment 466. The method of any embodiments 381-465, wherein the modification at the HLA-DM locus, the HLA-DO locus, the HLA-DP locus, the HLA-DQ locus, the HLA-DR locus, or a combination thereof is a heterozygous modification.

Embodiment 467. The method of any embodiments 381-466, wherein the modification at the HLA-DM locus, the HLA-DO locus, the HLA-DP locus, the HLA-DQ locus, the HLA-DR locus, or a combination thereof is a homozygous modification.

Embodiment 468. The method of any embodiments 381-467, wherein the method comprises modifying an HLA-DM locus, an HLA-DO locus, an HLA-DP locus, an HLA-DQ locus, an HLA-DR locus, or a combination thereof.

Embodiment 469. The method of any embodiments 381-468, wherein the modification at the HLA-DM locus, the HLA-DO locus, the HLA-DP locus, the HLA-DQ locus, the HLA-DR locus, or a combination thereof comprises a knock-out of the HLA-DM locus, the HLA-DO locus, the HLA-DP locus, the HLA-DQ locus, the HLA-DR locus, or a combination thereof.

Embodiment 470. The method of any embodiments 381-469, wherein the method comprises knocking out an HLA-DM locus, an HLA-DO locus, an HLA-DP locus, an HLA-DQ locus, an HLA-DR locus, or a combination thereof.

Embodiment 471. The method of any embodiments 381-470, wherein the one or more genetic modifications comprise a modification at a B2M locus.

Embodiment 472. The method of any embodiments 381-471, wherein the modification at the B2M locus is a heterozygous modification.

Embodiment 473. The method of any embodiments 381-472, wherein the modification at the B2M locus is a homozygous modification.

Embodiment 474. The method of any embodiments 381-473, wherein the method comprises modifying a B2M locus.

Embodiment 475. The method of any embodiments 381-474, wherein the modification at the B2M locus comprises a knock-out of the B2M locus.

Embodiment 476. The method of any embodiments 381-475, wherein the method comprises knocking out the B2M locus.

Embodiment 477. The method of any of claims 381-476, wherein the one or more genetic modifications comprise a modification at a CIITA locus.

Embodiment 478. The method of any embodiments 381-477, wherein the modification at the CIITA locus is a heterozygous modification.

Embodiment 479. The method of any one of the preceding embodiments, wherein the modification at the CIITA locus is a homozygous modification.

Embodiment 480. The method of any embodiments 381-478, wherein the method comprises modifying a CIITA locus.

Embodiment 481. The method of any embodiments 381-479, wherein the modification at the CIITA locus comprises a knock-out of the CIITA locus.

Embodiment 482. The method of any embodiments 381-481, wherein the method comprises knocking out the CIITA locus.

Embodiment 483. The method of any embodiments 381-482, wherein the level of one or more markers on the cell surface comprises a level of an MHC I molecule, an MHC II molecule, or both, on the cell surface of the one or more genetically engineered cells.

Embodiment 484. The method of any embodiments 381-483, wherein the method comprises detecting a level of the MHC I molecule, the MHC II molecule, or both, on the cell surface of the one or more genetically engineered cells.

Embodiment 485. The method of any embodiments 381-484, wherein the one or more genetically engineered cells are selected if the MHC I molecule, the MHC II molecule, or both, are not present at a detectable level on the cell surface of the one or more genetically engineered cells.

Embodiment 486. The method of any embodiments 381-485, wherein the one or more genetic modifications comprise a knock-out of: ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRBC1, TRBC2, or a combination thereof.

Embodiment 487. The method of any of claims 381-486, wherein the protein that is involved in oxidative or ER stress is selected from the group consisting of TXNIP, PERK, IRE1α, and DJ-1 (PARK7).

Embodiment 488. The method of any embodiments 381-487, wherein the level of one or more markers on the cell surface comprises a level of ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRBC1, TRBC2, or a combination thereof, on the cell surface of the one or more genetically engineered cells.

Embodiment 489. The method of any embodiments 381-488, wherein the method comprises detecting a level of ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRBC1, TRBC2, or a combination thereof, on the cell surface of the one or more genetically engineered cells.

Embodiment 490. The method of any embodiments 381-489, wherein the one or more genetically engineered cells are selected if ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRBC1, TRBC2, or a combination thereof, are not present at a detectable level on the cell surface of the one or more genetically engineered cells.

Embodiment 491. The method of any embodiments 381-490, wherein the one or more genetic modifications comprise a second inserted transgene.

Embodiment 492. The method of any embodiments 381-491, wherein the second transgene encodes a chimeric antigen receptor (CAR).

Embodiment 493. The method of any embodiments 381-492, wherein the method comprises inserting a transgene encoding a CAR in the genome of one or more cells in the population.

Embodiment 494. The method of any embodiments 381-493, wherein the transgene encoding a CAR is inserted at a safe harbor locus.

Embodiment 495. The method of any embodiments 381-494, wherein the transgene encoding a CAR is inserted at a TRAC locus, a TRBC1 locus, a TRBC2 locus, a B2M locus, a CIITA locus, a MICA locus, a MICB locus, or a safe harbor locus.

Embodiment 496. The method of any embodiments 381-495, wherein the transgene encoding a CAR is inserted at an AAVS1 locus, an ABO locus, a CCR5 locus, a CLYBL locus, a CXCR4 locus, a F3 locus, a FUT1 locus, a HMGB1 locus, a KDM5D locus, a LRP1 locus, a RHD locus, a ROSA26 locus, or a SHS231 locus.

Embodiment 497. The method of any embodiments 381-496, wherein the second transgene is inserted into same site as the transgene encoding the first tolerogenic factor.

Embodiment 498. The method of any embodiments 381-497, wherein the second transgene and the first tolerogenic factor are encoded by two separate constructs.

Embodiment 499. The method of any embodiments 381-498, wherein the second transgene and the first tolerogenic factor are encoded by a bicistronic construct.

Embodiment 500. The method of any embodiments 381-499, wherein the CAR comprises a CD5-specific CAR, a CD19-specific CAR, a CD20-specific CAR, a CD22-specific CAR, a CD23-specific CAR, a CD30-specific CAR, a CD33-specific CAR, CD38-specific CAR, a CD70-specific CAR, a CD123-specific CAR, a CD138-specific CAR, a Kappa, Lambda, B cell maturation agent (BCMA)-specific CAR, a G-protein coupled receptor family C group 5 member D (GPRC5D)-specific CAR, a CD123-specific CAR, a LeY-specific CAR, a NKG2D ligand-specific CAR, a WT1-specific CAR, a GD2-specific CAR, a HER2-specific CAR, a EGFR-specific CAR, a EGFRvIII-specific CAR, a B7H3-specific CAR, a PSMA-specific CAR, a PSCA-specific CAR, a CAIX-specific CAR, a CD171-specific CAR, a CEA-specific CAR, a CSPG4-specific CAR, a EPHA2-specific CAR, a FAP-specific CAR, a FRα-specific CAR, a IL-13Rα-specific CAR, a Mesothelin-specific CAR, a MUC1-specific CAR, a MUC16-specific CAR, a ROR1-specific CAR, a C-Met-specific CAR, a CD133-specific CAR, a Ep-CAM-specific CAR, a GPC3-specific CAR, a HPV16-E6-specific CAR, a IL13Ra2-specific CAR, a MAGEA3-specific CAR, a MAGEA4-specific CAR, a MART1-specific CAR, a NY-ESO-1-specific CAR, a VEGFR2-specific CAR, a α-Folate receptor-specific CAR, a CD24-specific CAR, a CD44v7/8-specific CAR, a EGP-2-specific CAR, a EGP-40-specific CAR, a erb-B2-specific CAR, a erb-B 2,3,4-specific CAR, a FBP-specific CAR, a Fetal acethylcholine e receptor-specific CAR, a G_{D2}-specific CAR, a G_{D3}-specific CAR, a HMW-MAA-specific CAR, a IL-11Rα-specific CAR, a KDR-specific CAR, a Lewis Y-specific CAR, a L1-cell adhesion molecule-specific CAR, a MAGE-A1-specific CAR, a Oncofetal antigen (h5T4)-specific CAR, a TAG-72-specific CAR, or a CD19/CD22-bispecific CAR.

Embodiment 501. The method of any embodiments 381-500, wherein the CAR comprises a CD19-specific CAR, a CD20-specific CAR, a CD22-specific CAR, a CD38-specific CAR, a CD123-specific CAR, a CD138-specific CAR, a BCMA-specific CAR, or a CD19/CD22-bispecific CAR.

Embodiment 502. The method of any embodiments 381-501, wherein the level of one or more markers on the cell surface comprises a level of the CAR on the cell surface of the one or more genetically engineered cells.

Embodiment 503. The method of any embodiments 381-502, wherein the method comprises detecting a level of the CAR on the cell surface of the one or more genetically engineered cells.

Embodiment 504. The method of any embodiments 381-503, wherein the one or more genetically engineered cells are selected if the CAR is detected on the cell surface of the one or more genetically engineered cells.

Embodiment 505. The method of any embodiments 381-504, wherein the second transgene encodes a second tolerogenic factor.

Embodiment 506. The method of any embodiments 381-505, wherein the second transgene encoding the second tolerogenic factor is inserted at a TRAC locus, a TRBC1 locus, a TRBC2 locus, a B2M locus, a CIITA locus, a MICA locus, a MICB locus, a safe harbor locus, an AAVS1 locus, an ABO locus, a CCR5 locus, a CLYBL locus, a CXCR4 locus, a F3 locus, a FUT1 locus, a HMGB1 locus, a KDM5D locus, a LRP1 locus, a RHD locus, a ROSA26 locus, or a SHS231 locus.

Embodiment 507. The method of any embodiments 381-506, wherein the second tolerogenic factor is or comprises A20/TNFAIP3, B2M-HLA-E, C1-Inhibitor, CCL21, CCL22, CD16, CD16 Fc receptor, CD24, CD27, CD35, CD39, CD46, CD47, CD52, CD55, CD59, CD200, CR1, CTLA4-Ig, DUX4, FasL, H2-M3 (HLA-G), HLA-C, HLA-E, HLA-E heavy chain, HLA-F, HLA-G, IDO1, IL-10, IL15-RF, IL-35, IL-39, MANF, Mfge8, PD-L1, or Serpinb9.

Embodiment 508. The method of any embodiments 381-507, wherein the first tolerogenic factor and the second tolerogenic factor are the same tolerogenic factor.

Embodiment 509. The method of any embodiments 381-508, wherein the first tolerogenic factor and the second tolerogenic factor are different tolerogenic factors.

Embodiment 510. The method of any embodiments 381-509, wherein the method comprises detecting a level of the second tolerogenic factor on the cell surface of the one or more genetically engineered cells, wherein the second tolerogenic factor is expressed at a higher level than endogenous expression levels of the second tolerogenic factor in a comparable cell that does not comprise the second transgene.

Embodiment 511. The method any embodiments 381-510, wherein the one or more genetically engineered cells are selected if the second tolerogenic factor is detected on the cell surface of the one or more genetically engineered cells at a higher level of expression than endogenous expression levels of the second tolerogenic factor in a comparable cell that does not comprise the second transgene.

Embodiment 512. The method of any embodiments 381-511, wherein the one or more genetically engineered cells are selected from a population of cells based on a level of two or more markers on the cell surface of the one or more genetically engineered cells.

Embodiment 513. The method of any embodiments 381-512, wherein the one or more genetically engineered cells are selected from a population of cells based on a level of three or more markers on the cell surface of the one or more genetically engineered cells.

Embodiment 514. The method of any embodiments 381-513, wherein the one or more genetically engineered cells are selected from a population of cells based on a level of four or more markers on the cell surface of the one or more genetically engineered cells.

Embodiment 515. The method of any embodiments 381-514, wherein each of the one or more markers on the cell surface of the one or more genetically engineered cells is associated with at least one of the one or more genetic modifications.

Embodiment 516. The method of any embodiments 381-515, wherein each of the one or more genetic modifications impacts the level of at least one of the one or more markers on the cell surface of the one or more genetically engineered cells.

Embodiment 517. The method of any embodiments 381-516, wherein one or more of: (i) the transgene encoding the first tolerogenic factor, (ii) the transgene encoding the CAR, or (iii) the transgene encoding the second tolerogenic factor comprise a promoter, an insulator, an enhancer, a polyadenylation (poly(A)) tail, a ubiquitous chromatin opening element, or a combination thereof.

Embodiment 518. The method of any embodiments 381-517, wherein one or more of: (i) the transgene encoding the first tolerogenic factor, (ii) the transgene encoding the CAR, or (iii) the transgene encoding the second tolerogenic factor comprise a promoter and the promoter is a constitutive promoter.

Embodiment 519. The method of any embodiments 381-518, wherein the constitutive promoter is an EF1α, EF1α short, CMV, SV40, PGK, adenovirus late, vaccinia virus 7.5K, SV40, HSV tk, mouse mammary tumor virus (MMTV), HIV LTR, moloney virus, Esptein Barr virus (EBV), Rous sarcoma virus (RSV), UBC CAG, MND, SSFV, or ICOS promoter.

Embodiment 520. The method of any embodiments 381-519, wherein the population of cells are human cells or non-human animal cells.

Embodiment 521. The method of any embodiments 381-520, wherein non-human animal cells are porcine, bovine or ovine cells.

Embodiment 522. The method of any embodiments 381-521, wherein the population of cells are human cells.

Embodiment 523. The method of any embodiments 381-522, wherein the population of cells are differentiated cells derived from stem cells or progenitor cells.

Embodiment 524. The method of any embodiments 381-523, wherein the stem cells are pluripotent stem cells.

Embodiment 525. The method of any embodiments 381-524, wherein the pluripotent stem cells are induced pluripotent stem cells.

Embodiment 526. The method of any embodiments 381-525, wherein the pluripotent stem cells are embryonic stem cells.

Embodiment 527. The method of any embodiments 381-526, wherein the population of cells are primary cells isolated from a donor.

Embodiment 528. The method of any embodiments 381-527, wherein the donor is a single donor or multiple donors.

Embodiment 529. The method of any embodiments 381-528, wherein the donor is healthy and/or is not suspected of having a disease or condition at the time the primary cells are obtained from the donor.

Embodiment 530. The method of any embodiments 381-529, wherein the population of cells are islet cells, beta islet cells, pancreatic islet cells, immune cells, B cells, T cells, natural killer (NK) cells, natural killer T (NKT) cells, macrophage cells, endothelial cells, muscle cells, cardiac muscle cells, smooth muscle cells, skeletal muscle cells, dopaminergic neurons, retinal pigmented epithelium cells, optic cells, hepatocytes, thyroid cells, skin cells, glial progenitor cells, neural cells, cardiac cells, stem cells, hematopoietic stem cells, induced pluripotent stem cells (iPSCs), mesenchymal stem cells (MSCs), embryonic stem cells (ESCs), pluripotent stem cell (PSCs), blood cells, or a combination thereof.

Embodiment 531. The method of any embodiments 381-530, wherein the population of cells are T-cells.

Embodiment 532. The method of any embodiments 381-531, wherein the T-cells are CD3+ T cells, CD4+ T cells, CDS+ T cells, naive T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T cells, effector memory T cells, effector memory T cells expressing CD45RA (TEMRA cells), tissue-resident memory (Trm) cells, virtual memory T cells, innate memory T cells, memory stem cell (Tse), 76 T cells, or a combination thereof.

Embodiment 533. The method of any embodiments 381-532, wherein the T cells are cytotoxic T-cells, helper T-cells, memory T-cells, regulatory T-cells, tumor infiltrating lymphocytes, or a combination thereof.

Embodiment 534. The method of any embodiments 381-533, wherein the population of cells are human T-cells.

Embodiment 535. The method of any embodiments 381-534, wherein the population of cells are autologous T-cells.

Embodiment 536. The method of any embodiments 381-535, wherein the population of cells are allogenic T-cells.

Embodiment 537. The method of any embodiments 381-536, wherein the allogeneic T cells are primary T cells.

Embodiment 538. The method of any embodiments 381-537, wherein the allogeneic T cells have been differentiated from embryonic stem cells (ESCs) or an induced pluripotent stem cells (iPSCs).

Embodiment 539. The method of any embodiments 381-538, wherein the population of cells are T-cells, and wherein, after the steps of inserting the transgene encoding the first tolerogenic factor and modifying an HLA-A locus, an HLA-B locus, an HLA-C locus, or a combination thereof, at least 30% of the population of T-cells each have (a) reduced cell surface expression of MHC I and/or MHC II molecules as compared to comparable T-cells that have not been genetically engineered, and (b) increased expression of the first tolerogenic factor encoded by the first transgene as compared to comparable T-cells that have not been genetically engineered.

Embodiment 540. The method of any embodiments 381-539, wherein the population of cells are T-cells and the tolerogenic factor is CD47, and wherein, after the steps of inserting the transgene encoding the first tolerogenic factor and knocking out the B2M locus and/or the CIITA locus, at least 30% of the population of T-cells each have (a) a B2M locus and/or a CIITA locus knocked-out, and (b) increased expression of CD47 as compared to comparable T-cells that have not been genetically engineered.

Embodiment 541. The method of any embodiments 381-540, wherein the population of cells are T-cells and the first tolerogenic factor is CD47, and wherein, after the steps of inserting the transgene encoding the first tolerogenic factor and knocking out the B2M locus and/or the CIITA locus, at least 30% of the population of T-cells each have (a) reduced cell surface expression of MHC I and/or MHC II molecules as compared to T-cells that have not been genetically engineered, and (b) increased expression of CD47 as compared to comparable T-cells that have not been genetically engineered.

Embodiment 542. The method of any embodiments 381-541, wherein at least 35% of the population of T-cells each have (a) and (b).

Embodiment 543. The method of any embodiments 381-542, wherein the population of cells are T-cells and the tolerogenic factor is CD47, and wherein, after the steps of inserting the transgene encoding the first tolerogenic factor and knocking out the B2M locus and/or the CIITA locus, at least 20% of the population of T-cells each have (a) reduced expression of B2M as compared to comparable T-cells that have not been genetically engineered, (b) reduced expression of CIITA as compared to comparable T-cells that have not been genetically engineered, and (c) increased expression of CD47 as compared to comparable T-cells that have not been genetically engineered.

Embodiment 544. The method of any embodiments 381-543, wherein at least 35% of the T-cells each have (a) and (b).

Embodiment 545. The method of any embodiments 381-544, wherein at least 35% of the population of T-cells each have (a), (b), and (c).

Embodiment 546. The method of any embodiments 381-545, further comprising freezing the cells.

Embodiment 547. The method of any embodiments 381-546, wherein the one or more genetically engineered cells are frozen after being selected based on a level of one or more markers on the cell surface of the one or more genetically engineered cells.

Embodiment 548. The method of any embodiments 381-547, wherein the one or more genetically engineered cells are frozen after one or more genetic modifications are introduced.

Embodiment 549. The method of any of claims 381-548, further comprising thawing the cells.

Embodiment 550. The method of any embodiments 381-549, wherein the one or more genetically engineered cells are thawed prior to one or more genetic modifications being introduced.

Embodiment 551. The method of any embodiments 381-550, wherein the one or more genetically engineered cells are formulated in the composition after thawing.

Embodiment 552. The method of any embodiments 381-551, wherein the one or more genetically engineered cells are formulated in the composition before thawing.

Embodiment 553. The method of any embodiments 381-552, wherein the composition is suitable for use in a subject, Embodiment 554. The method of any embodiments 381-553, wherein the composition is a therapeutic composition.

Embodiment 555. The method of any embodiments 381-554, wherein the composition is a cell therapy composition.

Embodiment 556. The method of any embodiments 381-555, wherein the composition comprises a pharmaceutically acceptable additive, carrier, diluent, or excipient.

Embodiment 557. The method of any embodiments 381-556, wherein the composition comprises a buffered solution.

Embodiment 558. The method of any embodiments 381-557, wherein the composition comprises a pharmaceutically acceptable buffer.

Embodiment 559. The method of any embodiments 381-558, wherein the pharmaceutically acceptable buffer comprises neutral buffer saline or phosphate buffered saline.

Embodiment 560. The method of any embodiments 381-559, wherein the composition comprises Plasma-Lyte A®, dextrose, dextran, sodium chloride, human serum albumin (HSA), dimethylsulfoxide (DMSO), or a combination thereof.

Embodiment 561. The method of any embodiments 381-560, wherein the composition comprises a cryoprotectant.

Embodiment 562. A population of genetically engineered cells produced by the method of any of claims 381-561.

Embodiment 563. A population of cells that have been genetically engineered to comprise a transgene encoding a first tolerogenic factor, wherein at least 30% of the cells have increased cell surface expression of a first tolerogenic factor as compared to a comparable cell that has not been genetically engineered.

Embodiment 564. The population of cells of any embodiments 381-563, wherein the transgene encoding the first tolerogenic factor is inserted at an insertion site at a T-cell receptor (TCR) gene locus.

Embodiment 565. The population of cells of any embodiments 381-564, wherein the insertion site is in an exon.

Embodiment 566. The population of cells of any embodiments 381-565, wherein the insertion site is in an intron.

Embodiment 567. The population of cells of any embodiments 381-566, wherein the insertion site is between an intron and an exon.

Embodiment 568. The population of cells of any embodiments 381-567, wherein the insertion site is in a regulatory region.

Embodiment 569. The population of cells of any embodiments 381-568, wherein at least 35% of the cells have increased cell surface expression of a first tolerogenic factor as compared to a comparable cell that has not been genetically engineered.

Embodiment 570. The population of cells of any embodiments 381-569, wherein the tolerogenic factor is CD47.

Embodiment 571. The population of cells of any embodiments 381-570, wherein at least 30% of the cells have decreased cell surface expression of a TCR as compared to a comparable cell that has not been genetically engineered.

Embodiment 572. The population of cells of any embodiments 381-571, wherein at least 35% of the cells have decreased cell surface expression of a TCR as compared to a comparable cell that has not been genetically engineered.

Embodiment 573. The population of cells of any embodiments 381-572, wherein the cells have been genetically engineered to knock-out an HLA-A locus, an HLA-B locus, an HLA-C locus, or a combination thereof.

Embodiment 574. The population of cells of any embodiments 381-573, wherein the cells have been genetically engineered to knock-out an HLA-DM locus, an HLA-DO locus, an HLA-DP locus, an HLA-DQ locus, an HLA-DR locus, or a combination thereof.

Embodiment 575. The population of cells of any embodiments 381-574, wherein the cells have been genetically engineered to knock-out a B2M locus.

Embodiment 576. The population of cells of any embodiments 381-575, wherein the cells have been genetically engineered to knock-out a CIITA locus.

Embodiment 577. The population of cells of any embodiments 381-576, wherein at least 30% of the cells have decreased cell surface expression of an MHC I molecule, an MHC II molecule, or both, as compared to a comparable cell that has not been genetically engineered.

Embodiment 578. The population of cells of any embodiments 381-577, wherein at least 35% of the cells have decreased cell surface expression of an MHC I molecule, an MHC II molecule, or both, as compared to a comparable cell that has not been genetically engineered.

Embodiment 579. The population of cells of any embodiments 381-578, wherein the cells have been genetically engineered to comprise a transgene encoding a CAR.

Embodiment 580. The population of cells of any embodiments 381-579, wherein at least 35% of the cells have cell surface expression of the CAR.

Embodiment 581. A composition comprising a population of cells according to any of claims 381-580.

Embodiment 582. A pharmaceutical composition comprising (i) a population of cells according to any of embodiments 381-581, and (ii) a pharmaceutically acceptable excipient.

Embodiment 583. A method comprising administering to a subject a population of cells of any of embodiments 381-582, a composition of any of embodiments 381-582, or a pharmaceutical composition of any of embodiments 381-582.

Embodiment 584. The method of any embodiments 381-583, wherein the method is a method of treating a disease in a subject.

Embodiment 585. A population of cells of any embodiments 381-584 for use in treating a disease in a subject.

Embodiment 586. A composition of any embodiments 381-585 for use in treating a disease in a subject.

Embodiment 587. A pharmaceutical composition of any embodiments 381-586 for use in treating a disease in a subject.

Embodiment 588. Use of a population of cells of any embodiments 381-587, a composition of embodiments 381-587, or a pharmaceutical composition of any embodiments 381-587 for use in treating a disease in a subject.

Embodiment 589. Use of a population of cells of any embodiments 381-588, a composition of any embodiments 381-588, or a pharmaceutical composition of any embodiments 381-588 in the manufacture of a medicament for the treatment of a disease.

Embodiment 590. The method of any of embodiments 381-589, the population of cells of any of embodiments 381-589, the composition of any of embodiments 381-589, the pharmaceutical composition of any of embodiments 381-589, or the use of any of embodiments 381-589, wherein the disease is cancer.

Embodiment 591. The method, the population of cells, the composition, the pharmaceutical composition or the use of any of embodiments 381-590, wherein the cancer is associated with CD5, CD19, CD20, CD22, CD23, CD30, CD33, CD70, Kappa, Lambda, B cell maturation agent (BCMA), G-protein coupled receptor family C group 5 member D (GPRC5D), CD123, LeY, NKG2D ligand, WT1, GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, ROR1, C-Met, CD133, Ep-CAM, GPC3, HPV16-E6, IL13Ra2, MAGEA3, MAGEA4, MART1, NY-ESO-1, VEGFR2, α-Folate receptor, CD24, CD44v7/8, EGP-2, EGP-40, erb-B2, erb-B 2,3,4, FBP, Fetal acethylcholine e receptor, G_{D2}, G_{D3}, HMW-MAA, IL-11Rα, KDR, Lewis Y, L1-cell adhesion molecule, MAGE-A1, Oncofetal antigen (h5T4), and/or TAG-72 expression.

Embodiment 592. The method, the population of cells, the composition, the pharmaceutical composition or the use of any of embodiments 381-591, wherein the cancer is a hematologic malignancy.

Embodiment 593. The method, the population of cells, the composition, the pharmaceutical composition or the use of any of embodiments 381-592, wherein the hematologic malignancy is selected from the group consisting of myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), T-cell lymphoma, and B-cell lymphoma.

Embodiment 594. The method, the population of cells, the composition, the pharmaceutical composition or the use of any of embodiments 381-593, wherein the cancer is solid malignancy.

Embodiment 595. The method, the population of cells, the composition, the pharmaceutical composition or the use of any of embodiments 381-594, wherein the solid malignancy is selected breast cancer, ovarian cancer, colon cancer, prostate cancer, epithelial cancer, renal-cell carcinoma, pancreatic adenocarcinoma, cervical carcinoma, colorectal cancer, glioblastoma, rhabdomyosarcoma, neuroblastoma, melanoma, Ewing sarcoma, osteosarcoma, mesothelioma and adenocarcinoma.

Embodiment 596. The method of any of embodiments 381-595, the population of cells of any of embodiments 381-595, the composition of any of embodiments 381-595, the pharmaceutical composition of any of embodiments 381-595, or the use of any of embodiments 381-595, wherein the disease is an autoimmune disease.

Embodiment 597. The method, the population of cells, the composition, the pharmaceutical composition or the use of any of embodiments 381-596, wherein the autoimmune disease is selected from the group consisting of lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

Embodiment 598. The method of any of embodiments 381-597, the population of cells of any of embodiments 381-597, the composition of any of embodiments 381-597, the pharmaceutical composition of any of embodiments 381-597, or the use of any of embodiments 381-597, wherein the disease is diabetes mellitus.

Embodiment 599. The method, the population of cells, the composition, the pharmaceutical composition or the use of any of embodiments 381-598, wherein the diabetes is selected from the group consisting Type I diabetes, Type II diabetes, prediabetes, and gestational diabetes.

Embodiment 600. The method of any of embodiments 381-599, the population of cells of any of embodiments 381-599, the composition of any of embodiments 381-599, the pharmaceutical composition of any of embodiments 381-599, or the use of any of embodiments 381-599, wherein the disease is a neurological disease.

Embodiment 601. The method, the population of cells, the composition, the pharmaceutical composition or the use of any of embodiments 381-600, wherein the neurological disease is selected from the group consisting of catalepsy, epilepsy, encephalitis, meningitis, migraine, Huntington's, Alzheimer's, Parkinson's, Pelizaeus-Merzbacher disease, and multiple sclerosis.

Embodiment 602. A method of identifying a site for inserting a first transgene at a TCR gene locus, comprising the steps of:
(a) identifying a protospacer adjacent motif (PAM) sequence or target adjacent motif (TAM) sequence in (i) the TCR gene locus, (ii) the 100 bp upstream of the 5′ end of TCR gene locus, or (iii) the 100 bp downstream of the 3′ end of TCR gene locus, and
(b) generating a gRNA comprising a complementary region, wherein the complementary region comprises a nucleic acid sequence that is complementary to a target nucleic acid sequence within the TCR gene locus, wherein the target nucleic acid sequence comprises the first insertion site, and wherein the first insertion site is 25 nucleotides or less from a PAM sequence or a TAM sequence.

### EQUIVALENTS

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known components and functions have not been shown or described in detail to avoid unnecessarily obscuring the description of the embodiments of the technology. Where the context permits, singular or plural terms may also include the plural or singular term, respectively. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1-223. (canceled)

224. A method of producing a composition comprising genetically engineered cells, the method comprising:
selecting one or more genetically engineered cells from a population of cells based on a level of one or more markers on the cell surface of the one or more genetically engineered cells, and
formulating the composition comprising the selected one or more genetically engineered cells for treating a disease in a subject,
wherein the one or more genetically engineered cells comprise one or more genetic modifications, and the one or more genetic modifications comprise a transgene encoding a first tolerogenic factor at an insertion site at a T-cell receptor (TCR) gene locus, and
wherein the level of the one or more markers on the cell surface comprise a level of CD3 and the one or more genetically engineered cells are selected if CD3 is not present at a detectable level on the cell surface of the one or more genetically engineered cells.

225. The method of claim 224, wherein the step of inserting comprises homology-directed repair (HDR)-mediated insertion using a genome-modifying protein.

226. The method of claim 225, wherein the step of inserting using a genome modifying protein comprises insertion by a CRISPR-associated transposase, prime editing, a TnpB polypeptide, or Programmable Addition via Site-specific Targeting Elements (PASTE).

227. The method of claim 225, wherein the step of inserting using a genome modifying protein comprises insertion by a site-directed nuclease selected from the group consisting of: Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, and a TnpB polypeptide.

228. The method of claim 224, wherein the TCR locus is or comprises: a TRAC locus, a TRBC1 locus, or a TRBC2 locus.

229. The method of claim 224, wherein the step of inserting comprises using an hTRAC gRNA comprising the nucleic acid sequence TCAGGGTTCTGGATATCTGT (SEQ ID NO: 124).

230. The method of claim 224, wherein the method further comprises detecting a level of the first tolerogenic factor on the cell surface of the one or more genetically engineered cells and the one or more genetically engineered cells are selected if the first tolerogenic factor is detected on the cell surface of the one or more genetically engineered cells.

231. The method of claim 224, wherein the first tolerogenic factor is or comprises CD47, A20/TNFAIP3, B2M-HLA-E, C1-Inhibitor, CCL21, CCL22, CD16, CD16 Fc receptor, CD24, CD27, CD35, CD39, CD46, CD52, CD55, CD59, CD200, CR1, CTLA4-Ig, DUX4, FasL, H2-M3 (HLA-G), HLA-C, HLA-E, HLA-E heavy chain, HLA-F, HLA-G, IDO1, IL-10, IL15-RF, IL-35, IL-39, MANF, Mfge8, PD-L1, or Serpinb9.

232. The method of claim 231, wherein the CD47 comprises an amino acid sequence at least 80% identical to an amino acid sequence set forth in SEQ ID NO:1 or SEQ ID NO:2.

233. The method of claim 224, wherein the one or more genetic modifications further comprise a modification at a B2M locus, a TAP I locus, a NLRC5 locus, a CIITA locus, an HLA-A locus, an HLA-B locus, an HLA-C locus, an HLA-DP locus, an HLA-DM locus, an HLA-DOA locus, an HLA-DOB locus, an HLA-DQ locus, an HLA-DR locus, a RFX5 locus, a RFXANK locus, a RFXAP locus, an NFY-A locus, an NFY-B locus, an NFY-C locus, or any combination thereof.

234. The method of claim 224, wherein the method further comprises inserting a second transgene encoding a CAR in the genome of one or more cells in the population.

235. The method of claim 234, wherein the second transgene encoding a CAR is inserted at a TRAC locus, a TRBC1 locus, a TRBC2 locus, a B2M locus, a CIITA locus, a MICA locus, a MICB locus, or a safe harbor locus.

236. The method of claim 234, wherein the second transgene and the first tolerogenic factor are encoded by a bicistronic construct.

237. The method of claim 234, wherein the CAR comprises a CD5-specific CAR, a CD19-specific CAR, a CD20-specific CAR, a CD22-specific CAR, a CD23-specific CAR, a CD30-specific CAR, a CD33-specific CAR, CD38-specific CAR, a CD70-specific CAR, a CD123-specific CAR, a CD138-specific CAR, a Kappa, Lambda, B cell maturation agent (BCMA)-specific CAR, a G-protein coupled receptor family C group 5 member D (GPRC5D)-specific CAR, a CD123-specific CAR, a LeY-specific CAR, a NKG2D ligand-specific CAR, a WT1-specific CAR, a GD2-specific CAR, a HER2-specific CAR, a EGFR-specific CAR, a EGFRvIII-specific CAR, a B7H3-specific CAR, a PSMA-specific CAR, a PSCA-specific CAR, a CAIX-specific CAR, a CD171-specific CAR, a CEA-specific CAR, a CSPG4-specific CAR, a EPHA2-specific CAR, a FAP-specific CAR, a FRα-specific CAR, a IL-13Rα-specific CAR, a Mesothelin-specific CAR, a MUC1-specific CAR, a MUC16-specific CAR, a ROR1-specific CAR, a C-Met-specific CAR, a CD133-specific CAR, a Ep-CAM-specific CAR, a GPC3-specific CAR, a HPV16-E6-specific CAR, a IL13Ra2-specific CAR, a MAGEA3-specific CAR, a MAGEA4-specific CAR, a MART1-specific CAR, a NY-ESO-1-specific CAR, a VEGFR2-specific CAR, a α-Folate receptor-specific CAR, a CD24-specific CAR, a CD44v7/8-specific CAR, a EGP-2-specific CAR, a EGP-40-specific CAR, a erb-B2-specific CAR, a erb-B 2,3,4-specific CAR, a FBP-specific CAR, a Fetal acethylcholine e receptor-specific CAR, a G_{D2}-specific CAR, a G_{D3}-specific CAR, a HMW-MAA-specific CAR, a IL-11Rα-specific CAR, a KDR-specific CAR, a Lewis Y-specific CAR, a L1-cell adhesion molecule-specific CAR, a MAGE-A1-specific CAR, a Oncofetal antigen (h5T4)-specific CAR, a TAG-72-specific CAR, or a CD19/CD22-bispecific CAR.

238. The method of claim 234, wherein the method further comprises detecting a level of the CAR on the cell surface of the one or more genetically engineered cells and the one or more genetically engineered cells are selected if the CAR is detected on the cell surface of the one or more genetically engineered cells.

239. The method of claim 224, wherein the population of cells are T cells.

240. The method of claim 239, wherein the T-cells are CD3+ T cells, CD4+ T cells, CDS+ T cells, naive T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T cells, effector memory T cells, effector memory T cells expressing CD45RA (TEMRA cells), tissue-resident memory (Trm) cells, virtual memory T cells, innate memory T cells, memory stem cell (Tse), 76 T cells, or any combination thereof.

241. The method of claim 239, wherein the T cells are primary T cells, or the T cells have been differentiated from embryonic stem cells (ESCs) or an induced pluripotent stem cells (iPSCs).

242. A population of genetically engineered cells produced by the method of claim 224.

243. A method treating a disease in a subject, comprising administering to a subject a population of cells according to claim 242.
